SEQUENCE LISTING (26 sequences)

SEQ ID NO: 1 - protein, 7 aa, Artificial Sequence
  MET Receptor Binding Peptide
ASVHFPP

SEQ ID NO: 2 - protein, 7 aa, Artificial Sequence
  MET Receptor Binding Peptide
TATFWFQ

SEQ ID NO: 3 - protein, 7 aa, Artificial Sequence
  MET Receptor Binding Peptide
TSPVALL

SEQ ID NO: 4 - protein, 7 aa, Artificial Sequence
  MET Receptor Binding Peptide
IPLKVHP

SEQ ID NO: 5 - protein, 7 aa, Artificial Sequence
  MET Receptor Binding Peptide
WPRLTNM

SEQ ID NO: 6 - protein, 12 aa, Artificial Sequence
  Free SP94 peptide
SFSIILTPILPL

SEQ ID NO: 7 - protein, 15 aa, Artificial Sequence
  Cys-SP94 peptide
SFSIILTPILPLGGC

SEQ ID NO: 8 - protein, 18 aa, Artificial Sequence
  SP94 peptide
SFSIILTPILPLEEEGGC

SEQ ID NO: 9 - protein, 42 aa, Artificial Sequence
  Nuclear localization peptides
GNQSSNFGPMKGGNFGGRSSGPYGGGGQYFAKPRNQGGYGGC

SEQ ID NO: 10 - protein, 7 aa, Artificial Sequence
  Nuclear localization peptides
RRMKWKK

SEQ ID NO: 11 - protein, 7 aa, Artificial Sequence
  Nuclear localization peptides
PKKKRKV

SEQ ID NO: 12 - protein, 16 aa, Artificial Sequence
  Nuclear localization peptides
KRPAATKKAGQAKKKK

SEQ ID NO: 13 - protein, 25 aa, Artificial Sequence
  H5WYG fusogenic peptide
GLFHAIAHFIHGGWHGLIHGWYGGC

SEQ ID NO: 14 - protein, 8 aa, Artificial Sequence
  Fusogenic peptide - polyarginine
RRRRRRRR

SEQ ID NO: 15 - protein, 12 aa, Artificial Sequence
  MET Receptor Binding Peptide 12-mer
YLFSVHWPPLKA

SEQ ID NO: 16 - protein, 7 aa, Artificial Sequence
  target un-related peptide
HAIYPRH

SEQ ID NO: 17 - protein, 7 aa, Artificial Sequence
  irrelevant phage clone
TPDWLFP

SEQ ID NO: 18 - protein, 7 aa, Artificial Sequence
  primary receptor for NiV entry via macropinocytosis
TGAILHP

SEQ ID NO: 19 - DNA, 64 bp, Artificial Sequence
  cyclin B1-specific siRNA precursor DNA
ggatccgaaatgtaccctccagaaattgaattcgtttctggagggtacatttctttttgaattc

SEQ ID NO: 20 - DNA, 64 bp, Artificial Sequence
  cyclin B1-specific siRNA precursor DNA
cctaggctttacatgggaggtctttaacttaagcaaagacctcccatgtaaagaaaaagaattc

SEQ ID NO: 21 - RNA, 21 nt, Artificial Sequence
  Cyclin B1-specific siRNA
gaaauguacccuccagaaauu

SEQ ID NO: 22 - RNA, 21 nt, Artificial Sequence
  Cyclin B1-specific siRNA
uucuuuacaugggaggucuuu

SEQ ID NO: 23 - protein, 7 aa, Artificial Sequence
  MET Receptor Binding Peptide
FSAHAHL

SEQ ID NO: 24 - protein, 7 aa, Artificial Sequence
  MET Receptor Binding Peptide
GNTPSRA

SEQ ID NO: 25 - DNA, 22 bp, Artificial Sequence
  oligonucleotide with 5′ amino modifier C12
aaacatgtggattacccatgtc

SEQ ID NO: 26 - protein, 10 aa, Artificial Sequence
  Targeting Peptide (MC40)
ASVHFPPGGC

(19) United States
(12) Patent Application Publication
(10) Pub. No.: US 2015/0272885 A1
(43) Pub. Date: Oct. 1, 2015

(54) POROUS NANOPARTICLE-SUPPORTED LIPID BILAYERS (PROTOCELLS) FOR TARGETED DELIVERY INCLUDING TRANSDERMAL DELIVERY OF CARGO AND METHODS THEREOF

(71) Applicants: STC.UNM, Albuquerque, NM (US); SANDIA CORPORATION, Albuquerque, NM (US)

(72) Inventors: Carlee Erin Ashley, Albuquerque, NM (US); C. Jeffrey Brinker, Albuquerque, NM (US); Eric C. Carnes, Albuquerque, NM (US); Mohammad Houman Fekrazad, Albuquerque, NM (US); Linda A. Felton, Albuquerque, NM (US); Oscar Negrete, Pleasanton, CA (US); David Patrick Padilla, Albuquerque, NM (US); Brian S. Wilkinson, Albuquerque, NM (US); Dan C. Wilkinson, Albuquerque, NM (US); Cheryl L. Willman, Albuquerque, NM (US)

(21) Appl. No.: 14/350,674

(22) PCT Filed: Oct. 12, 2012
(86) PCT No.: PCT/US12/60072

§ 371 Date: May 20, 2014

Related U.S. Application Data
(60) Provisional application No. 61/547,402, filed on Oct. 14, 2011.
(60) Provisional application No. 61/577,410, filed on Dec. 19, 2011.
(60) Provisional application No. 61/578,463, filed on Dec. 21, 2011.

(51) Int. Cl. A61K 9/127; A61K 45/06; A61K 31/704; A61K 31/513; A61K 31/713; A61K 38/17

(52) U.S. Cl. CPC A61K 9/1271; A61K 31/713; A61K 38/17; A61K 31/704; A61K 31/513; A61K 45/06

(57) ABSTRACT

The present invention is directed to protocells for specific targeting of hepatocellular and other cancer cells which comprise a nanoporous silica core with a supported lipid bilayer; at least one agent which facilitates cancer cell death (such as a traditional small molecule, a macromolecular cargo (e.g. siRNA or a protein toxin such as ricin toxin A-chain or diphtheria toxin A-chain) and/or a histone-packaged plasmid DNA disposed within the nanoporous silica core (preferably supercoiled in order to more efficiently package the DNA into protocells) which is optionally modified with a nuclear localization sequence to assist in localizing protocells within the nucleus of the cancer cell and the ability to express peptides involved in therapy (apoptosis/cell death) of the cancer cell or as a reporter, a targeting peptide which targets cancer cells in tissue to be treated such that binding of the protocell to the targeted cells is specific and enhanced and a fusogenic peptide that promotes endosomal escape of protocells and encapsulated DNA. Protocells according to the present invention may be used to treat cancer, especially including hepatocellular (liver) cancer using novel binding peptides (c-MET peptides) which selectively bind to hepatocellular tissue or to function in diagnosis of cancer, including cancer treatment and drug discovery.

### RELATED APPLICATIONS

This invention claims the benefit of priority of U.S. Provisional Application Ser. No. 61/547,402, filed Oct. 14, 2011, entitled “Engineering Nanoporous Particle-Supported Lipid Bilayers (‘Protocells’) for Transdermal Cargo Delivery”, and U.S. Provisional Application Ser. No. 61/578,463, filed Dec. 21, 2011, entitled “Engineering Nanoporous Particle-Supported Lipid Bilayers (Protocells′) for Transdermal Cargo Delivery”, the entire contents of which are incorporated by reference herein.

This invention also claims the benefit of priority of U.S. Provisional Application Ser. No. 61/577,410, filed Dec. 19, 2011, entitled “Delivery of Therapeutic Macromolecular Cargos by Targeted Protocells”, the entire contents of which are incorporated by reference herein.

### GOVERNMENT SUPPORT

This invention was made with government support under grant no. PHS 2 PN2 EY016570B of the National Institutes of Health; grant no. awarded by 1U01CA151792-01 of the National Cancer Institute; grant no. FA 9550-07-1-0054/9550-10-1-0054 of the Air Force Office of Scientific Research; 1U19ES019528-01 of NIEHS; NSF:EF-0820117 of the National Science Foundation and DGE-0504276 of the National Science Foundation. The government has certain rights in the invention.

### FIELD OF THE INVENTION

Embodiments of the present invention are directed to protocells for specific targeting of cells within a patient's body, especially including hepatocellular and other cancer cells which comprise a 1) a nanoporous silica or metal oxide core; 2) a supported lipid bilayer; 3) at least one agent which facilitates cancer cell death (such as a traditional small molecule, a macromolecular cargo (e.g. siRNA, shRNA other micro RNA, or a protein toxin such as ricin toxin A-chain or diphtheria toxin A-chain) and/or DNA, including double stranded or linear DNA, plasmid DNA which may be supercoiled and/or packaged such as with histones and disposed within the nanoporous silica core (preferably supercoiled in order to more efficiently package the DNA into protocells) which is optionally modified with a nuclear localization sequence to assist in localizing protocells within the nucleus of the cancer cell and the ability to express peptides involved in therapy (apoptosis/cell death) of the cancer cell or as a reporter, a targeting peptide which targets cancer cells in tissue to be treated such that binding of the protocell to the targeted cells is specific and enhanced and a fusogenic peptide that promotes endosomal escape of protocells and encapsulated cargo, including DNA. Protocells according to the present invention may be used to treat cancer, especially including hepatocellular (liver) cancer using novel binding peptides (c-MET peptides) which selectively bind to hepatocellular tissue or to function in diagnosis of cancer, including cancer treatment and drug discovery.

In certain embodiments, protocells of the invention facilitate the delivery of a wide variety of active ingredients. Significantly, these protocells effectively enhance stratum corneum permeability and enable transdermal delivery of active ingredients including macromolecules.

In another embodiment, the invention provides stable, hydrophobic and super-hydrophobic porous nanoparticles useful in the delivery of a wide variety of active ingredients in environments such as the stomach.

In certain other embodiments, the invention provides transdermal protocells that are useful in delivering a wide-variety of active ingredients, protocells comprising a plurality of mesoporous, nanoparticulate silica cores that are loaded with a siRNA that induces sequence-specific degradation of NiV nucleocapsid protein (NiV-N) mRNA, and gastrically-buoyant protocells that enable delivery of a wide variety of active ingredients in the stomach.

### BACKGROUND OF THE INVENTION

Targeted delivery of drugs encapsulated within nanocarriers can potentially ameliorate a number of problems exhibited by conventional Tree′ drugs, including poor solubility, limited stability, rapid clearing, and, in particular, lack of selectivity, which results in non-specific toxicity to normal cells and prevents the dose escalation necessary to eradicate diseased cells. Passive targeting schemes, which rely on the enhanced permeability of the tumor vasculature and decreased draining efficacy of tumor lymphatics to direct accumulation of nanocarriers at tumor sites (the so-called enhanced permeability and retention, or EPR effect), overcome many of these problems, but the lack of cell-specific interactions needed to induce nanocarrier internalization decreases therapeutic efficacy and can result in drug expulsion and induction of multiple drug resistance.

One of the challenges in nanomedicine is to engineer nanostructures and materials that can efficiently encapsulate cargo, for example, drugs, at high concentration, cross the cell membrane, and controllably release the drugs at the target site over a prescribed period of time. Recently, inorganic nanoparticles have emerged as a new generation of drug or therapy delivery vehicles in nanomedicine. More recently, gating methods that employ coumarin, azobenzene, rotaxane, polymers, or nanoparticles have been developed to seal a cargo within a particle and allow a triggered release according to an optical or electrochemical stimulus.

While liposomes have been widely used in drug delivery due to their low immunogenicity and low toxicity, they still need to be improved in several aspects. First, the loading of cargo can only be achieved under the condition in which liposomes are prepared. Therefore, the concentration and category of cargo may be limited. Second, the stability of liposomes is relatively low. The lipid bilayer of the liposomes often tends to age and fuse, which changes their size and size distribution. Third, the release of cargo in liposomes is instantaneous upon rupture of the liposome which makes it difficult to control the release.

A porous nanoparticle-supported lipid bilayer (protocell), formed via fusion of liposomes to nanoporous silica particles, is a novel type of nanocarrier that addresses multiple challenges associated with targeted delivery of cancer therapeutics and diagnostics. Like liposomes, protocells are biocompatible, biodegradable, and non-immunogenic, but their nanoporous silica core confers a drastically enhanced cargo capacity and prolonged bilayer stability when compared to similarly-sized liposomal delivery agents. The porosity and surface chemistry of the core can, furthermore, be modulated to promote encapsulation of a wide variety of therapeutic agents, such as drugs, nucleic acids, and protein toxins. The rate of cargo release can be controlled by pore size, chemical composition and the overall degree of silica condensation of the core, making protocells useful in applications requiring either burst or controlled release profiles. Finally, the protocell's supported lipid bilayer (SLB) can be modified with variously with ligands to promote selective delivery and with PEG to extend circulation times.

The need to improve the activity of chemotherapeutic agents and to enhance cancer therapy is ongoing. The use of protocells in conjunction with alternative approaches to targeting, binding, enhancing invasion of cancer and depositing chemotherapeutic agents in proximity to their site of activity are important facets of cancer therapy. The present invention is undertaken to advance the art of cancer therapy and to improve the delivery of agents which can influence therapeutic outcome, whether by enhancing the administration of cancer therapeutic agents or in diagnostics, to facilitate approaches to diagnosing cancer and monitoring cancer therapy.

There is also a need for transdermal delivery vehicles which are designed to permeate the stratum corneum optimally to enable delivery of active ingredients previously restricted to administration through other, less advantageous routes.

### OBJECTS OF THE INVENTION

Objects of the invention are directed to providing improvements to protocell technology, to the protocells themselves, to pharmaceutical compositions which comprise such protocells and methods of using protocells and pharmaceutical compositions according to the invention for therapy and diagnostics, including monitoring therapy.

Additional objects of embodiments of the invention relate to novel MET binding peptides, their use in pharmaceutical compositions and methods according to other embodiments the present invention.

These and/or other objects of the invention may be readily gleaned from a review of a description as presented in the specification.

### BRIEF DESCRIPTION OF THE INVENTION

Embodiments of the present invention are directed to protocells for specific targeting of cells, in particular aspects, hepatocellular and other cancer cells.

In certain aspects, the present invention is directed to a cell-targeting porous protocell comprising a nanoporous silica or metal oxide core with a supported lipid bilayer, and at least one further component selected from the group consisting of a cell targeting species;
a fusogenic peptide that promotes endosomal escape of protocells and encapsulated DNA, and other cargo comprising at least one cargo component selected from the group consisting of double stranded linear DNA or a plasmid DNA;
a drug;
an imaging agent,
small interfering RNA, small hairpin RNA, microRNA, or a mixture thereof,
wherein one of said cargo components is optionally conjugated further with a nuclear localization sequence.

In certain embodiments, protocells according to embodiments of the invention comprise a nanoporous silica core with a supported lipid bilayer; a cargo comprising at least one therapeutic agent which optionally facilitates cancer cell death such as a traditional small molecule, a macromolecular cargo (e.g. siRNA such as S565, S7824 and/or s10234, among others, shRNA or a protein toxin such as a ricin toxin A-chain or diphtheria toxin A-chain) and/or a packaged plasmid DNA (in certain embodiments—histone packaged) disposed within the nanoporous silica core (preferably supercoiled as otherwise described herein in order to more efficiently package the DNA into protocells as a cargo element) which is optionally modified with a nuclear localization sequence to assist in localizing/presenting the plasmid within the nucleus of the cancer cell and the ability to express peptides involved in therapy (e.g., apoptosis/cell death of the cancer cell) or as a reporter (fluorescent green protein, fluorescent red protein, among others, as otherwise described herein) for diagnostic applications. Protocells according to the present invention include a targeting peptide which targets cells for therapy (e.g., cancer cells in tissue to be treated) such that binding of the protocell to the targeted cells is specific and enhanced and a fusogenic peptide that promotes endosomal escape of protocells and encapsulated DNA. Protocells according to the present invention may be used in therapy or diagnostics, more specifically to treat cancer and other diseases, including viral infections, especially including hepatocellular (liver) cancer. In other aspects of the invention, proctocells use novel binding peptides (MET binding peptides as otherwise described herein) which selectively bind to cancer tissue (including hepatocellular, ovarian and cervical cancer tissue, among other tissue) for therapy and/or diagnosis of cancer, including the monitoring of cancer treatment and drug discovery.

In one aspect, protocells according to embodiments of the present invention comprise a porous nanoparticle protocell which often comprises a nanoporous silica core with a supported lipid bilayer. In this aspect of the invention, the protocell comprises a targeting peptide which is often a MET receptor binding peptide as otherwise described herein, often in combination with a fusogenic peptide on the surface of the protocell. The protocell may be loaded with various therapeutic and/or diagnostic cargo, including for example, small molecules (therapeutic and/or diagnostic, especially including anticancer and/or antiviral agents (for treatment of HBV and/or HCV), macromolecules including polypeptides and nucleotides, including RNA (shRNA and siRNA) or plasmid DNA which may be supercoiled and histone-packaged including a nuclear localization sequence, which may be therapeutic and/or diagnostic (including a reporter molecule such as a fluorescent peptide, including fluorescent green protein/FGP, fluorescent red protein/FRP, among others).

Transdermal embodiments of the invention include protocells comprised of porous nanoparticulates that (a) are loaded with one or more pharmaceutically-active agents and (b) that are encapsulated by and that support a lipid bilayer, wherein the lipid bilayer comprises one or more stratum corneum permeability-enhancers selected form the group consisting of monosaturated omega-9 fatty acids (oleic acid, elaidic acid, eicosenoic acid, mead acid, erucic acid, and nervonic acid, most preferably oleic acid), an alcohol, a diol (most preferably polyethylene glycol (PEG)), R8 peptide, and edge activators such as bile salts, polyoxyethylene esters and polyoxyethylene ethers, a single-chain surfactant (e.g. sodium deoxycholate), and wherein the protocell has an average diameter of between about 50 nm to about 300 nm, more preferably between about 55 nm to about 270 nm, more preferably between about 60 nm to about 240 nm, more preferably between about 65 nm to about 210 nm, more preferably between about 65 nm to about 190 nm, more preferably between about 65 nm to about 160 nm, more preferably between about 65 nm to about 130 nm, more preferably between about 65 nm to about 100 nm, more preferably between about 65 nm to about 90 nm, more preferably between about 65 nm to about 80 nm, more preferably between about 65 nm to about 75 nm, more preferably between about 65 nm to about 66, 67, 68, 69, 70, 71, 72, 73, 74 or 75 nm, most preferably around 70 nm.

Thus, the invention in one aspect provides a transdermal protocell comprising a plurality of porous nanoparticulates that (a) are loaded with one or more pharmaceutically-active agents and (b) that are encapsulated by and that support a lipid bilayer, wherein the lipid bilayer comprises one or more stratum corneum permeability-enhancers selected from the group consisting of a monosaturated omega-9 fatty acid, an alcohol, a diol, a solvent, a co-solvent, permeation promoting peptides and nucleotides, and an edge activator, wherein the protocell has an average diameter of between about 50 nm to about 300 nm. The monosaturated omega-9 fatty acid can be selected from the group consisting of oleic acid, elaidic acid, eicosenoic acid, mead acid, erucic acid, and nervonic acid, most preferably oleic acid, and mixtures thereof. The alcohol can be selected from the group consisting of methanol, ethanol, proponal, and butanol, and mixtures thereof, and the solvent and co-solvent are selected from the group consisting of PEG 400 and DMSO. The diol can be selected from the group consisting of ethylene glycol and polyethylene glycol, and mixtures thereof. The edge activator can be selected from the group consisting of bile salts, polyoxyethylene esters and polyoxyethylene ethers, and a single-chain surfactant, and mixtures thereof. In a preferred embodiment, the edge activator is sodium deoxycholate.

The transdermal route of administration is a superior route in comparison to the oral and parenteral routes. Orally administered drugs are subject to first-pass metabolism, and can have adverse interactions with food and the broad pH-range of the digestive tract. Parenteral administration is painful, generates bio-hazardous waste, and cannot be self-administered. Transdermal drug delivery addresses all of the fore-mentioned issues associated with both the oral and parenteral routes. Additionally, transdermal delivery systems (TDDS) allow for a controlled release profile that is sustained over several days. However, the main challenge associated with transdermal drug delivery lies in the skin's outermost layer of the epidermis, the stratum corneum. It confers the skin's barrier function due to its structure that is analogous to a “brick and mortar”. The “bricks” are composed of flattened corneocytes enriched with proteins, glycoproteins, fatty acids, and cholesterol. The intercellular space, that comprises the “mortars”, is rich in bilayers composed of ceramides, cholesterol, fatty acids, and exhibits a polarity similar to that of butanol. In the past four decades three generations of TDDS have been developed. First-generation systems utilize diffusion of low molecular weight, lipophilic compounds. Second- and third-generation systems recognize that permeability of the stratum corneum is key. These strategies ablate/bypass the stratum corneum or utilize chemical enhancers, biochemical enhancers, and electromotive forces to increase permeability of the stratum corneum. Amongst different enhancement strategies, liposomes have been shown to disrupt the highly ordered structure of the stratum corneum and subsequently increase the skin's permeability.

In one embodiment herein, we describe the development of nanoporous particle-supported lipid bilayers (“protocells”) to serve as a TDDS. Protocells are formed by electrostatically fusing a liposome to a nanoporous silica-particle core. They synergistically combine the advantages of both inorganic nanoparticles and liposomes, such as tunable porosity, high surface area that is amenable to high capacity loading of disparate types of cargo, and a supported lipid bilayer (SLB) with tunable fluidity that can be modified with various molecules. These biophysical and biochemical properties allow the protocell to be modified for different applications. In our preliminary studies, using inductively coupled plasma mass spectroscopy, we have shown that 0.1-0.5 wt % of our standard protocell formulation (55% DOPE, 30% Cholesterol, 15% PEG-2000) dosed at 8.125 mg was able to cross full-thickness patient-derived abdominal skins. Additionally, we demonstrated that 0.3-2.4 wt % of protocells were able to cross partial thickness skin from which the stratum corneum was removed.

The nanoporous silica-particle core of the transdermal protocells has a high surface area, readily variable porosity, and surface chemistry that is easily modified. These properties make the protocell-core amenable to high-capacity loading of many different types of cargo. The protocell's supported lipid bilayer (SLB) has an inherently low immunogenicity. Additionally, the SLB provides a fluid surface to which peptides, polymers and other molecules can be conjugated in order to facilitate targeted cellular uptake. These biophysical and biochemical properties allow for the protocell to be optimized for a specific environment, facilitate penetration into the stratum corneum, and subsequently deliver disparate types of cargo via the transdermal route. Methods of treating a cancer are one example of a therapeutic use of the transdermal protocells of the invention. Related pharmaceutical compositions are also described.

In one embodiment, the invention provides a protocell comprising a plurality of negatively-charged, nanoporous, nanoparticulate silica cores that are modified with an amine-containing silane such as N-(2-aminoethyl)-3-aminopropyltrimethoxysilane (AEPTMS) and that (a) are loaded with a siRNA or ricin toxin A-chain and (b) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bilayer comprises a cationic lipid and one or more zwitterionic phospholipids.

In the embodiment of the preceding paragraph, the lipid is preferably selected from the group consisting of 1,2-dioleoyl-3-trimethylammonium-propane (18:1 DOTAP) or 1,2-dioleoyl-sn-glycero-3-phospho-(1′-rac-glycerol) (DOPG), 1,2-dioleoyl-sn-glycero-3-phosphoethanolamine (DOPE) and mixtures thereof, and the protocell has at least one of the following characteristics: a BET surface area of greater than about 600 m²/g, a pore volume fraction of between about 60% to about 70%, a multimodal pore morphology composed of pores having an average diameter of between about 20 nm to about 30 nm, surface-accessible pores interconnected by pores having an average diameter of between about 5 nm to about 15 nm. Preferably, the protocell encapsulates around 10 nM of siRNA per 10¹⁰ nanoparticulate silica cores.

In still another embodiment, the invention provides a protocell comprising a plurality of negatively-charged, nanoporous, nanoparticulate silica cores that are modified with an amine-containing silane such as AEPTMS and that:

(a) are loaded with one or more siRNAs that target members of the cyclin superfamily selected from the group consisting of cyclin A2, cyclin B1, cyclin D1, and cyclin E; and
(b) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein (1) the lipid bilayer is loaded with SP94 and an endosomolytic peptide, and (2) the protocell selectively binds to a hepatocellular carcinoma cell.

In the embodiment of the preceding paragraph, the lipid bilayer preferably comprises DOPC/DOPE/cholesterol/PEG-2000 in an approximately 55:5:30:10 mass ratio.

Methods of treating a cancer such as liver cancer are one example of a therapeutic use of the AEPTMS-modified protocells of the invention. Related pharmaceutical compositions are also described.

In another embodiment, the invention provides a protocell comprising a plurality of mesoporous, nanoparticulate silica cores that (a) are loaded with a siRNA that induces sequence-specific degradation of Nipah virus (NiV) nucleocapsid protein (NiV-N) mRNA and (b) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yeamino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

In certain embodiments of the protocells of the preceding paragraph, the lipid bilayer comprises 1,2-dioleoyl-sn-glycero-3-phosphocholine (DOPC), 1,2-dioleoyl-sn-glycero-3-phosphoethanolamine (DOPE) a polyethylene glycol (PEG), a targeting peptide, and R8, and the mesoporous, nanoparticulate silica cores each have an average diameter of around 100 nm, an average surface area of greater than 1,000 m²/g and surface-accessible pores having an average diameter of between about 20 nm to about 25 nm, and have a siRNA load of around 1 μM per 10¹⁰ particles or greater. The targeting peptide preferably is a peptide that binds to ephrin B2 (EB2), and most preferably is TGAILHP (SEQ ID NO:18). Most preferably, the protocell comprises around 0.01 to around 0.02 wt % of TGAILHP, around 10 wt % PEG-2000 and around 0.500 wt % of R8.

Methods of treating a subject who is infected by, or at risk of infection with Nipah virus (NiV) are one example of a therapeutic use of protocells of the invention comprising a siRNA that induces sequence-specific degradation of Nipah virus (NiV) nucleocapsid protein (NiV-N) mRNA. Related pharmaceutical compositions are also described.

Other aspects of embodiments of the present invention are directed to pharmaceutical compositions. Pharmaceutical compositions according to the present invention comprise a population of protocells which may be the same or different and are formulated in combination with a pharmaceutically acceptable carrier, additive or excipient. The protocells may be formulated alone or in combination with another bioactive agent (such as an additional anti-cancer agent or an antiviral agent) depending upon the disease treated and the route of administration (as otherwise described herein). These compositions comprise protocells as modified for a particular purpose (e.g. therapy, including cancer therapy, or diagnostics, including the monitoring of cancer therapy). Pharmaceutical compositions comprise an effective population of protocells for a particular purpose and route of administration in combination with a pharmaceutically acceptable carrier, additive or excipient.

An embodiment of the present invention also relates to methods of utilizing the novel protocells as described herein. Thus, in alternative embodiments, the present invention relates to a method of treating a disease and/or condition comprising administering to a patient or subject in need an effective amount of a pharmaceutical composition as otherwise described herein. The pharmaceutical compositions according to the present invention are particularly useful for the treatment of a number disease states, especially including cancer, and disease states or conditions which occur secondary to cancer or are the cause of cancer (in particular, HBV and/or HCV infections).

In further alternative aspects, the present invention relates to methods of diagnosing cancer, the method comprising administering a pharmaceutical composition comprising a population of protocells which have been modified to deliver a diagnostic agent or reporter imaging agent selectively to cancer cells to identify cancer in the patient. In this method, protocells according to the present invention may be adapted to target cancer cells through the inclusion of at least one targeting peptide which binds to cancer cells which express polypeptides or more generally, surface receptors ro cell membrane components, which are the object of the targeting peptide and through the inclusion of a reporter component (including an imaging agent) of the protocell targeted to the cancer cell, may be used to identify the existence and size of cancerous tissue in a patient or subject by comparing a signal from the reporter with a standard. The standard may be obtained for example, from a population of healthy patients or patients known to have a disease for which diagnosis is made. Once diagnosed, appropriate therapy with pharmaceutical compositions according to the present invention, or alternative therapy may be implemented.

In still other aspects of the invention, the compositions according to the present invention may be used to monitor the progress of therapy of a particular disease state and/or condition, including therapy with compositions according to the present invention. In this aspect of the invention, a composition comprising a population of protocells which are specific for cancer cell binding and include a reporter component may be administered to a patient or subject undergoing therapy such that progression of the therapy of the disease state can be monitored.

Alternative aspects of the invention relate to five (5) novel MET binding peptides as otherwise described herein, which can be used as targeting peptides on protocells of certain embodiments of the present invention, or in pharmaceutical compositions for their benefit in binding MET protein in a variety of cancer cells, including hepatocellular, cervical and ovarian cells, among numerous other cells in cancerous tissue. One embodiment of the invention relates to five (5) different 7 mer peptides which show activity as novel binding peptides for MET receptor (a.k.a. hepatocyte growth factor receptor, expressed by gene c-MET). These five (5) 7 mer peptides are as follows:

SEQ ID NO: 1
ASVHFPP (Ala-Ser-Val-His-Phe-Pro-Pro)
SEQ ID NO: 2
TATFWFQ (Thr-Ala-Thr-Phe-Trp-Phe-Gln)
SEQ ID NO: 3
TSPVALL (Thr-Ser-Pro-Val-Ala-Leu-Leu)
SEQ ID NO: 4
IPLKVHP (Ile-Pro-Leu-Lys-Val-His-Pro)
SEQ ID NO: 5
WPRLTNM (Trp-Pro-Arg-Leu-Thr-Asn-Met)

Each of these peptides may be used alone or in combination with other MET binding peptides within the above group or with a spectrum of other targeting peptides (e.g., SP94 peptides as described herein) which may assist in binding protocells according to an embodiment of the present invention to cancer cells, including hepatocellular cancer cells, ovarian cancer cells, breast cancer cells and cervical cancer cells, among numerous others. These binding peptides may also be used in pharmaceutical compounds alone as MET binding peptides to treat cancer and otherwise inhibit hepatocyte growth factor binding receptor. These peptides may be formulated alone or in combination with other bioactive agents for purposes of providing an intended result. Pharmaceutical compositions comprise an effective amount of at least one of the five (5) MET-binding peptides identified above, in combination with a pharmaceutically acceptable carrier, additive or excipient optionally in combination with an additional bioactive agent, which may include an anticancer agent, antiviral agent or other bioactive agent.

### BRIEF DESCRIPTION OF THE FIGURES

FIG. 1 shows that the nanoparticles according to one embodiment used in the present invention which are prepared by an aerosol-assisted EISA process can be altered to control particle size and distribution.

FIG. 2 shows the pore size and framework designed to be tailorable for multiple types of cargo and that aerosolized auxiliary components are easily incorporated according to one embodiment.

FIG. 2A shows that that a, b c, and e of FIG. 2 are templated by CTAB, B58, P123 and PS+B56. A, B, C, D and E are templated by CTAP+NaCl, 3% wt P123, 3% wt P123+poly(propylene glycol acrylate), microemulsion and CTAB(NH₄)₂S0₄.

FIG. 3 shows that pore surface chemistry (i.e., charge and hydrophobicity) and pore size is controlled principally by co-condensation of organo-silanes and silicic acids either by co-self-assembly or post-self-assembly derivatization according to one embodiment. See Lin, et al., Chem. Mater. 15, 4247-56 2003; Liu, J. et al., J. Phys. Chem., 104, 8328-2339, 2000; Fan, H. et al., Nature, 405, 56-60, 2000 and Lu, Y. et al., J. Am. Chem. Soc., 122, 5258-5261, 2000.

FIG. 4 depicts the packaging of the CB1 plasmid with histone proteins. (A) Schematic depicting the process used to supercoil the CB1 plasmid @CB1), package supercoiled pCB1 with histones H1, H2A, H2B, H3, and H4, and modify the resulting pCB1-histone complex with a nuclear localization sequence (NLS) that promotes translocation through nuclear pores. (B) and (D) Atomic force microscopy (AFM) images of the CB1 plasmid (B) and histone-packaged pCB1 (D). Scale bars=100 nm. (C) and (E) Height profiles that correspond to the red lines in (B) and (D), respectively.

FIG. 5 depicts the synthesis of MC40-targeted mesoporous silica nanoparticle-supported lipid bilayers (protocells) loaded with histone-packaged pCB1. (A) Schematic depicting the process used to generate DNA-loaded, peptide-targeted protocells. Histone-packaged pCB1 is loaded into the mesoporous silica nanoparticles that form the core of the protocell by simply soaking the particles in a solution of the pCB1-histone complex. PEGylated liposomes are then fused to DNA-loaded cores to form a supported lipid bilayer (SLB) that is further modified with a targeting peptide (MC40) that binds to HCC and a endosomolytic peptide (H5WYG) that promotes endosomal escape of internalized protocells. A sulfhydryl-to-amine crosslinker (spacer arm=9.5 nm) was used to conjugate peptides, modified with a C-terminal cysteine residue, to DOPE moieties in the SLB. (B) Transmission electron microscopy (TEM) image of the mesoporous silica nanoparticles that are used as the core of the protocell. Scale bar=200 nm. Inset=scanning electron microscopy (SEM) image, which demonstrates that the 15-25 nm pores are surface-accessible. Inset scale bar=50 nm. (C) Size distribution for the mesoporous silica nanoparticles, as determined by dynamic light scattering (DLS). (D, left axis) Cumulative pore volume plot for the mesoporous silica nanoparticles, calculated from the adsorption branch of the nitrogen sorption isotherm shown in Figure S-4A using the Barrett-Joyner-Halenda (BJH) model. (D, right axis) Size distribution for the pCB1-histone complex, as determined by DLS.

FIG. 6 shows that mesoporous silica nanoparticles have a high capacity for histone-packaged pCB1, and the resulting protocells release encapsulated DNA only under conditions that mimic the endosomal environment according to one embodiment. (A) The concentration of pCB1 or histone-packed pCB1 (‘complex’) that can be encapsulated within unmodified mesoporous silica nanoparticles (ζ=−38.5 mV) or mesoporous silica nanoparticles modified with APTES, an amine-containing silane (ζ=+11.5 mV). (B) The percentage of Hep3B that become positive for ZsGreen, a green fluorescent protein encoded by pCB1, when 1×10⁶ cells/mL are incubated with 1×10⁹ MC40-targeted, pCB1-loaded protocells for 24 hours at 37° C. The x-axis specifies whether the protocell core was modified with APTES and whether pCB1 was pre-packaged with histones. pCB1 packaged with a mixture of DOTAP and DOPE (1:1 w/w) was included as a control in (A) and (B). (C) and (D) The time-dependent release of histone-packaged pCB1 from unmodified mesoporous silica nanoparticles and corresponding protocells upon exposure to a simulated body fluid (C) or a pH 5 buffer (D). The protocell SLB was composed of DOPC with 5 wt % DOPE, 30 wt % cholesterol, and 10 wt % PEG-2000 and, for (B), was modified with 0.015 wt % MC40 and 0.500 wt % H5WYG. All error bars represent 95% confidence intervals (1.96σ) for n=3.

FIG. 7 provides a schematic depicting the process by which MC40-targeted protocells deliver histone-packaged pCB1 to HCC. [1] MC40-targeted protocells bind to Hep3B cells with high affinity due to the recruitment of targeting peptides to Met, which is over-expressed by a variety of HCC lines. The fluid DOPC SLB promotes peptide mobility and, therefore, enables protocells modified with a low MC40 density to retain a high specific affinity for Hep3B (see FIG. 8A). [2] MC40-targeted protocells become internalized by Hep3B via receptor-mediated endocytosis (see FIG. 8B and FIG. 15A). [3] Endosomal conditions destabilize the SLB [insert Nature Materials ref] and cause protonation of the H5WYG endosomolytic peptide, both of which enable histone-packaged pCB1 to become dispersed in the cytosol of Hep3B cells (see FIG. 16B). [4] pCB1-histone complexes, when modified with a nuclear localization sequence (NLS), become concentrated in the nuclei of Hep3B cells within ˜24 hours (see FIG. 16C), which enables efficient transfection of both dividing and non-dividing cancer cells (see FIG. 17).

FIG. 8 shows that MC40-targeted protocells bind to HCC with high affinity and are internalized by Hep3B but not by normal hepatocytes. (A) Apparent dissociation constants (K_{d}) for MC40-targeted protocells when exposed to Hep3B or hepatocytes; K_{d} values are inversely related to specific affinity and were determined from saturation binding curves (see Figure S-11). Error bars represent 95% confidence intervals (1.96σ) for n=5. (B) and (C) Confocal fluorescence microscopy images of Hep3B (B) and hepatocytes (C) that were exposed to a 1000-fold excess MC40-targeted protocells for 1 hour at 37° C. Met was stained with an Alexa Fluor® 488-labeled monoclonal antibody (green), the protocell core was labeled with Alexa Fluor® 594 (red), and cell nuclei were stained with Hoechst 33342 (blue). Scale bars=20 μm Protocell SLBs were composed of DOPC with 5 wt % DOPE, 30 wt % cholesterol, and 10 wt % PEG-2000 (18:1) and were modified with either 0.015 wt % (A-C) or 0.500 wt % (A) of the MC40 targeting peptide.

FIG. 9 shows MC40-targeted, pCB1-loaded protocells induce apoptosis of HCC at picomolar concentrations but have a minimal impact on the viability of normal hepatocytes. Dose (A) and time (B) dependent decreases in expression of cyclin B1 mRNA and cyclin B1 protein upon continual exposure of Hep3B to MC40-targeted, pCB1-loaded protocells at 37° C. Cells were exposed to various pCB1 concentrations for 48 hours in (A) and to 5 pM of pCB1 for various periods of time in (B). Expression of cyclin B1 protein in hepatocytes and ZsGreen in Hep3B are included as controls. Real-time PCR and immunofluorescence were employed to determine cyclin B1 mRNA and protein concentrations, respectively. (C) The percentage of Hep3B that become arrested in G₂/M phase after continual exposure to MC40-targeted, pCB1-loaded protocells ([pCB1]=5 pM) for various periods of time at 37° C. The percentage of hepatocytes in G₂/M phase and Hep3B in S phase are included for comparison. Cells were stained with Hoechst 33342 prior to cell cycle analysis. (D) The percentage of Hep3B that become apoptotic upon continual exposure to MC40-targeted, pCB1-loaded protocells ([pCB1]=5 pM) for various periods of time at 37° C. The percentage of hepatocytes positive for markers of apoptosis was included as a control. Cells positive for Alexa Fluor® 647-labeled annexin V were considered to be in the early stages of apoptosis, while cells positive for both annexin V and propidium iodide were considered to be in the late stages of apoptosis. The total number of apoptotic cells was determined by adding the numbers of single- and double-positive cells. In all experiments, protocell SLBs were composed of DOPC with 5 wt % DOPE, 30 wt % cholesterol, and 10 wt % PEG-2000 (18:1) and were modified with 0.015 wt % MC40 and 0.500 wt % H5WYG. All error bars represent 95% confidence intervals (1.96σ) for n=3.

FIG. 10 shows MC40-targeted, pCB1-loaded protocells induce selective apoptosis of HCC 2500-fold more effectively than corresponding lipoplexes. (A) Zeta potential values for DOPC protocells, DOPC protocells modified with 10 wt % PEG-2000 (18:1), lipoplexes composed of pCB1 and a mixture of DOTAP and DOPE (1:1 w/w), and DOTAP/DOPE lipoplexes modified with 10 wt % PEG-2000. All zeta potential measurements were conducted in 0.5×PBS (pH 7.4). (B, left axis) The percentage of Hep3B and hepatocytes that become apoptotic upon continual exposure to 5 pM of pCB1, delivered via MC40-targeted protocells or lipoplexes, for 48 hours at 37° C. (B, right axis) The number of MC40-targeted, pCB1-loaded protocells or lipoplexes necessary to induce apoptosis in 90% of 1×10⁶ Hep3B cells within 48 hours at 37° C. For (B), cells were stained with Alexa Fluor® 647-labeled annexin V and propidium iodide; single- and double-positive cells were considered to be apoptotic. Protocell SLBs were composed of DOPC with 5 wt % DOPE, 30 wt % cholesterol, and 10 wt % PEG-2000 (when indicated) and were modified with 0.015 wt % MC40 and 0.500 wt % H5WYG. DOTAP/DOPE lipoplexes were modified with 10 wt % PEG-2000 (when indicated), 0.015 wt % MC40, and 0.500 wt % H5WYG. pCB1 was modified with the NLS in all experiments. All error bars represent 95% confidence intervals (1.96σ) for n=3.

FIG. 11 shows that MC40-targeted protocells selectively deliver high concentrations of taxol, Bcl-2-specific siRNA, and pCB1 to HCC without affecting the viability of hepatocytes. (A) Concentrations of taxol, siRNA that silences expression of Bcl-2, and the CB1 plasmid that can be encapsulated within 10¹² protocells, liposomes, or lipoplexes. Red bars indicate how taxol and pCB1 concentrations change when both are loaded within protocells. Blue bars indicate how taxol, siRNA, and pCB1 concentrations change when all three are loaded within protocells or when siRNA and pCB1 are loaded within lipoplexes. (B) Confocal fluorescence microscopy image showing the intracellular distributions of Oregon Green® 488-labeled taxol (green), Alexa Fluor® 594-labeled siRNA (red), and Cy5-labeled pDNA (white) upon delivery to Hep3B via MC40-targeted protocells. Cells were incubated with a 1000-fold excess of MC40-targeted protocells for 24 hours at 37° C. prior to being fixed and stained with Hoechst 33342 (blue). Scale bars=10 μm. (C) Fractions of Hep3B, SNU-398, and hepatocyte cells that become arrested in G₂/M phase upon exposure to 10 nM of taxol and/or 5 pM of pCB1 for 48 hours at 37° C. Fractions were normalized against the percentage of logarithmically-growing cells in G₂/M. (D) The percentage of Hep3B, SNU-398, and hepatocyte cells that become positive for Alexa Fluor® 647-labeled annexin V and propidium iodide (PI) upon exposure to 10 nM of taxol, 250 pM of Bcl-2-specific siRNA, and/or 5 pM of pCB1 for 48 hours at 37° C. In (C) and (D), ‘pCB1’ refers to pCB1 that was packaged and delivered non-specifically to cells using a mixture of DOTAP and DOPE (1:1 w/w). In all experiments, protocell SLBs were composed of DOPC with 5 wt % DOPE, 30 wt % cholesterol, and 10 wt % PEG-2000 (18:1) and were modified with 0.015 wt % MC40 and 0.500 wt % H5WYG. Liposomes were composed of DSPC with 5 wt % DMPE, 30 wt % cholesterol, and 10 wt % PEG-2000 (16:0) and were modified with 0.015 wt % MC40 and 0.500 wt % H5WYG. Lipoplexes were composed of a DOTAP:DOPE (1:1 w/w) mixture and were modified with 10 wt % PEG-2000, 0.015 wt % MC40, and 0.500 wt % H5WYG. pCB1 was modified with the NLS in all experiments. All error bars represent 95% confidence intervals (1.96σ) for n=3.

FIG. 12 provides a vector map for the CB1 plasmid. The CB1 plasmid (pCB1) was constructed from the RNAi-Ready pSIREN-RetroQ-ZsGreen vector (Clontech Laboratories, Inc.; Mountain View, Calif.) and the pNEB193 vector (New England BioLabs, Inc.; Ipswich, Mass.). pCB1 encodes a cyclin B1-specific small hairpin RNA (shRNA) and a Zoanthus sp. green fluorescent protein (ZsGreen). Constitutive shRNA expression is driven by the RNA Pol III-dependent human U6 promoter (P_{U6}), while constitutive ZsGreen expression is driven by the immediate early promoter of cytomegalovirus (P_{CMV IE}). The ori and Amp^{R} elements enable propagation of the plasmid in E. coli. The DNA sequences that encode the sense and antisense strands of the cyclin B1-specific shRNA are underlined and are flanked by the restriction enzyme sites (BamHI in red and EcoRI in blue) that were employed to introduce the dsDNA oligonucleotide into the pSIREN vector.

FIG. 13 depicts the characterization of histone-packaged pCB1. (A) Electrophoretic mobility shift assays for pCB1 exposed to increasing concentrations of histones (H1, H2A, H2B, H3, and H4 in a 1:2:2:2:2 molar ratio). The pCB1:histone molar ratio is given for lanes 3-6. Lane 1 contains a DNA ladder, and lane 2 contains pCB1 with no added histones. (B) TEM image of histone-packaged pCB1 (1:50 pCB1:histone molar ratio). Scale bar=50 nm.

FIG. 14 shows nitrogen sorption analysis of unloaded and pCB1-loaded mesoporous silica nanoparticles. (A) Nitrogen sorption isotherms for mesoporous silica nanoparticles before and after loading with histone-packaged pCB1. (B) Brunauer-Emmett-Teller (BET) surface area of mesoporous silica nanoparticles, before and after loading with histone-packaged pCB1. Error bars represent 95% confidence intervals (1.96σ) for n=3.

FIG. 15 shows the small-angle neutron scattering (SANS) data for DOPC protocells. The data fit was obtained using a model for polydisperse porous silica spheres with a conformal shell of constant thickness and shows the presence of a 36-A bilayer at the surface of the silica particles that spans pore openings. Simulated SANS data for bilayer thicknesses of 0, 20, and 60 A are included for comparison. The measured bilayer thickness of 36 A is consistent with other neutron studies (33-38 A) performed on planar supported lipid bilayers and, under these contrast conditions, primarily represents scattering from the hydrogen-rich hydrocarbon core of the lipid bilayer.

FIG. 16 shows that protocells protect encapsulated DNA from nuclease degradation. Agarose gel electrophoresis of DNase I-treated pCB1 (lane 3), histone-packaged pCB1 (lane 5), pCB1 packaged with a 1:1 (w/w) mixture of DOTAP and DOPE (lane 7), pCB1 loaded in protocells with cationic cores (lane 9), and histone-packaged pCB1 loaded in protocells with anionic cores (lane 11). Naked pCB1 (lane 2), pCB1 released from histones (lane 4), pCB1 released from DOTAP/DOPE lipoplexes (lane 6), pCB1 released from protocells with cationic cores (lane 8), and histone-packaged pCB1 released from protocells with anionic cores (lane 10) are included for comparison. Lane 1 contains a DNA ladder. Samples were incubated with DNase I (1 unit per 50 ng of DNA) for 30 minutes at room temperature, and pCB1 release was stimulated using 1% SDS.

FIG. 17 shows zeta potential (ζ) values for mesoporous silica nanoparticles (‘unmodified cores’), mesoporous silica nanoparticles that were soaked in 20% (v/v) APTES for 12 hours at room temperature (‘APTES-modified cores’), the CB1 plasmid (‘pCB1’), histone-packaged pCB1 (‘pCB1-histone complex’), and pCB1 packaged with a 1:1 (w/w) mixture of DOTAP and DOPE (‘DOTAP/DOPE Lipoplexes’). Zeta potential measurements were conducted in 0.5×PBS (pH 7.4). Error bars represent 95% confidence intervals (1.96σ) for n=3.

FIG. 18 shows the representative forward scatter-side scatter (FSC-SSC) plots and FL-1 histograms used to determine the percentage of cells positive for ZsGreen expression in FIGS. 6 and 24. (A)-(D) FSC-SSC plots (A and C) and the corresponding FL-1 histograms (B and D, respectively) for ZsGreen-negative cells that were (A) or were not (C) gated to exclude cellular debris. Mean fluorescence intensity (MFI) values for the FL-1 channel are given in (B) and (D). (E)-(H) FSC-SSC plots (E and G) and the corresponding FL-1 histograms (F and H, respectively) for ZsGreen-positive cells that were (E) or were not (G) gated to exclude cellular debris. Gates on (F) and (H) correspond to the percentage of cells with MFI≦282, i.e. 100× the MFI of ZsGreen-negative cells (see panel D).

FIG. 19 shows the identification of the MC40 targeting peptide. Schematic set forth in the figure depicts the process used to select the MC40 targeting peptide. Peptides at 1×10¹¹ pfu/mL were incubated with 100 nM of recombinant human Met (rhMet), fused to the Fc domain of human IgG, for 1 hour at room temperature. Protein A or protein G-coated magnetic particles were used to affinity capture Met-phage complexes and were subsequently washed 10 times with TBS (50 mM Tris-HCl with 150 mM NaCl, pH 7.4) to remove unbound phage. Bound phage clones were eluted with a low-pH buffer (0.2 M glycine with 1 mg/mL BSA, pH 2.2), and elutants were amplified via infection of the host bacterium (E. coli ER2738).

FIG. 20 shows the characterization of the MC40 targeting peptide. (A) Peptide sequence alignment after the 5^{th} round of selection; the predominant sequence, ASVHFPP (SEQ ID NO:1), is similar to the emboldened portion of a previously-identified Met-specific 12-mer, YLFSVHWPPLKA, SEQ ID NO:15, Zhao, et al. ClinCancerRes 2007; 13(20 6049-6055). Phage clones displaying the target-unrelated HAIYPRH peptide (˜10%) (SEQ ID NO:16, Brammer, et al., Anal. Biochem. 373(2008)88-98) were omitted from the sequence alignment. (B) and (C) The degree to which affinity-selected phage clones bound to rhMet was determined via enzyme-linked immunosorbent assay (ELISA). The ELISA scheme, depicted in (B), is described in the Materials and Methods section. ELISA results are shown in (C). (D) Sequence alignment after peptides that do not bind to Met were removed. The consensus sequence depicted in Figure S-9 was determined from this alignment. (E) and (F) Flow cytometry scatter plots for Hep3B (E) and hepatocytes (F) exposed to either (1) an Alexa Fluor® 488-labeled monoclonal antibody against Met AND an irrelevant phage clone (TPDWLFP) (SEQ ID NO:17) and an Alexa Fluor® 546-labeled monoclonal antibody against M13 phage (blue dots) or (2) an Alexa Fluor® 488-labeled monoclonal antibody against Met AND the MC40 clone AND an Alexa Fluor® 546-labeled monoclonal antibody against M13 phage (orange dots). Untreated cells (red dots) were used to set voltage parameters for the FL-1 (Alexa Fluor® 488 fluorescence) and FL-2 (Alexa Fluor® 546 fluorescence) channels.

FIG. 21 shows sample binding curves for MC40-targeted protocells exposed to Hep3B. To determine the dissociation constants in FIG. 5A, 1×10⁶ Hep3B or hepatocytes were pre-treated with cytochalasin D to inhibit endocytosis and incubated with various concentrations of Alexa Fluor® 647-labeled, MC40-targeted protocells for 1 hour at 37° C. Flow cytometry was used to determine mean fluorescence intensities for the resulting cell populations, which were plotted against protocell concentrations to obtain total binding curves. Non-specific binding was determined by incubating cells with Alexa Fluor® 647-labeled, MC40-targeted protocells in the presence of a saturating concentration of unlabeled hepatocyte growth factor. Specific binding curves were obtained by subtracting non-specific binding curves from total binding curves; K_{d} values were calculated from specific binding curves. In the experiments depicted in this figure, protocell SLBs were composed of DOPC with 5 wt % DOPE, 30 wt % cholesterol, and 10 wt % PEG-2000 (18:1) and were modified with 0.015 wt % (˜6 peptides/particle) of the MC40 targeting peptide; the corresponding K_{d} value is 1050±142 pM. All error bars represent 95% confidence intervals (1.96σ) for n=5.

FIG. 22 shows that MC40-targeted protocells are internalized via receptor-mediated endocytosis and, in the absence of the H5WYG peptide, are directed to lysosomes. (A) The average number of MC40-targeted protocells internalized by each Hep3B or hepatocyte cell within one hour at 37° C. 1×10⁶ cells were incubated with various concentrations of protocells in the absence (−) or presence (+) of a saturating concentration (100 μg/mL) of human hepatocyte growth factor (HGF), and flow cytometry was used to determine the average number of particles associated with each cell. Protocells were labeled with NBD and pHrodo™ to distinguish surface-bound particles from those internalized into acidic intracellular compartments (respectively). Error bars represent 95% confidence intervals (1.96σ) for n=3. (B) Pearson's correlation coefficients (r-values) between protocells and: (1) Rab5, (2) Rab7, (3) Lysosomal-Associated Membrane Protein 1 (LAMP-1), or (4) Rab11a. Hep3B cells were incubated with a 1000-fold excess of Alexa Fluor® 594-labeled protocells for 1 hour at 37° C. before being fixed, permeabilized, and incubated with Alexa Fluor® 488-labeled antibodies against Rab5, Rab7, LAMP-1, or Rab11a. SlideBook software was used to determine r-values, which are expressed as the mean value±the standard deviation for n=3×50 cells. Differential Interference Contrast (DIC) images were employed to define the boundaries of Hep3B cells so that pixels outside of the cell boundaries could be disregarded when calculating r-values. Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG.

FIG. 23 shows that histone-packaged pCB1, when modified with a NLS and delivered via MC40-targeted protocells, becomes concentrated in the nuclei of HCC cells in a time-dependent manner. (A)-(C) Confocal fluorescence microscopy images of Hep3B cells exposed to a 1000-fold excess of MC40-targeted, pCB1-loaded protocells for 15 minutes (A), 12 hours (B), or 24 hours (C) at 37° C. For (B), endosomal escape of protocells and cytosolic dispersion of pCB1 was evident after ˜2 hours; ZsGreen expression was not detectable until 12-16 hours, however. At 24 hours, Cy5-labeled pCB1 remained distributed throughout the cells; cytosolic staining is not visible in (C), however, since the gain of the Cy5 channel was reduced to avoid saturation of pixels localized within the nuclei. Silica cores were labeled with Alexa Fluor® 594 (red), pCB1 was labeled with Cy5 (white), and cell nuclei were counterstained with Hoechst 33342 (blue). Scale bars=20 μm. (D) Pearson's correlation coefficients (r-values) versus time for Cy5-labeled pCB1 and Hoechst 33342-labeled Hep3B nuclei. SlideBook software was used to determine r-values, which are expressed as the mean value±the standard deviation for n=3×50 cells. Differential Interference Contrast (DIC) images were employed to define the boundaries of Hep3B cells so that pixels outside of the cell boundaries could be disregarded when calculating r-values. Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG.

FIG. 24 shows that histone-packaged pCB1, when modified with a NLS and delivered via MC40-targeted protocells, selectively transfects both dividing and non-dividing HCC cells with nearly 100% efficacy. (A), (C), and (E) Confocal fluorescence microscopy images of Hep3B cells exposed to a 1000-fold excess of MC40-targeted, pCB1-loaded protocells for 24 hours at 37° C. Hep3B cells were dividing in (A) and ˜95% confluent in (C) and (E); pCB1 was pre-packaged with histones in all images, and the pCB1-histone complex was further modified with a NLS in (E). Silica cores were labeled with Alexa Fluor® 594 (red), pCB1 was labeled with Cy5 (white), and cell nuclei were counterstained with Hoechst 33342 (blue). Scale bars=20 μm. (B), (D), and (F) The percentage of 1×10⁶ Hep3B and hepatocytes that become positive for ZsGreen expression upon continual exposure to 1×10⁹ MC40-targeted, pCB1-loaded protocells (‘PC’) for 24 hours at 37° C. Cells were dividing in (B) and ˜95% confluent in (D) and (F); the x-axes indicate whether CB1 plasmids (‘pCB1’) and pCB1-histone complexes (‘complex’) were modified with the NLS. pCB1 alone, as well as pCB1 packaged with a 1:1 (w/w) mixture of DOTAP and DOPE were employed as controls. Cells were exposed to 20 mg/mL of wheat germ agglutinin (WGA) to block translocation of NLS-modified pCB1 through the nuclear pore complex. Error bars represent 95% confidence intervals (1.96σ) for n=3. (G)-(I) Cell cycle histograms for cells employed in (A), (C), and (E), respectively. The percentage of cells in G₀/G₁ phase is given for each histogram. In all experiments, protocell SLBs were composed of DOPC with 5 wt % DOPE, 30 wt % cholesterol, and 10 wt % PEG-2000 (18:1) and were modified with 0.015 wt % MC40 and 0.500 wt % H5WYG.

FIG. 25 shows confocal fluorescence microscopy images of Hep3B (A) and hepatocytes (B) that were exposed to MC40-targeted, pCB1-loaded protocells for either 1 hour or 72 hours at 37° C.; the pCB1 concentration was maintained at 5 pM in all experiments. The arrows in (B) indicate mitotic cells. Cyclin B1 was labeled with an Alexa Fluor® 594-labeled monoclonal antibody (red), and cell nuclei were stained with Hoechst 33342 (blue). Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG. All scale bars=20 μm.

FIG. 26 shows confocal fluorescence microscopy images of Hep3B (A) and hepatocytes (B) that were exposed to MC40-targeted, pCB1-loaded protocells for either 1 hour or 72 hours at 37° C.; the pCB1 concentration was maintained at 5 pM in all experiments. Cells were stained with Alexa Fluor® 647-labeled annexin V (white) and propidium iodide (red) to assay for early and late apoptosis, respectively, and cell nuclei were counterstained with Hoechst 33342 (blue). Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG. All scale bars=20 μm.

FIG. 27 shows that protocells with a SLB composed of zwitterionic lipids induce minimal non-specific cytotoxicity. The percentage of 1×10⁶ Hep3B that become apoptotic upon continual exposure to 1×10⁹ APTES-modified mesoporous silica nanoparticles, DOPC protocells with APTES-modified cores, DOPC protocells loaded with a plasmid that encodes a scrambled shRNA sequence (‘scrambled pCB1’), or DOTAP/DOPE (1:1 w/w) lipoplexes loaded with scrambled pCB1 for 48 hours at 37° C. Protocells and lipoplexes were modified with 10 wt % PEG-2000, 0.015 wt % MC40, and 0.500 wt % H5WYG. Positively- and negatively-charged polystyrene nanoparticles (′amine-PS' and ‘Carboxyl-PS’, respectively) were employed as positive controls, while Hep3B exposed to 10 mM of the antioxidant, N-acetylcysteine (NAC), or to 1 pmol of free pCB1 were used as negative controls. All error bars represent 95% confidence intervals (1.96σ) for n=3.

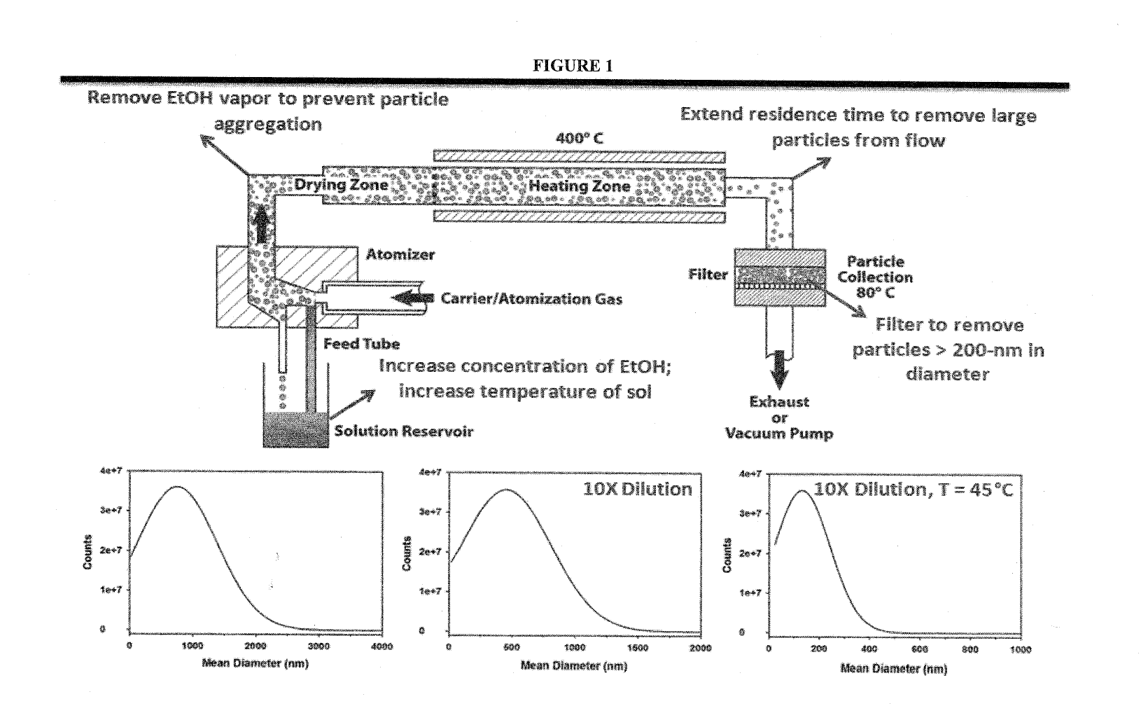

FIG. 1X2 shows the aqueous solubility of Imatinib as a function pH. The solubility of the drug increased as the pH decreased due to ionization of the weakly basic functional groups on the chemical structure.

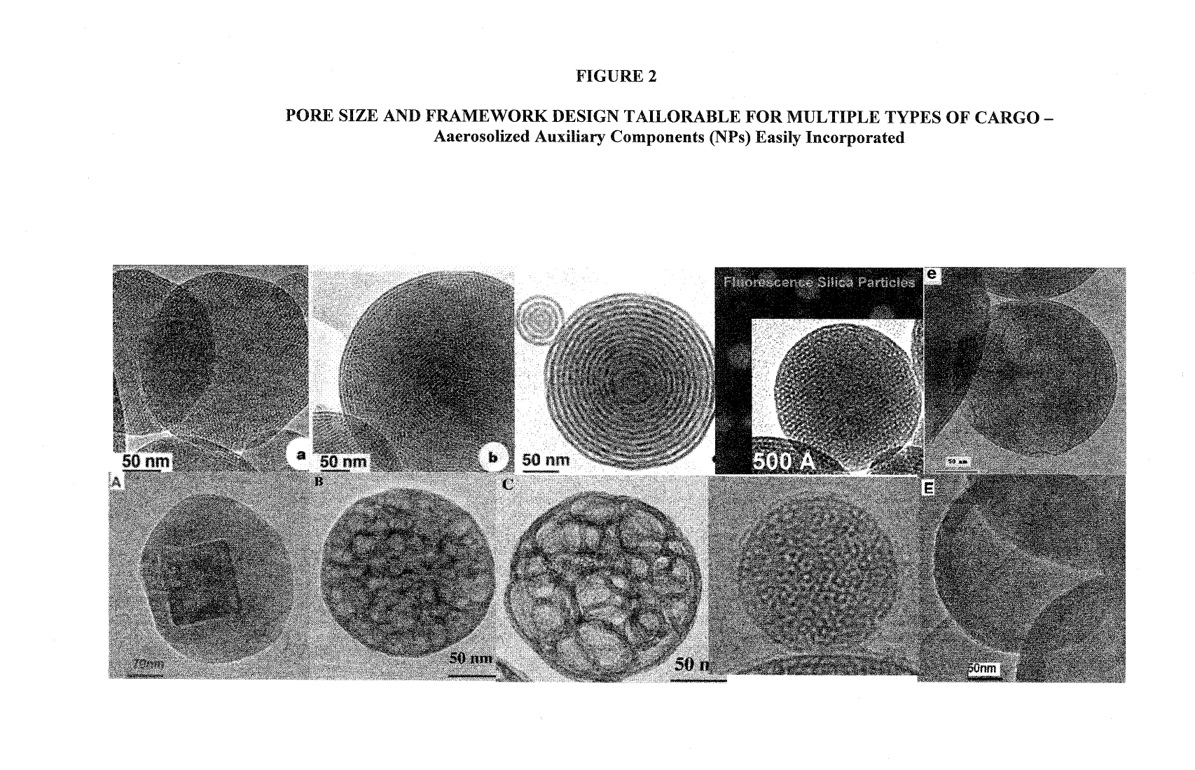

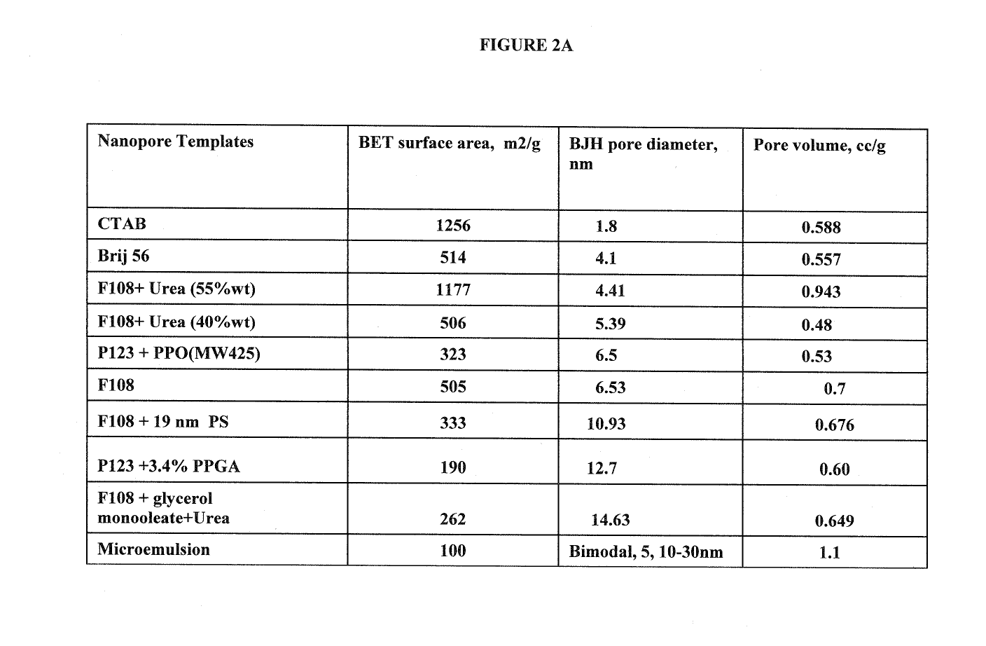

FIG. 2X2 shows the solubility of Imatinib in different formulations at pH 7. The formulation with 10% ethanol exhibited the highest solubility compared to the other formulations. Imatinib was also found to be highly soluble in DMSO.

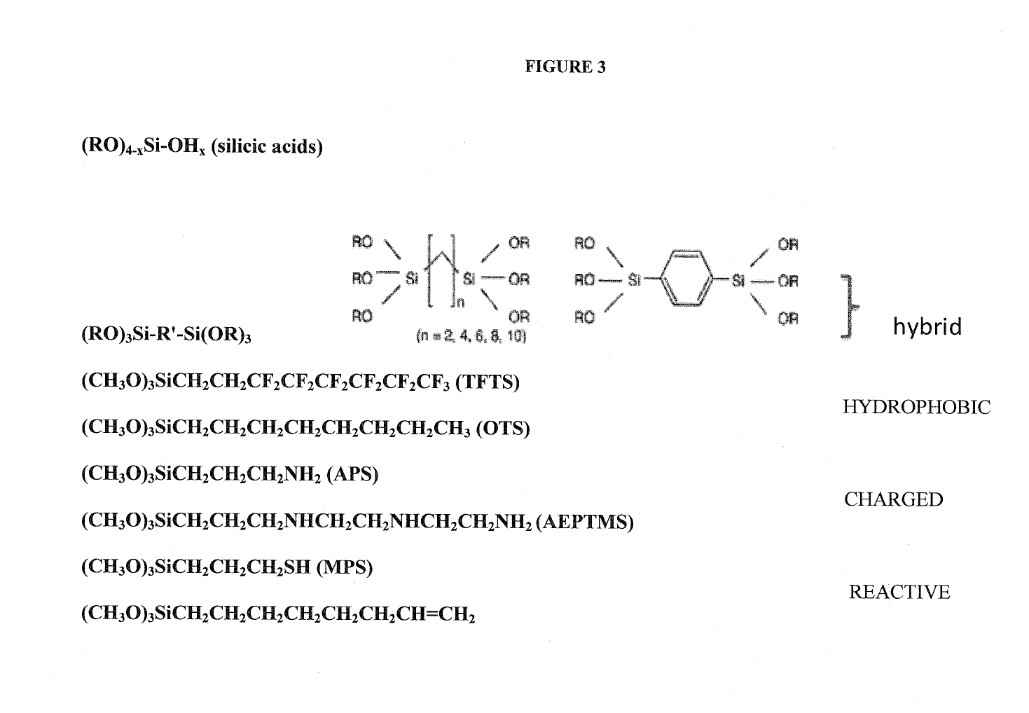

FIG. 3X2 shows the influence of solvent system on the permeation of imatinih over 24 hours. All the formulations containing cosolvents showed higher penetration through the skin compared to the control (water, pH 7). DMSO exhibited the highest permeation. (N12-186PCT 2012-032-01 Provisional.PDF).

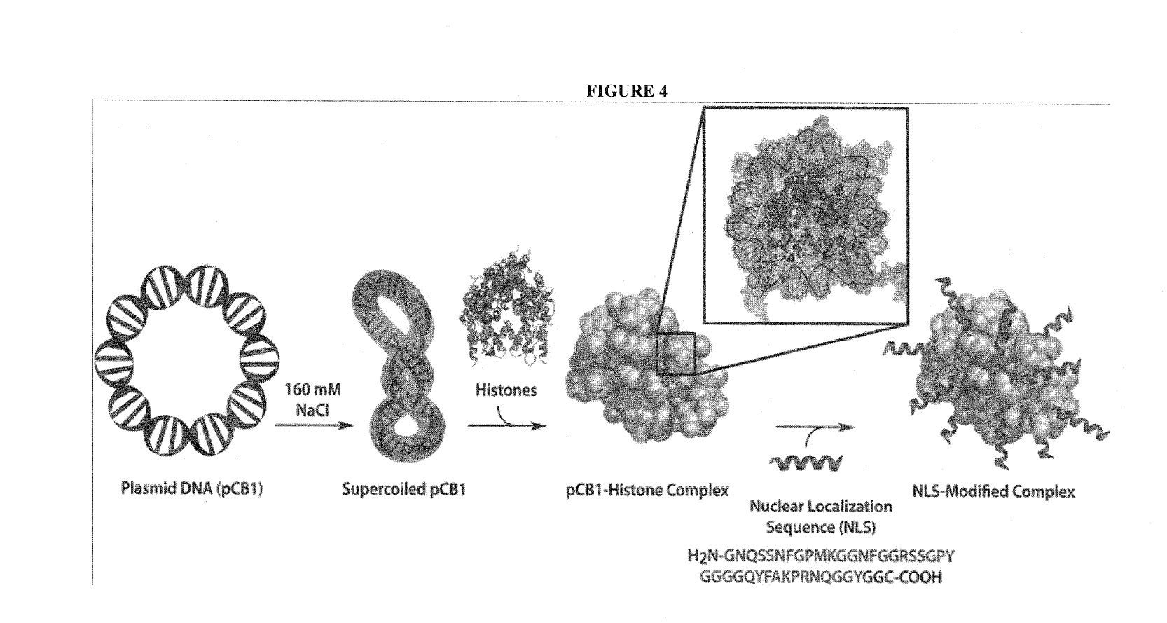

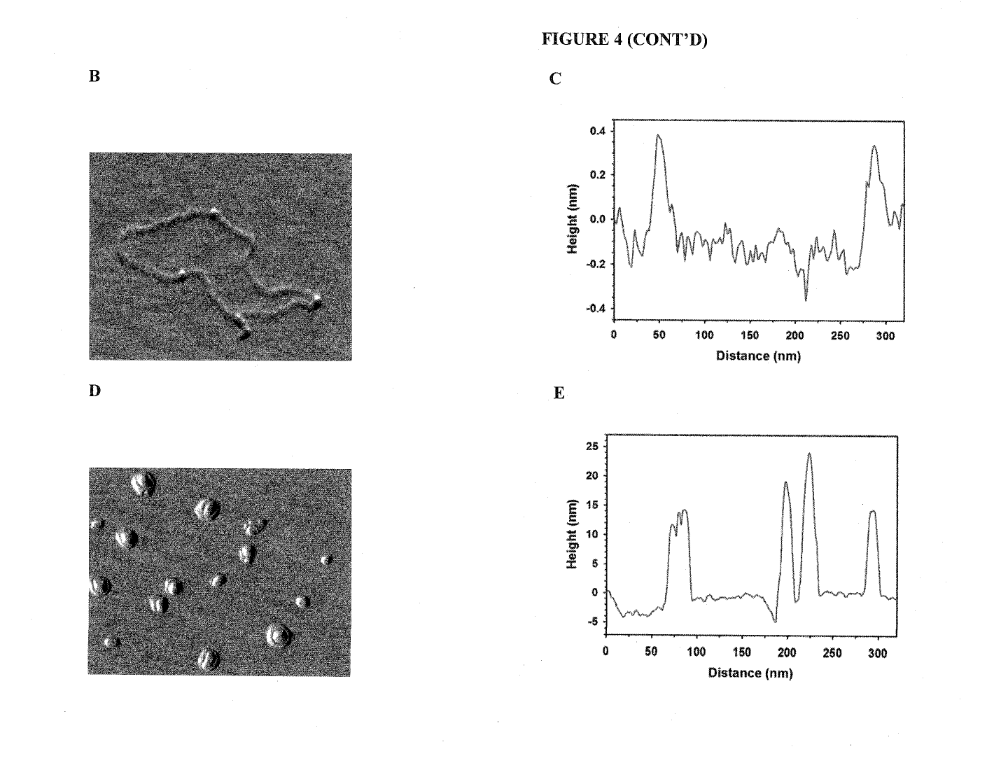

FIG. 4X2 shows the effect of solvent system on flux (rate of transdermal permeation) of imatinib. The formulation containing DMSO exhibited the highest flux of the formulations investigated.

FIG. 1X3. Schematic depicting the process used to synthesize siRNA or protein toxin-loaded nanoporous particle-supported lipid bilayers (protocells). To form protocells loaded with macromolecular therapeutic agents and targeted to hepatocellular carcinoma (HCC), nanoporous silica cores modified with an amine-containing silane (AEPTMS) were first soaked in a solution of small interfering RNA (siRNA) or a protein-based toxin (e.g. ricin toxin A-chain). Liposomes composed of DOPC, DOPE, cholesterol, and 18:1 PEG-2000 PE (55:5:30:10 mass ratio) were then fused to cargo-loaded cores. The resulting supported lipid bilayer (SLB) was modified with a targeting peptide (SP94) that binds to HCC and an endosomolytic peptide (H5WYG) that promotes endosomal/lysosomal escape of internalized protocells. Peptides, modified with glycine-glycine (GG) spacers and C-terminal cysteine residues, were conjugated to primary amines present in DOPE moieties via a heterobifunctional crosslinker (SM(PEG)₂₄) with a 9.5-nm polyethylene glycol (PEG) spacer. The SP94 and H5WYG sequences reported by Lo, et al.⁶⁵ and Moore, et al.⁶⁶ cited in Example 3 are given in red.

FIG. 2X3. Characterization of the nanoporous silica particles that form the protocell core. (A) Dynamic light scattering (DLS) of multimodal silica particles, before and after size-based separation. Particles have an average particle diameter of ˜165 nm after separation. (B) Nitrogen sorption isotherm for multimodal particles. The presence of hysteresis is consistent with a network of larger pores interconnected by smaller pores. (C) A plot of pore diameter vs. pore volume, calculated from the adsorption isotherm in (e), demonstrates the presence of large (20-30 nm) pores and small (6-12 nm) pores.

FIG. 3X3. Protocells have a high capacity for siRNA, the release of which is triggered by acidic pH. (A) The concentrations of siRNA that can be loaded within 10¹⁰ protocells and lipoplexes. Zeta potential values for unmodified and AEPTMS-modified silica cores in 0.5×PBS (pH 7.4) are ˜−32 mV and +12 mV, respectively. (B) and (C) The rates at which siRNA is released from DOPC protocells with AEPTMS-modified cores, DOPC lipoplexes, and DOTAP lipoplexes upon exposure to a pH 7.4 simulated body fluid (B) or a pH 5.0 buffer (C) at 37° C. The average diameters of siRNA-loaded protocells, DOPC lipoplexes, and DOTAP lipoplexes were 178-nm, 135-nm, and 144-nm, respectively. Error bars represent 95% confidence intervals (1.96σ) for n=3.

FIG. 4X3. siRNA-loaded, SP94-targeted protocells silence various cyclin family members in HCC but not hepatocytes. (A) and (B) Dose (A) and time (B) dependent decreases in the expression of cyclin A2, B1, D1, and E protein upon exposure of Hep3B to siRNA-loaded, SP94-targeted protocells. 1×10⁶ cells were continually exposed to various concentrations of siRNA for 48 hours in (A) and to 125 pM of siRNA for various periods of time in (B). (C, left axis) Percentages of initial cyclin A2 protein concentrations that remain upon exposure of 1×10⁶ Hep3B or heaptocytes to 125 pM of siRNA for 48 hours. (C, right axis) The number of siRNA-loaded, SP94-targeted DOPC protocells, DOPC lipoplexes, and DOTAP lipoplexes that must be incubated with 1×10⁶ Hep3B cells to reduce expression of cyclin A2 protein to 10% of the initial concentration. Protocell SLBs were composed of DOPC with 5 wt % DOPE, 30 wt % cholesterol, and 10 wt % PEG-2000 and were modified with 0.015 wt % SP94 and 0.500 wt % H5WYG. DOPC (and DOTAP) lipoplexes were prepared using a 55:5:30:10 ratio of DOPC (or DOTAP):DOPE:cholesterol:PEG-2000 PE and were modified with 0.015 wt % SP94, and 0.500 wt % H5WYG. All experiments were conducted in complete growth medium at 37° C. Error bars represent 95% confidence intervals (1.96σ) for n=3.

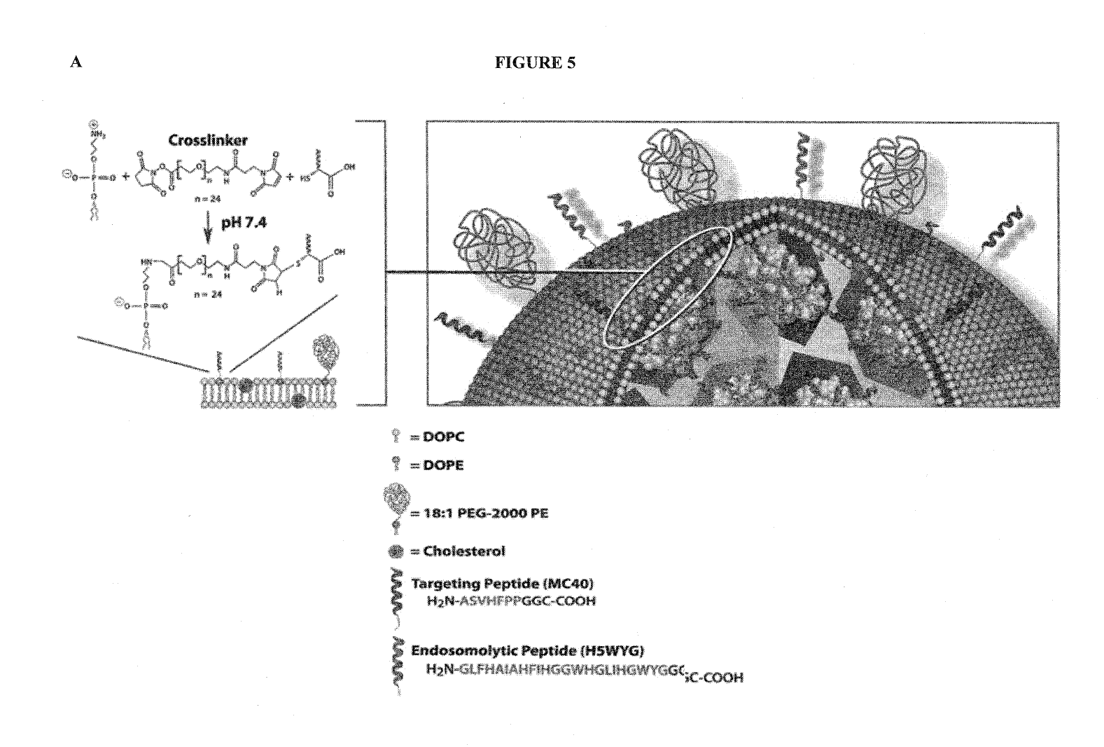

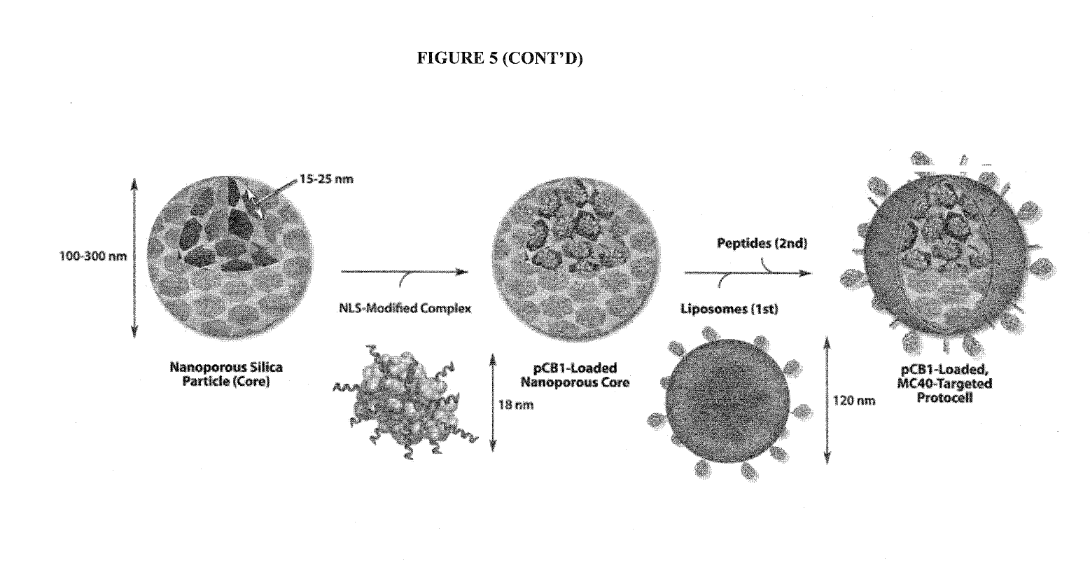

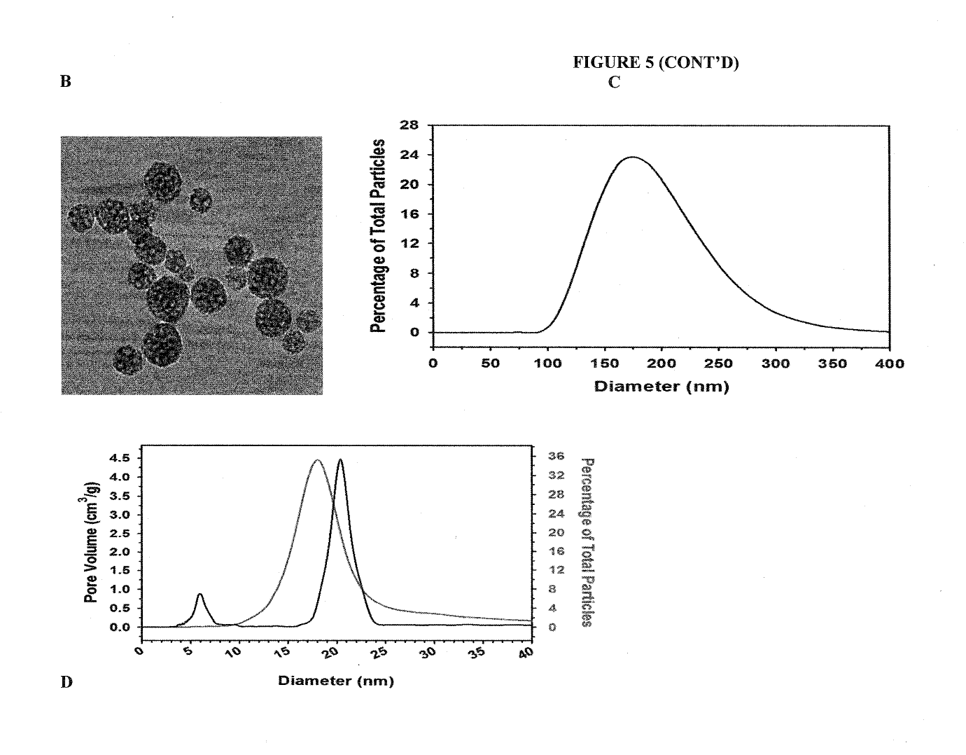

FIG. 5X3. Confocal fluorescence microscopy images of Hep3B (A) and hepatocytes (B) after exposure to siRNA-loaded, SP94-targeted protocells for 1 hour or 48 hours at 37° C. Cells were incubated with a 10-fold excess of Alexa Fluor 647-labeled protocells (white) prior to being fixed, permeablized, and stained with Hoechst 33342 (blue) and Alexa Fluor 488-labeled antibodies against cyclin A2, cyclin B1, cyclin D1, or cyclin E (green). Protocell SLBs were composed of DOPC with 5 wt % DOPE, 30 wt % cholesterol, and 10 wt % PEG-2000 and were modified with 0.015 wt % SP94 and 0.500 wt % H5WYG. Scale bars=20 μm.

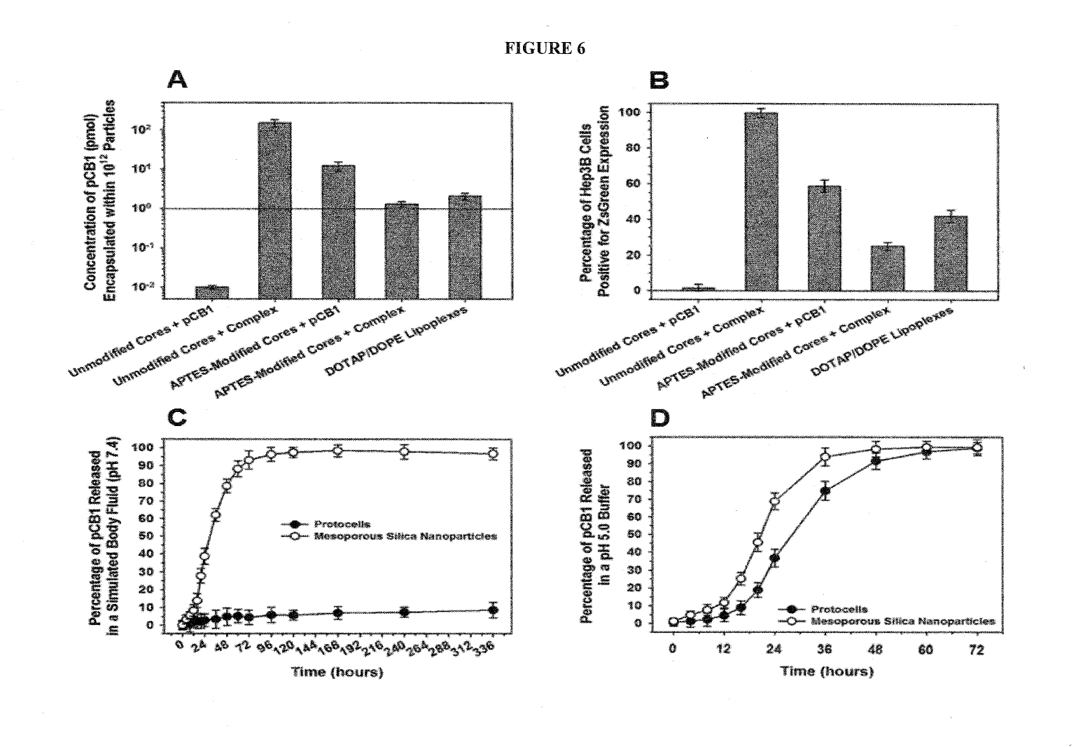

FIG. 6X3. SP94-targeted protocells loaded with the cyclin-specific siRNA cocktail induce apoptosis in HCC without affecting hepatocyte viability. (A) The percentage of 1×10⁶ Hep3B and hepatocytes that become positive for Alexa Fluor 488-labeled annexin V and/or propidium iodide (PI) upon exposure to SP94-targeted protocells loaded with the cyclin-specific siRNA cocktail for various periods of time at 37° C. Cells positive for annexin V were considered to be in the early stages of apoptosis, while cells positive for both annexin V and PI were considered to be in the late stages of apoptosis; the total number of apoptotic cells was determined by adding the numbers of cells in early and late apoptosis. The total siRNA concentration was maintained at ˜125 pM. Error bars represent 95% confidence intervals (1.96σ) for n=3. (B) and (C) Confocal fluorescence microscopy images of Hep3B (B) and hepatocytes (C) after exposure to siRNA-loaded, SP94-targeted protocells for 1 hour or 48 hours at 37° C. Cells were incubated with a 10-fold excess of Alexa Fluor 647-labeled protocells (white) prior to being stained with Hoechst 33342 (blue), Alexa Fluor 488-labeled annexin V (green), and propidium iodide (red). Differential Interference Contrast (DIC) images are included to show cell morphology. Scale bars=20 μm. In all experiments, protocell SLBs were composed of DOPC with 5 wt % DOPE, 30 wt % cholesterol, and 10 wt % PEG-2000 and were modified with 0.015 wt % SP94 and 0.500 wt % H5WYG.

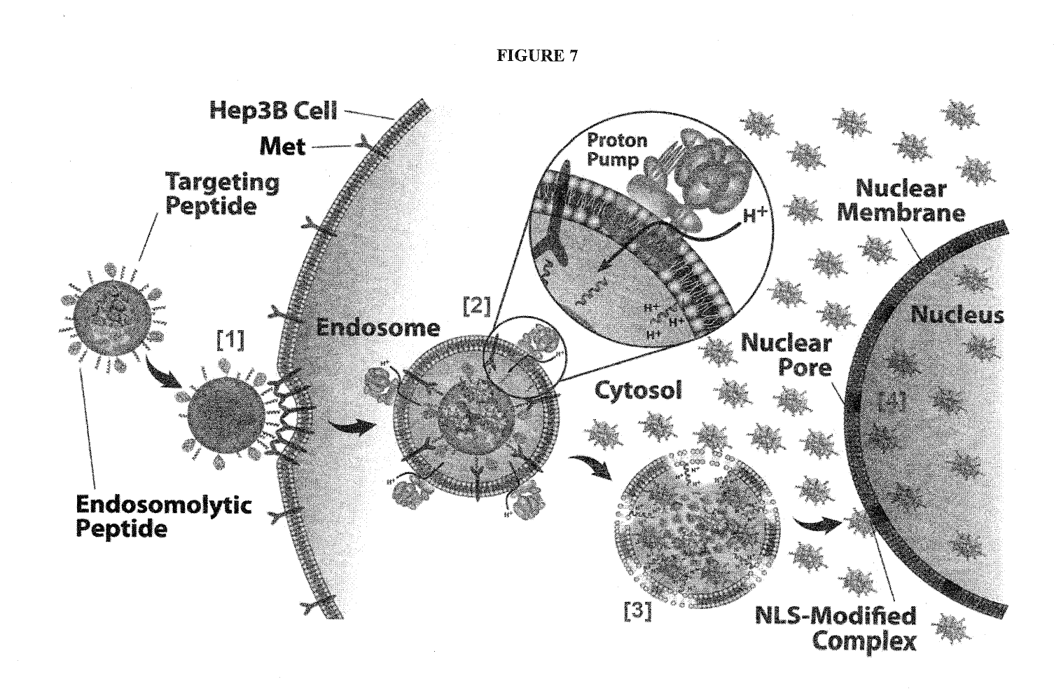

FIG. 7X3. Protocells encapsulate a high concentration of ricin toxin A-chain (RTA) and release it only at acidic pH. (A) The concentrations of RTA that can be encapsulated within 10¹⁰ protocells and liposomes. Zeta potential values for unmodified and AEPTMS-modified silica cores in 0.5×PBS (pH 7.4) are ˜32 mV and +12 mV, respectively. The isoelectric point (pI) of deglycosolated RTA is ˜7. (B) and (C) Time-dependent release of RTA upon exposure of DOPC protocells with AEPTMS-modified cores and DOPC liposomes to a pH 7.4 simulated body fluid (B) or a pH 5.0 buffer (C) at 37° C. Average diameters of RTA-loaded protocells and liposomes were 184-nm and 140-nm, respectively. Error bars represent 95% confidence intervals (1.96σ) for n=3.

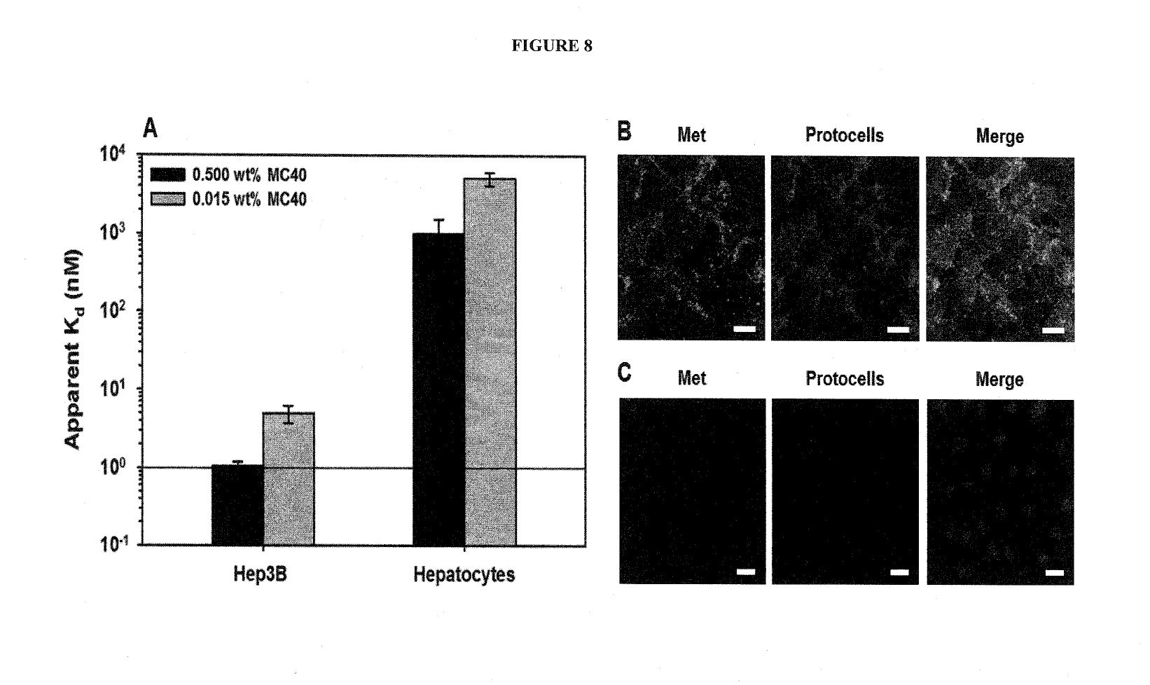

FIG. 8X3. RTA-loaded, SP94-targeted protocells inhibit protein biosynthesis in HCC but not hepatocytes. (A) and (B) Dose (A) and time (B) dependent decreases in nascent protein synthesis upon exposure of Hep3B to RTA-loaded, SP94-targeted protocells. 1×10⁶ cells were continually exposed to various concentrations of RTA for 48 hours in (A) and to 25 pM of RTA for various periods of time in (B). Nascent protein synthesis was quantified using an Alexa Fluor 488-labeled derivative of methionine. (C, left axis) Percentages of initial nascent protein concentrations that remain upon exposure of 1×10⁶ Hep3B or heaptocytes to 25 pM of RTA for 48 hours. (C, right axis) The number of RTA-loaded, SP94-targeted DOPC protocells and liposomes that must be incubated with 1×10⁶ Hep3B cells to reduce protein biosynthesis by 90%. Protocell and liposome bilayers were composed of DOPC with 5 wt % DOPE, 30 wt % cholesterol, and 10 wt % PEG-2000 and were modified with 0.015 wt % SP94 and 0.500 wt % H5WYG. All experiments were conducted in complete growth medium at 37° C. Error bars represent 95% confidence intervals (1.96σ) for n=3.

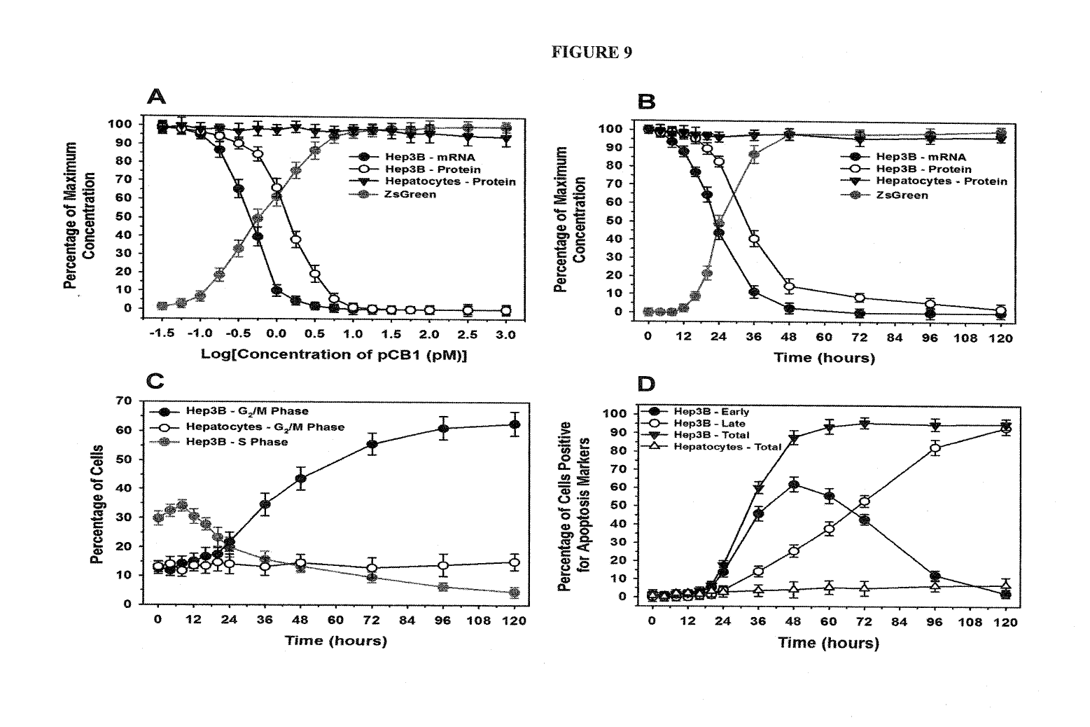

FIG. 9X3. Confocal fluorescence microscopy images of Hep3B (A) and hepatocytes (B) after exposure to RTA-loaded, SP94-targeted protocells for 1 hour or 48 hours at 37° C. Cells were incubated with a 10-fold excess of Alexa Fluor 647-labeled protocells (white) prior to being stained with Hoechst 33342 (blue) and the Click-iT AHA Alexa Fluor 488 Protein Synthesis Kit (green). Protocell SLBs were composed of DOPC with 5 wt % DOPE, 30 wt % cholesterol, and 10 wt % PEG-2000 and were modified with 0.015 wt % SP94 and 0.500 wt % H5WYG. Scale bars=20 μm.

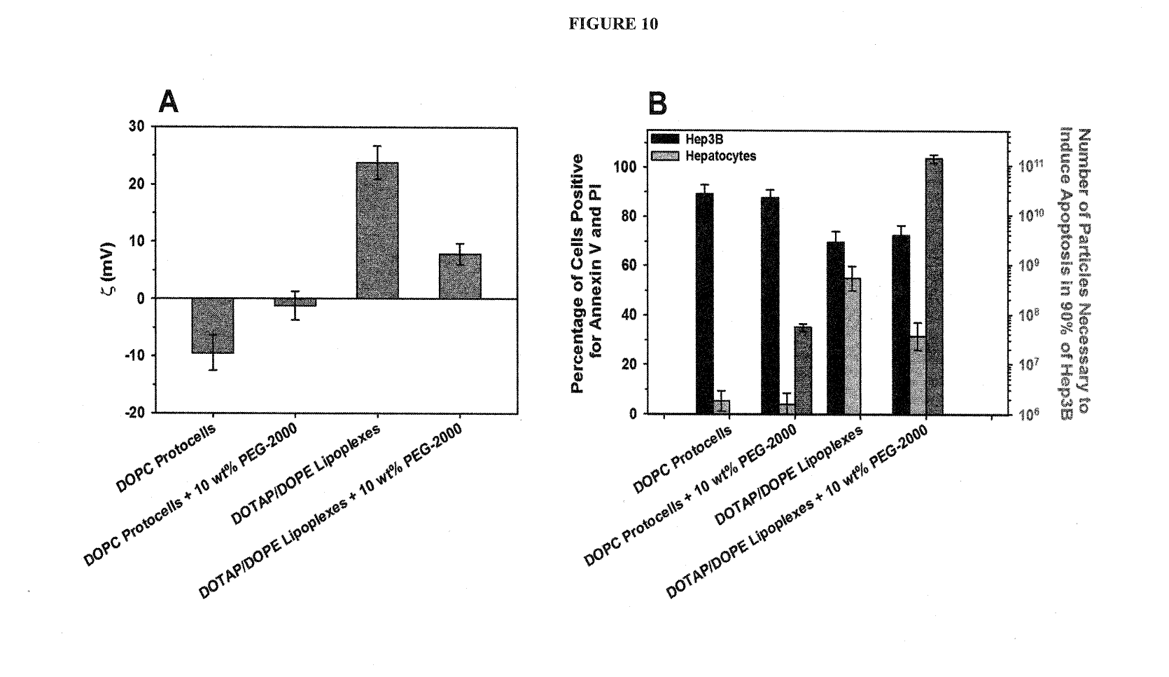

FIG. 10X3. SP94-targeted protocells loaded with RTA induce selective apoptosis of HCC. (A) The percentage of 1×10⁶ Hep3B and hepatocytes that become positive for caspase-9 or caspase-3 activation upon exposure to RTA-loaded, SP94-targeted protocells for various periods of time at 37° C. The total RTA concentration was maintained at ˜25 pM. Error bars represent 95% confidence intervals (1.96σ) for n=3. (B) and (C) Confocal fluorescence microscopy images of Hep3B (B) and hepatocytes (C) after exposure to RTA-loaded, SP94-targeted protocells for 1 hour or 48 hours at 37° C. Cells were incubated with a 10-fold excess of Alexa Fluor 647-labeled protocells (white) prior to being stained with Hoechst 33342 (blue), CaspGLOW Fluorescein Active Caspase-9 Staining Kit (green), and CaspGLOW Red Active Caspase-3 Staining Kit (red). Differential Interference Contrast (DIC) images are included to show cell morphology. Scale bars=20 μm. In all experiments, protocell SLBs were composed of DOPC with 5 wt % DOPE, 30 wt % cholesterol, and 10 wt % PEG-2000 and were modified with 0.015 wt % SP94 and 0.500 wt % H5WYG.

FIG. 1X5 is a schematic that depicts the “brick and mortar” structure of the SC along with the three routes of passive transdermal diffusion. Intercellular diffusion is widely accepted as the primary route, however it usually occurs in parallel with transcellular diffusion, and are both influenced by the strategy of permeation enhancement employed. Transappendageal diffusion is often neglected since sweat glands and hair follicles only account for about 1% of the body's surface-area.

FIG. 2X5 is a schematic that illustrates the protocell and is representative of the various modifications to its core and SLB that can be made in order to optimize it for a specific application. Starting in the bottom left, the protocell is composed of a nanoporous silica core that is encapsulated by a supported lipid bilayer. The core has a high-surface area, controllable particle diamter, tunable pore size, modifiable surface chemistry, and can be engineered to facilitate high-capacity loading of disparate types of cargo (i.e. nanoparticles, protein toxins, therapeutic nucleic acids, drugs). The supported lipid bilayer provides a fluid surface to which various molecules (i.e. peptides, polyethylene glycol-PEG) can be conjugated using heterobifunctional crosslinkers to affect specific binding, internalization, and permeation.

FIG. 3X5. Preliminary results illustrate that Protocells can interact with the SC, penetrate the SC, and diffuse across the skin, and that the transdermal kinetics of these interactions are influenced by SLB composition and formulation. a.) ICP-MS results showing the total amount (ug) of SiO₂ in the receptacles of skin samples (n=3) treated with DOPC/Chol/PEG, where the SC was left intact or removed. b.) A schematic illustrating core functionalization with fluorophores, along with the necessary characterizations that must be made after each step. c.) DOPC/Chol formulated Protocells showed nearly 2× the amount of SiO₂ in the receptacle after 24 hours with respect to the DSPC/Chol formulation. However, the same Protocells formulated with PEG show significantly decreased kinetics with respect to their non-pegylated formulations.

FIG. 1X6. Schematic depicting the protocell that we propose to develop for targeted delivery of anti-viral agents to potential host cells and already infected cells. The MSNP core is shown in blue, and the SLB is shown in yellow.

FIG. 2X6. Preliminary in vivo characterization of non-targeted, PEGylated protocells. (A) The time-dependent weight of Balb/c mice that were injected with protocells or saline. (B) Balb/c mice injected with DyLight 633-labeled protocells or 100 μL, of saline (control) and imaged with an IVIS Lumina II. In all experiments, protocells were modified with 10 wt % of PEG-2000 and were injected into the tail vein.

FIG. 1X7. General biodistribution and toxicity of the protocell in vivo. (A) Systemic circulation of particles immediately following intravenous injection and (B) localization to liver and bone 48 hours following injection. (C) Three times weekly dosing of protocells results in no gross signs of toxicity including by whole animal weight. (D) Fluorescence from particles are observed to accumulate in the liver of mice injected with protocells (D1, D3, D4—30 mg total over 4 weeks) with no effect on liver anatomy.

FIG. 2X7. 3D rendering of particle distribution in thick section of the liver. Particles are found to accumulate within defined, but currently unidentified, areas of the liver over time. No gross or histological toxicity have been observed at doses up to 30 mg per mouse over 4 weeks. Scale 20 μm.

FIGS. 1X8, 2X8, 3X8. Protocell diffusion through full and partial skin thickness as determined in the experiment of Example 8.

FIG. 4X8. ICP mass spec of donor cap samples as determined in the experiment of Example 8.

FIG. 5X8. Core functionalization as determined in the experiment of Example 8.

FIG. 6X8, 7X8, 8X8, 9X8. Spectrafluorimetry used to determine the concentration of SiO₂ in the receptacle fluid as determined in the experiment of Example 8.

FIG. 10X8. Positive control showed that fluorescently-tagged particles in the skin can be imaged while taking advantage of the skin's autofluorescence as determined in the experiment of Example 8.

FIG. 1X9. Various protocell formulations were administered (500 μl of 16 mg/ml in 0.5×PBS) with n=4 for each formulation. 1 skin (51) from each experiment was treated with 0.5×PBS. Standard curves were generated within the concentration range of 0.16 mg/ml-1.953125E-5 mg/ml using a 1:2 dilution from the S1 24 hour receptacle fluid, as determined in the experiment of Example 9.

FIG. 2X9. Liner regression analysis in conjunction with spectrafluorimetry as determined in the experiment of Example 9.

FIG. 3X9. SLB formulation can drastically affect transdermal diffusion as determined in the experiment of Example 9.

FIG. 4X9. The addition of PEG to DOPC/chol and DSPC/chol formulations significantly decreased transdermal diffusion as determined in the experiment of Example 9.

FIG. 5X9, 6X9. The individual increase in the corrected mean fluorescence intensities as a function of time as determined in the experiment of Example 9.

FIGS. 7X9, 8X9 and 9X9 illustrate the effect of formulation on kinetics as determined in the experiment of Example 9.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diasteromers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment of the present invention. Exemplary bioactive agents include the compounds according to the present invention which are used to treat cancer or a disease state or condition which occurs secondary to cancer and may include antiviral agents, especially anti-HIV, anti-HBV and/or anti-HCV agents (especially where hepatocellular cancer is to be treated) as well as other compounds or agents which are otherwise described herein.

The terms “treat”, “treating”, and “treatment”, are used synonymously to refer to any action providing a benefit to a patient at risk for or afflicted with a disease, including improvement in the condition through lessening, inhibition, suppression or elimination of at least one symptom, delay in progression of the disease, prevention, delay in or inhibition of the likelihood of the onset of the disease, etc. In the case of viral infections, these terms also apply to viral infections and preferably include, in certain particularly favorable embodiments the eradication or elimination (as provided by limits of diagnostics) of the virus which is the causative agent of the infection.

Treatment, as used herein, encompasses both prophylactic and therapeutic treatment, principally of cancer, but also of other disease states, including viral infections, especially including HBV and/or HCV. Compounds according to the present invention can, for example, be administered prophylactically to a mammal in advance of the occurrence of disease to reduce the likelihood of that disease. Prophylactic administration is effective to reduce or decrease the likelihood of the subsequent occurrence of disease in the mammal, or decrease the severity of disease (inhibition) that subsequently occurs, especially including metastasis of cancer. Alternatively, compounds according to the present invention can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds according to the present invention is effective to decrease the severity of the disease or lengthen the lifespan of the mammal so afflicted, as in the case of cancer, or inhibit or even eliminate the causative agent of the disease, as in the case of hepatitis B virus (HBV) and/or hepatitis C virus infections (HCV) infections.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions according to the present invention, alone or in combination with another agent. It is noted that prophylaxis will rarely be 100% effective; consequently the terms prevention and reducing the likelihood are used to denote the fact that within a given population of patients or subjects, administration with compounds according to the present invention will reduce the likelihood or inhibit a particular condition or disease state (in particular, the worsening of a disease state such as the growth or metastasis of cancer) or other accepted indicators of disease progression from occurring.

The term “protocell” is used to describe a porous nanoparticle which is made of a material comprising silica, polystyrene, alumina, titania, zirconia, or generally metal oxides, organometallates, organosilicates or mixtures thereof.

Porous nanoparticulates used in protocells of the invention include mesoporous silica nanoparticles and core-shell nanoparticles.

The porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

A porous spherical silica nanoparticle is used for the preferred protocells and is surrounded by a supported lipid or polymer bilayer or multilayer. Various embodiments according to the present invention provide nanostructures and methods for constructing and using the nanostructures and providing protocells according to the present invention. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from SkySpring Nanomaterials, Inc., Houston, Tex., USA or from Discovery Scientific, Inc., Vancouver, British Columbia. Multimodal silica nanoparticles may be readily prepared using the procedure of Carroll, et al., Langmuir, 25, 13540-13544 (2009). Protocells can be readily obtained using methodologies known in the art. The examples section of the present application provides certain methodology for obtaining protocells which are useful in the present invention. Protocells according to the present invention may be readily prepared, including protocells comprising lipids which are fused to the surface of the silica nanoparticle. See, for example, Liu, et al., Chem. Comm., 5100-5102 (2009), Liu, et al., J. Amer. Chem. Soc., 131, 1354-1355 (2009), Liu, et al., J. Amer. Chem. Soc., 131, 7567-7569 (2009) Lu, et al., Nature, 398, 223-226 (1999), Preferred protocells for use in the present invention are prepared according to the procedures which are presented in Ashley, et al., Nature Materials, 2011, May; 10(5):389-97, Lu, et al., Nature, 398, 223-226 (1999), Caroll, et al., Langmuir, 25, 13540-13544 (2009), and as otherwise presented in the experimental section which follows.

The terms “nanoparticulate” and “porous nanoparticulate” are used interchangeably herein and such particles may exist in a crystalline phase, an amorphous phase, a semi-crystalline phase, a semi amorphous phase, or a mixture thereof.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, or a whisker. A nanoparticle may include particles having two or more of the aforementioned shapes. In one embodiment, a cross-sectional geometry of the particle may be one or more of circular, ellipsoidal, triangular, rectangular, or polygonal. In one embodiment, a nanoparticle may consist essentially of non-spherical particles. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

The phrase “effective average particle size” as used herein to describe a multiparticulate (e.g., a porous nanoparticulate) means that at least 50% of the particles therein are of a specified size. Accordingly, “effective average particle size of less than about 2,000 nm in diameter” means that at least 50% of the particles therein are less than about 2000 nm in diameter. In certain embodiments, nanoparticulates have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. “D₅₀” refers to the particle size below which 50% of the particles in a multiparticulate fall. Similarly, “D₉₀” is the particle size below which 90% of the particles in a multiparticulate fall.

In certain embodiments, the porous nanoparticulates are comprised of one or more compositions selected from the group consisting of silica, a biodegradable polymer, a solgel, a metal and a metal oxide.

In an embodiment of the present invention, the nanostructures include a core-shell structure which comprises a porous particle core surrounded by a shell of lipid preferably a bilayer, but possibly a monolayer or multilayer (see Liu, et al., JACS, 2009, Id). The porous particle core can include, for example, a porous nanoparticle made of an inorganic and/or organic material as set forth above surrounded by a lipid bilayer. In the present invention, these lipid bilayer surrounded nanostructures are referred to as “protocells” or “functional protocells,” since they have a supported lipid bilayer membrane structure. In embodiments according to the present invention, the porous particle core of the protocells can be loaded with various desired species (“cargo”), including small molecules (e.g. anticancer agents as otherwise described herein), large molecules (e.g. including macromolecules such as RNA, including small interfering RNA or siRNA or small hairpin RNA or shRNA or a polypeptide which may include a polypeptide toxin such as a ricin toxin A-chain or other toxic polypeptide such as diphtheria toxin A-chain DTx, among others) or a reporter polypeptide (e.g. fluorescent green protein, among others) or semiconductor quantum dots, or metallic nanoparticles, or metal oxide nanoparticles or combinations thereof. In certain preferred aspects of the invention, the protocells are loaded with super-coiled plasmid DNA, which can be used to deliver a therapeutic and/or diagnostic peptide(s) or a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anticancer agents and antiviral agents, including anti-HIV, anti-HBV and/or anti-HCV agents, nucleic acids (DNA and RNA, including siRNA and shRNA and plasmids which, after delivery to a cell, express one or more polypeptides or RNA molecules), such as for a particular purpose, such as a therapeutic application or a diagnostic application as otherwise disclosed herein.

In embodiments, the lipid bilayer of the protocells can provide biocompatibility and can be modified to possess targeting species including, for example, targeting peptides including antibodies, aptamers, and PEG (polyethylene glycol) to allow, for example, further stability of the protocells and/or a targeted delivery into a bioactive cell.

The protocells particle size distribution, according to the present invention, depending on the application, may be monodisperse or polydisperse. The silica cores can be rather monodisperse (i.e., a uniform sized population varying no more than about 5% in diameter e.g., ±10-nm for a 200 nm diameter protocell especially if they are prepared using solution techniques) or rather polydisperse (i.e., a polydisperse population can vary widely from a mean or medium diameter, e.g., up to ±200-nm or more if prepared by aerosol. See FIG. 1, attached. Polydisperse populations can be sized into monodisperse populations. All of these are suitable for protocell formation. In the present invention, preferred protocells are preferably no more than about 500 nm in diameter, preferably no more than about 200 nm in diameter in order to afford delivery to a patient or subject and produce an intended therapeutic effect.

In certain embodiments, protocells according to the present invention generally range in size from greater than about 8-10 nm to about 5 μm in diameter, preferably about 20-nm-3 μm in diameter, about 10 nm to about 500 nm, more preferably about 20-200-nm (including about 150 nm, which may be a mean or median diameter). As discussed above, the protocell population may be considered monodisperse or polydisperse based upon the mean or median diameter of the population of protocells. Size is very important to therapeutic and diagnostic aspects of the present invention as particles smaller than about 8-nm diameter are excreted through kidneys, and those particles larger than about 200 nm are trapped by the liver and spleen. Thus, an embodiment of the present invention focuses in smaller sized protocells for drug delivery and diagnostics in the patient or subject.

In certain embodiments, protocells according the present invention are characterized by containing mesopores, preferably pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Preferred pore sizes of mesopores range from about 2-30 nm; they can be monosized or bimodal or graded—they can be ordered or disordered (essentially randomly disposed or worm-like). See FIG. 2, attached.

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g. if a templating moiety in the aerosol process is not used. They could also be enlarged to micropores, i.e., 50-nm in diameter.

Pore surface chemistry of the nanoparticle material can be very diverse—all organosilanes yielding cationic, anionic, hydrophilic, hydrophobic, reactive groups—pore surface chemistry, especially charge and hydrohobicity, affect loading capacity. See FIG. 3, attached. Attractive electrostatic interactions or hydrophobic interactions control/enhance loading capacity and control release rates. Higher surface areas can lead to higher loadings of drugs/cargos through these attractive interactions. See below.

In certain embodiments, the surface area of nanoparticles, as measured by the N2 BET method, ranges from about 100m2/g to >about 1200 m2/g. In general, the larger the pore size, the smaller the surface area. See table FIG. 2A. The surface area theoretically could be reduced to essentially zero, if one does not remove the templating agent or if the pores are sub-0.5-nm and therefore not measurable by N2 sorption at 77K due to kinetic effects. Howeveer, in this case, they could be measured by CO2 or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells according to the present invention are loaded with cargo to a capacity up to over 100 weight %: defined as (cargo weight/weight of protocell)×100. The optimal loading of cargo is often about 0.01 to 30% but this depends on the drug or drug combination which is incorporated as cargo into the protocell. This is generally expressed in μM per 10¹⁰ particles where we have values ranging from 2000-100 μM per 10¹⁰ particles. Preferred protocells according to the present invention exhibit release of cargo at pH about 5.5, which is that of the endosome, but are stable at physicological pH of 7 or higher (7.4).

The surface area of the internal space for loading is the pore volume whose optimal value ranges from about 1.1 to 0.5 cubic centimeters per gram (cc/g). Note that in the protocells according to one embodiment of the present invention, the surface area is mainly internal as opposed to the external geometric surface area of the nanoparticle.

The lipid bilayer supported on the porous particle according to one embodiment of the present invention has a lower melting transition temperature, i.e. is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

In the present invention, the lipid bilayer may vary significantly in composition. Ordinarily, any lipid or polymer which is may be used in liposomes may also be used in protocells. Preferred lipids are as otherwise described herein. Particularly preferred lipid bilayers for use in protocells according to the present invention comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bilayer, the zeta-potential is reduced to between about −10 mV and +5 mV, which is important for maximizing circulation time in the blood and avoiding non-specific interactions.

Depending on how the surfactant template is removed, e.g. calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to an embodiment of the present invention are that they are stable at pH 7, i.e. they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. The protocell core particle and surface can also be modified to provide non-specific release of cargo over a specified, prolonged period of time, as well as be reformulated to release cargo upon other biophysical changes, such as the increased presence of reactive oxygen species and other factors in locally inflamed areas. Quantitative experimental evidence has shown that targeted protocells illicit only a weak immune response, because they do not support T-Cell help required for higher affinity IgG, a favorable result.

Protocells according to the present invention exhibit at least one or more a number of characteristics (depending upon the embodiment) which distinguish them from prior art protocells:
1) In contrast to the prior art, an embodiment of the present invention specifies nanoparticles whose average size (diameter) is less than about 200-nm—this size is engineered to enable efficient cellular uptake by receptor mediated endocytosis and to minimize binding and uptake by non-target cells and organs;
2) An embodiment of the present invention can specify both monodisperse and/or polydisperse sizes to enable control of biodistribution.
3) An embodiment of the present invention is directed to targeted nanoparticles that induce receptor mediated endocytosis.
4) An embodiment of the present invention induces dispersion of cargo into cytoplasm through the inclusion of fusogenic or endosomolytic peptides.
5) An embodiment of the present invention provides particles with pH triggered release of cargo.
6) An embodiment of the present invention exhibits controlled time dependent release of cargo (via extent of thermally induced crosslinking of silica nanoparticle matrix).
7) An embodiment of the present invention can exhibit time dependent pH triggered release.
8) An embodiment of the present invention can contain and provide cellular delivery of complex multiple cargoes.
9) An embodiment of the present invention shows the killing of target cancer cells.
10) An embodiment of the present invention shows diagnosis of target cancer cells.
11) An embodiment of the present invention shows selective entry of target cells.
12) An embodiment of the present invention shows selective exclusion from off-target cells (selectivity).
13) An embodiment of the present invention shows enhanced enhanced fluidity of the supported lipid bilayer.
14) An embodiment of the present invention exhibits sub-nanomolar and controlled binding affinity to target cells.
15) An embodiment of the present invention exhibits sub-nanomolar binding affinity with targeting ligand densities below concentrations found in the prior art.
16) An embodiment of the present invention can further distinguish the prior art with with finer levels of detail unavailable in the prior art.

The term “lipid” is used to describe the components which are used to form lipid bilayers on the surface of the nanoparticles which are used in the present invention. Various embodiments provide nanostructures which are constructed from nanoparticles which support a lipid bilayer(s). In embodiments according to the present invention, the nanostructures preferably include, for example, a core-shell structure including a porous particle core surrounded by a shell of lipid bilayer(s). The nanostructure, preferably a porous silica nanostructure as described above, supports the lipid bilayer membrane structure. In embodiments according to the invention, the lipid bilayer of the protocells can provide biocompatibility and can be modified to possess targeting species including, for example, targeting peptides, fusogenic peptides, antibodies, aptamers, and PEG (polyethylene glycol) to allow, for example, further stability of the protocells and/or a targeted delivery into a bioactive cell, in particular a cancer cell. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used and the PEG component which is generally conjugated to phospholipid through an amine group comprises about 1% to about 20%, preferably about 5% to about 15%, about 10% by weight of the lipids which are included in the lipid bilayer.

Numerous lipids which are used in liposome delivery systems may be used to form the lipid bilayer on nanoparticles to provide protocells according to the present invention. Virtually any lipid or polymer which is used to form a liposome or polymersome may be used in the lipid bilayer which surrounds the nanoparticles to form protocells according to an embodiment of the present invention. Preferred lipids for use in the present invention include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the present invention given the fact that cholesterol may be an important component of the lipid bilayer of protocells according to an embodiment of the invention. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles used in the present invention can be, for example, mesoporous silica nanoparticles and core-shell nanoparticles. The nanoparticles may incorporate an absorbing molecule, e.g. an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be e.g. from around 5 nm to around 500 nm in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

The core-shell nanoparticles comprise a core and shell. The core comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but is not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙ—SiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosure of such processes therein are incorporated herein by reference.

“Amine-containing silanes” include, but are not limited to, a primary amine, a secondary amine or a tertiary amine functionalized with a silicon atom, and may be a monoamine or a polyamine such as diamine. Preferably, the amine-containing silane is N-(2-aminoethyl)-3-aminopropyltrimethoxysilane (AEPTMS). Non-limiting examples of amine-containing silanes also include 3-aminopropyltrimethoxysilane (APTMS) and 3-aminopropyltriethoxysilane (APTS), as well as an amino-functional trialkoxysilane. Protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, or combinations thereof, can also be used.

In certain embodiments of a protocell of the invention, the lipid bilayer is comprised of one or more lipids selected from the group consisting of phosphatidyl-cholines (PCs) and cholesterol.

In certain embodiments, the lipid bilayer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1].

In other embodiments:

(a) the lipid bilayer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (A9-Cis)], POPC [16:0-18:1] and DOTAP [18:1]; and wherein
(b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid bilayer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglykol)-5-soy bean sterol, and PEG-(polyethyleneglykol)-5 rapeseed sterol. In certain embodiments, the phytosterol comprises a mixture of at least two of the following compositions: sistosterol, camposterol and stigmasterol.

In still other illustrative embodiments, the lipid bilayer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the lipid bilayer is comprised of phospholipid selected from a monoacyl or diacylphosphoglyceride.

In still other illustrative embodiments, the lipid bilayer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bilayer is comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), and dimyristoylphosphatidylglycerol (DMPG).

In one illustrative embodiment of a protocell of the invention:

(a) the one or more pharmaceutically-active agents include at least one anticancer agent; (b) less than around 10% to around 20% of the anticancer agent is released from the porous nanoparticulates in the absence of a reactive oxygen species; and
(c) upon disruption of the lipid bilayer as a result of contact with a reactive oxygen species, the porous nanoparticulates release an amount of anticancer agent that is approximately equal to around 60% to around 80%, or around 61% to around 79%, or around 62% to around 78%, or around 63% to around 77%, or around 64% to around 77%, or around 65% to around 76%, or around 66% to around 75%, or around 67% to around 74%, or around 68% to around 73%, or around 69% to around 72%, or around 70% to around 71%, or around 70% of the amount of anticancer agent that would have been released had the lipid bilayer been lysed with 5% (w/v) Triton X-100.

One illustrative embodiment of a protocell of the invention comprises a plurality of negatively-charged, nanoporous, nanoparticulate silica cores that:

(a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof;
(b) are loaded with a siRNA or ricin toxin A-chain; and
(c) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bilayer comprises a cationic lipid and one or more zwitterionic phospholipids.

Protocells of the invention can comprise a wide variety of pharmaceutically-active ingredients.

The term “reporter” is used to describe an imaging agent or moiety which is incorporated into the phospholipid bilayer or cargo of protocells according to an embodiment of the present invention and provides a signal which can be measured. The moiety may provide a fluorescent signal or may be a radioisotope which allows radiation detection, among others. Exemplary fluorescent labels for use in protocells (preferably via conjugation or adsorption to the lipid bilayer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moities which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of cancer (cancer tissue) in a patient and or the progress of therapy in a patient or subject.

The term “histone-packaged supercoiled plasmid DNA” is used to describe a preferred component of protocells according to the present invention which utilize a preferred plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e. complexing DNA with cationic lipid mixtures). In addition, DNA has also been packaged with cationic proteins (including proteins other than histones), as well as gold nanoparticles (e.g. NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins are preferably used. In certain aspects of the invention, a combination of human histone proteins H1, H2A, H2B, H3 and H4 in a preferred ratio of 1:2:2:2:2, although other histone proteins may be used in other, similar ratios, as is known in the art or may be readily practiced pursuant to the teachings of the present invention. The DNA may also be double stranded linear DNA, instead of plasmid DNA, which also may be optionally supercoiled and/or packaged with histones or other packaging components.

Other histone proteins which may be used in this aspect of the invention include, for example, H1F, H1F0, H1FNT, H1FOO, H1FX H1H1 HIST1H1A, HIST1H1B, HIST1H1C, HIST1H1D, HIST1H1E, HIST1H1T; H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, H44 and HIST4H4.

The term “nuclear localization sequence” refers to a peptide sequence incorporated or otherwise crosslinked into histone proteins which comprise the histone-packaged supercoiled plasmid DNA. In certain embodiments, protocells according to the present invention may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Preferred nuclear localization sequences include H₂N-GNQSSNFGPMKGGNFGGRS SGPYGGGGQYFAKPRNQGGYGGC—COOH SEQ I.D NO: 9, RRMKWKK (SEQ ID NO:10), PKKKRKV (SEQ ID NO: 11), and KR[PAATKKAGQA]KKKK (SEQ ID NO:12), the NLS of nucleoplasmin, a prototypical bipartite signal comprising two clusters of basic amino acids, separated by a spacer of about 10 amino acids. Numerous other nuclear localization sequences are well known in the art. See, for example, LaCasse, et al., Nuclear localization signals overlap DNA-or RNA-binding domains in nucleic acid-binding proteins. NucL Acids Res., 23, 1647-1656 1995); Weis, K. Importins and exportins: how to get in and out of the nucleus [published erratum appears in Trends Biochem Sci 1998 July; 23(7):235]. TIBS, 23, 185-9 (1998); and Murat Cokol, Raj Nair & Burkhard Rost, “Finding nuclear localization signals”, at the website ubic.bioc.columbia.edu/papers/2000 nls/paper.html#tab2.

The term “cancer” is used to describe a proliferation of tumor cells (neoplasms) having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. It is noted that certain tumors including hepatocellular and cervical cancer, among others, are shown to exhibit increased levels of MET receptors specifically on cancer cells and are a principal target for compositions and therapies according to embodiments of the present invention which include a MET binding peptide complexed to the protocell.

The terms “coadminister” and “coadministration” are used synonymously to describe the administration of at least one of the protocell compositions according to the present invention in combination with at least one other agent, often at least one additional anti-cancer agent (as otherwise described herein), which are specifically disclosed herein in amounts or at concentrations which would be considered to be effective amounts at or about the same time. While it is preferred that coadministered compositions/agents be administered at the same time, agents may be administered at times such that effective concentrations of both (or more) compositions/agents appear in the patient at the same time for at least a brief period of time. Alternatively, in certain aspects of the present invention, it may be possible to have each coadministered composition/agent exhibit its inhibitory effect at different times in the patient, with the ultimate result being the inhibition and treatment of cancer, especially including hepatoccellular or cellular cancer as well as the reduction or inhibition of other disease states, conditions or complications. Of course, when more than disease state, infection or other condition is present, the present compounds may be combined with other agents to treat that other infection or disease or condition as required.

The term “anti-cancer agent” is used to describe a compound which can be formulated in combination with one or more compositions comprising protecells according to the present invention and optionally, to treat any type of cancer, in particular hepatocellular or cervical cancer, among numerous others. Anti-cancer compounds which can be formulated with compounds according to the present invention include, for example, Exemplary anti-cancer agents which may be used in the present invention include, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, NO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 345-(methylsulfonylpiperadinemethyl)-indolylj-quinolone, vatalanib, AG-013736, AVE-0005, the acetate salt of [D-Ser(But) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “antihepatocellular cancer agent” is used throughout the specification to describe an anticancer agent which may be used to inhibit, treat or reduce the likelihood of hepatocellular cancer, or the metastasis of that cancer. Anticancer agents which may find use in the present invention include for example, nexavar (sorafenib), sunitinib, bevacizumab, tarceva (erlotinib), tykerb (lapatinib) and mixtures thereof. In addition, other anticancer agents may also be used in the present invention, where such agents are found to inhibit metastasis of cancer, in particular, hepatocellular cancer.

The term “antiviral agent” is used to describe a bioactive agent/drug which inhibits the growth and/or elaboration of a virus, including mutant strains such as drug resistant viral strains. Preferred antiviral agents include anti-HIV agents, anti-HBV agents and anti-HCV agents. In certain aspects of the invention, especially where the treatment of hepatocellular cancer is the object of therapy, the inclusion of an anti-hepatitis C agent or anti-hepatitis B agent may be combined with other traditional anticancer agents to effect therapy, given that hepatitis B virus (HBV) and/or hepatitis C virus (HCV) is often found as a primary or secondary infection or disease state associated with hepatocellular cancer. Anti-HBV agents which may be used in the present invention, either as a cargo component in the protocell or as an additional bioactive agent in a pharmaceutical composition which includes a population of protocells includes such agents as Hepsera (adefovir dipivoxil), lamivudine. entecavir, telbivudine, tenofovir, emtricitabine, clevudine, valtoricitabine, amdoxovir, pradefovir, racivir, BAM 205, nitazoxanide, UT 231-13, Bay 41-4109. EHT899, zadaxin (thymosin alpha-1) and mixtures thereof. Typical anti-HCV agents for use in the invention include such agents as boceprevir, daclatasvir, asunapavir, INX-189, FV-100, NM 283, VX-950 (telaprevir), SCH 50304, TMC435, VX-500, BX-813, SCHSO3034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 and mixtures thereof

The term “anti-HIV agent” refers to a compound which inhibits the growth and/or elaboration of HIV virus (I and/or II) or a mutant strain thereof. Exemplary anti-HIV agents for use in the present invention which can be included as cargo in protocells according to the present invention include, for example, including nucleoside reverse transcriptase inhibitors (NRTI), other non-nucleoside reverse transcriptase inhibitors (i.e., those which are not representative of the present invention), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof

The term “targeting active species” is used to describe a compound or moiety which is complexed or preferably covalently bonded to the surface of a protocell according to the present invention which binds to a moiety on the surface of a cell to be targeted so that the protocell may selectively bind to the surface of the targeted cell and deposit its contents into the cell. The targeting active species for use in the present invention is preferably a targeting peptide as otherwise described herein, a polypeptide including an antibody or antibody fragment, an aptamer, or a carbohydrate, among other species which bind to a targeted cell.

The term “targeting peptide” is used to describe a preferred targeting active species which is a peptide of a particular sequence which binds to a receptor or other polypeptide in cancer cells and allows the targeting of protocells according to the present invention to particular cells which express a peptide (be it a receptor or other functional polypeptide) to which the targeting peptide binds. In the present invention, exemplary targeting peptides include, for example, SP94 free peptide (H₂N-SFSIILTPILPL-COOH, SEQ ID NO: 6), SP94 peptide modified with a C-terminal cysteine for conjugation with a crosslinking agent (H₂N-GLFHAIAHFIHGGWHGLIHGWYGGC—COOH (SEQ ID. NO: 13) or an 8 mer polyarginine (H₂N—RRRRRRRR—COOH, SEQ ID NO:14),), a modified SP94 peptide (H₂N-SFSIILTPILPLEEEGGC—COOH, SEQ ID NO: 8) or a MET binding peptide as otherwise disclosed herein. Other targeting peptides are known in the art. Targeting peptides may be complexed or preferably, covalently linked to the lipid bilayer through use of a crosslinking agent as otherwise described herein.

The term “MET binding peptide” or “MET receptor binding peptide” is used to five (5) 7-mer peptides which have been shown to bind MET receptors on the surface of cancer cells with enhanced binding efficiency. Pursuant to the present invention, several small peptides with varying amino acid sequences were identified which bind the MET receptor (a.k.a. hepatocyte growth factor receptor, expressed by gene c-MET) with varying levels of specificity and with varying ability to activate MET receptor signaling pathways. 7-mer peptides were identified using phage display biopanning, with examples of resulting sequences which evidence enhanced binding to MET receptor and consequently to cells such as cancer cells (e.g. hepatocellular, ovarian and cervical) which express high levels of MET receptors, which appear below. Binding data for several of the most commonly observed sequences during the biopanning process is also presented in the examples section of the present application. These peptides are particularly useful as targeting ligands for cell-specific therapeutics. However, peptides with the ability to activate the receptor pathway may have additional therapeutic value themselves or in combination with other therapies. Many of the peptides have been found bind not only hepatocellular carcinoma, which was the original intended target, but also to bind a wide variety of other carcinomas including ovarian and cervical cancer. These peptides are believed to have wide-ranging applicability for targeting or treating a variety of cancers and other physiological problems associated with expression of MET and associated receptors.

The following five 7mer peptide sequences show substantial binding to MET receptor and are particularly useful as targeting peptides for use on protocells according to the the present invention.

SEQ ID NO: 1
ASVHFPP (Ala-Ser-Val-His-Phe-Pro-Pro)
SEQ ID NO: 2
TATFWFQ (Thr-Ala-Thr-Phe-Trp-Phe-Gln)
SEQ ID NO: 3
TSPVALL (Thr-Ser-Pro-Val-Ala-Leu-Leu)
SEQ ID NO: 4
IPLKVHP (Ile-Pro-Leu-Lys-Val-His-Pro)
SEQ ID NO: 5
WPRLTNM (Trp-Pro-Arg-Leu-Thr-Asn-Met)

Each of these peptides may be used alone or in combination with other MET peptides within the above group or with other targeting peptides which may assist in binding protocells according to the present invention to cancer cells, including hepatocellular cancer cells, ovarian cancer cells and cervical cancer cells, among numerous others. These binding peptides may also be used in pharmaceutical compounds alone as MET binding peptides to treat cancer and otherwise inhibit hepatocyte growth factor binding.

The terms “fusogenic peptide” and “endosomolytic peptide” are used synonymously to describe a peptide which is optionally and preferred crosslinked onto the lipid bilayer surface of the protocells according to the present invention. Fusogenic peptides are incorporated onto protocells in order to facilitate or assist escape from endosomal bodies and to facilitate the introduction of protocells into targeted cells to effect an intended result (therapeutic and/or diagnostic as otherwise described herein). Representative and preferred fusogenic peptides for use in protocells according to the present invention include H5WYG peptide, H₂N-GLFHAIAHFIHGGWHGLIHGWYGGC—COOH (SEQ ID. NO: 13) or an 8 mer polyarginine (H₂N—RRRRRRRR—COOH, SEQ ID NO:14), among others known in the art.

The term “crosslinking agent” is used to describe a bifunctional compound of varying length containing two different functional groups which may be used to covalently link various components according to the present invention to each other. Crosslinking agents according to the present invention may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility required. Crosslinking agents are used to anchor targeting and/or fusogenic peptides to the phospholipid bilayer, to link nuclear localization sequences to histone proteins for packaging supercoiled plasmid DNA and in certain instances, to crosslink lipids in the lipid bilayer of the protocells. There are a large number of crosslinking agents which may be used in the present invention, many commercially available or available in the literature. Preferred crosslinking agents for use in the present invention include, for example, 1-Ethyl-3-[3-dimethylaminopropyl]carbodiimide hydrochloride (EDC), succinimidyl 41N-maleimidomethyl]cyclohexane-1-carboxylate (SMCC), N-[β-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido]hexanoate (LC-SPDP), among others.

As discussed in detail above, the porous nanoparticle core of the present invention can include porous nanoparticles having at least one dimension, for example, a width or a diameter of about 3000 nm or less, about 1000 nm or less, about 500 nm or less, about 200 nm or less. Preferably, the nanoparticle core is spherical with a preferred diameter of about 500 nm or less, more preferably about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, protocells according to the present invention are biocompatible. Drugs and other cargo components are often loaded by adsorption and/or capillary filling of the pores of the particle core up to approximately 50% by weight of the final protocell (containing all components). In certain embodiments according to the present invention, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

In the present invention, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In preferred aspects of the invention, the lipid bilayer is fused onto the porous particle core to form the protocell. Protocells according to the present invention can include various lipids in various weight ratios, preferably including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bilayer which is used to prepare protocells according to the present invention can be prepared, for example, by extrusion of hydrated lipid films through a filter with pore size of, for example, about 100 nm, using standard protocols known in the art or as otherwise described herein. The filtered lipid bilayer films can then be fused with the porous particle cores, for example, by pipette mixing. In certain embodiments, excess amount of lipid bilayer or lipid bilayer films can be used to form the protocell in order to improve the protocell colloidal stability.

In certain diagnostic embodiments, various dyes or fluorescent (reporter) molecules can be included in the protocell cargo (as expressed by as plasmid DNA) or attached to the porous particle core and/or the lipid bilayer for diagnostic purposes. For example, the porous particle core can be a silica core or the lipid bilayer and can be covalently labeled with FITC (green fluorescence), while the lipid bilayer or the particle core can be covalently labeled with FITC Texas red (red fluorescence). The porous particle core, the lipid bilayer and the formed protocell can then be observed by, for example, confocal fluorescence for use in diagnostic applications. In addition, as discussed herein, plasmid DNA can be used as cargo in protocells according to the present invention such that the plasmid may express one or more fluorescent proteins such as fluorescent green protein or fluorescent red protein which may be used in diagnostic applications.

In various embodiments, the protocell is used in a synergistic system where the lipid bilayer fusion or liposome fusion (i.e., on the porous particle core) is loaded and sealed with various cargo components with the pores (mesopores) of the particle core, thus creating a loaded protocell useful for cargo delivery across the cell membrane of the lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final protocell

A fusion and synergistic loading mechanism can be included for cargo delivery. For example, cargo can be loaded, encapsulated, or sealed, synergistically through liposome fusion on the porous particles. The cargo can include, for example, small molecule drugs (e.g. especially including anticancer drugs and/or antiviral drugs such as anti-HBV or anti-HCV drugs), peptides, proteins, antibodies, DNA (especially plasmid DNA, including the preferred histone-packaged super coiled plasmid DNA), RNAs (including shRNA and siRNA (which may also be expressed by the plasmid DNA incorporated as cargo within the protocells) fluorescent dyes, including fluorescent dye peptides which may be expressed by the plasmid DNA incorporated within the protocell.

In embodiments according to the present invention, the cargo can be loaded into the pores (mesopores) of the porous particle cores to form the loaded protocell. In various embodiments, any conventional technology that js developed for liposome-based drug delivery, for example, targeted delivery using PEGylation, can be transferred and applied to the protocells of the present invention.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bilayer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, a cancer cell in a subject or patient. Due to the internalization of the cargo-loaded protocells in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which is preferably formulated as histone-packaged supercoiled plasmid DNA preferably modified with a nuclear localization sequence can be directly delivered or internalized by the targeted cells. Thus, the DNA or RNA can be loaded first into a protocell and then into then through the target cells through the internalization of the loaded protocells.

As discussed, the cargo loaded into and delivered by the protocell to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally and preferably modified with a nuclear localization sequence which can localize and concentrate the delivered plasmid DNA into the nucleus of the target cell. As such, loaded protocells can deliver their cargo into targeted cells for therapy or diagnostics.

In various embodiments according to the present invention, the protocells and/or the loaded protocells can provide a targeted delivery methodology for selectively delivering the protocells or the cargo components to targeted cells (e.g., cancer cells). For example, a surface of the lipid bilayer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In preferred aspects of the invention, the targeting active species is a targeting peptide as otherwise described herein. In certain embodiments, preferred peptide targeting species include a MET binding peptide as otherwise described herein.

For example, by providing a targeting active species (preferably, a targeting peptide) on the surface of the loaded protocell, the protocell selectively binds to the targeted cell in accordance with the present teachings. In one embodiment, by conjugating an exemplary targeting peptide SP94 or analog or a MET binding peptide as otherwise described herein that targets cancer cells, including cancer liver cells to the lipid bilayer, a large number of the cargo-loaded protocells can be recognized and internalized by this specific cancer cells due to the specific targeting of the exemplary SP94 or MET binding peptide with the cancer (including liver) cells. In most instances, if the protocells are conjugated with the targeting peptide, the protocells will selectively bind to the cancer cells and no appreciable binding to the non-cancerous cells occurs.

Once bound and taken up by the target cells, the loaded protocells can release cargo components from the porous particle and transport the released cargo components into the target cell. For example, sealed within the protocell by the liposome fused bilayer on the porous particle core, the cargo components can be released from the pores of the lipid bilayer, transported across the protocell membrane of the lipid bilayer and delivered within the targeted cell. In embodiments according to the present invention, the release profile of cargo components in protocells can be more controllable as compared with when only using liposomes as known in the prior art. The cargo release can be determined by, for example, interactions between the porous core and the lipid bilayer and/or other parameters such as pH value of the system. For example, the release of cargo can be achieved through the lipid bilayer, through dissolution of the porous silica; while the release of the cargo from the protocells can be pH-dependent.

In certain embodiments, the pH value for cargo is often less than 7, preferably about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or span for several days to about 20-30 days or longer. Thus, the present invention may accommodate immediate release and/or sustained release applications from the protocells themselves.

In certain embodiments, the inclusion of surfactants can be provided to rapidly rupture the lipid bilayer, transporting the cargo components across the lipid bilayer of the protocell as well as the targeted cell. In certain embodiments, the phospholipid bilayer of the protocells can be ruptured by the application/release of a surfactant such as sodium dodecyl sulfate (SDS), among others to facilitate a rapid release of cargo from the protocell into the targeted cell. Other than surfactants, other materials can be included to rapidly rupture the bilayer. One example would be gold or magnetic nanoparticles that could use light or heat to generate heat thereby rupturing the bilayer. Additionally, the bilayer can be tuned to rupture in the presence of discrete biophysical phenomena, such as during inflammation in response to increased reactive oxygen species production. In certain embodiments, the rupture of the lipid bilayer can in turn induce immediate and complete release of the cargo components from the pores of the particle core of the protocells. In this manner, the protocell platform can provide an increasingly versatile delivery system as compared with other delivery systems in the art. For example, when compared to delivery systems using nanoparticles only, the disclosed protocell platform can provide a simple system and can take advantage of the low toxicity and immunogenicity of liposomes or lipid bilayers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile and provide increased cargo capacity.

In addition, the lipid bilayer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bilayer of the protocells can provide a fluidic interface for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells.

Pharmaceutical compositions according to the present invention comprise an effective population of protocells as otherwise described herein formulated to effect an intended result (e.g. therapeutic result and/or diagnostic analysis, including the monitoring of therapy) formulated in combination with a pharmaceutically acceptable carrier, additive or excipient. The protocells within the population of the composition may be the same or different depending upon the desired result to be obtained. Pharmaceutical compositions according to the present invention may also comprise an addition bioactive agent or drug, such as an anticancer agent or an antiviral agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method of the invention include humans, companion animals, laboratory animals, and the like. The invention contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds according to the present invention may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Preferably, the composition is about 0.1% to about 85%, about 0.5% to about 75% by weight of a compound or compounds of the invention, with the remainder consisting essentially of suitable pharmaceutical excipients.

An injectable composition for parenteral administration (e.g. intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject according to the present invention.

Methods of treating patients or subjects in need for a particular disease state or infection (especially including cancer and/or a HBV, HCV or HIV infection) comprise administration an effective amount of a pharmaceutical composition comprising therapeutic protocells and optionally at least one additional bioactive (e.g. antiviral) agent according to the present invention.

Diagnostic methods according to the present invention comprise administering to a patient in need (a patient suspected of having cancer) an effective amount of a population of diagnostic protocells (e.g., protocells which comprise a target species, such as a targeting peptide which binds selectively to cancer cells and a reporter component to indicate the binding of the protocells to cancer cells if the cancer cells are present) whereupon the binding of protocells to cancer cells as evidenced by the reporter component (moiety) will enable a diagnosis of the existence of cancer in the patient.

An alternative of the diagnostic method of the present invention can be used to monitor the therapy of cancer or other disease state in a patient, the method comprising administering an effective population of diagnostic protocells (e.g., protocells which comprise a target species, such as a targeting peptide which binds selectively to cancer cells or other target cells and a reporter component to indicate the binding of the protocells to cancer cells if the cancer cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

The following non-limiting examples are illustrative of the invention and its advantageous properties, and are not to be taken as limiting the disclosure or claims in any way. In the examples, as well as elsewhere in this application, all parts and percentages are by weight unless otherwise indicated.

### Example 1

### Ligand-Targeting Protocells

As provided in the following examples, the porous nanoparticle-supported lipid bilayer (protocell), formed via fusion of liposomes to nanoporous silica particles, is a novel type of nanocarrier that addresses multiple challenges associated with targeted delivery of cancer therapeutics and diagnostics. Like liposomes, protocells are biocompatible, biodegradable, and non-immunogenic, but their nanoporous silica core confers a drastically enhanced cargo capacity and prolonged bilayer stability when compared to similarly-sized liposomal delivery agents. The porosity and surface chemistry of the core can, furthermore, be modulated to promote encapsulation of a wide variety of therapeutic agents, such as drugs, nucleic acids, and protein toxins. The rate of cargo release can be controlled by pore size and the overall degree of silica condensation, making protocells useful in applications requiring either burst or controlled release profiles. Finally, the protocell's supported lipid bilayer (SLB) can be modified with ligands to promote selective delivery and with PEG to extend circulation times. In the examples, the inventors report the use of peptidetargeted protocells to achieve highly specific delivery of a plasmid that encodes small hairpin RNA (snRNA), which induces growth arrest and apoptosis of transfected cells by silencing cyclin B1. As set forth in the examples section below, the inventors have prepared synthesized silica nanoparticles with pores large enough to accommodate histone-packaged plasmids using a dual surfactant approach. A non-ionic surfactant (Pluronic® F-127), when employed in conjunction with a swelling agent (1,3,5-trimethylbenzene) served as the template for large pores, while a fluorocarbon surfactant (FC-4) promoted growth of the silica core. Resulting particles had diameters ranging from 100-nm to 300-nm and contained an ordered network of 20-nm pores with 17.3-nm pore entrances. Supercoiled plasmid DNA was packaged with histones, and the resulting complex (about 15-nm in diameter) was modified with a nuclear localization sequence (NLS) prior to being loaded into the silica core. Fusion of liposomes to the nanoporous core promoted long-term retention (>1 month) of encapsulated DNA upon exposure to simulated body fluids at 37° C. Using phage display, the inventors identified a targeting peptide with nanomolar affinity for hepatocyte growth factor receptor (c-Met), which is known to be overexpressed by various types of hepatocellular carcinoma (HCC). Protocells loaded with the DNA-histone-NLS complex and modified with “240 copies each of the targeting peptide and a fusogenic peptide that promotes endosomal escape of protocells and encapsulated DNA were capable of transfecting both dividing and non-dividing HCC. Furthermore, targeted protocells effectively induced GJM arrest and apoptosis of HCC (LC, =25 nM) without affecting the viability of non-cancerous cells, including hepatocytes, endothelial cells, and immune cells (PBMCs, B cells, and T cells).

### Methods

The nanoporous silica particles that form the core of the protocell are prepared, as previously described”² (see also Ashley, et al., Nature Materials, 2011, May; 10(5):389-97) from a homogenous mixture of water-soluble silica precursor(s) and amphipathic surfactant(s) using either aerosol-assisted evaporation-induced self-assembly (EISA) or solvent extraction-driven self-assembly within water-in-oil emulsion droplets¹. Solvent evaporation or extraction concentrates the aerosol or emulsion droplets in surfactant(s), which directs the formation of periodic, ordered structures, around which silica assembles and condenses. Surfactants are removed via thermal calcination, which results in porous nanoparticles with well-defined, uniform pore sizes and topologies. Particles formed via aerosol-assisted EISA (‘unimodal’ particles) possess an average diameter of approximately 120-nm (after size exclusion-based separation), a Brunauer-Emmer-Teller (BET) surface area in excess of 1200 m²/g, a pore volume fraction of about 50%, and a unimodal pore diameter of 2.5-nm. Particles formed within emulsion droplets (‘multimodal’ particles) have an average diameter of ˜150 nm (after size exclusion-based separation), a BET surface area of >600 m²/g, a pore volume fraction of ˜65%, and a multimodal pore morphology composed of large (20-30 nm), surface-accessible pores interconnected by 6-12 nm pores. The liquid-vapor or liquid-liquid interfacial tensions associated with aerosol or emulsion processing (respectively) enforce a spherical shape with minimal surface roughness. Both types of particles, additionally, have fully accessible three-dimensional pore networks, as evidenced by analysis of nitrogen sorption isotherms.

The high pore volume, surface area, and accessibility of the nanoporous silica cores imparts a high cargo capacity and enables rapid loading of multiple types of therapeutic and diagnostic agents. Unimodal nanoporous cores have a high capacity for low molecular weight chemotherapeutic agents, while multimodal cores possess the large, surface-accessible pores necessary for encapsulation of siRNA, protein toxins, and other high molecular weight cargos (e.g. plasmid DNA). The rate of cargo release can be precisely controlled by the degree to which the silica core is condensed. Incorporating various amounts of AEPTMS, an amine-containing silane, into the sol used to form the nanoporous silica cores reduces the level of achievable condensation and promotes more rapid dissolution of the cores under neutral pH, high ionic strength (i.e. cytosolic) conditions. Particles that contain no AEPTMS dissolve over the course of 2 weeks in a simulated body fluid, while particles that contain 30 mol % AEPTMS dissolve within 24 hours. Protocells can, therefore, be adapted for applications requiring continuous or burst release profiles.

Incorporating AEPTMS into the precursor sol used to form nanoporous silica particles accelerates particle dissolution under cytosolic conditions and promotes more rapid release of encapsulated cargo than can be achieved via simple diffusion. AEPTMS-modified particles also have a reduced capacity for weakly basic chemotherapeutic drugs (e.g. doxorubicin), however. Therefore, in order to maximize both capacity and intracellular release, we characterized zeta potential, cargo (e.g. drug (Doxorubicin/DOX)/chemotherapy) capacity, silica dissolution rates, and cargo release rates as a function of AEPTMS concentration. As previously demonstrated, unmodified unimodal particles (ζ=−104.5±5.6) have a high capacity for cargo (in the case of DOX ˜1.8 mM per 10¹⁰ particles) but release only 20% of their encapsulated cargo (drug) within 24 hours (i.e. the typical doubling time of HCC). Conversely, unimodal particles modified with 30 wt % AEPTMS (ζ=88.9±5.5) release all of their encapsulated cargo (drug) within 6 hours but have a reduced drug (DOX) capacity (˜0.15 mM per 10¹⁰ particles). Unimodal particles that contain 15 wt % AEPTMS (ζ=−21.3±5.1) retain their high capacity for drug (DOX) (˜1.1 mM per 10¹⁰ particles) and release nearly all of their encapsulated (drug) within 24 hours when exposed to a simulated body fluid; therefore these particles are selected for all experiments involving delivery of cargo. It is important to note that, while the zeta potential of unimodal silica particles increases as a function of AEPTMS concentration, the pore volume fraction of AEPTMS-modified particles (˜45% for particles that contain 30 wt % AEPTMS) is not substantially different from that of unmodified particles (˜50%). Therefore, we attribute the decreased cargo capacity of AEPTMS-modified unimodal particles to electrostatic repulsion rather than decreased pore volume. Multimodal particles are included as a control to demonstrate the effect of pore size on cargo capacity and the kinetics of cargo release.

### General Reagents

Absolute ethanol, hydrochloric acid (37%), tetraethyl orthosilicate (TEOS, 98%), 3-aminopropyltriethoxysilane (APTES, ≧98%), 3-[2-(2-aminoethylamino)ethylamino]propyltrimethoxysilane (AEPTMS, technical grade), 2-cyanoethyl triethoxysilane (CETES, ≧97.0%), hexadecyltrimethylammonium bromide (CTAB, ≧99%), Brij®-56, sodium dodecyl sulfate (SDS, ≧98.5%), Triton® X-100, hexadecane (≧99%), doxorubicin hydrochloride (≧98%), 5-fluorouracil (≧99%), cis-diammineplatinum(II) dichloride (cisplatin, ≧99.9%), diphtheria toxin from Corynebacterium diphtheriae, cyclosporin A from Tolypocladium inflatum (CsA, ≧95%), N-Acetyl-L-cysteine (NAC, ≧99%), human epidermal growth factor, L-α-phosphatidylethanolamine, thymidine (≧99%), hypoxanthine (≧99%), bovine fibronectin, bovine collagen type I, gelatin, soybean trypsin inhibitor (≧98%), 2-mercaptoethanol (≧99.0%), DL-dithiothreitol (≧99.5%), dimethyl sulfoxide (≧99.9%), pH 5 citric acid buffer, ethylenediaminetetraacetic acid (EDTA, 99.995%), 4-(2-Hydroxyethyl)piperazine-1-ethanesulfonic acid (HEPES, ≧99.5%), ammonium phosphate dibasic (≧99.99%), and Sepharose® CL-4B were purchased from Sigma-Aldrich (St. Louis, Mo.). ABIL® EM 90 (cetyl PEG/PPG-10/1 dimethicone) was purchased from Evonik Industries (Essen, Germany). Ultra pure, EM-grade formaldehyde (16%, methanol-free) was purchased from Polysciences, Inc. (Warrington, Pa.). Hellmanex II was purchased from Hellma (Müllheim, Germany).

### Lipids

1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N4methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and cholesterol were purchased from Avanti Polar Lipids, Inc. (Alabaster, Ala.).

### Cell Lines and Growth Media

Human Hep3B (HB-8064), human hepatocytes (CRL-11233), human peripheral blood mononuclear cells (CRL-9855), human umbilical cord vein endothelial cells (CRL-2873), T lymphocytes (CRL-8293), B lymphocytes (CCL-156), Eagle's Minimum Essential Medium (EMEM), Dulbecco's Modified Eagle's Medium (DMEM), Iscove's Modified Dulbecco's Medium (IMDM), RPMI 1640 medium, fetal bovine serum (FBS), and 1× trypsin-EDTA solution (0.25% trypsin with 0.53 mM EDTA) were purchased from American Type Culture Collection (ATCC; Manassas, Va.). BEGM Bullet Kits were purchased from Lonza Group Limited (Clonetics; Walkersville, Md.). DMEM without phenol red was purchased from Sigma-Aldrich (St. Louis, Mo.).

### Fluorescent Stains and Microscopy Reagents

Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580), Alexa Fluor® 532 carboxylic acid, succinimidyl ester (532/554), propidium iodide (535/617), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670), Alexa Fluor® 647 conjugate of annexin V (650/665), SlowFade® Gold antifade reagent (with and without DAPI), Image-iT® FX signal enhancer, 1× Dulbecco's phosphate-buffered saline (D-PBS), bovine albumin fraction V solution (BSA, 7.5%), and transferrin were purchased from Invitrogen Life Sciences (Carlsbad, Calif.). Red Fluorescent Protein (RFP, 557/585), CaspGLOW™ Fluorescein Active Caspase-3 Staining Kit (485/535), and CaspGLOW™ Red Active Caspase-8 Staining Kit (540/570) were purchased from BioVision, Inc. (Mountain View, Calif.). Water soluble CdSe/ZnS quantum dots, CZWD640 (640/660), were purchased from NN-Labs (Fayetteville, Ark.).

### Crosslinkers

1-Ethyl-3-[3-dimethylaminopropyl]carbodiimide hydrochloride (EDC), succinimidyl 4-[N-maleimidomethyl]cyclohexane-1-carboxylate (SMCC), N-[β-Maleimidopropionic acid] hydrazide (BMPH), succinimidyl-[(N-maleimidopropionamido)-tetracosaethyleneglycol] ester (SM(PEG)₂₄), succinimidyl 6-[3′-(2-pyridyldithio)-propionamido]hexanoate (LC-SPDP), and the Sulfhydryl Addition Kit were purchased from Pierce Protein Research Products (Thermo Fisher Scientific LSR; Rockford, Ill.).

### Other Silica Nanoparticles

Sub-5-nm silicon nanoparticles were purchased from Melorium Technologies, Inc. (Rochester, N.Y.). 10-20 nm silicon oxide nanoparticles were purchased from SkySpring Nanomaterials, Inc. (Houston, Tex.). 30-nm, 40-nm, 50-nm, 60-nm, 70-nm, 80-nm, 90-nm, 100-nm, 150-nm, 200-nm, and 10-mm silica particles were purchased from Discovery Scientific, Inc. (Vancouver, British Columbia).
Synthetic siRNA and Peptides
Silencer select siRNAs (siRNA IDs for EGFR, VEGFR-2, and PDGFR-α are s565, s7824, and s10234, respectively) were purchased from Ambion, Inc. (Austin, Tex.). The double stranded-DNA oligonucleotide (5′-AAACATGTGGATTACCCATGTC-3′) with 5′ amino modifier C12 was purchased from Integrated DNA Technologies (IDT; Coralville, Iowa). ‘Free’ SP94 peptide (H₂N-SFSIILTPILPL-COOH, SEQ ID NO: 6), SP94 peptide modified with C-terminal Cys for conjugation (H₂N-SFSIILTPILPLGGC—COOH, SEQ ID NO: 7), and SP94 peptide used in the FIG. 2d recruitment experiments (H₂N-SFSIILTPILPLEEEGGC—COOH, SEQ ID NO: 8) were synthesized by New England Peptide (Gardner, Mass.). The H5WYG peptide (H₂N-GLFHAIAHFIHGGWHGLIHGWYGGGC—COOH) and nuclear localization sequence (H₂N-NQSSNFGPMKGGNFGGRSSGPYGGGGQYFAKPRNQGGYGGC—COOH) were synthesized by Biopeptide Co., Inc. (San Diego, Calif.). The emboldened portions of peptides are the original sequences; additional amino acid residues were added for conjugation or labeling purposes. All antibodies (CHALV-1, anti-Rab11a, anti-LAMP-1, anti-EGFR, anti-VEGFR-2, anti-PDGFR-a) were purchased from Abcam, Inc. (Cambridge, Mass.).

### Cell Culture Conditions

Hep3B, hepatocytes, PBMCs, T-lymphocytes, and B-lymphocytes were obtained from ATCC and grown per manufacturer's instructions. Briefly, Hep3B was maintained in EMEM with 10% FBS. Hepatocytes were grown in flasks coated with BSA, fibronectin, and bovine collagen type I; the culture medium used was BEGM (gentamycin, amphotericin, and epinephrine were discarded from the BEGM Bullet kit) with 5 ng/mL epidermal growth factor, 70 ng/mL phosphatidylethanolamine, and 10% FBS. HUVECs were grown in DMEM with 20% FBS; gelatin-coated flasks were used to promote adhesion. PBMCs, T lymphocytes, and B lymphocytes were maintained in suspension flasks (Greiner Bio-One; Monroe, N.C.). PBMCs were grown in IMDM supplemented with 0.02 mM thymidine, 0.1 mM hypoxanthine, 0.05 mM 2-mercaptoethanol, and 10% FBS. T and B lymphocytes were grown in IMDM with 20% FBS and RPMI 1640 medium with 20% FBS, respectively. All cells were maintained at 37° C. in a humidified atmosphere (air supplemented with 5% CO₂). Adherent cells were passaged with 0.05% trypsin at a sub-cultivation ratio of 1:3, while non-adherent cells were seeded at a density of 2×10⁵ cells/mL and maintained at 1-5×10⁶ cells/mL.

### Synthesis and Characterization of Nanoporous Silica Particles

### Synthesis of Unimodal Silica Nanoparticles

The aerosol-assisted evaporation-induced self-assembly method employed to prepare nanoporous silica particles with unimodal porosity has been described by Lu, et al.². Briefly, a homogenous sol containing a silica precursor (TEOS), a structure-directing surfactant (CTAB, initially at a concentration much less than the critical micelle concentration, or CMC), and HCl dissolved in a solution of water and ethanol was aerosolized using a modified commercial atomizer (Model 9302A; TSI, Inc.; St Paul, Minn.). Nitrogen was used as the carrier gas, and all heating zones were maintained at 400° C. to evaporate the solvent and increase the effective surfactant concentration. Pressure drop at the pinhole was 20 psi. Particles were collected on a Durapore membrane filter (Millipore; Billerica, Mass.) maintained at 80° C. A typical reaction mixture contained 55.9 mL of deionized H₂O, 43 mL of 200-proof ethanol, 1.10 mL of 1.0 N HCl, 4.0 g of CTAB, and 10.32 g of TEOS. To prepare nanoporous silica particles that dissolve more rapidly under intracellular (neutral pH, relatively high salt concentrations) conditions, various amounts of TEOS and AEPTMS, an amine-containing silane, were incorporated into the precursor sol, and the pH of the system was adjusted to 2.0 using concentrated HCl. For example, to prepare particles with 15 wt % AEPTMS, 9.36 g of TEOS and 1.33 g of AEPTMS were used.

### Synthesis of Multimodal Silica Nanoparticles

The emulsion processing used to synthesize nanoporous silica particles with multimodal porosity has been described by Carroll, et al.¹. Briefly, 1.82 g of CTAB (soluble in the aqueous phase) was added to 20 g of deionized water, stirred at 40° C. until dissolved, and allowed to cool to 25° C. 0.57 g of 1.0 N HCl, 5.2 g of TEOS, and 0.22 g of NaCl were added to the CTAB solution, and the resulting sol was stirred for 1 hour. An oil phase composed of hexadecane with 3 wt % Abil EM 90 (a non-ionic emulsifier soluble in the oil phase) was prepared. The precursor sol was combined with the oil phase (1:3 volumetric ratio of sol:oil) in a 1000-mL round-bottom flask, stirred vigorously for 2 minutes to promote formation of a water-in-oil emulsion, affixed to a rotary evaporator (R-205; Buchi Laboratory Equipment; Switzerland), and placed in an 80° C. water bath for 30 minutes. The mixture was then boiled under a reduced pressure of 120 mbar (35 rpm for 3 hours) to remove the solvent. Particles were the centrifuged (Model Centra MP4R; International Equipment Company; Chattanooga, Tenn.) at 3000 rpm for 20 minutes, and the supernatant was decanted. Finally, the particles were calcined at 500° C. for 5 hours to remove surfactants and other excess organic matter. As described by Carroll, et al., solvent extraction enriches the aqueous phase in CTAB (>CMC), and the resulting micelles template 6-12 nm pores upon condensation of silica particles (in the aqueous phase). Additionally, adsorption of two surfactants (CTAB and Abil EM 90) at the water-oil interface synergistically decreases the interfacial tension, which results in the spontaneous formation of 20-30 nm microemulsion droplets that template large, surface-accessible pores.

### Characterization of Silica Nanoparticles

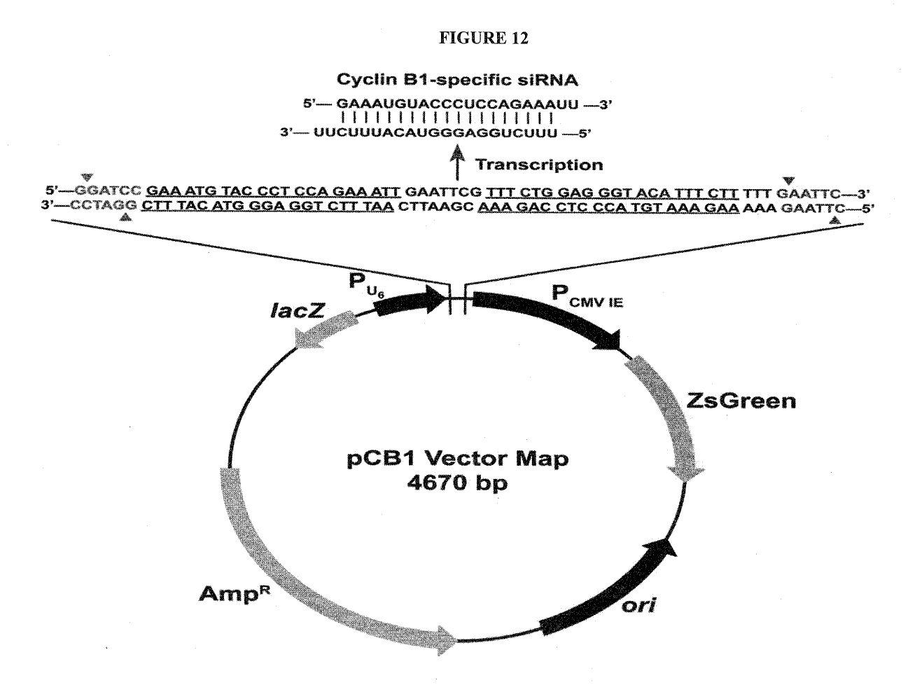

Dynamic light scattering of nanoporous silica particles was performed using a Zetasizer Nano (Malvern; Worcestershire, United Kingdom). Samples were prepared by diluting 484 of silica particles (25 mg/mL) in 2.4 ml of 1×D-PBS. Solutions were transferred to 1 mL polystyrene cuvettes (Sarstedt; Nümbrecht, Germany) for analysis. Nitrogen sorption was performed using an ASAP 2020 Surface Area and Porosity Analyzer (Micromeritics Instrument Corporation; Norcross, Ga.). Zeta potential measurements were made using a Zetasizer Nano (Malvern; Worcestershire, United Kingdom). In a typical experiment, silica particles, liposomes, or protocells were diluted 1:50 in a simulated body fluid (pH 7.4) or citric acid buffer (pH 5.0), both of which were adjusted to contain 150 mM NaCl, and transferred to 1-mL folded capillary cells (Malvern; Worcestershire, United Kingdom) for analysis. See Supplementary FIG. 1 for DLS and nitrogen sorption data and Supplementary FIG. 12 for zeta potential values of silica nanoparticles, liposomes, and protocells.

### Synthesis, Loading, and Surface Functionalization of Protocells Liposome Fusion to Nanoporous Silica Particles

The procedure used to synthesize protocells has been described by Liu, et al.²⁵⁻²⁷ and will be mentioned only briefly. Lipids were ordered from Avanti Polar Lipids pre-dissolved in chloroform and stored at −20° C. Immediately prior to protocell synthesis, 2.5 mg of lipid was dried under a stream of nitrogen and placed in a vacuum oven (Model 1450M, VWR International, West Chester, Pa.) overnight to remove residual solvent. Lipids were re-hydrated in 0.5×D-PBS at a concentration of 2.5 mg/mL and were passed through a 100-nm filter at least 10 times using a Mini-Extruder set (Avanti Polar Lipids, Inc.; Alabaster, Ala.). DPPC and DSPC were dissolved in 0.5×D-PBS pre-warmed to their respective transition temperatures (41° C. and 55° C.) and maintained at 60° C. during the extrusion process. Resulting liposomes (˜120-nm in diameter) were stored at 4° C. for no more than one week. Nanoporous silica cores were dissolved in 0.5×D-PBS (25 mg/mL) and exposed to an excess of liposomes (1:2-1:4 volumetric ratio of lipid:silica) for 30-90 minutes at room temperature. Protocells were stored in the presence of excess lipid for up to 3 months at 4° C. To remove excess lipid, protocells were centrifuged at 10,000 rpm for 5 minutes, washed twice, and re-suspended in 0.5×D-PBS.

### Optimization of the Supported Lipid Bilayer Composition

The composition of the SLB was optimized to minimize non-specific binding and toxicity to control cells; see Supplementary FIG. 4 for structures of the various lipids that were used. The protocells used in all surface binding, internalization, and delivery experiments had SLBs composed of DOPC (or DPPC) with 5 wt % DOPE (or DPPE), 30 wt % cholesterol, and 5 wt % 18:1 (or 16:0) PEG-2000 PE. If necessary, fluorescent lipids (18:1-12:0 NBD-PC, 16:0-12:0 NBD-PC, or Texas Red® DHPE) were incorporated into the SLB at 1-5 wt %. Lipids were lyophilized together prior to rehydration and extrusion; for example 75 μL of DOPC (25 mg/mL), 5 pL of DOPE (25 mg/mL), 10 μL of cholesterol (75 mg/mL), 5 μL of 18:1 PEG-2000 PE (25 mg/mL), and 5 μL of 18:1-12:0 NBD-PC (5 mg/mL) were combined and dried to form liposomes composed of DOPC with 5 wt % DOPE, 30 wt % cholesterol, 5 wt % PEG-2000, and 1 wt % NBD-PC.

Modification of the Supported Lipid Bilayer with Various Types of Targeting Ligands

The specific affinity of protocells for HCC was optimized by conjugating various types of targeting ligands in various densities to the SLB. The SP94 and H5WYG peptides (synthesized with C-terminal cysteine residues) were conjugated to primary amines present in the head groups of PE via the heterobifunctional crosslinker, NHS-(PEG)ₙ-maleimide, which is reactive toward sulfhydryl and amine moieties and possesses a PEG spacer arm, the length of which can be altered to optimize specific affinity. SM(PEG)₂₄ was used in most studies (spacer arm=9.52 nm). Amine moieties present in transferrin, anti-EGFR, and CHALV-1 were converted to free sulfhydryls using the Sulfhydryl Addition Kit (per manufacturer's instructions). Functionalized transferrin and antibodies were conjugated to PE in the SLB using SM(PEG)₂₄. Ligand density was controlled by both reaction stoichiometry and incubation time. For example, protocells were incubated with a 10-fold molar excess of SP94 for 2 hours at room temperature to attain a peptide density of 0.015 wt % (˜6 peptides/protocell), whereas protocells were incubated with a 5000-fold molar excess of SP94 overnight at 4° C. to attain a peptide density of 5.00 wt % (˜2048 peptides/protocell). Average ligand density was determined by Tricine-SDS-PAGE (SP94 and H5WYG peptides) or Laemmli-SDS-PAGE (transferrin, anti-EGFR, and CHALV-1)²⁸. Briefly, protocells were modified with various ligand densities using LC-SPDP (spacer arm=1.57 nm), a heterobifunctional crosslinker that reacts with primary amine and sulfhydryl moieties and is cleavable via reduction. Protocells were exposed to 10 mM dithiothreitol (DTT) for 30 minutes and centrifuged at 10,000 rpm for 5 minutes; the resulting supernatant contained free ligands, the concentration of which was determined via SDS-PAGE by comparing the band intensity of each sample to a standard curve using Image J Image Processing and Analysis software (National Institutes of Health; Bethesda, Md.). 20% gels (with 6% bis-acrylamide and 6 M urea) were used to analyze the SP94 and H5WYG peptide densities. 10% gels were employed to analyze antibody (anti-EGFR and CHALV-1) densities, while 15% gels were used to analyze the density of transferrin.

### Preparation of Fluorescently-Labeled Nanoporous Cores

Nanoporous cores were fluorescently-labeled by adding 100 μL of particles (25 mg/mL) to 900 μL of 20% APTES in 0.5×D-PBS; the particles were incubated in APTES overnight at room temperature, centrifuged (10,000 rpm, 5 minutes) to remove unreacted APTES, and re-suspended in 1 mL of 0.5×D-PBS. An amine-reactive fluorophore (e.g. Alexa Fluor® 647 carboxylic acid, succinimidyl ester; 1 mg/mL in DMSO) was added (5 μL of dye per mL of particles), and the particles were kept at room temperature for 2 hours prior to being centrifuged to remove unreacted dye. Fluorescently-labeled particles were stored in 0.5×D-PBS at 4° C.

Loading of Unimodal Cores and Liposomes with Chemotherapeutic Drugs

Prior to liposome fusion, unimodal nanoporous cores modified to contain 15 wt % AEPTMS (25 mg/mL) were soaked in doxorubicin (5 mM) or a mixture of doxorubicin, cisplatin, and 5-fluorouracil (5 mM of each drug) for 1 hour at room temperature. Excess drug was removed via centrifugation of the particles at 10,000 rpm for 5 minutes. 120-nm liposomes were loaded with DOX using an ammonium phosphate gradient-based method that has been described previously²⁹. Briefly, lipid films were re-hydrated with 300 mM (NH₄)₂HPO₄, and the liposome solution was extruded through a 100-nm membrane at least 10 times. Liposomes were equilibrated with an isotonic buffer solution (140 mM NaCl, 10 mM HEPES, pH 7.4) via dialysis (Float-A-Lyzer G2 dialysis units, 3.5-5 kDa MWCO; Spectrum Laboratories, Inc.; Rancho Dominguez, Calif.) and incubated with doxorubicin HCl (1:3 drug:lipid molar ratio) overnight at 4° C. Excess DOX was removed via size-exclusion chromatography on a 0.7 cm×10 cm Sepharose CL-4B column. Liposomes were loaded with 5-FU or cisplatin as described previously^{30,31}.

Loading of Multimodal Cores with the Multicomponent Mixture, siRNA, and Diphtheria Toxin A-Chain

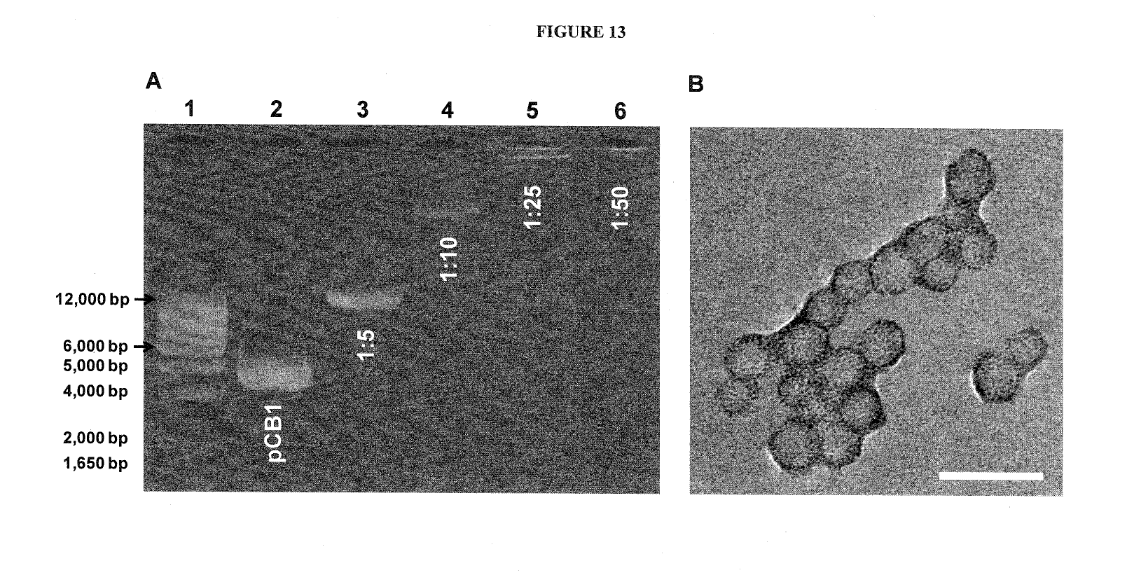

Multimodal nanoporous cores modified to contain 20 wt % AEPTMS (25 mg/mL) were soaked in a solution of calcein (5 mM), Alexa Fluor® 647-labeled dsDNA oligonucleotides (100 μM), RFP (100 μM), and CdSe/ZnS quantum dots (10 μM) for 4 hours; the concentration of each cargo was varied in order to attain the optimal fluorescence intensity for hyperspectral imaging. Calcein was modified with the NLS (synthesized with a C-terminal cysteine residue) by dissolving 1 mg each of calcein and the NLS in 850 μL of 1×D-PBS; 100 μL of EDC (10 mg/mL in deionized water) and 50 μL of BMPH (10 mg/mL in DMSO) were added, and the mixture was incubated for 2 hours at room temperature. Excess calcein was removed via dialysis (Slide-A-Lyzer mini dialysis units, 2 kDa MWCO; Thermo Fisher Scientific LSR; Rockford, Ill.). The dsDNA oligonucleotide was labeled using the Ulysis™ Alexa Fluor® 647 Nucleic Acid Labeling Kit (per manufacturer's instructions) and modified with the NLS by combining 50 μL of dsDNA (2 mM in deionized water) with 50 μL of the NLS (1 mM in DMSO) and 10 μL of SMCC (10 mg/mL in DMSO); the mixture was incubated at room temperature for 2 hours, and excess NLS was removed via dialysis (Slide-A-Lyzer mini dialysis units, 7 kDa MWCO; Thermo Fisher Scientific LSR; Rockford, Ill.). For the delivery experiments described in Supplementary FIGS. 13-16, multimodal nanoporous cores modified with 20 wt % AEPTMS (25 mg/mL) were soaked in siRNA (100 μM) or diphtheria toxin A-chain (100 μM) for 2 hours at 4° C. Unencapsulated cargo was removed via centrifugation at 10,000 rpm for 5 minutes, and liposomes were immediately fused to cargo-loaded cores.

Packaging of the CB1 Plasmid with Histone Proteins.

The process used to supercoil the CB1 plasmid (pCB1) is depicted in FIG. 4. The schematic depicts the process used to supercoil the CB1 plasmid (pCB1) (the CB1 plasmid vector is presented below and in attached FIG. 12) using a highly saturated salt solution, package supercoiled pCB1 with histones H1, H2A, H2B, H3, and H4, and modifying the resulting pCB1-histone complex with a nuclear localization sequence (NLS) that promotes translocation through nuclear pores by conjugation to histone protein. FIGS. 4 (B) and (D) show atomic force microscopy (AFM) images of the CB1 plasmid (B) and histone-packaged pCB1 (D). Scale bars=100 nm (C) and (E) Height profiles that correspond to the red lines in (B) and (D), respectively.

Synthesis of MC40-Targeted Mesoporous Silica Nanoparticle-Supported Lipid Bilayers (Protocells) Loaded with Histone-Packaged pCB1.

As depicted in FIG. 5, 5(A) provides a schematic depicting the process used to generate DNA-loaded, peptide-targeted protocells. Pursuant to this method Histone-packaged pCB1 is loaded into the mesoporous silica nanoparticles that form the core of the protocell by simply soaking the particles in a solution of the pCB1-histone complex. PEGylated liposomes are then fused to DNA-loaded cores to form a supported lipid bilayer (SLB) that is further modified with a targeting peptide (MC40) that binds to HCC and a endosomolytic peptide (H5WYG) that promotes endosomal escape of internalized protocells. A sulfhydryl-to-amine crosslinker (spacer arm=9.5 nm) was used to conjugate peptides, modified with a C-terminal cysteine residue, to DOPE moieties in the SLB. FIG. 5(B) shows the transmission electron microscopy (TEM) image of the mesoporous silica nanoparticles that are used as the core of the protocell. Scale bar=200 nm. Inset=scanning electron microscopy (SEM) image, which demonstrates that the 15-25 nm pores are surface-accessible. Inset scale bar=50 nm. 5(C) shows the size distribution for the mesoporous silica nanoparticles, as determined by dynamic light scattering (DLS). (5D, left axis) Cumulative pore volume plot for the mesoporous silica nanoparticles, calculated from the adsorption branch of the nitrogen sorption isotherm shown in Figure S-4A using the Barrett-Joyner-Halenda (BJH) model. (5D, right axis) Size distribution for the pCB1-histone complex, as determined by DLS.

Mesoporous Silica Nanoparticles have a High Capacity for Histone-Packaged pCB1, and the Resulting Protocells Release Encapsulated DNA Only Under Conditions that Mimic the Endosomal Environment.

As depicted in FIG. 6(A), the concentration of pCB1 or histone-packed pCB1 (‘complex’) that can be encapsulated within unmodified mesoporous silica nanoparticles (ζ=−38.5 mV) or mesoporous silica nanoparticles modified with APTES, an amine-containing silane (ζ+11.5 mV). FIG. 6(B) shows the percentage of Hep3B that become positive for ZsGreen, a green fluorescent protein encoded by pCB1, when 1×10⁶ cells/mL are incubated with 1×10⁹ MC40-targeted, pCB1-loaded protocells for 24 hours at 37° C. The x-axis specifies whether the protocell core was modified with APTES and whether pCB1 was pre-packaged with histones. pCB1 packaged with a mixture of DOTAP and DOPE (1:1 w/w) was included as a control in (A) and (B). FIGS. 6(C) and (D) show the time-dependent release of histone-packaged pCB1 from unmodified mesoporous silica nanoparticles and corresponding protocells upon exposure to a simulated body fluid (C) or a pH 5 buffer (D). The protocell SLB was composed of DOPC with 5 wt % DOPE, 30 wt % cholesterol, and 10 wt % PEG-2000 and, for (B), was modified with 0.015 wt % MC40 and 0.500 wt % H5WYG. All error bars represent 95% confidence intervals (1.96σ) for n=3.

The Process by which MC40-Targeted Protocells Deliver Histone-Packaged pCB1 to HCC.

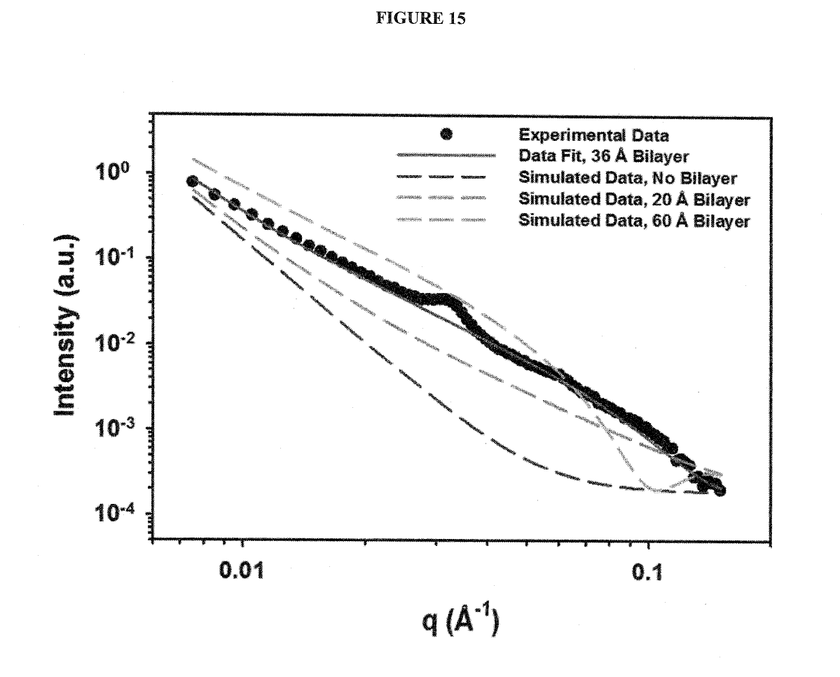

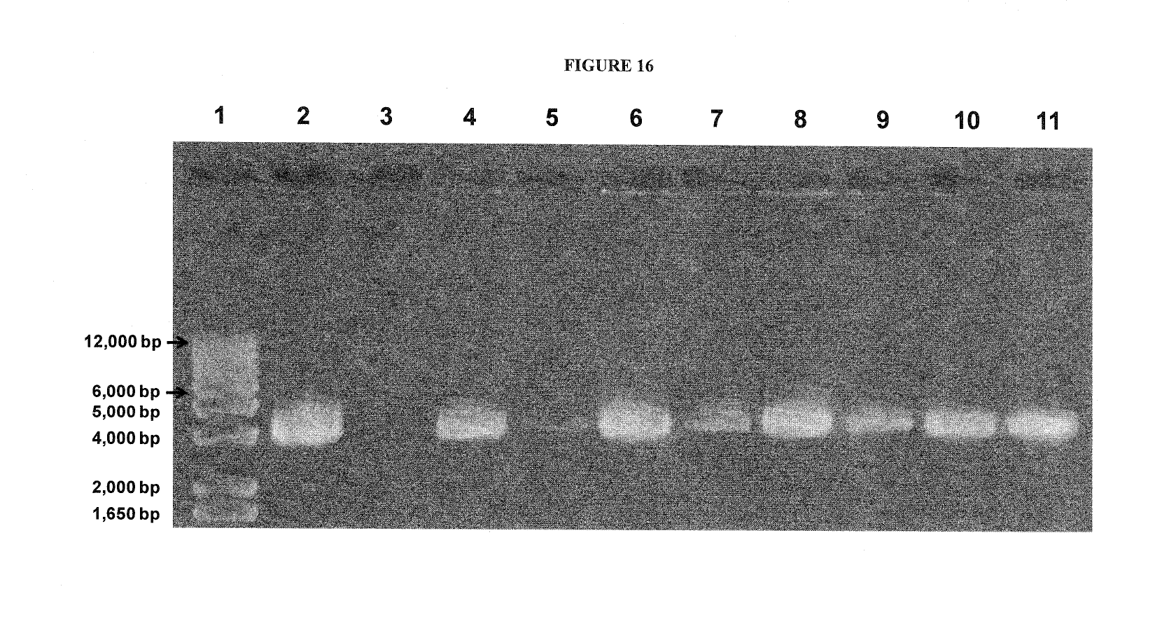

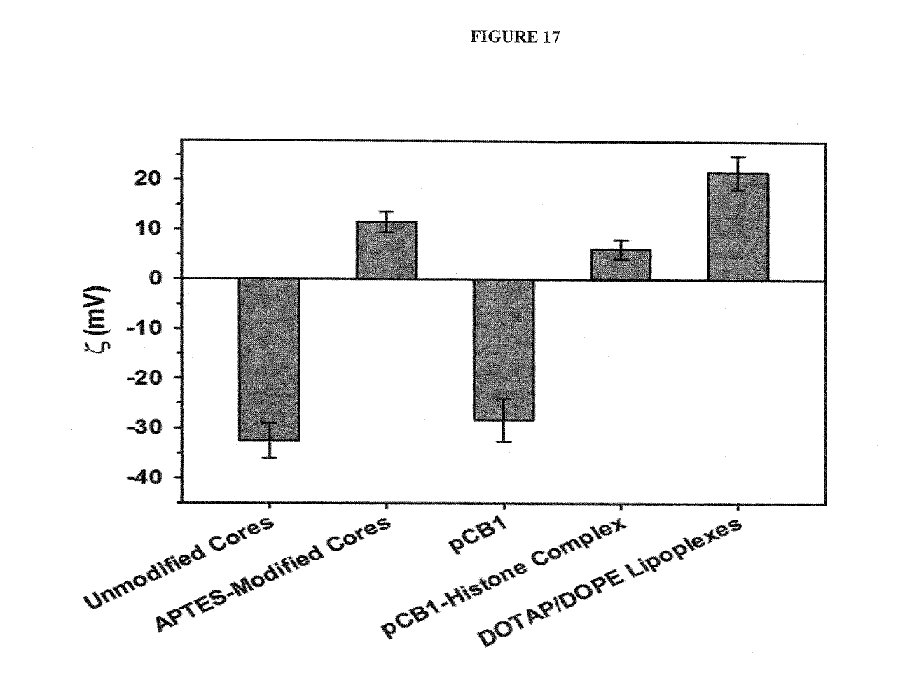

As depicted in the schematic presented in attached FIG. 7 [1] MC40-targeted protocells bind to Hep3B cells with high affinity due to the recruitment of targeting peptides to Met, which is over-expressed by a variety of HCC lines. The fluid DOPC SLB promotes peptide mobility and, therefore, enables protocells modified with a low MC40 density to retain a high specific affinity for Hep3B (see FIG. 8A). [2] MC40-targeted protocells become internalized by Hep3B via receptor-mediated endocytosis (see FIG. 8B and FIG. 15A). [3] Endosomal conditions destabilize the SLB [insert Nature Materials ref] and cause protonation of the H5WYG endosomolytic peptide, both of which enable histone-packaged pCB1 to become dispersed in the cytosol of Hep3B cells (see FIG. 15B). [4] pCB1-histone complexes, when modified with a nuclear localization sequence (NLS), become concentrated in the nuclei of Hep3B cells within ˜24 hours (see FIG. 16C), which enables efficient transfection of both dividing and non-dividing cancer cells (see FIG. 17).

MC40-Targeted Protocells Bind to HCC with High Affinity and are Internalized by Hep3B but not by Normal Hepatocytes.

FIG. 8(A) shows the apparent dissociation constants (K_{d}) for MC40-targeted protocells when exposed to Hep3B or hepatocytes; K_{d} values are inversely related to specific affinity and were determined from saturation binding curves (see Figure S-11). Error bars represent 95% confidence intervals (1.96σ) for n=5. FIGS. 8(B) and (C) show the confocal fluorescence microscopy images of Hep3B (B) and hepatocytes (C) that were exposed to a 1000-fold excess MC40-targeted protocells for 1 hour at 37° C. Met was stained with an Alexa Fluor® 488-labeled monoclonal antibody (green), the protocell core was labeled with Alexa Fluor® 594 (red), and cell nuclei were stained with Hoechst 33342 (blue). Scale bars=Protocell SLBs were composed of DOPC with 5 wt % DOPE, 30 wt % cholesterol, and 10 wt % PEG-2000 (18:1) and were modified with either 0.015 wt % (A-C) or 0.500 wt % (A) of the MC40 targeting peptide.

MC40-Targeted, pCB1-Loaded Protocells Induce Apoptosis of HCC at Picomolar Concentrations but have a Minimal Impact on the Viability of Normal Hepatocytes.

FIGS. 9(A) and (B) shows the dose (A) and time (B) dependent decreases in expression of cyclin B1 mRNA and cyclin B1 protein upon continual exposure of Hep3B to MC40-targeted, pCB1-loaded protocells at 37° C. Cells were exposed to various pCB1 concentrations for 48 hours in (A) and to 5 pM of pCB1 for various periods of time in (B). Expression of cyclin B1 protein in hepatocytes and ZsGreen in Hep3B are included as controls. Real-time PCR and immunofluorescence were employed to determine cyclin B1 mRNA and protein concentrations, respectively. FIG. 9(C) shows the percentage of Hep3B that become arrested in G₂/M phase after continual exposure to MC40-targeted, pCB1-loaded protocells ([pCB1]=5 pM) for various periods of time at 37° C. The percentage of hepatocytes in G₂/M phase and Hep3B in S phase are included for comparison. Cells were stained with Hoechst 33342 prior to cell cycle analysis. FIG. 9(D) shows the percentage of Hep3B that become apoptotic upon continual exposure to MC40-targeted, pCB1-loaded protocells ([pCB1]=5 pM) for various periods of time at 37° C. The percentage of hepatocytes positive for markers of apoptosis was included as a control. Cells positive for Alexa Fluor® 647-labeled annexin V were considered to be in the early stages of apoptosis, while cells positive for both annexin V and propidium iodide were considered to be in the late stages of apoptosis. The total number of apoptotic cells was determined by adding the numbers of single- and double-positive cells. In all experiments, protocell SLBs were composed of DOPC with 5 wt % DOPE, 30 wt % cholesterol, and 10 wt % PEG-2000 (18:1) and were modified with 0.015 wt % MC40 and 0.500 wt % H5WYG. All error bars represent 95% confidence intervals (1.96 cy) for n=3.

MC40-Targeted, pCB1-Loaded Protocells Induce Selective Apoptosis of HCC 2500-Fold More Effectively than Corresponding Lipoplexes.

FIG. 10(A) shows the zeta potential values for DOPC protocells, DOPC protocells modified with 10 wt % PEG-2000 (18:1), lipoplexes composed of pCB1 and a mixture of DOTAP and DOPE (1:1 w/w), and DOTAP/DOPE lipoplexes modified with 10 wt % PEG-2000. All zeta potential measurements were conducted in 0.5×PBS (pH 7.4). FIG. 10 (B, left axis) shows the percentage of Hep3B and hepatocytes that become apoptotic upon continual exposure to 5 pM of pCB1, delivered via MC40-targeted protocells or lipoplexes, for 48 hours at 37° C. FIG. 10 (B, right axis) shows the number of MC40-targeted, pCB1-loaded protocells or lipoplexes necessary to induce apoptosis in 90% of 1×10⁶ Hep3B cells within 48 hours at 37° C. For (B), cells were stained with Alexa Fluor® 647-labeled annexin V and propidium iodide; single- and double-positive cells were considered to be apoptotic. Protocell SLBs were composed of DOPC with 5 wt % DOPE, 30 wt % cholesterol, and 10 wt % PEG-2000 (when indicated) and were modified with 0.015 wt % MC40 and 0.500 wt % H5WYG. DOTAP/DOPE lipoplexes were modified with 10 wt % PEG-2000 (when indicated), 0.015 wt % MC40, and 0.500 wt % H5WYG. pCB1 was modified with the NLS in all experiments. All error bars represent 95% confidence intervals (1.96σ) for n=3.

MC40-Targeted Protocells Selectively Deliver High Concentrations of Taxol, Bcl-2-Specific siRNA, and pCB1 to HCC without Affecting the Viability of Hepatocytes.

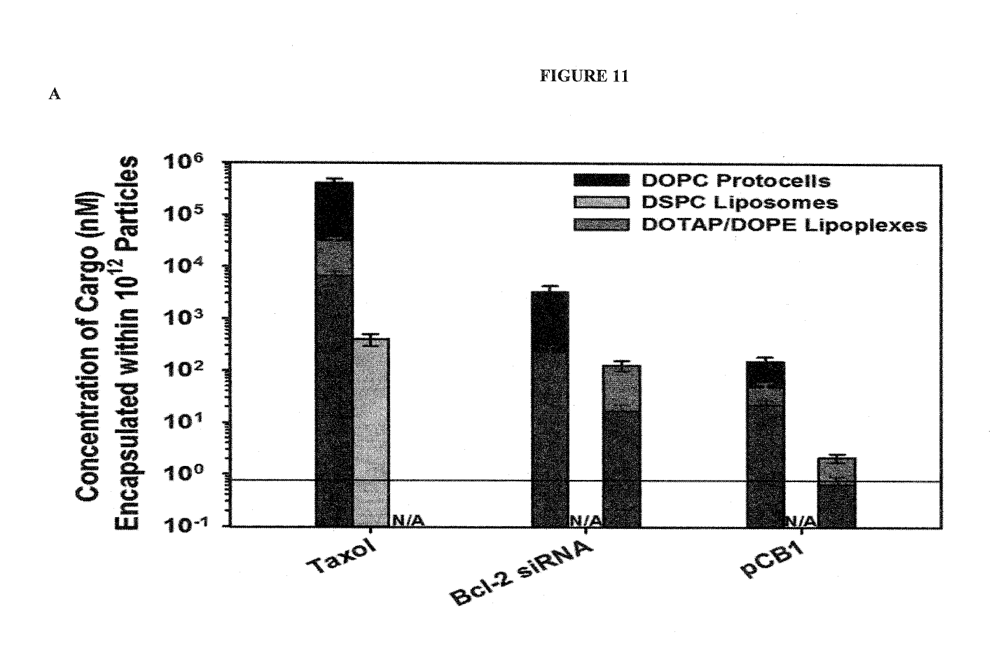

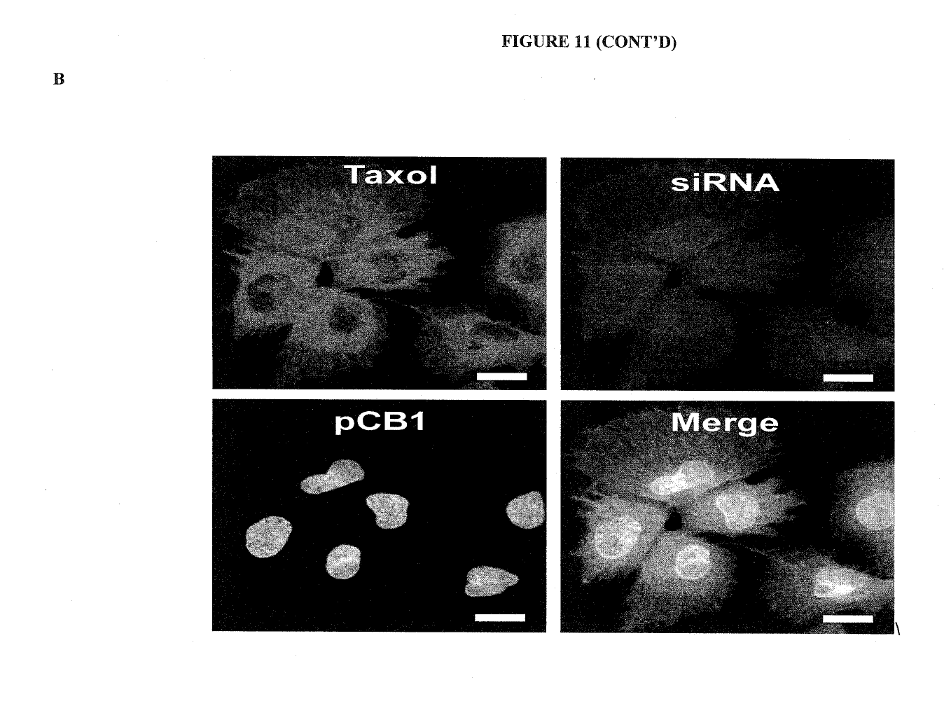

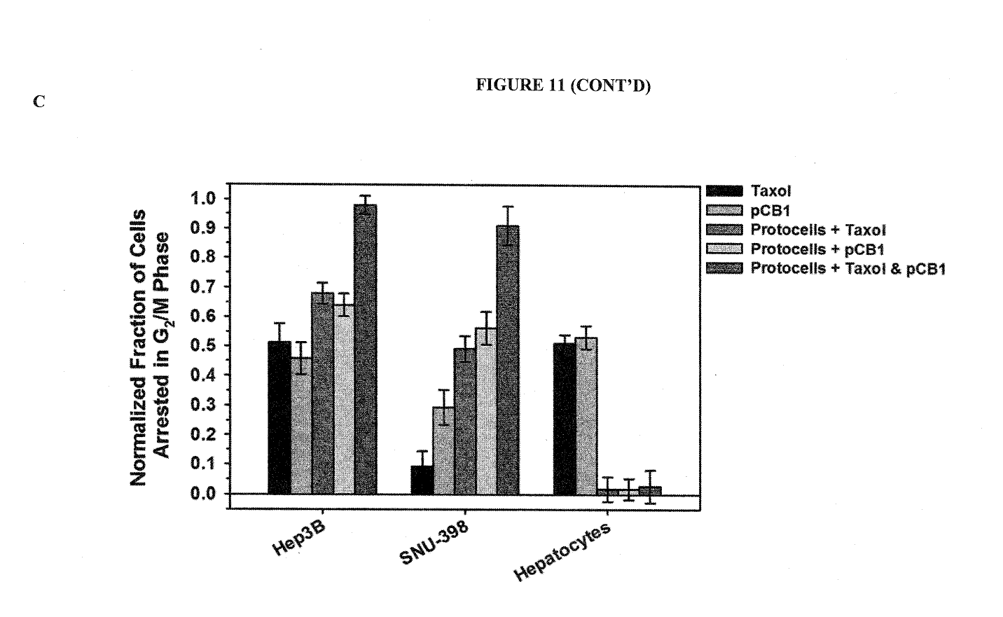

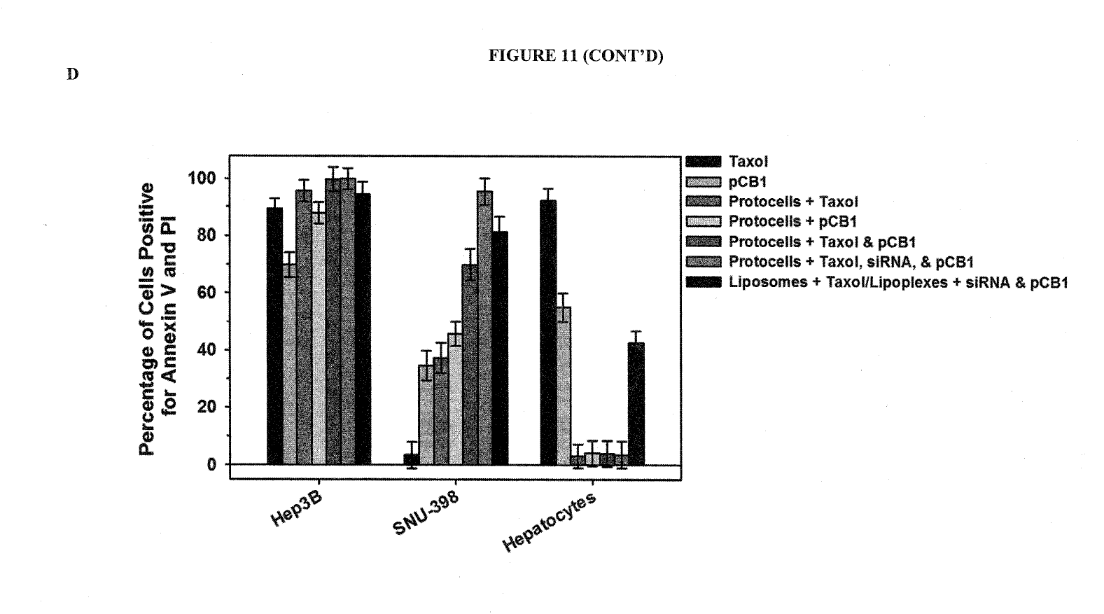

FIG. 11(A) shows the concentrations of taxol, siRNA that silence expression of Bcl-2, and the CB1 plasmid that can be encapsulated within 10¹² protocells, liposomes, or lipoplexes. Red bars in FIG. 11A indicate how taxol and pCB1 concentrations change when both are loaded within protocells. Blue bars indicate how taxol, siRNA, and pCB1 concentrations change when all three are loaded within protocells or when siRNA and pCB1 are loaded within lipoplexes. FIG. 11(B) provides a confocal fluorescence microscopy image showing the intracellular distributions of Oregon Green® 488-labeled taxol (green), Alexa Fluor® 594-labeled siRNA (red), and Cy5-labeled pDNA (white) upon delivery to Hep3B via MC40-targeted protocells. Cells were incubated with a 1000-fold excess of MC40-targeted protocells for 24 hours at 37° C. prior to being fixed and stained with Hoechst 33342 (blue). Scale bars=10 μm. FIG. 11(C) shows the fractions of Hep3B, SNU-398, and hepatocyte cells that become arrested in G₂/M phase upon exposure to 10 nM of taxol and/or 5 pM of pCB1 for 48 hours at 37° C. Fractions were normalized against the percentage of logarithmically-growing cells in G₂/M. FIG. 11(D) shows the percentage of Hep3B, SNU-398, and hepatocyte cells that become positive for Alexa Fluor® 647-labeled annexin V and propidium iodide (PI) upon exposure to 10 nM of taxol, 250 pM of Bcl-2-specific siRNA, and/or 5 pM of pCB1 for 48 hours at 37° C. In (C) and (D), ‘pCB1’ refers to pCB1 that was packaged and delivered non-specifically to cells using a mixture of DOTAP and DOPE (1:1 w/w). In all experiments, protocell SLBs were composed of DOPC with 5 wt % DOPE, 30 wt % cholesterol, and 10 wt % PEG-2000 (18:1) and were modified with 0.015 wt % MC40 and 0.500 wt % H5WYG. Liposomes were composed of DSPC with 5 wt % DMPE, 30 wt % cholesterol, and 10 wt % PEG-2000 (16:0) and were modified with 0.015 wt % MC40 and 0.500 wt % H5WYG. Lipoplexes were composed of a DOTAP:DOPE (1:1 w/w) mixture and were modified with 10 wt % PEG-2000, 0.015 wt % MC40, and 0.500 wt % H5WYG. pCB1 was modified with the NLS in all experiments. All error bars represent 95% confidence intervals (1.96σ) for n=3.

### Vector Map for the CB1 Plasmid

As shown in FIG. 12, the CB1 plasmid (pCB1) was constructed from the RNAi-Ready pSIREN-RetroQ-ZsGreen vector (Clontech Laboratories, Inc.; Mountain View, Calif.) and the pNEB193 vector (New England BioLabs, Inc.; Ipswich, Mass.). pCB1 encodes a cyclin B1-specific small hairpin RNA (shRNA) [Yuan, et al., Oncogene (2006) 25, 1753-1762] and a Zoanthus sp. green fluorescent protein (ZsGreen). Constitutive shRNA expression is driven by the RNA Pol III-dependent human U6 promoter (P_{U6}), while constitutive ZsGreen expression is driven by the immediate early promoter of cytomegalovirus (P_{CMV IE}). The on and Amp^{R} elements enable propagation of the plasmid in E. coli. The DNA sequences that encode the sense and antisense strands of the cyclin B1-specific shRNA are underlined and are flanked by the restriction enzyme sites (BamHI in red and EcoRI in blue) that were employed to introduce the dsDNA oligonucleotide into the pSIREN vector.

Characterization of Histone-Packaged pCB1.

FIG. 13(A) shows the electrophoretic mobility shift assays for pCB1 exposed to increasing concentrations of histones (H1, H2A, H2B, H3, and H4 in a 1:2:2:2:2 molar ratio). The pCB1:histone molar ratio is given for lanes 3-6. Lane 1 contains a DNA ladder, and lane 2 contains pCB1 with no added histones. FIG. 13(B) shows the TEM image of histone-packaged pCB1 (1:50 pCB1:histone molar ratio). Scale bar=50 nm

Nitrogen Sorption Analysis of Unloaded and pCB1-Loaded Mesoporous Silica Nanoparticles.

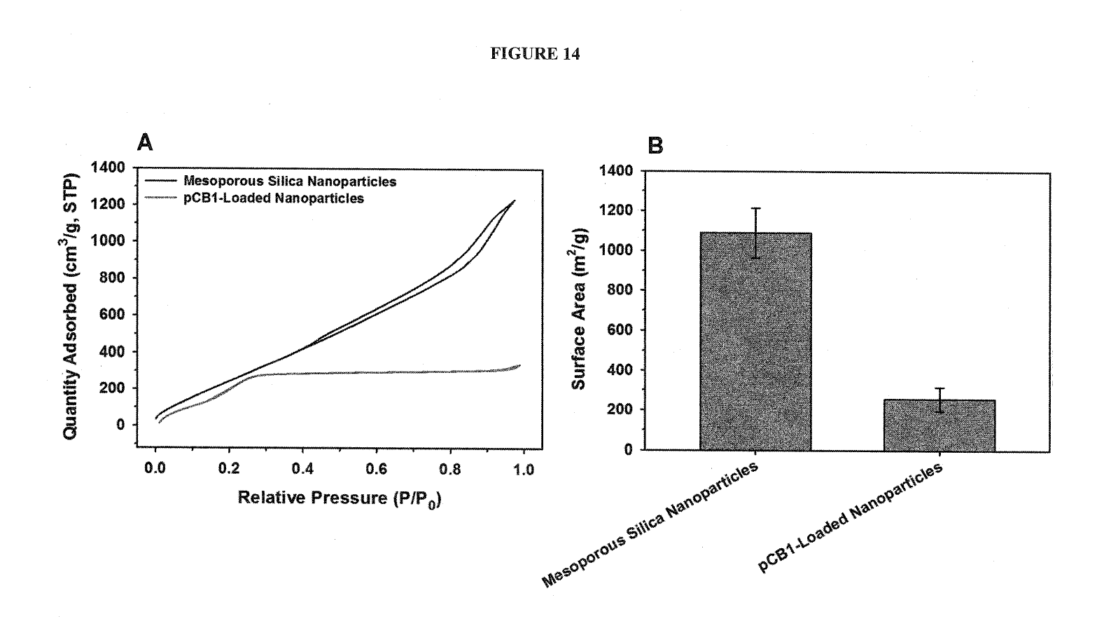

FIG. 14(A) Nitrogen sorption isotherms for mesoporous silica nanoparticles before and after loading with histone-packaged pCB1. FIG. 14(B) shows the Brunauer-Emmett-Teller (BET) surface area of mesoporous silica nanoparticles, before and after loading with histone-packaged pCB1. Error bars represent 95% confidence intervals (1.96σ) for n=3.

### Small-Angle Neutron Scattering (SANS) Data for DOPC Protocells.

FIG. 15 shows SANS data for DOPC protcells. The data fit was obtained using a model for polydisperse porous silica spheres with a conformal shell of constant thickness and shows the presence of a 36-Å bilayer at the surface of the silica particles that spans pore openings. Simulated SANS data for bilayer thicknesses of 0, 20, and 60 Å are included for comparison. The measured bilayer thickness of 36 A is consistent with other neutron studies (33-38 Å) [see, Ferrari, M. Cancer nanotechnology: Opportunities and challenges. Nature Reviews Cancer 5, 161-171 (2005)] performed on planar supported lipid bilayers and, under these contrast conditions, primarily represents scattering from the hydrogen-rich hydrocarbon core of the lipid bilayer. Experimental data also demonstrates the presence of 299.2-Δ pores, determined by dividing 0.0315 Å⁻¹ (i.e. the q-value for the peak in the experimental data, which is caused by scattering from pores) into 2π. SANS data were obtained on the LQD beam line at LANSCE (Los Alamos National Laboratories) using a 5% (v/v) protocell suspension in 100% D₂O PBS buffer. Data were fit using the NCNR SANS data analysis package (NIST).

Protocells Protect Encapsulated DNA from Nuclease Degradation.

FIG. 16 shows the results of agarose gel electrophoresis of DNase I-treated pCB1 (lane 3), histone-packaged pCB1 (lane 5), pCB1 packaged with a 1:1 (w/w) mixture of DOTAP and DOPE (lane 7), pCB1 loaded in protocells with cationic cores (lane 9), and histone-packaged pCB1 loaded in protocells with anionic cores (lane 11). Naked pCB1 (lane 2), pCB1 released from histones (lane 4), pCB1 released from DOTAP/DOPE lipoplexes (lane 6), pCB1 released from protocells with cationic cores (lane 8), and histone-packaged pCB1 released from protocells with anionic cores (lane 10) are included for comparison. Lane 1 contains a DNA ladder. Samples were incubated with DNase I (1 unit per 50 ng of DNA) for 30 minutes at room temperature, and pCB1 release was stimulated using 1% SDS.

FIG. 17 shows the Zeta potential (1) values for mesoporous silica nanoparticles (‘unmodified cores’), mesoporous silica nanoparticles that were soaked in 20% (v/v) APTES for 12 hours at room temperature (‘APTES-modified cores’), the CB1 plasmid (‘pCB1’), histone-packaged pCB1 (‘pCB1-histone complex’), and pCB1 packaged with a 1:1 (w/w) mixture of DOTAP and DOPE (‘DOTAP/DOPE Lipoplexes’). Zeta potential measurements were conducted in 0.5×PBS (pH 7.4). Error bars represent 95% confidence intervals (1.96σ) for n=3.

Representative Forward Scatter-Side Scatter (FSC-SSC) Plots and FL-1 Histograms Used to Determine the Percentage of Cells Positive for ZsGreen Expression in FIG. 6 and S-16 (A)-(D)

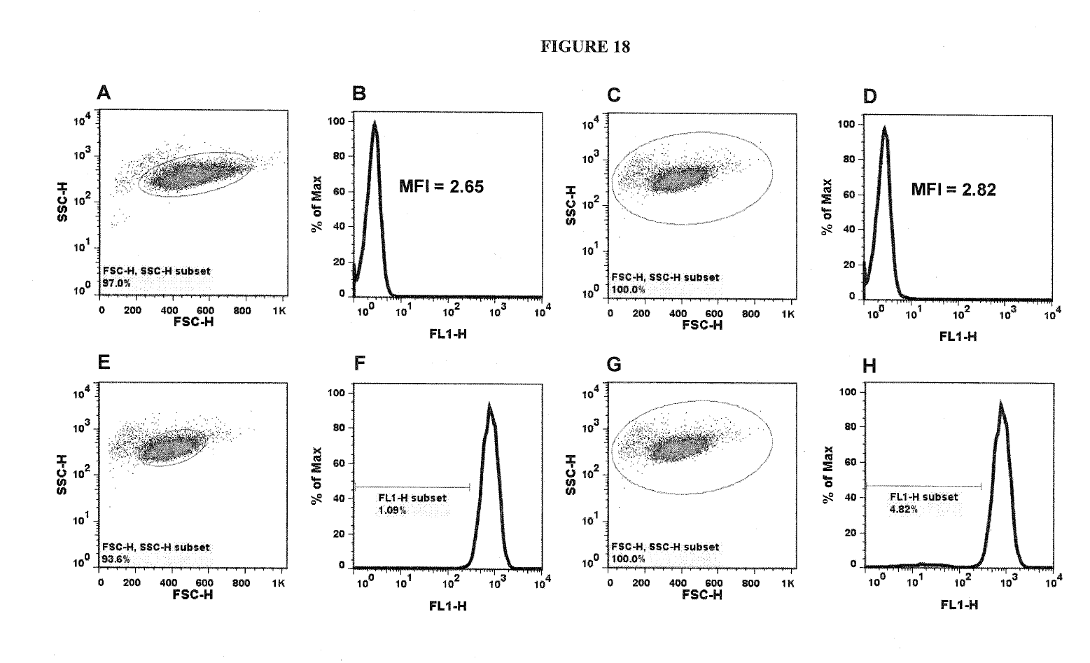

FIG. 18 shows the FSC-SSC plots (A and C) and the corresponding FL-1 histograms (B and D, respectively) for ZsGreen-negative cells that were (A) or were not (C) gated to exclude cellular debris. Mean fluorescence intensity (MFI) values for the FL-1 channel are given in (B) and (D). (E)-(H) FSC-SSC plots (E and G) and the corresponding FL-1 histograms (F and H, respectively) for ZsGreen-positive cells that were (E) or were not (G) gated to exclude cellular debris. Gates on (F) and (H) correspond to the percentage of cells with MFI≦282, i.e. 100× the MFI of ZsGreen-negative cells (see panel D).

### Identification of the MC40 Targeting Peptide.

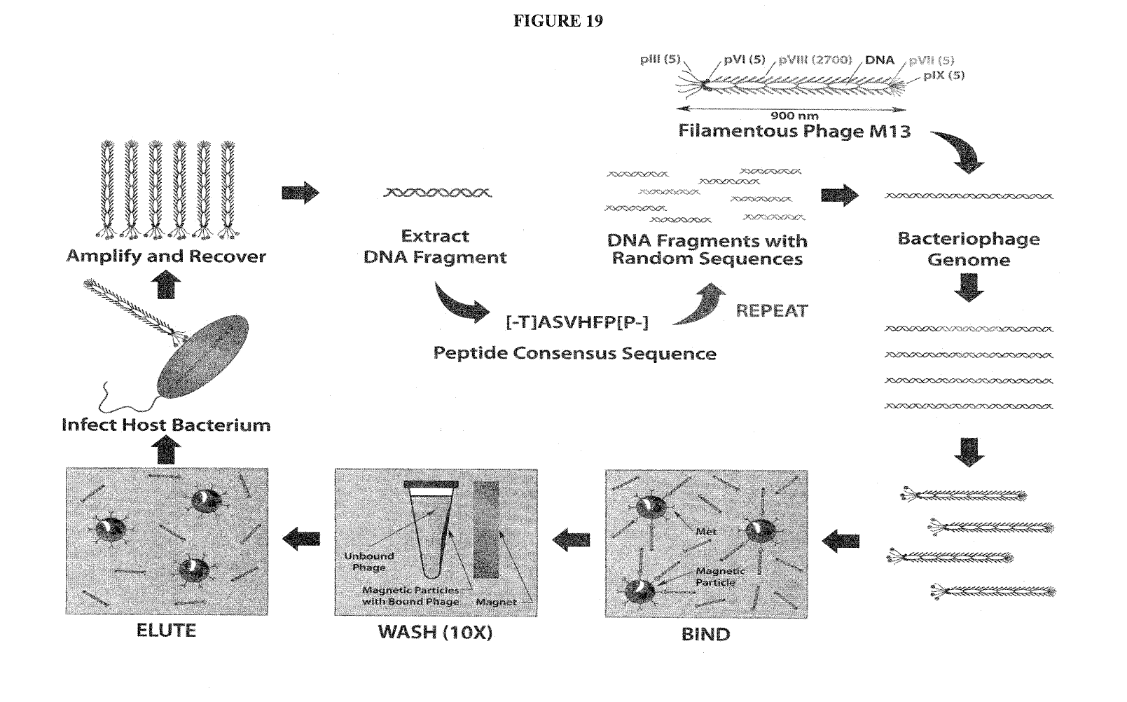

FIG. 19 provides a schematic depicting the process used to select the MC40 targeting peptide from a Ph.D.™-7 phage display library (New England BioLabs, Inc.; Ipswich, Mass.). 1×10¹¹ pfu/mL were incubated with 100 nM of recombinant human Met (rhMet), fused to the Fc domain of human IgG, for 1 hour at room temperature. Protein A or protein G-coated magnetic particles were used to affinity capture Met-phage complexes and were subsequently washed 10 times with TBS (50 mM Tris-HCl with 150 mM NaCl, pH 7.4) to remove unbound phage. Bound phage clones were eluted with a low-pH buffer (0.2 M glycine with 1 mg/mL BSA, pH 2.2), and elutants were amplified via infection of the host bacterium (E. coli ER2738). Pursuant to the schematic, five rounds of affinity selection were performed using increasingly stringent conditions: the Met concentration was decreased from 100 nM to 50 nM to 10 nM, the incubation time was reduced from 1 hour to 30 minutes to 15 minutes, and the concentration of Tween-20 added to the wash buffer was increased from 0% (v/v) to 0.1% to 0.5%. Peptides specific for protein A and protein G were avoided by alternating rounds of selection between protein A-coated magnetic particles and protein G-coated magnetic particles. After five rounds of selection, DNA was recovered from 40 individual clones and sequenced using the −96 gIII primer provided with the Ph.D.™-7 kit. The sequences which have the greatest binding activity against the MET receptor are presented as follows:

SEQ ID NO: 1
ASVHFPP (Ala-Ser-Val-His-Phe-Pro-Pro)
SEQ ID NO: 2
TATFWFQ (Thr-Ala-Thr-Phe-Trp-Phe-Gln)
SEQ ID NO: 3
TSPVALL (Thr-Ser-Pro-Val-Ala-Leu-Leu)
SEQ ID NO: 4
IPLKVHP (Ile-Pro-Leu-Lys-Val-His-Pro)
SEQ ID NO: 5
WPRLTNM (Trp-Pro-Arg-Leu-Thr-Asn-Met)

### Characterization of the MC40 Targeting Peptide.

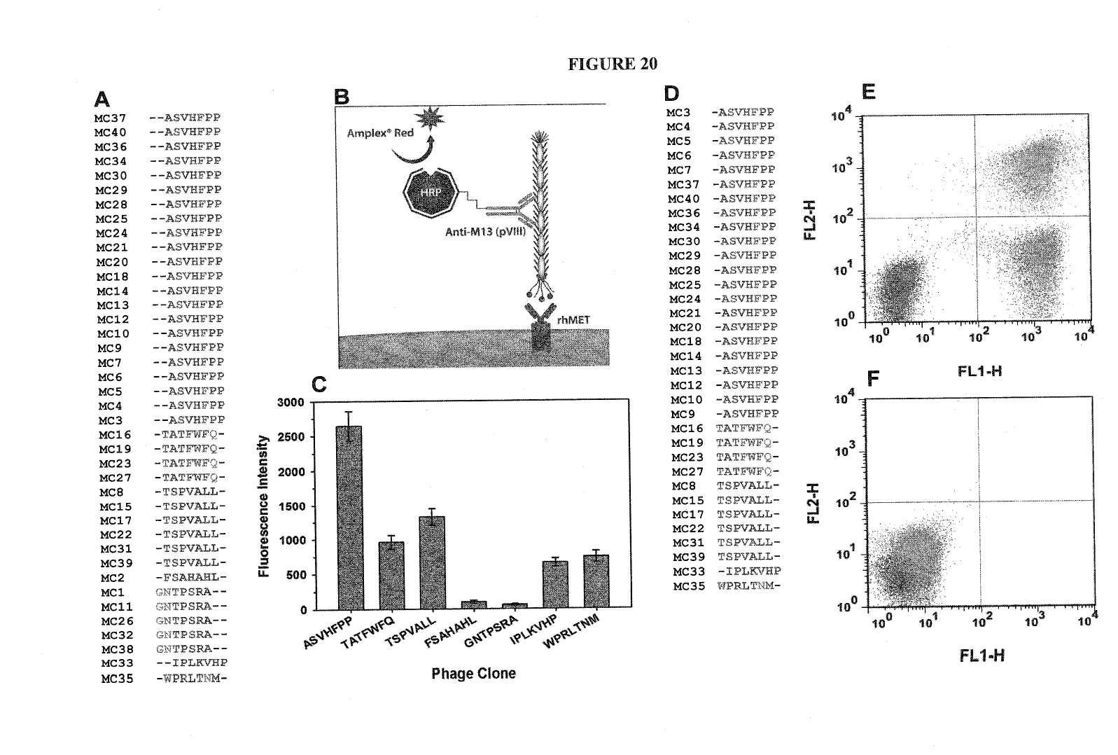

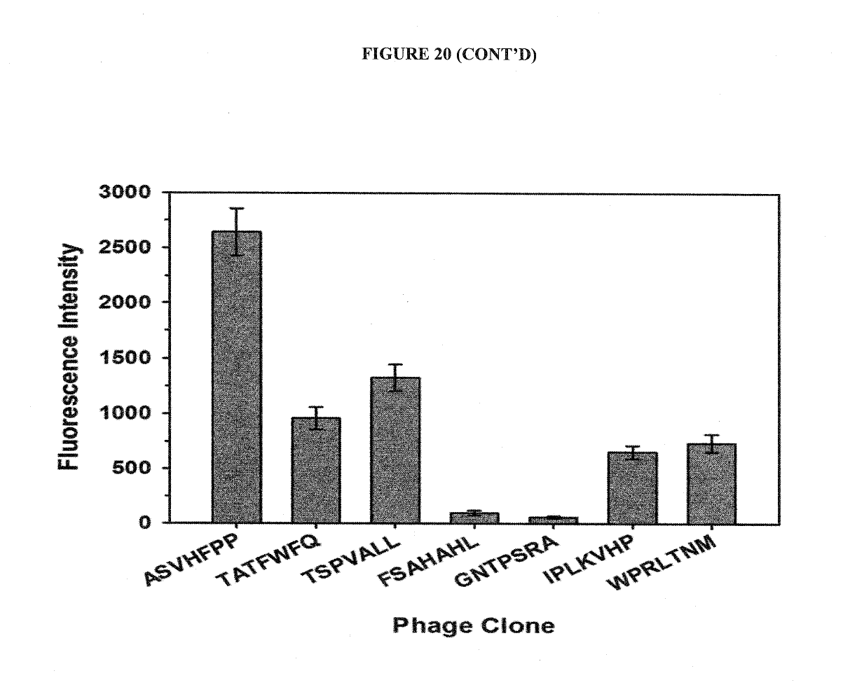

FIG. 20(A) shows the peptide sequence alignment after the 5^{th} round of selection; the predominant sequence, ASVHFPP, is similar to the emboldened portion of a previously-identified Met-specific 12-mer, YLFSVHWPPLKA SEQ ID N0:15). Phage clones displaying the target-unrelated HAIYPRH peptide (˜10%) (SEQ ID NO:16) were omitted from the sequence alignment. FIGS. 20(B) and (C) show the degree to which affinity-selected phage clones bound to rhMet was determined via enzyme-linked immunosorbent assay (ELISA). The ELISA scheme, depicted in (B), is described in the Materials and Methods section. ELISA results are shown in (C). FIG. 20(D) shows the sequence alignment after peptides that do not bind to Met were removed. The consensus sequence depicted in FIG. 20 was determined from this alignment. FIGS. 20(E) and (F) show the flow cytometry scatter plots for Hep3B (E) and hepatocytes (F) exposed to either (1) an Alexa Fluor® 488-labeled monoclonal antibody against Met AND an irrelevant phage clone (TPDWLFP, SEQ ID NO:17) AND an Alexa Fluor® 546-labeled monoclonal antibody against M13 phage (blue dots) or (2) an Alexa Fluor® 488-labeled monoclonal antibody against Met AND the MC40 clone AND an Alexa Fluor® 546-labeled monoclonal antibody against M13 phage (orange dots). Untreated cells (red dots) were used to set voltage parameters for the FL-1 (Alexa Fluor® 488 fluorescence) and FL-2 (Alexa Fluor® 546 fluorescence) channels.

### Sample Binding Curves for MC40-Targeted Protocells Exposed to Hep3B.

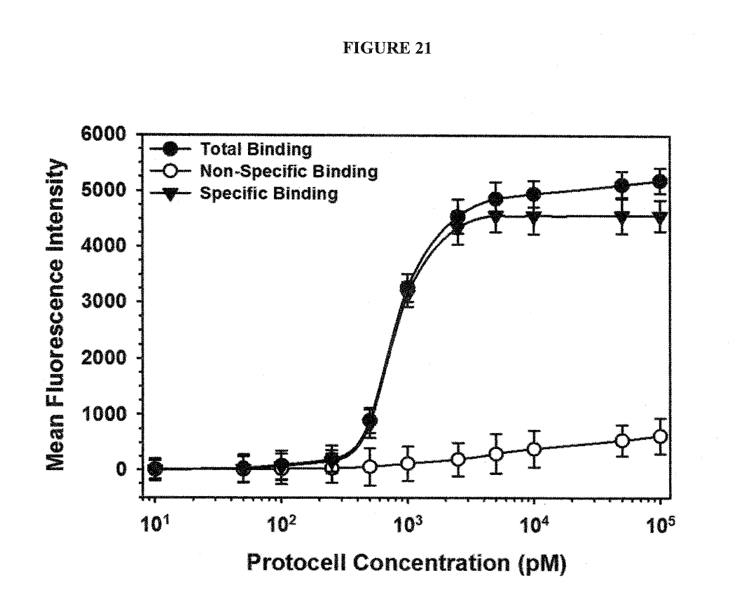

To determine the dissociation constants in FIG. 8A, 1×10⁶ Hep3B or hepatocytes were pre-treated with cytochalasin D to inhibit endocytosis and incubated with various concentrations of Alexa Fluor® 647-labeled, MC40-targeted protocells for 1 hour at 37° C. Flow cytometry was used to determine mean fluorescence intensities for the resulting cell populations, which were plotted against protocell concentrations to obtain total binding curves. Non-specific binding was determined by incubating cells with Alexa Fluor® 647-labeled, MC40-targeted protocells in the presence of a saturating concentration of unlabeled hepatocyte growth factor. Specific binding curves were obtained by subtracting non-specific binding curves from total binding curves; K_{d} values were calculated from specific binding curves. In the experiments which are depicted in FIG. 21, protocell SLBs were composed of DOPC with 5 wt % DOPE, 30 wt % cholesterol, and 10 wt % PEG-2000 (18:1) and were modified with 0.015 wt % (˜6 peptides/particle) of the MC40 targeting peptide; the corresponding K_{d} value is 1050±142 pM. All error bars represent 95% confidence intervals (1.96σ) for n=5.

MC40-Targeted Protocells are Internalized Via Receptor-Mediated Endocytosis and, in the Absence of the H5WYG Peptide, are Directed to Lysosomes.

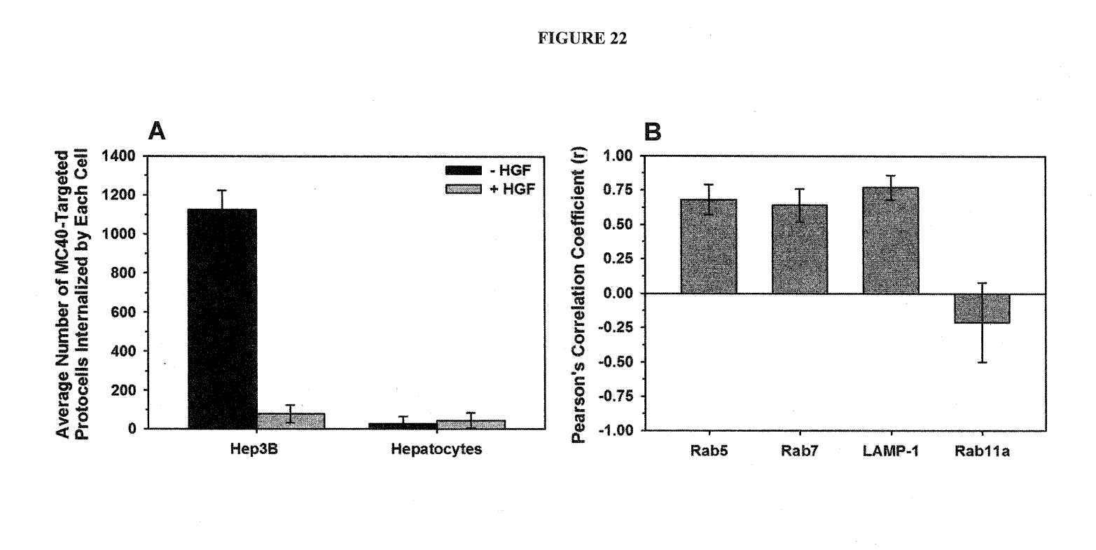

FIG. 22(A) shows the average number of MC40-targeted protocells internalized by each Hep3B or hepatocyte cell within one hour at 37° C. 1×10⁶ cells were incubated with various concentrations of protocells in the absence (−) or presence (+) of a saturating concentration (100 μg/mL) of human hepatocyte growth factor (HGF), and flow cytometry was used to determine the average number of particles associated with each cell, as described by Ashley, et al. Nature Materials, 2011, May; 10(5):389-97. Protocells were labeled with NBD and pHrodo™ to distinguish surface-bound particles from those internalized into acidic intracellular compartments (respectively). Error bars represent 95% confidence intervals (1.96σ) for n=3. (B) Pearson's correlation coefficients (r-values) between protocells and: (1) Rab5, (2) Rab7, (3) Lysosomal-Associated Membrane Protein 1 (LAMP-1), or (4) Rab11a. Hep3B cells were incubated with a 1000-fold excess of Alexa Fluor® 594-labeled protocells for 1 hour at 37° C. before being fixed, permeabilized, and incubated with Alexa Fluor® 488-labeled antibodies against Rab5, Rab7, LAMP-1, or Rab11a. SlideBook software was used to determine r-values, which are expressed as the mean value±the standard deviation for n=3×50 cells. Differential Interference Contrast (DIC) images were employed to define the boundaries of Hep3B cells so that pixels outside of the cell boundaries could be disregarded when calculating r-values. Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG.

Histone-Packaged pCB1, when Modified with a NLS and Delivered Via MC40-Targeted Protocells, Becomes Concentrated in the Nuclei of HCC Cells in a Time-Dependent Manner.

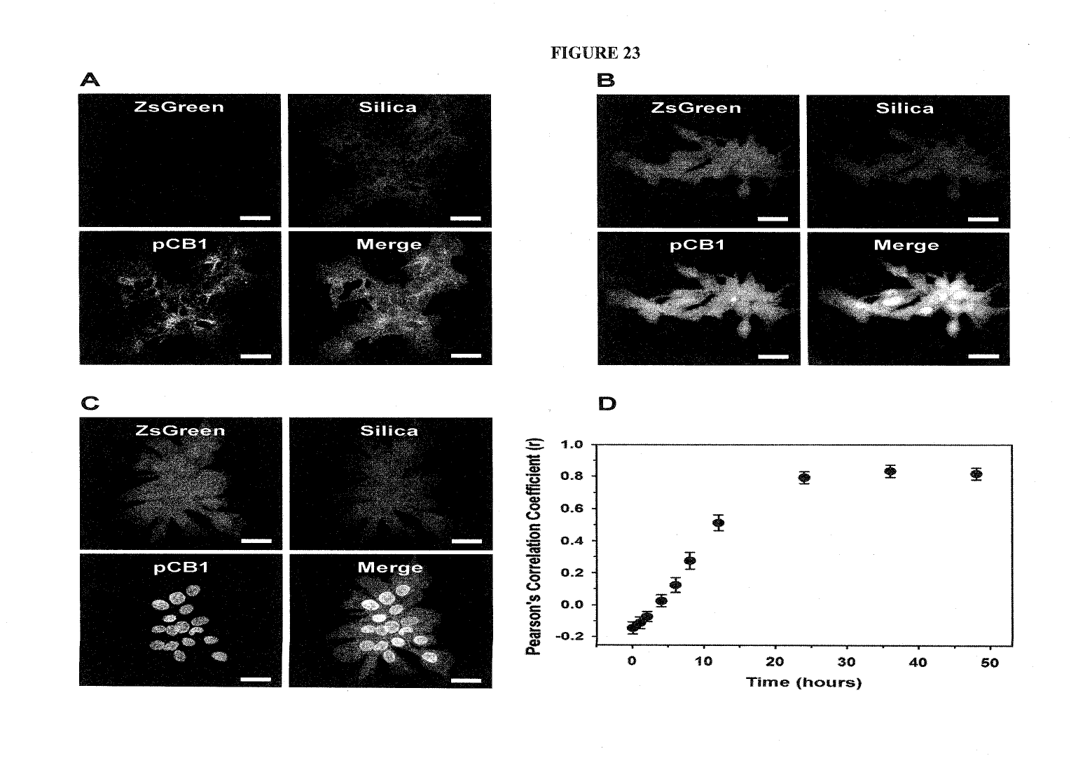

FIGS. 23(A)-(C) depict confocal fluorescence microscopy images of Hep3B cells exposed to a 1000-fold excess of MC40-targeted, pCB1-loaded protocells for 15 minutes (A), 12 hours (B), or 24 hours (C) at 37° C. For (B), endosomal escape of protocells and cytosolic dispersion of pCB1 was evident after ˜2 hours; ZsGreen expression was not detectable until 12-16 hours, however. At 24 hours, Cy5-labeled pCB1 remained distributed throughout the cells; cytosolic staining is not visible in (C), however, since the gain of the Cy5 channel was reduced to avoid saturation of pixels localized within the nuclei. Silica cores were labeled with Alexa Fluor® 594 (red), pCB1 was labeled with Cy5 (white), and cell nuclei were counterstained with Hoechst 33342 (blue). Scale bars=20 FIG. 23(D) shows Pearson's correlation coefficients (r-values) versus time for Cy5-labeled pCB1 and Hoechst 33342-labeled Hep3B nuclei. SlideBook software was used to determine r-values, which are expressed as the mean value±the standard deviation for n=3×50 cells. Differential Interference Contrast (DIC) images were employed to define the boundaries of Hep3B cells so that pixels outside of the cell boundaries could be disregarded when calculating r-values. Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG.

Histone-Packaged pCB1, when Modified with a NLS and Delivered Via MC40-Targeted Protocells, Selectively Transfects Both Dividing and Non-Dividing HCC Cells with Nearly 100% Efficacy.

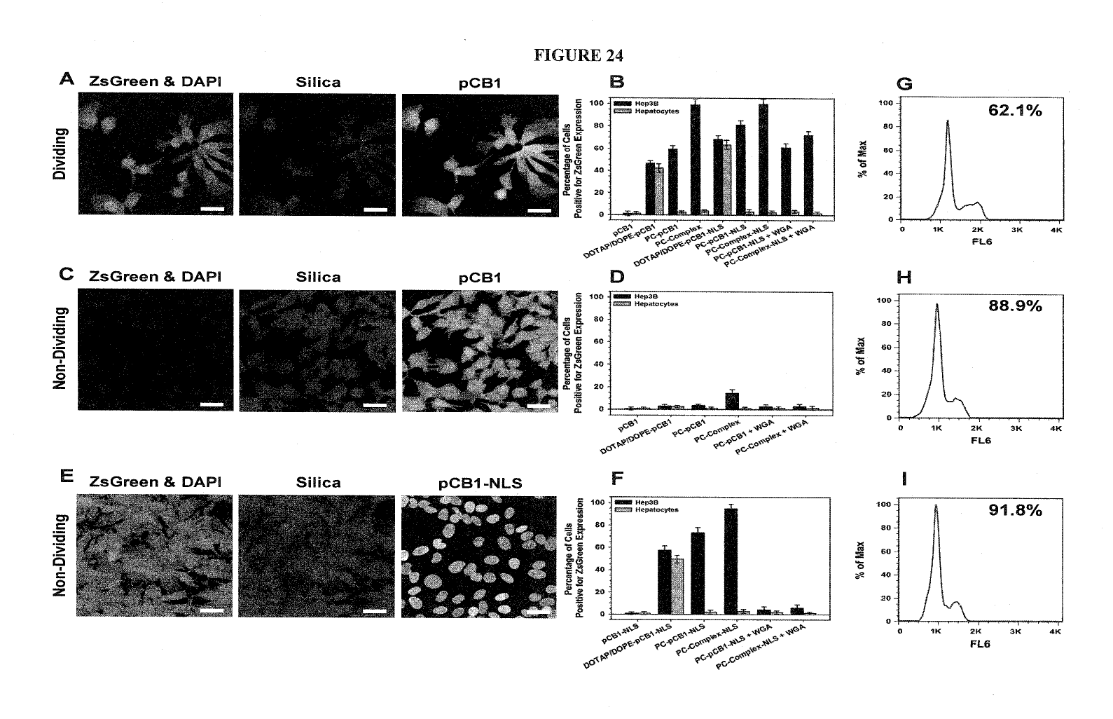

FIGS. 24 (A), (C), and (E) show confocal fluorescence microscopy images of Hep3B cells exposed to a 1000-fold excess of MC40-targeted, pCB1-loaded protocells for 24 hours at 37° C. Hep3B cells were dividing in (A) and ˜95% confluent in (C) and (E); pCB1 was pre-packaged with histones in all images, and the pCB1-histone complex was further modified with a NLS in (E). Silica cores were labeled with Alexa Fluor® 594 (red), pCB1 was labeled with Cy5 (white), and cell nuclei were counterstained with Hoechst 33342 (blue). Scale bars=20 μm. FIGS. 24(B), (D), and (F) show the percentage of 1×10⁶ Hep3B and hepatocytes that become positive for ZsGreen expression upon continual exposure to 1×10⁹ MC40-targeted, pCB1-loaded protocells (‘PC’) for 24 hours at 37° C. Cells were dividing in (B) and ˜95% confluent in (D) and (F); the x-axes indicate whether CB1 plasmids (‘pCB1’) and pCB1-histone complexes (‘complex’) were modified with the NLS. pCB1 alone, as well as pCB1 packaged with a 1:1 (w/w) mixture of DOTAP and DOPE were employed as controls. Cells were exposed to 20 mg/mL of wheat germ agglutinin (WGA) to block translocation of NLS-modified pCB1 through the nuclear pore complex. Error bars represent 95% confidence intervals (1.96σ) for n=3. FIGS. 24(G)-(I) Cell cycle histograms for cells employed in Figures (A), (C), and (E), respectively. The percentage of cells in G₀/G₁ phase is given for each histogram. In all experiments, protocell SLBs were composed of DOPC with 5 wt % DOPE, 30 wt % cholesterol, and 10 wt % PEG-2000 (18:1) and were modified with 0.015 wt % MC40 and 0.500 wt % H5WYG.

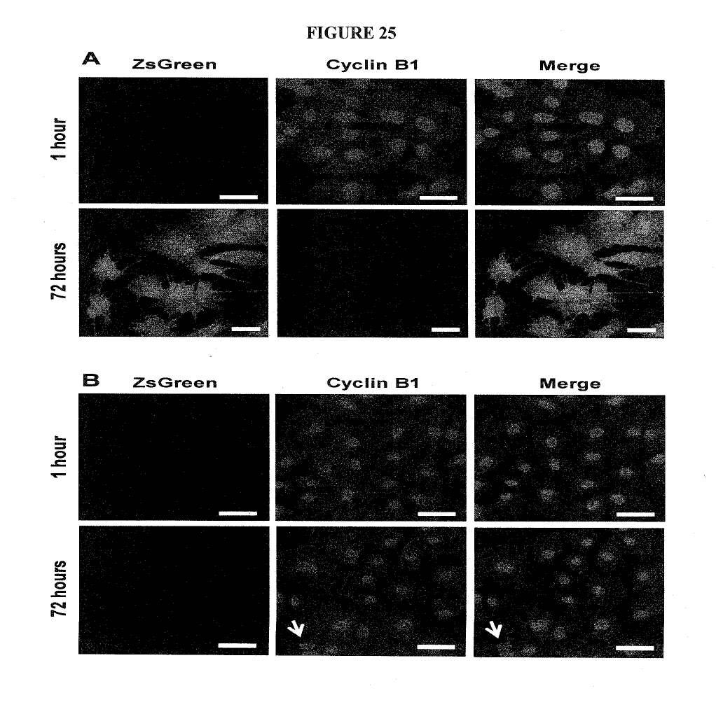

FIG. 25 shows the confocal fluorescence microscopy images of Hep3B (A) and hepatocytes (B) that were exposed to MC40-targeted, pCB1-loaded protocells for either 1 hour or 72 hours at 37° C.; the pCB1 concentration was maintained at 5 pM in all experiments. The arrows in (B) indicate mitotic cells. Cyclin B1 was labeled with an Alexa Fluor® 594-labeled monoclonal antibody (red), and cell nuclei were stained with Hoechst 33342 (blue). Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG. All scale bars=20 μm.

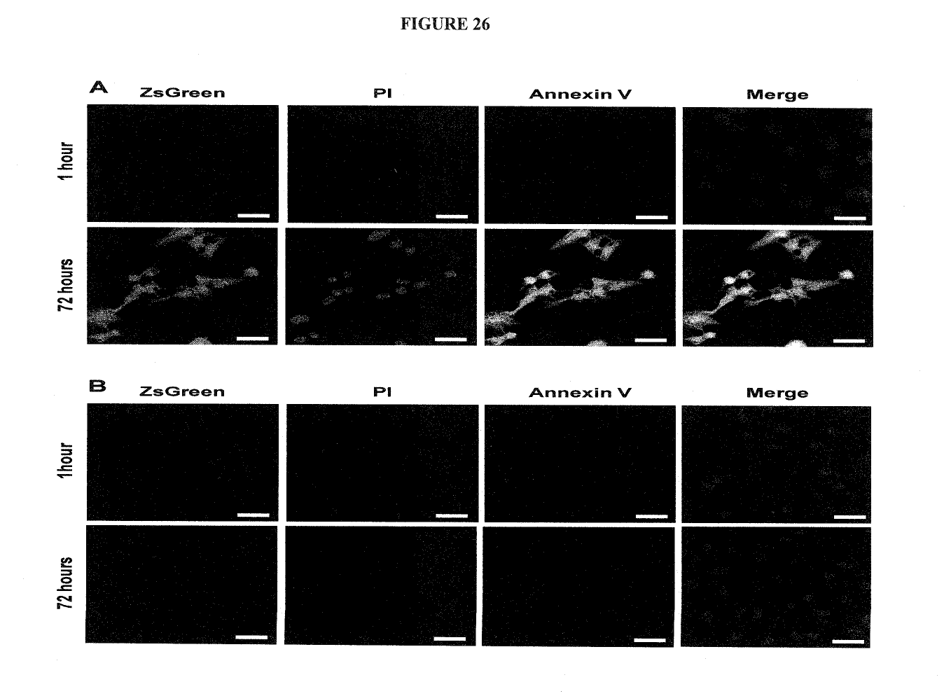

FIG. 26 shows the confocal fluorescence microscopy images of Hep3B (A) and hepatocytes (B) that were exposed. to MC40-targeted, pCB1-loaded protocells for either 1 hour or 72 hours at 37° C.; the pCB1 concentration was maintained at 5 pM in all experiments. Cells were stained with Alexa Fluor® 647-labeled annexin V (white) and propidium iodide (red) to assay for early and late apoptosis, respectively, and cell nuclei were counterstained with Hoechst 33342 (blue). Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG. All scale bars=20 μm.

Protocells with a SLB Composed of Zwitterionic Lipids Induce Minimal Non-Specific Cytotoxicity.

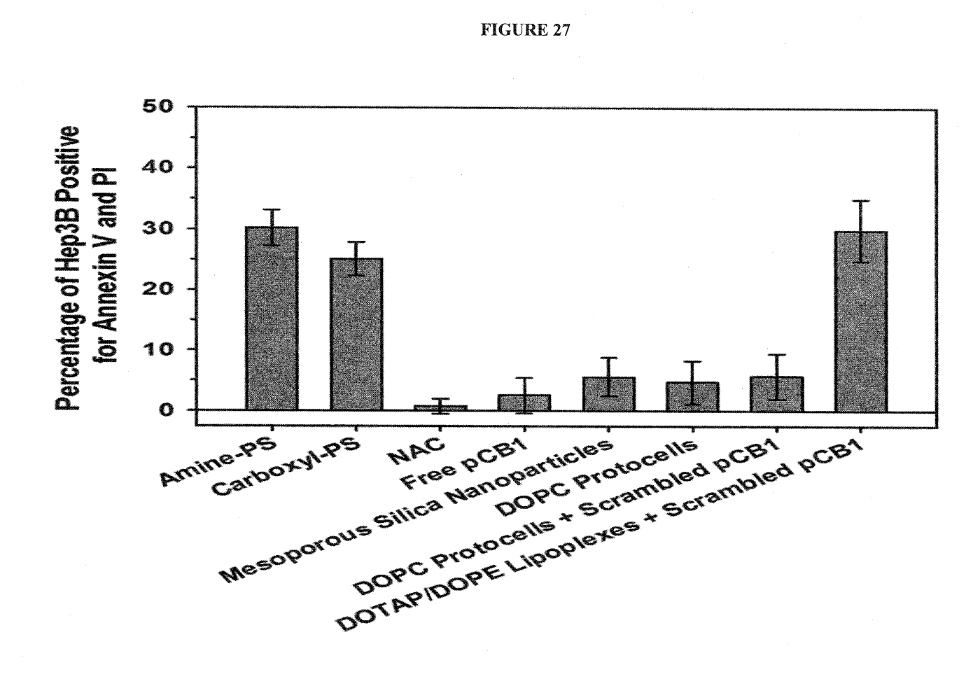

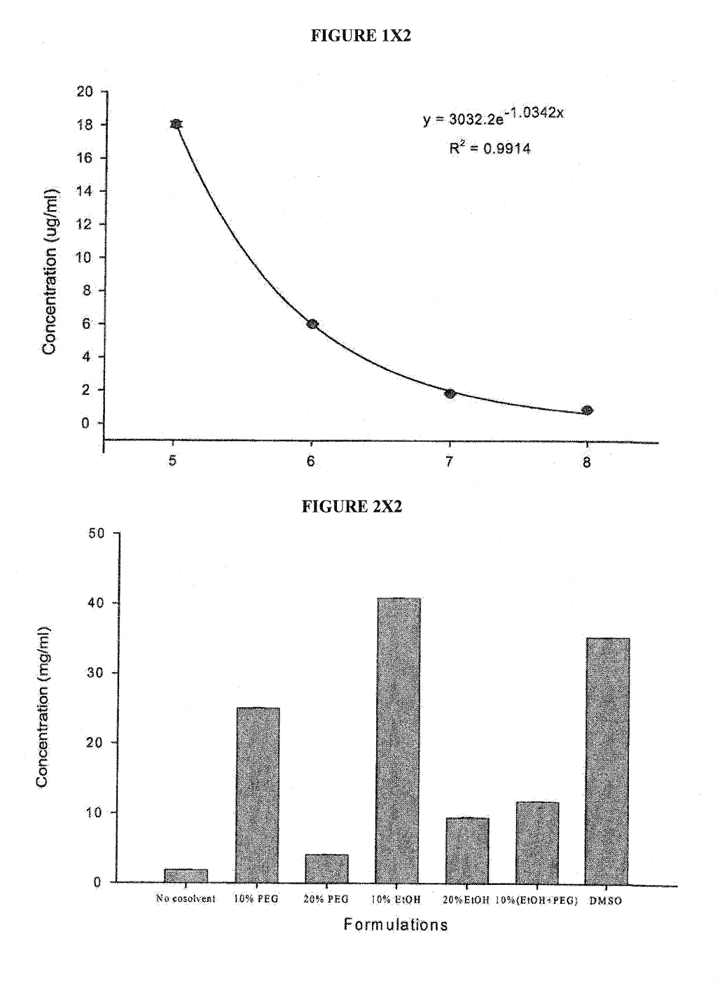

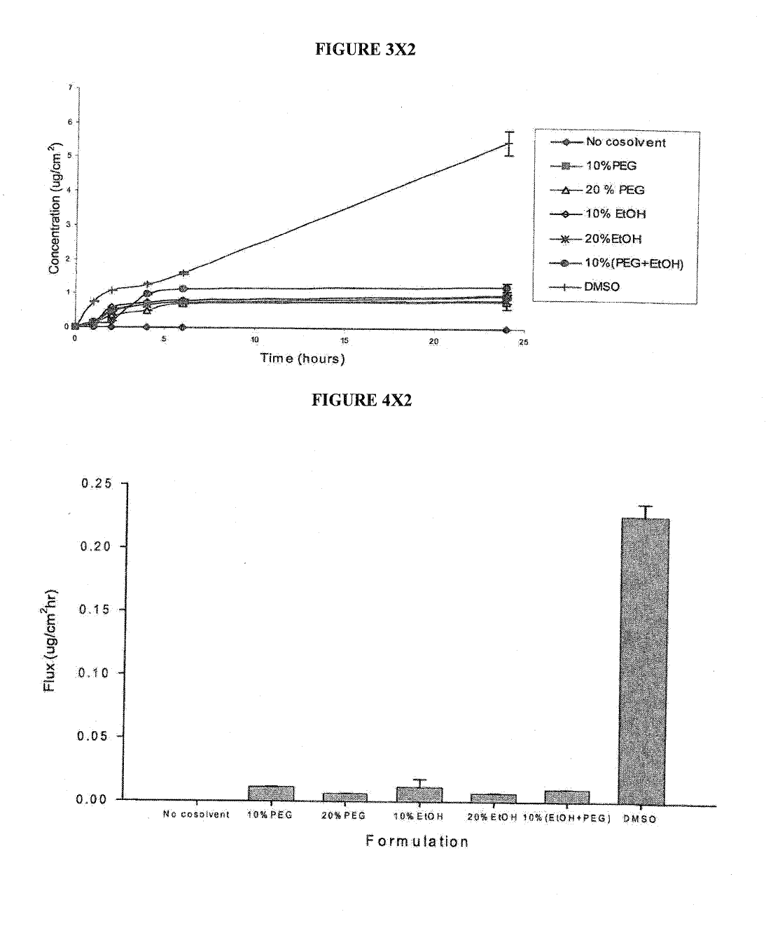

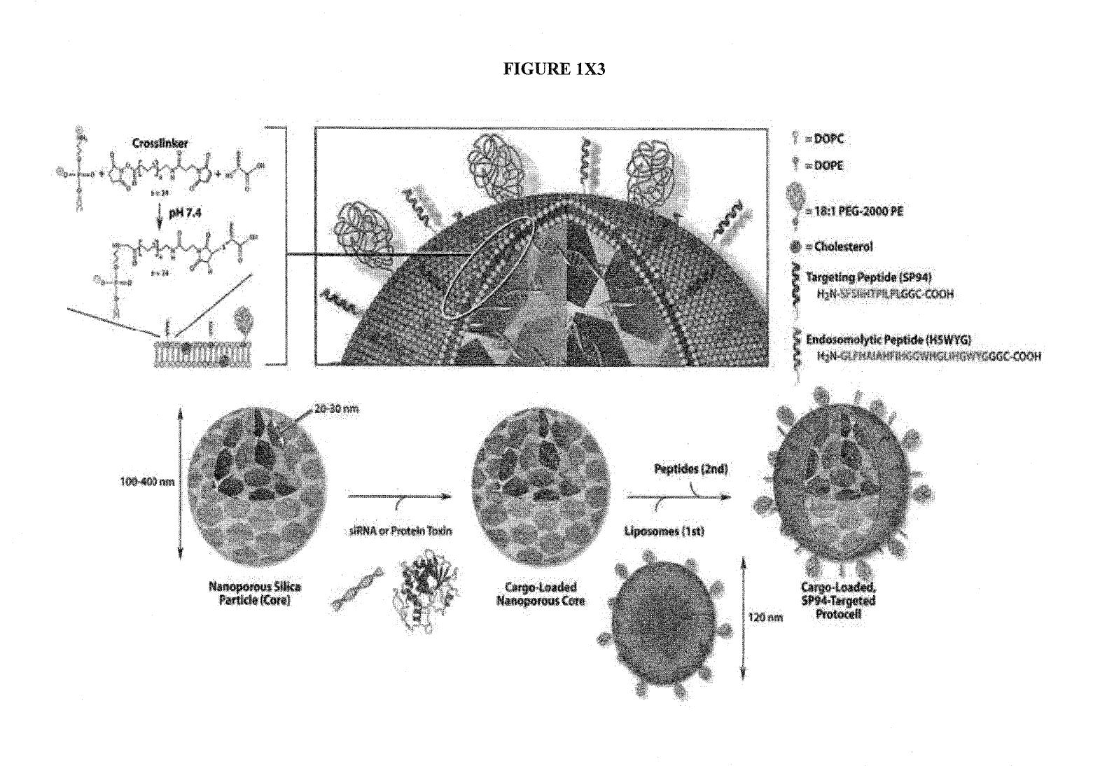

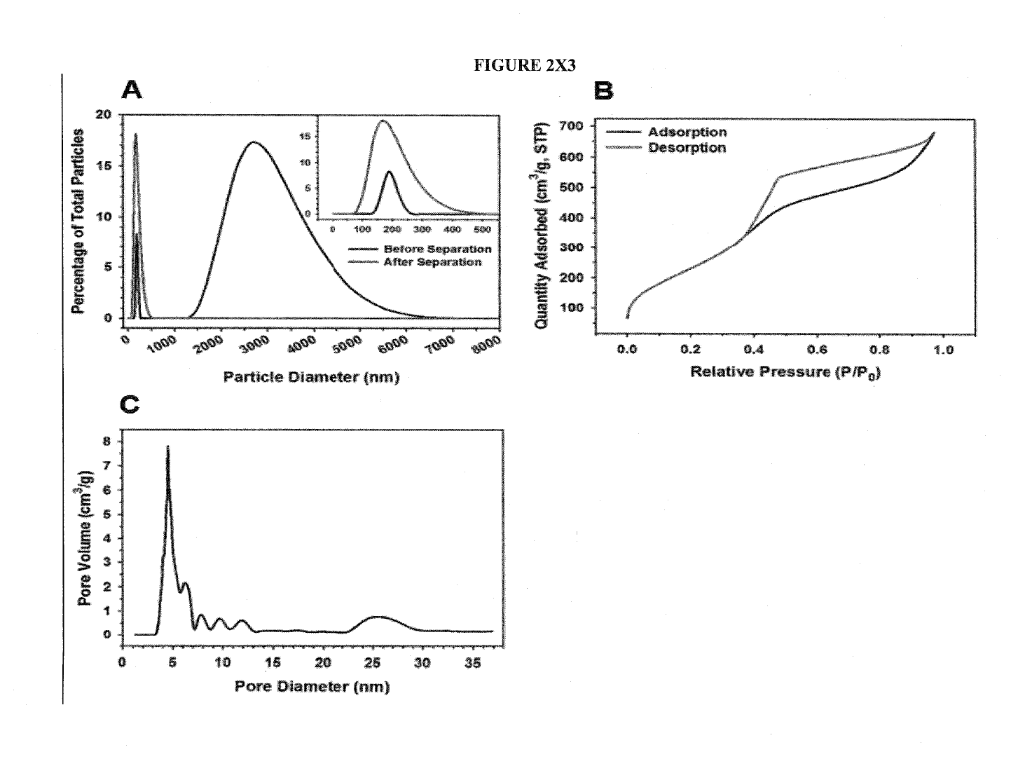

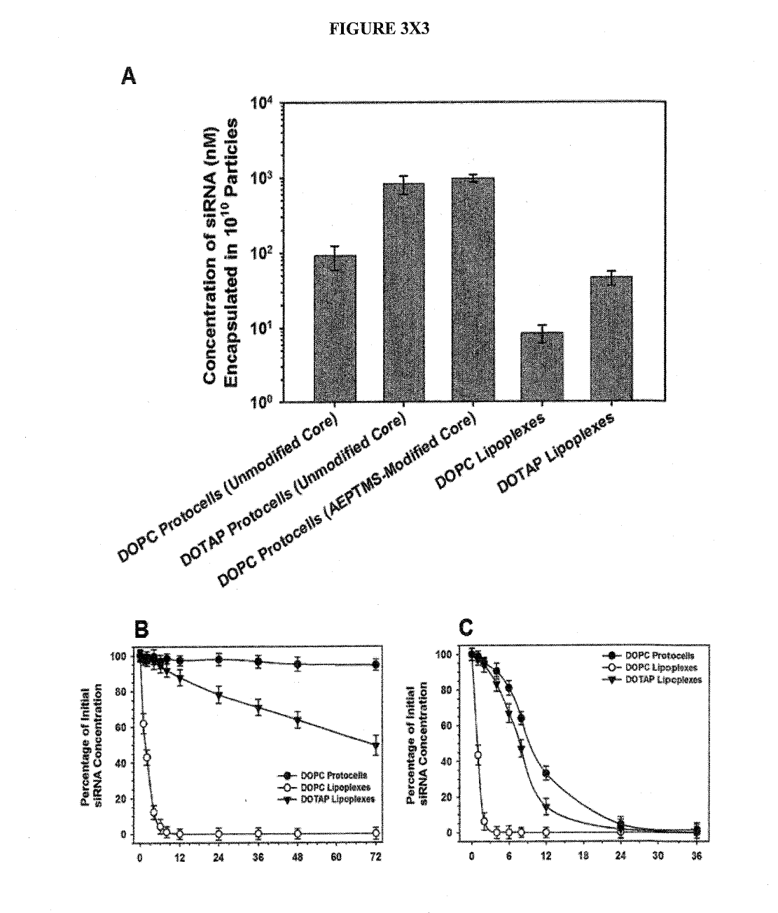

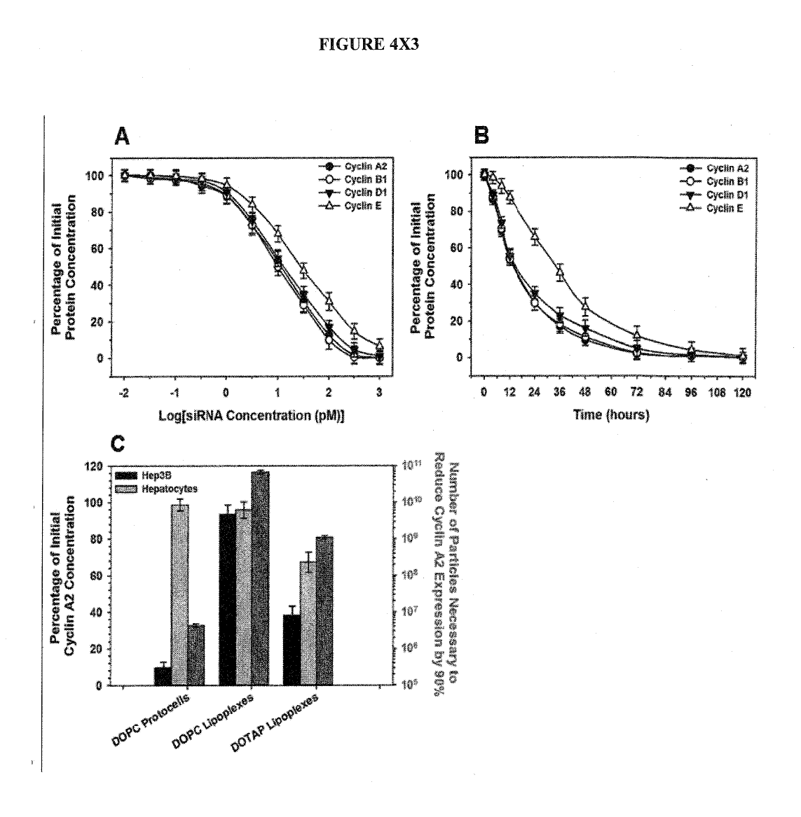

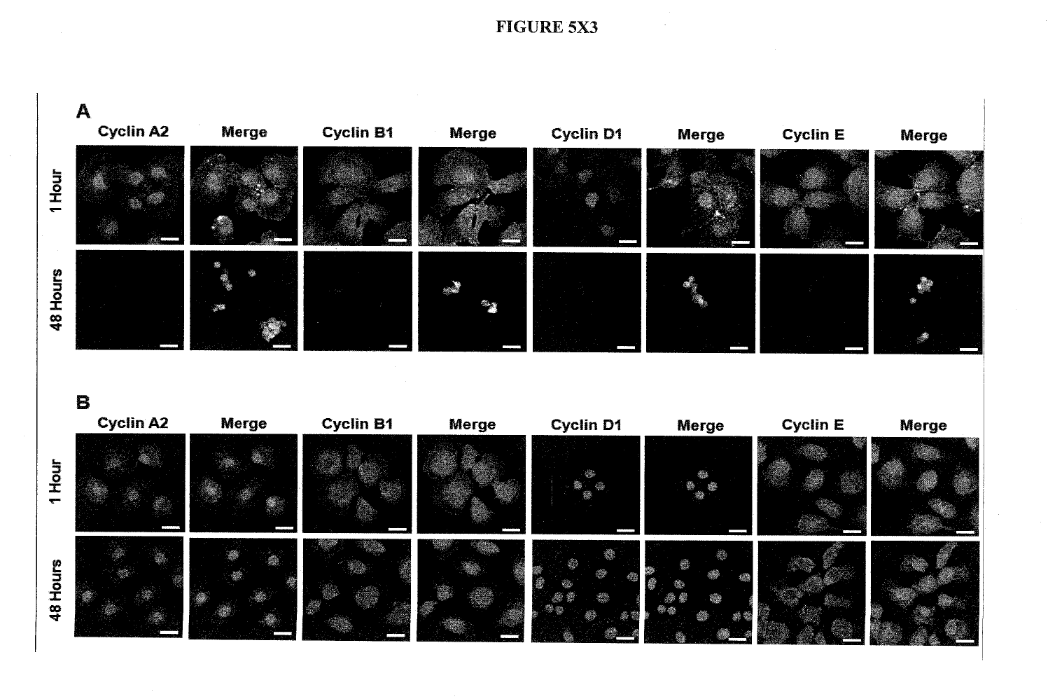

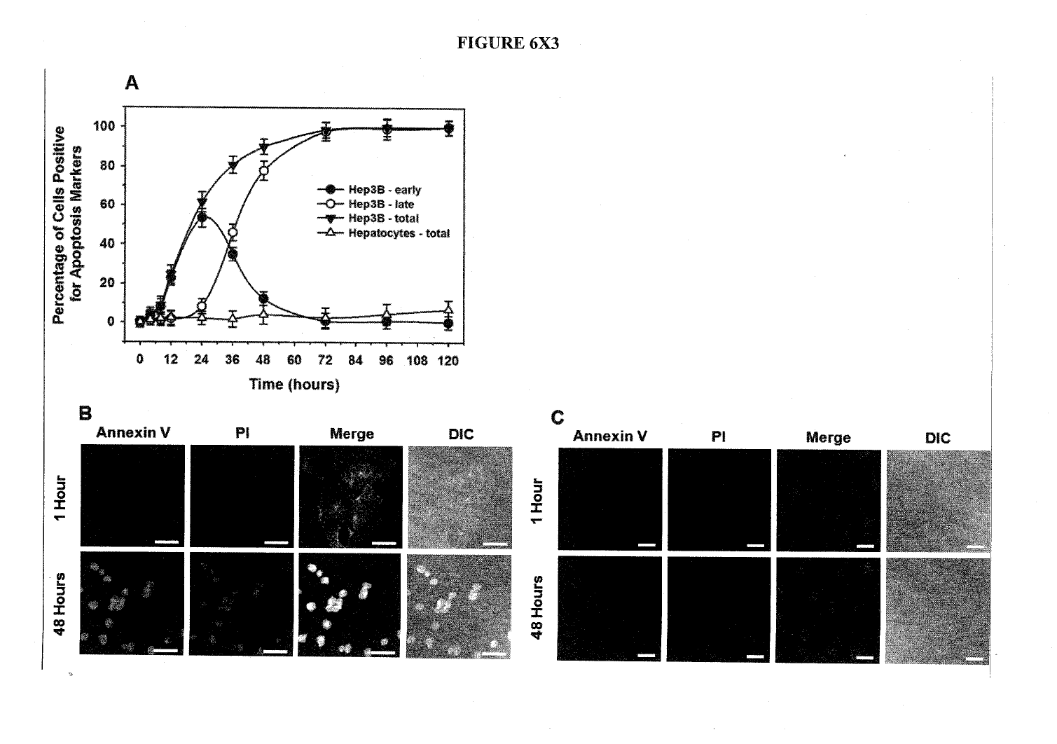

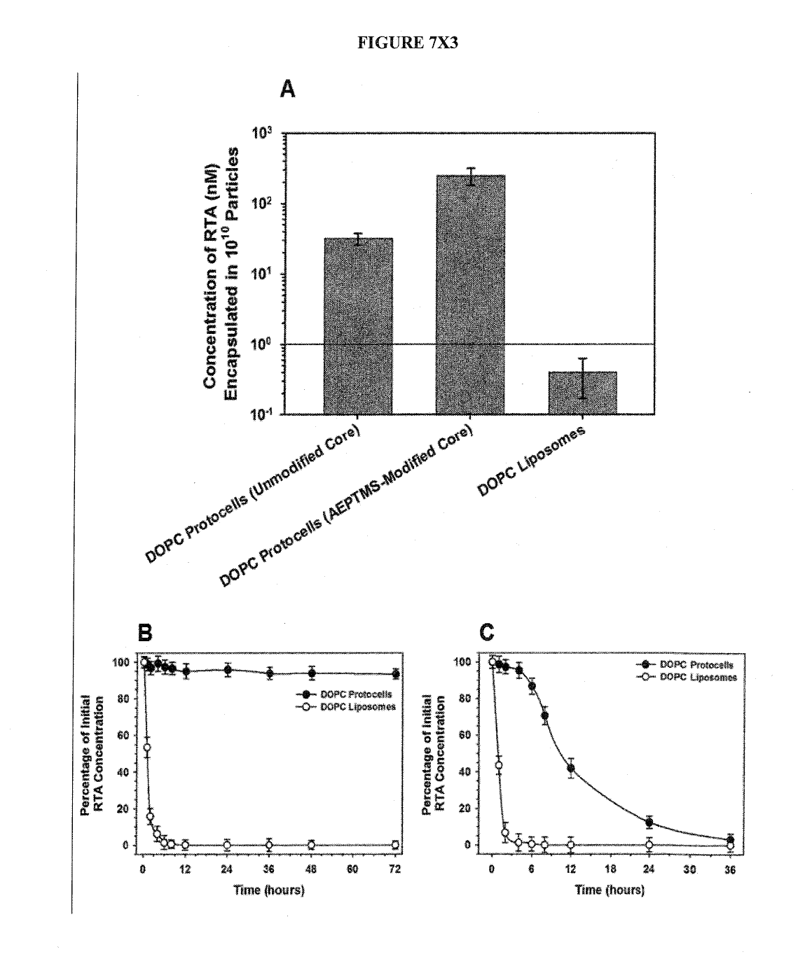

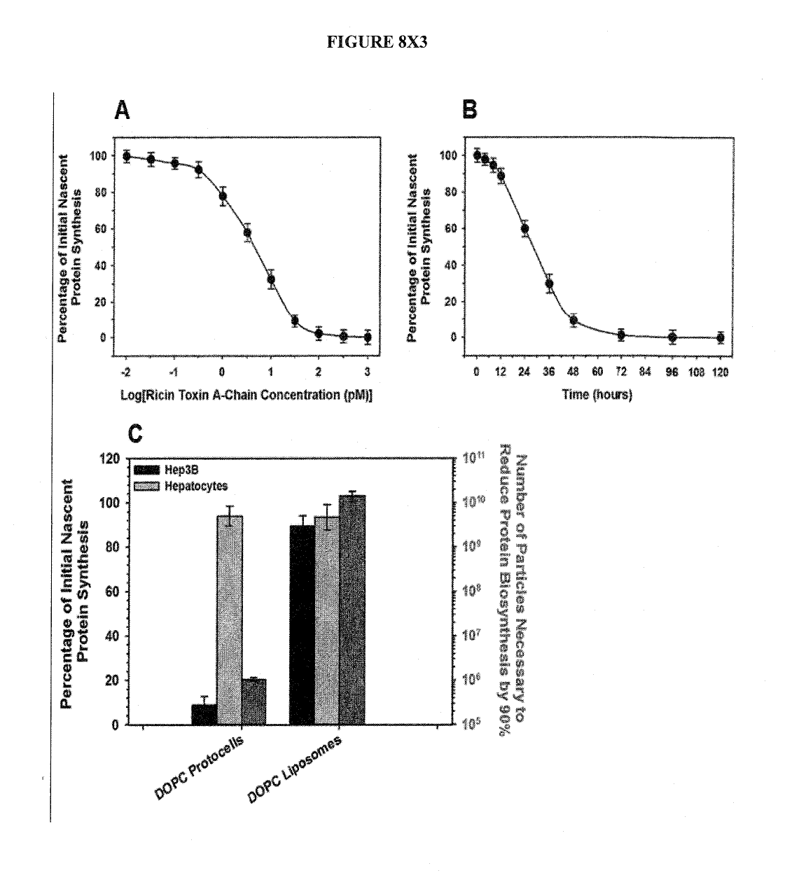

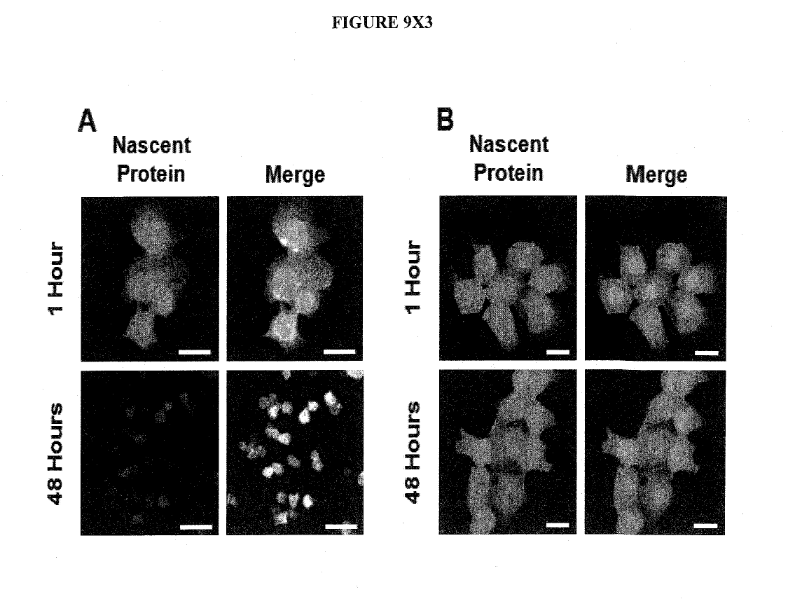

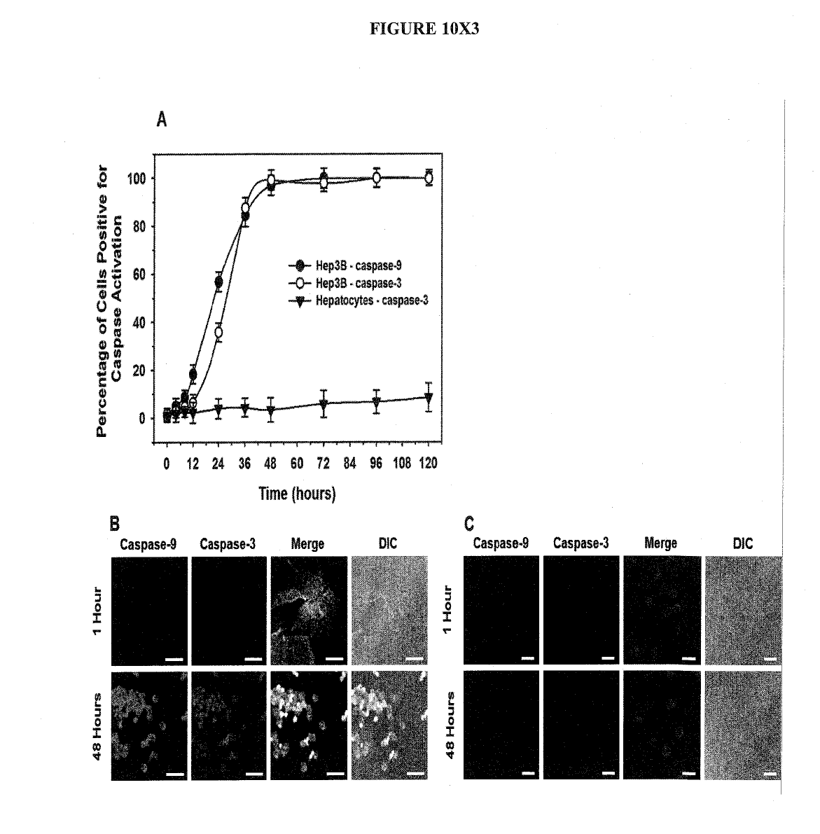

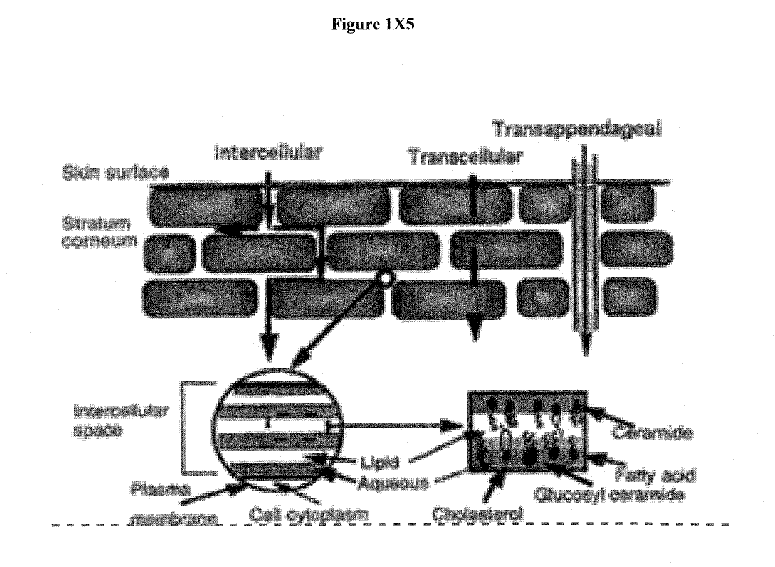

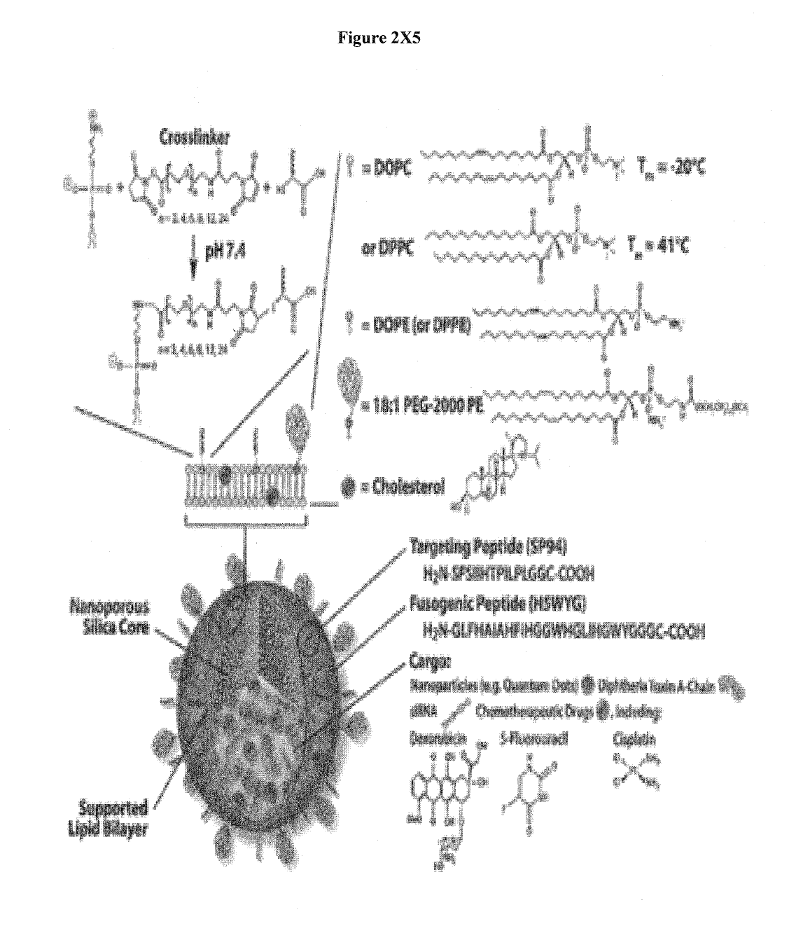

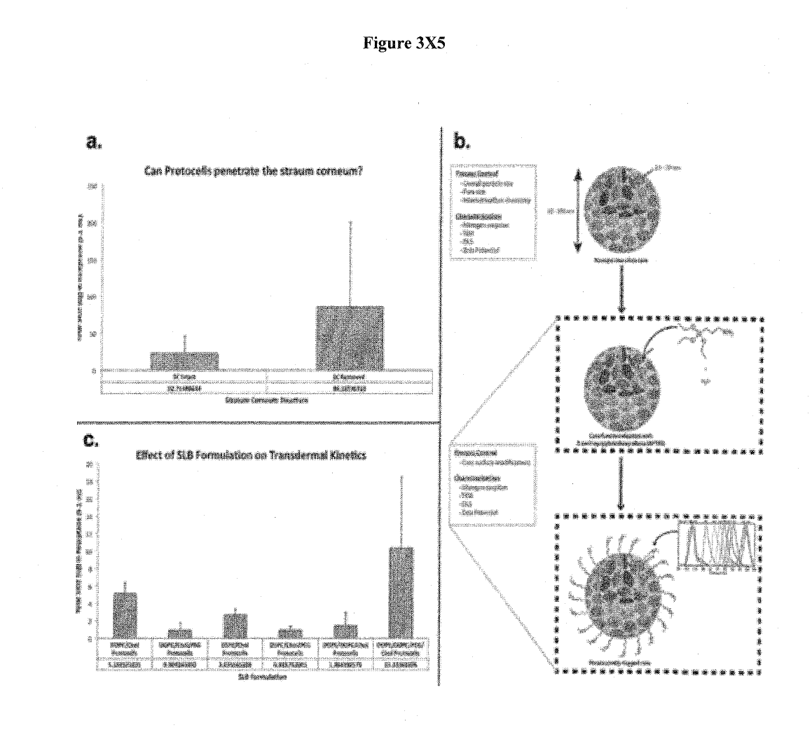

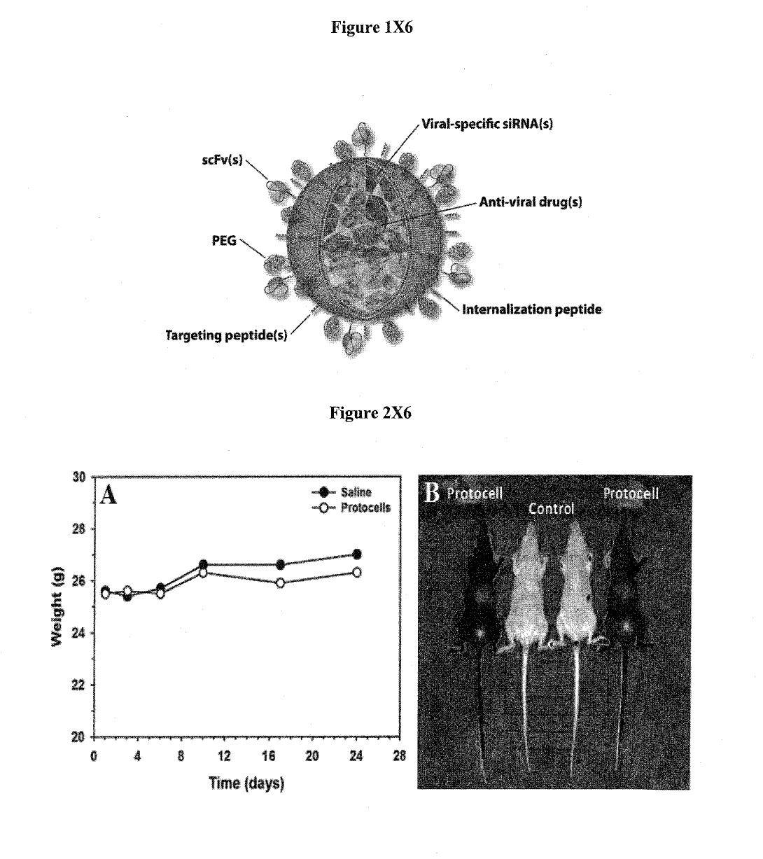

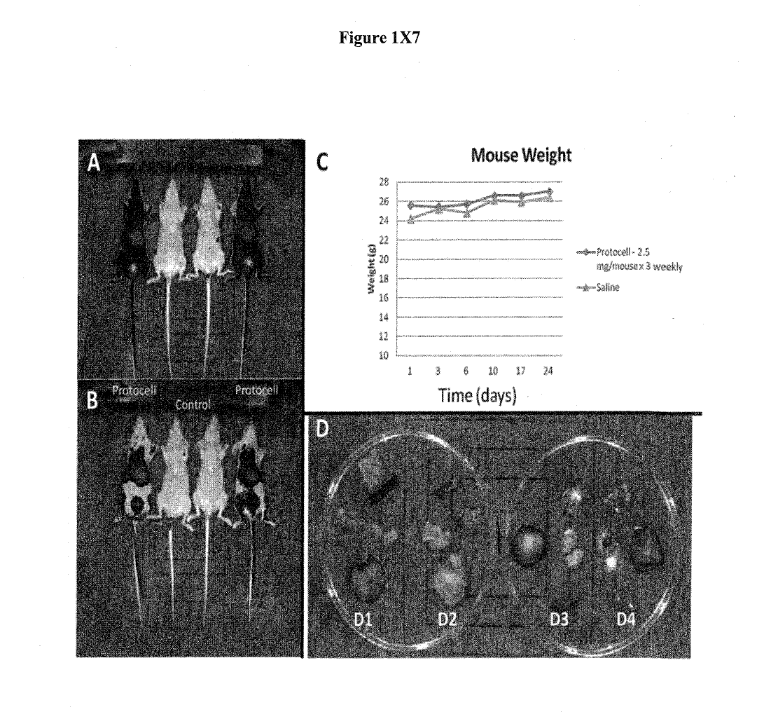

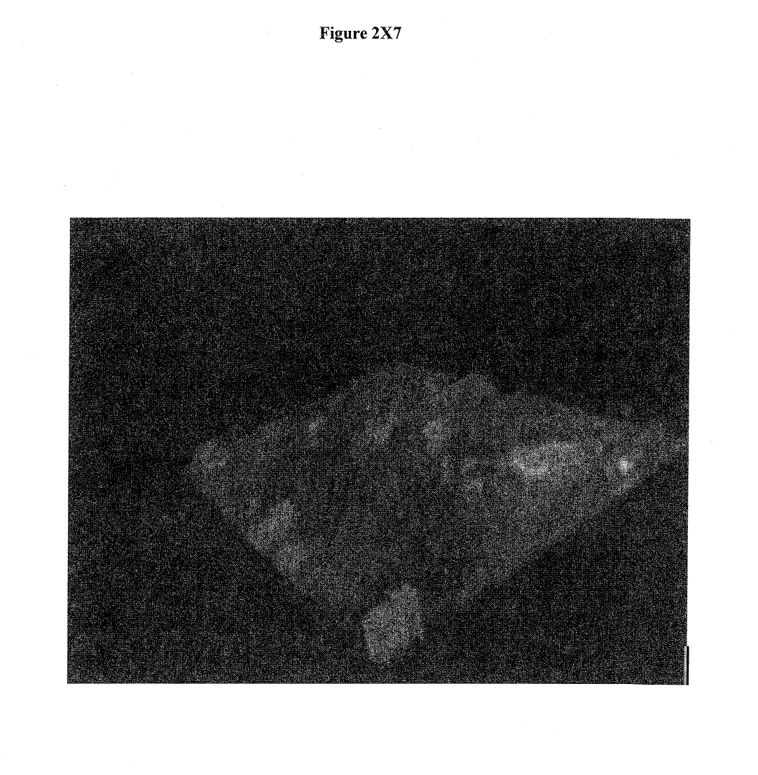

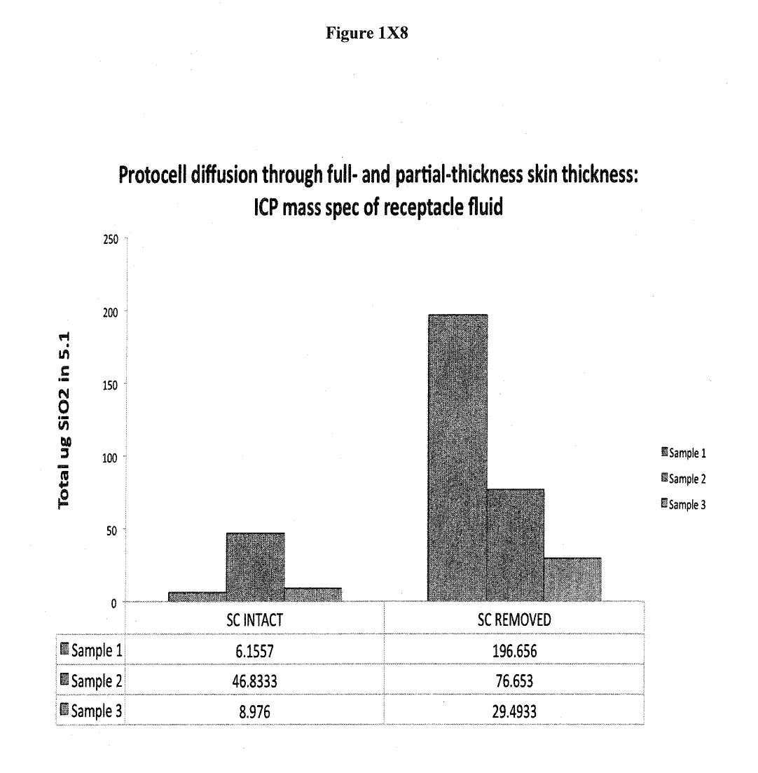

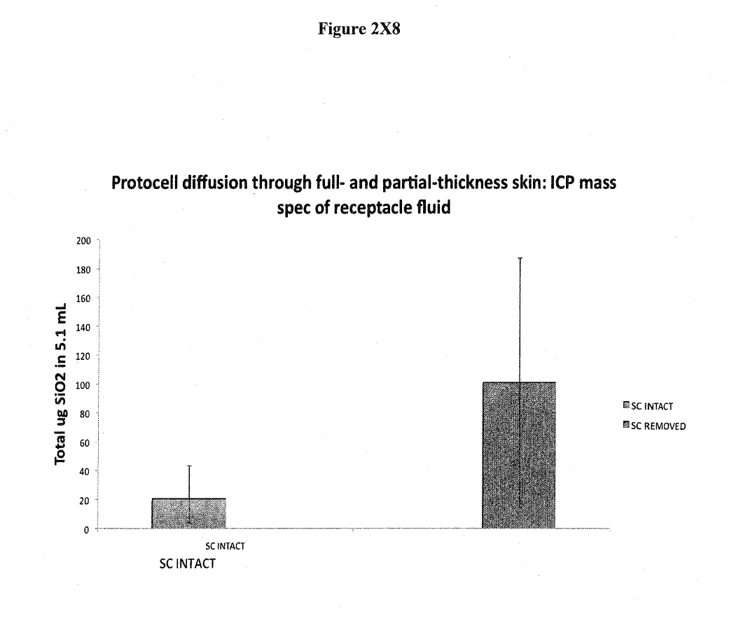

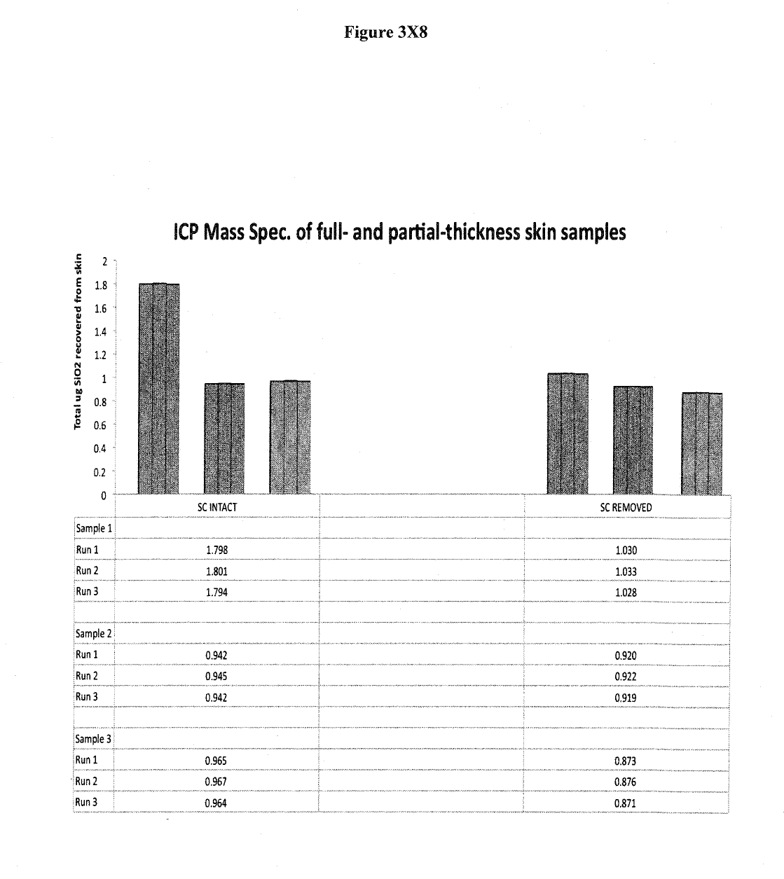

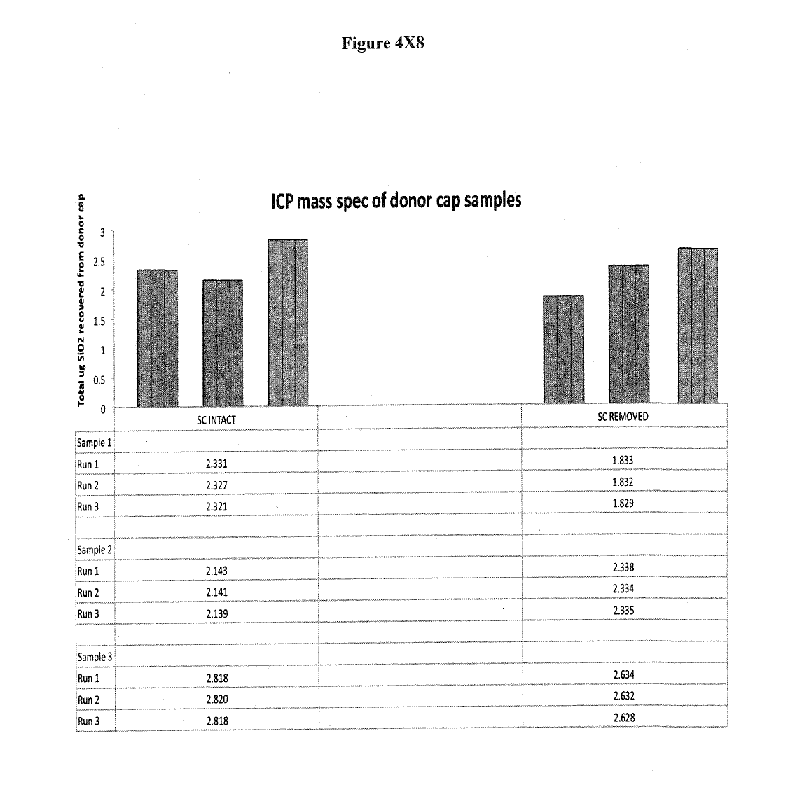

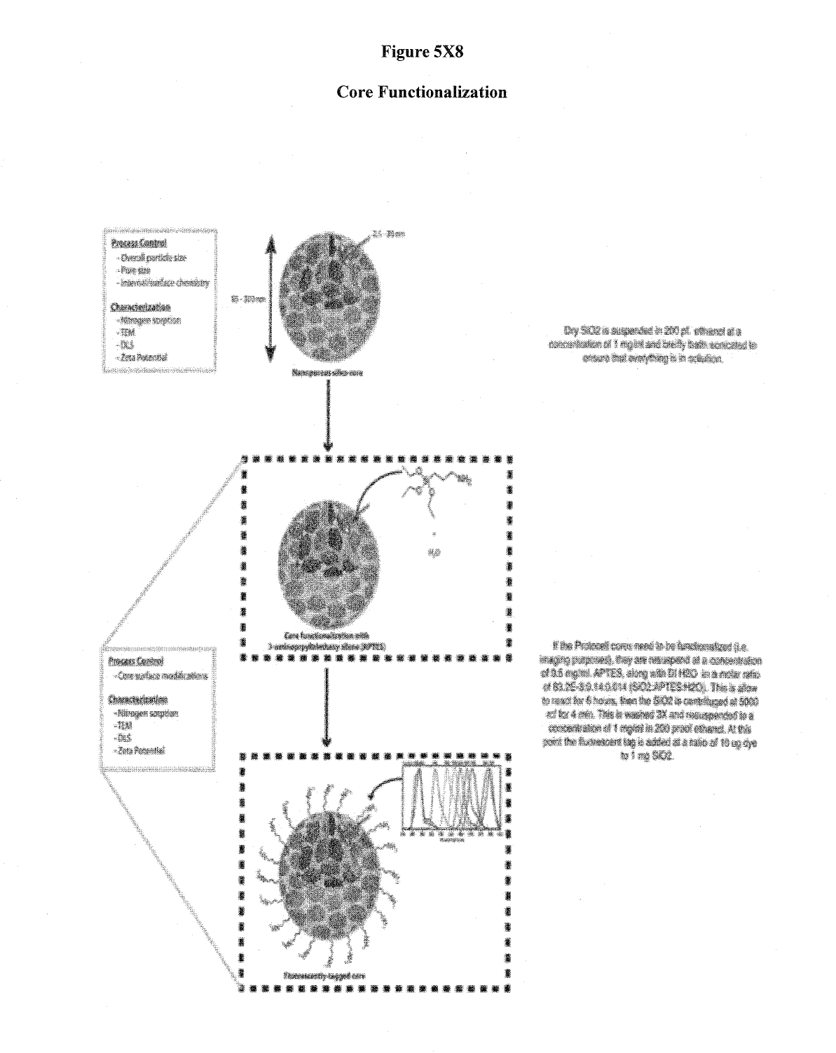

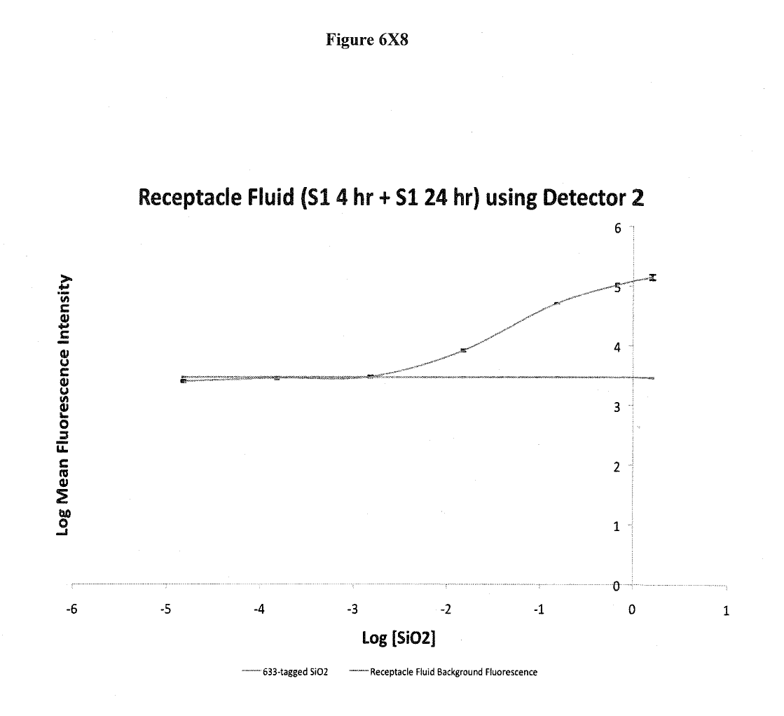

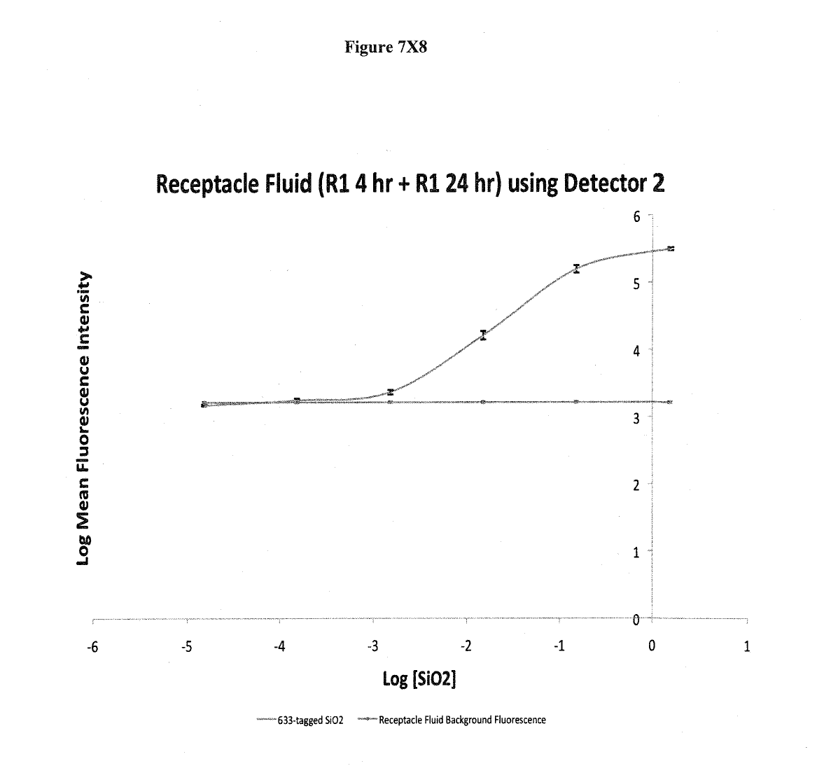

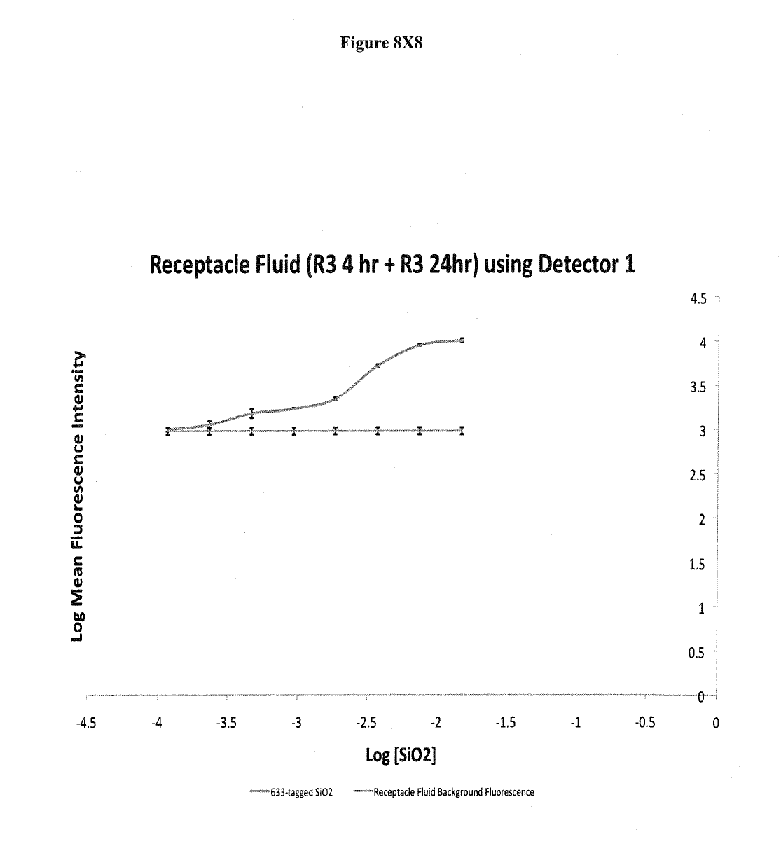

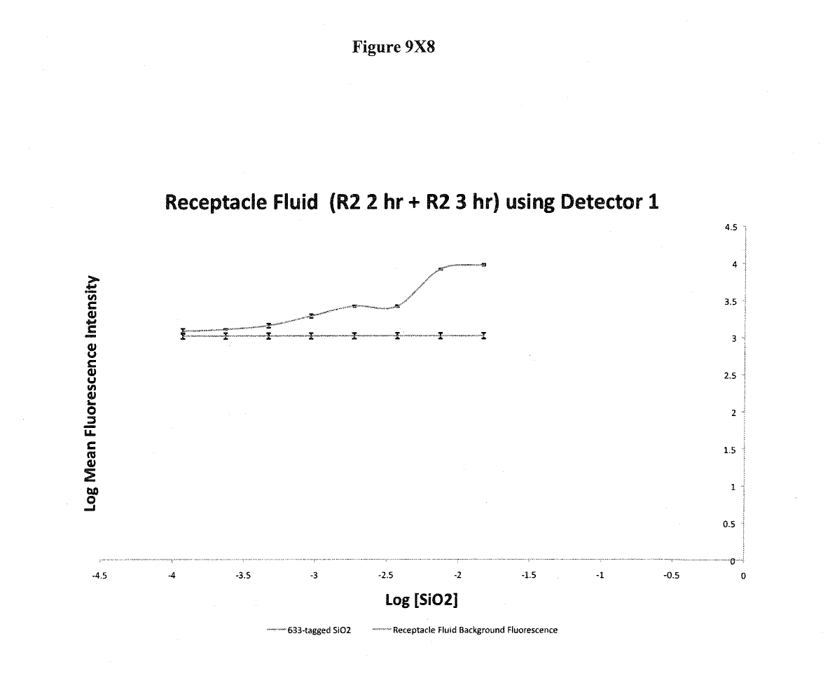

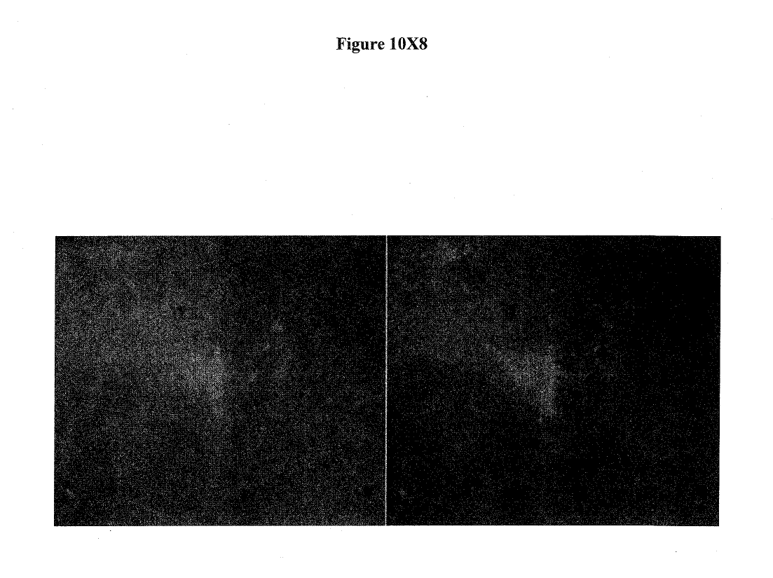

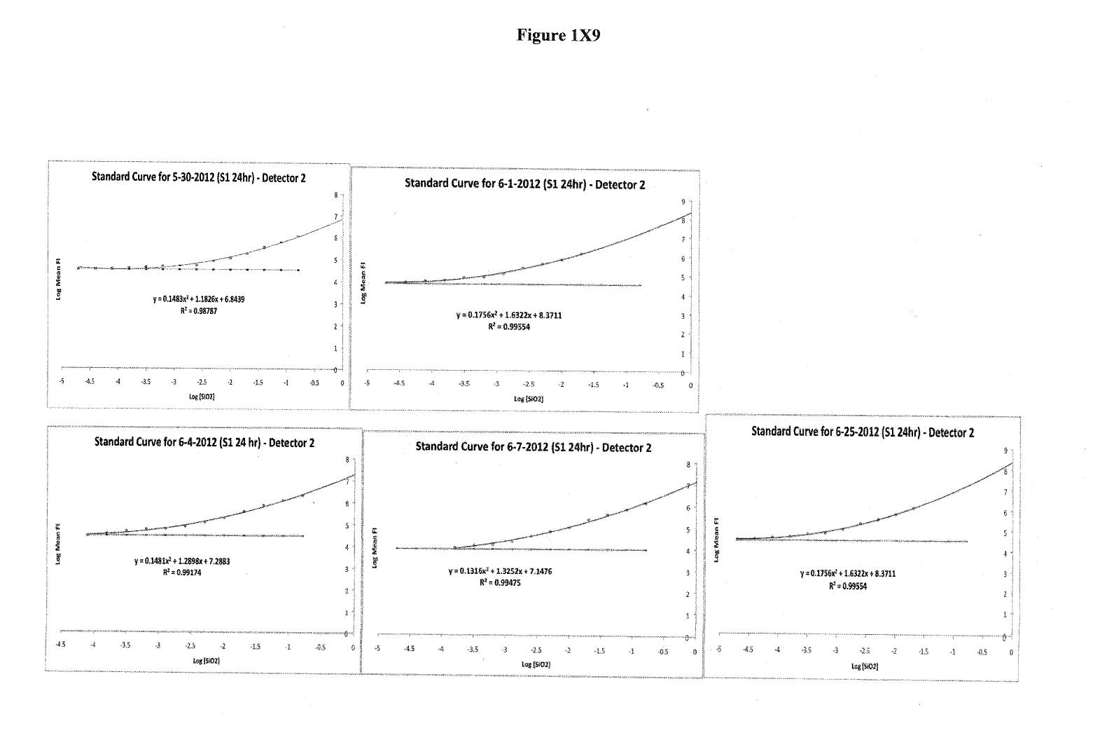

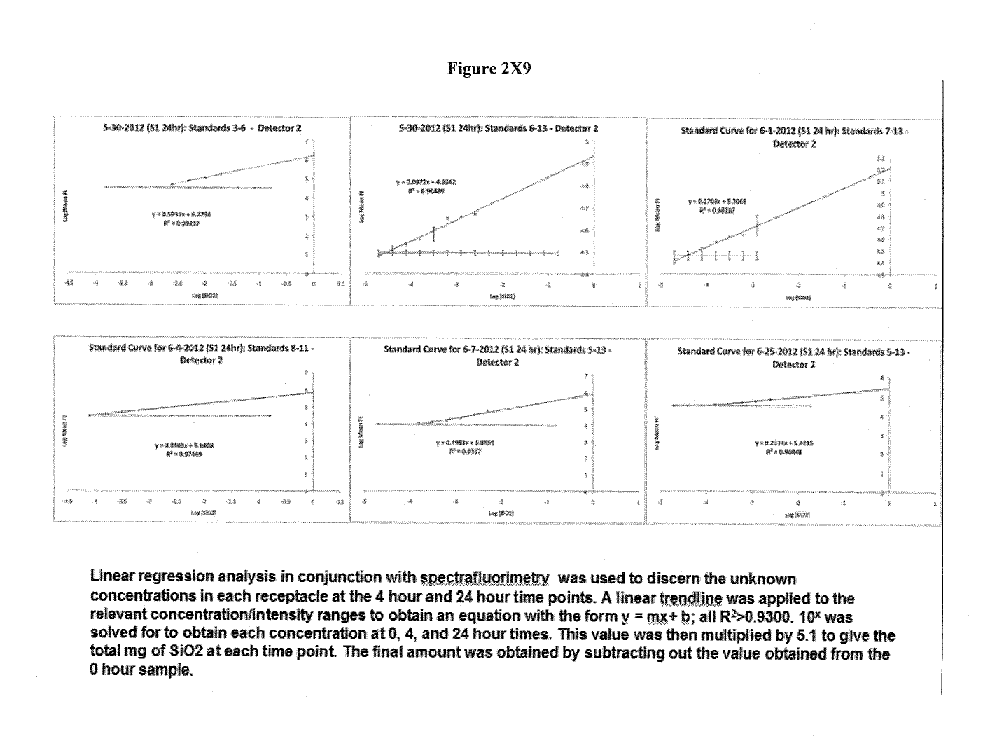

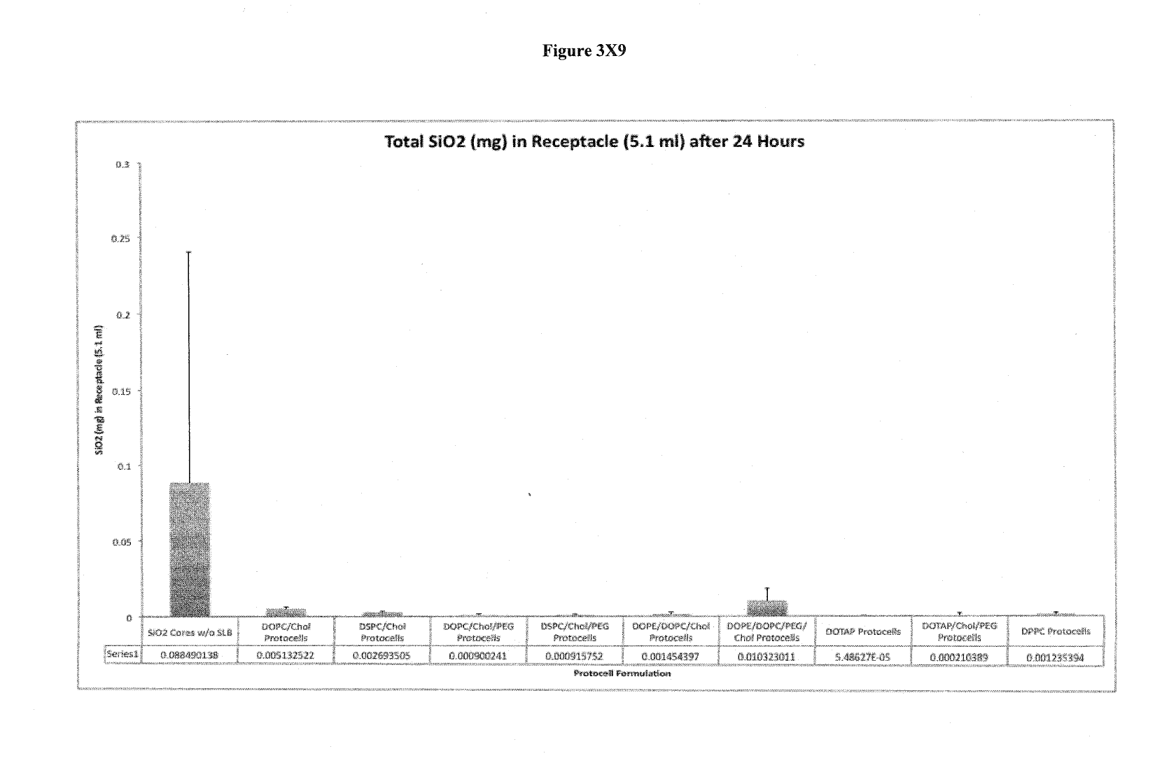

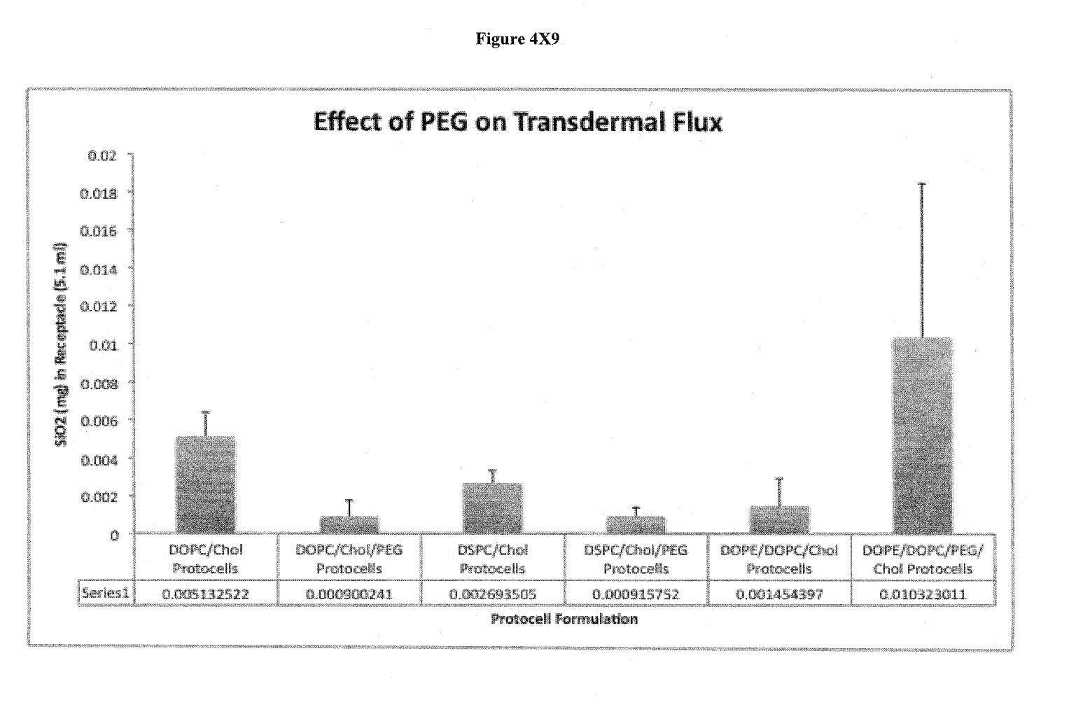

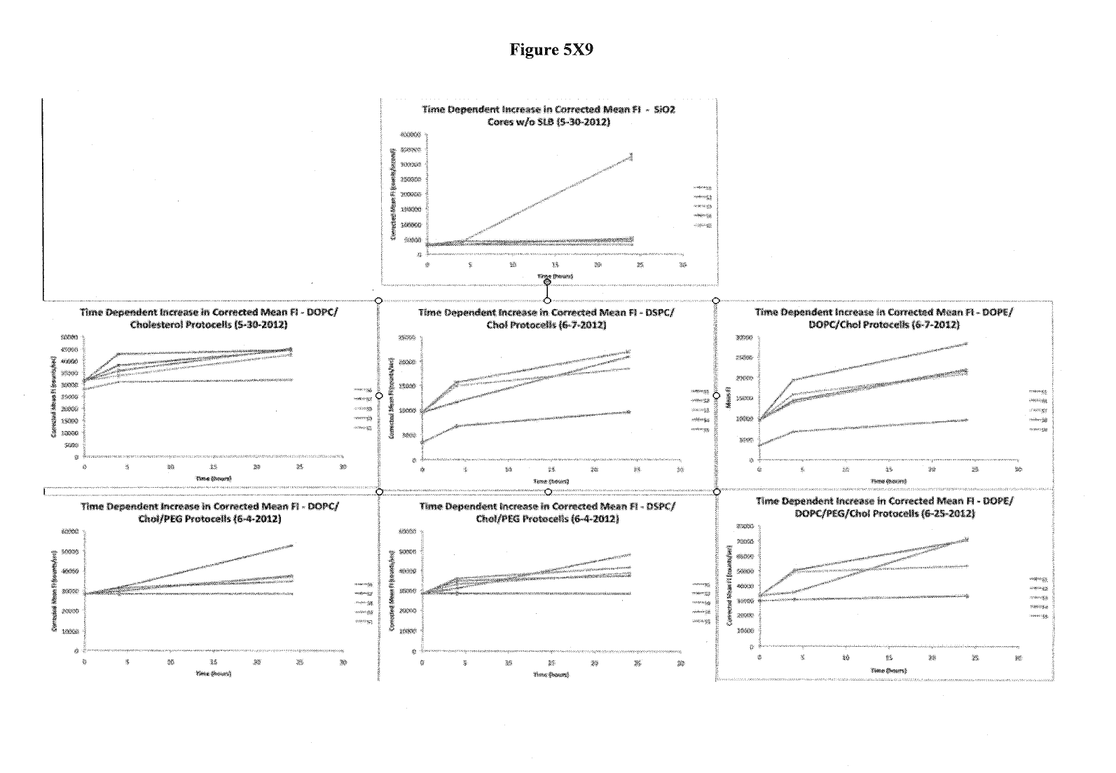

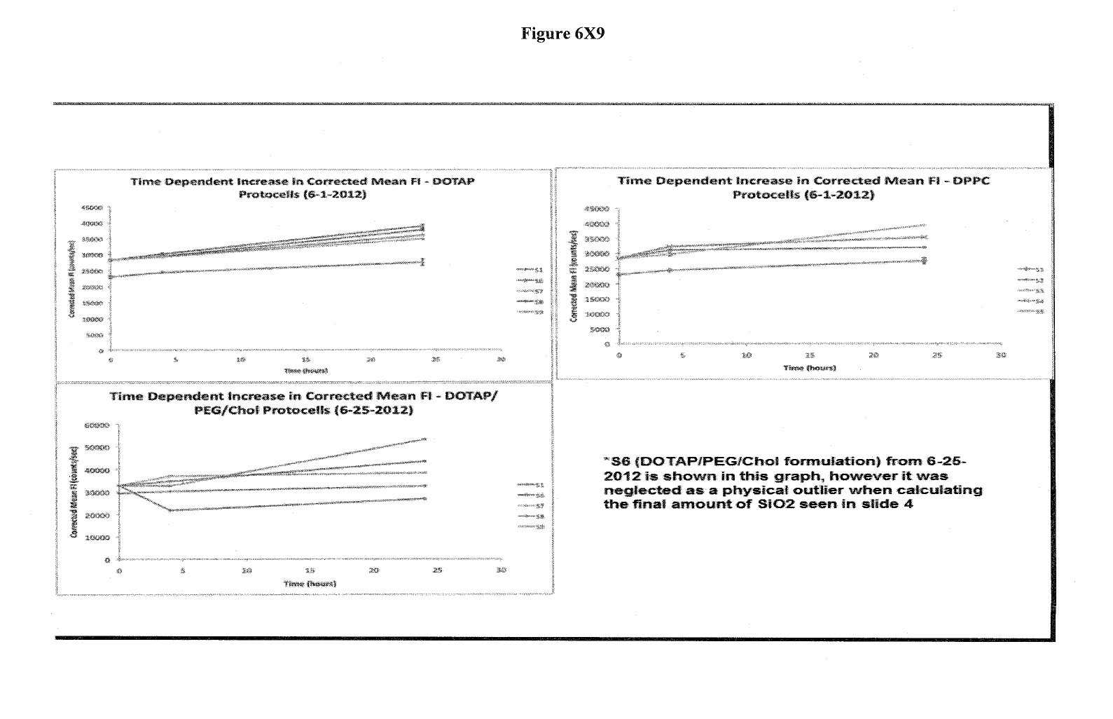

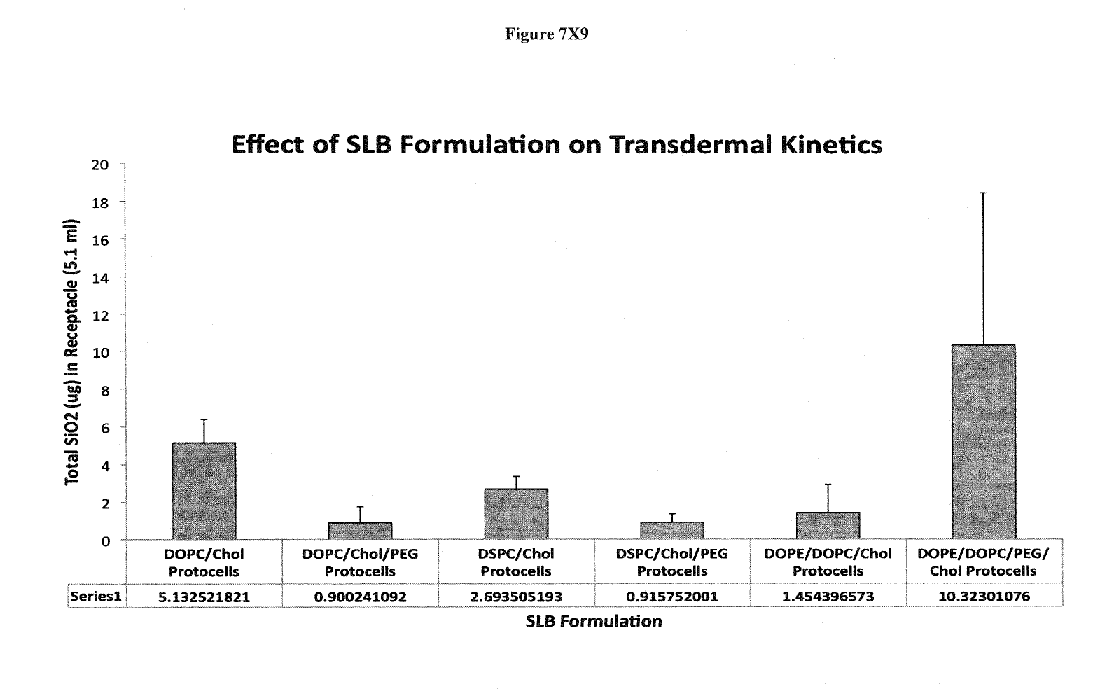

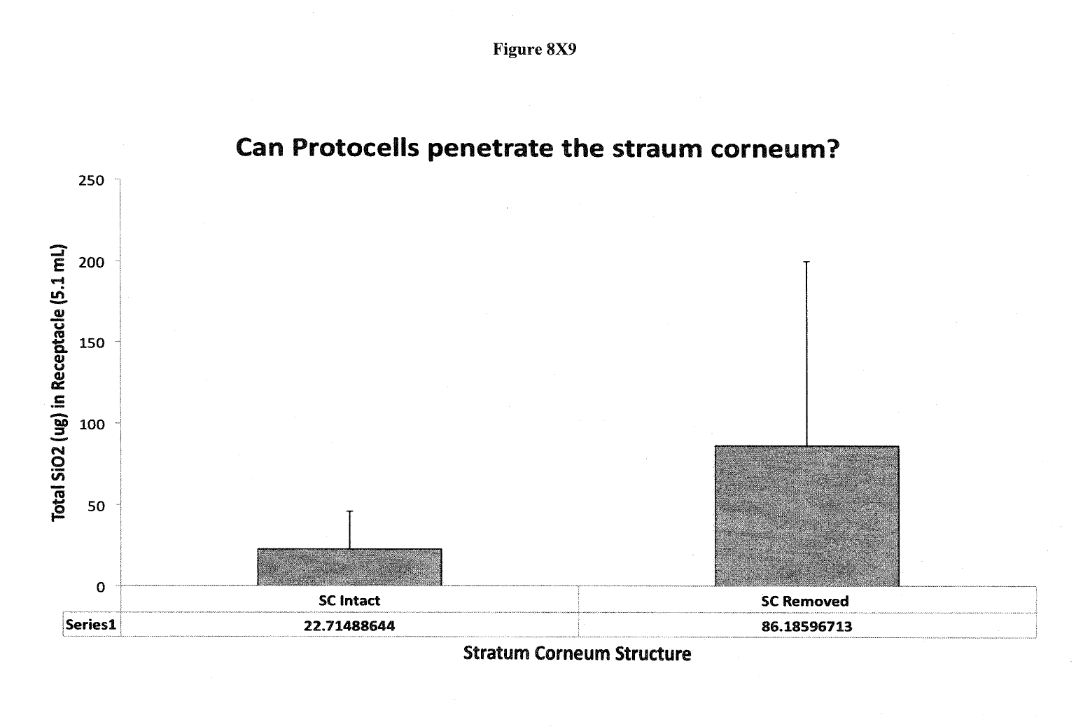

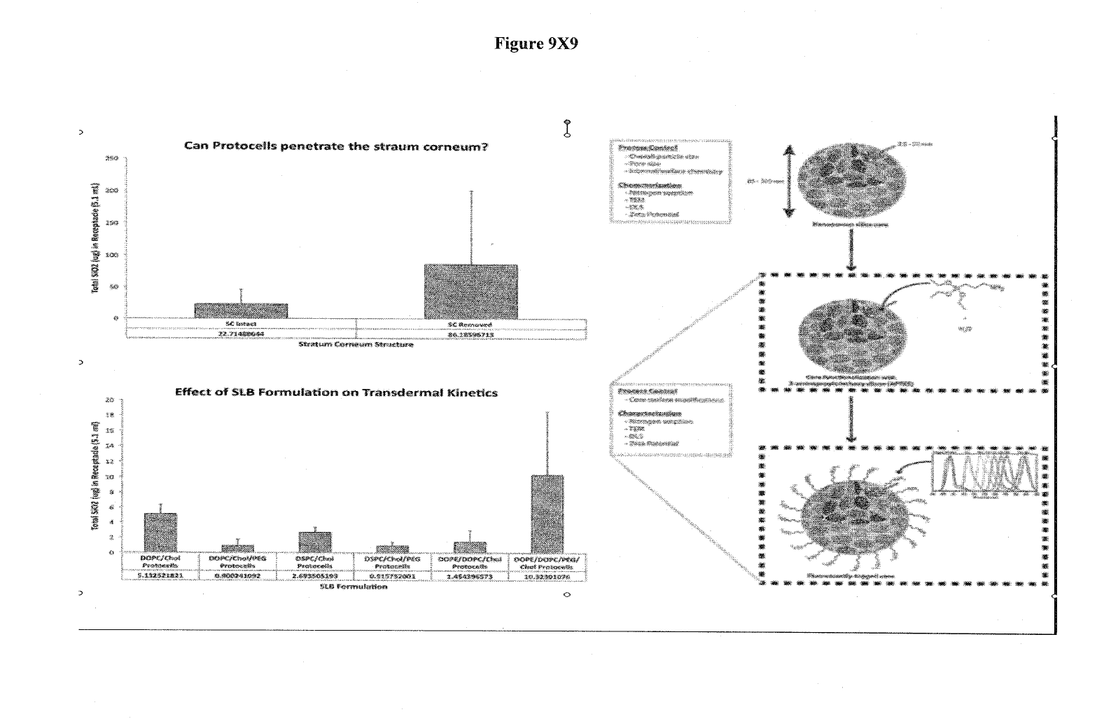

As depicted in attached FIG. 27, the percentage of 1×10⁶ Hep3B that become apoptotic upon continual exposure to 1×10⁹ APTES-modified mesoporous silica nanoparticles, DOPC protocells with APTES-modified cores, DOPC protocells loaded with a plasmid that encodes a scrambled shRNA sequence (‘scrambled pCB1’), or DOTAP/DOPE (1:1 w/w) lipoplexes loaded with scrambled pCB1 for 48 hours at 37° C. Protocells and lipoplexes were modified with 10 wt % PEG-2000, 0.015 wt % MC40, and 0.500 wt % H5WYG. Positively- and negatively-charged polystyrene nanoparticles (‘amine-PS’ and ‘Carboxyl-PS’, respectively) were employed as positive controls, while Hep3B exposed to 10 mM of the antioxidant, N-acetylcysteine (NAC), or to 1 pmol of free pCB1 were used as negative controls. All error bars represent 95% confidence intervals (1.96σ) for n=3.

All references which are disclosed herein are incorporated by reference where relevant.

### REFERENCES FOR EXAMPLE 1

1 Carroll, N. J., Pylypenko, S., Atanassov, P. B. & Petsev, D. N. Microparticles with Bimodal Nanoporosity Derived by Microemulsion Templating. Langmuir, doi:10.1021/1a900988j (2009).
2 Lu, Y. F. et al. Aerosol-assisted self-assembly of mesostructured spherical nanoparticles. Nature 398, 223-226 (1999).
3 Iler, R. K. The Chemistry of Silica: Solubility, Polymerization, Colloid and Surface Properties, and Biochemistry. (John Wiley and Sons, 1979).
4 Doshi, D. A. et al. Neutron Reflectivity Study of Lipid Membranes Assembled on Ordered Nanocomposite and Nanoporous Silica Thin Films, Langmuir 21, 2865-2870, doi:10.1021/1a0471240 (2005).
5 Bernhard, M. I. et al. Guinea Pig Line 10 Hepatocarcinoma Model: Characterization of Monoclonal Antibody and in Vivo Effect of Unconjugated Antibody and Antibody Conjugated to Diphtheria Toxin A Chain. Cancer Research 43, 4420-4428 (1983).
6 Lo, A., Lin, C. T. & Wu, H. C. Hepatocellular carcinoma cell-specific peptide ligand for targeted drug delivery. Molecular Cancer Therapeutics 7, 579-589, doi:10.1158/1535-7163.mct-07-2359 (2008).
7 Sciot, R. et al. Transferrin receptor expression in human hepatocellular carcinoma: an immunohistochemical study of 34 cases. Histopathology 12, 53-63 (1988).
8 Kannangai, R., Sahin, F. & Torbenson, M. S. EGFR is phosphorylated at Ty845 in hepatocellular carcinoma. Mod Pathol 19, 1456-1461 (2006).
9 Behr, J. P. The Proton Sponge: a Trick to Enter Cells the Viruses Did Not Exploit. CHIMIA International Journal for Chemistry 51, 34-36 (1997).
10 Jiang, W., KimBetty, Y. S., Rutka, J. T. & ChanWarren, C. W. Nanoparticle-mediated cellular response is size-dependent. Nat Nano 3, 145-150 (2008).
11 Zimmermann, R. et al. Charging and structure of zwitterionic supported bilayer lipid membranes studied by streaming current measurements, fluorescence microscopy, and attenuated total reflection Fourier transform infrared spectroscopy. Biointerphases 4, 1-6 (2009).
12 Ashihara, E., Kawata, E. & Maekawa, T. Future Prospect of RNA Interference for Cancer Therapies. Current Drug Targets 11, 345-360 (2010).
13 Pawitan, J. A. The possible use of RNA interference in diagnosis and treatment of various diseases. International Journal of Clinical Practice 63, 1378-1385 (2009).
14 Elbashir, S. M. et al. Duplexes of 21-nucleotide RNAs mediate RNA interference in cultured mammalian cells. Nature 411, 494-498 (2001).
15 Davis, M. E. et al. Evidence of RNAi in humans from systemically administered siRNA via targeted nanoparticles. Nature advance online publication (2010).
16 Oh, Y.-K. & Park, T. G. siRNA delivery systems for cancer treatment. Advanced Drug Delivery Reviews 61, 850-862 (2009).
17 Sou, K., Endo, T., Takeoka, S. & Tsuchida, E. Poly(ethylene glycol)-Modification of the Phospholipid Vesicles by Using the Spontaneous Incorporation of Poly(ethylene glycol)-Lipid into the Vesicles. Bioconjugate Chemistry 11, 372-379, doi:10.1021/bc990135y (2000).
18 Klein, E. et al. “HFP” Fluorinated Cationic Lipids for Enhanced Lipoplex Stability and Gene Delivery. Bioconjugate Chemistry 21, 360-371, doi:10.1021/bc900469z (2010).
19 Mínguez, B., Tovar, V., Chiang, D., Villanueva, A. & Llovet, J. M. Pathogenesis of hepatocellular carcinoma and molecular therapies. Current Opinion in Gastroenterology 25, 186-194 110.1097/MOG.1090b1013e32832962a32832961 (2009).
20 Li, S.-D., Chen, Y.-C., Hackett, M. J. & Huang, L. Tumor-targeted Delivery of siRNA by Self-assembled Nanoparticles. Mol Ther 16, 163-169, doi:http://www.nature.com/mt/journal/v16/n1/suppinfo/6300323s1.html (2007).
21 Landen, C. N. et al. Therapeutic EphA2 Gene Targeting In vivo Using Neutral Liposomal Small Interfering RNA Delivery. Cancer Research 65, 6910-6918 (2005).
22 Honjo, T., Nishizuka, Y., Hayaishi, O. & Kato, I. Diphtheria Toxin-dependent Adenosine Diphosphate Ribosylation of Aminoacyl Transferase II and Inhibition of Protein Synthesis. Journal of Biological Chemistry 243, 3553-3555 (1968).
23 Uchida, T., Kim, J. H., Yamaizumi, M., Miyake, Y. & Okada, Y. Reconstitution of lipid vesicles associated with HVJ (Sendai virus) spikes. Purification and some properties of vesicles containing non-toxic fragment A of diphtheria toxin. Journal of Cell Biology 80, 10-20 (1979).
Mizuguchi, H. et al. Application of fusogenic liposomes containing fragment A of diphtheria toxin to cancer therapy. British Journal of Cancer 73, 472-476 (1997).
25 Liu, J. W., Jiang, X. M., Ashley, C. & Brinker, C. J. Electrostatically Mediated Liposome Fusion and Lipid Exchange with a Nanoparticle-Supported Bilayer for Control of Surface Charge, Drug Containment, and Delivery. Journal of the American Chemical Society 131, 7567-+, doi:10.1021/ja902039y (2009).
26 Liu, J. W., Stace-Naughton, A. & Brinker, C. J. Silica nanoparticle supported lipid bilayers for gene delivery. Chemical Communications, 5100-5102, doi:10.1039/b911472f (2009).
27 Liu, J. W., Stace-Naughton, A., Jiang, X. M. & Brinker, C. J. Porous Nanoparticle Supported Lipid Bilayers (Protocells) as Delivery Vehicles. Journal of the American Chemical Society 131, 1354-+, doi:10.1021/ja808018y (2009).
28 Schagger, H. Tricine-SDS-PAGE. Nat. Protocols 1, 16-22 (2006).
29 Fritze, A., Hens, F, Kimpfler, A., Schubert, R. & Peschka-Süss, R. Remote loading of doxorubicin into liposomes driven by a transmembrane phosphate gradient. Biochimica et Biophysica Acta (BBA)—Biomembranes 1758, 1633-1640 (2006).
30 Elorza, B., Elorza, M. A., Frutos, G. & Chantres, J. R. Characterization of 5-fluorouracil loaded liposomes prepared by reverse-phase evaporation or freezing-thawing extrusion methods: study of drug release. Biochimica et Biophysica Acta 1153, 135-142 (1993).
31 Peleg-Shulman, T., Gibson, D., Cohen, R., Abra, R. & Barenholz, Y. Characterization of sterically stabilized cisplatin liposomes by nuclear magnetic resonance. Biochimica et Biophysica Acta 1510, 278-291 (2001).
32 Bogush, T., Smirnova, G., Shubina, L., Syrkin, A. & Robert, J. Direct evaluation of intracellular accumulation of free and polymer-bound anthracyclines. Cancer Chemotherapy and Pharmacology 35, 501-505, doi:10.1007/BF00686835 (1995).
33 Tong, A. W. et al. Chemosensitization of human hepatocellular carcinoma cells with cyclosporin A in post-liver transplant patient plasma. Clin. Cancer Res. 2, 531-539 (1996).
34 Minko, T., Kopeckova, P. & Kopecek, J. Chronic exposure to HPMA copolymer-bound adriamycin does not induce multidrug resistance in a human ovarian carcinoma cell line. Journal of Controlled Release 59, 133-148 (1999).

### Example 2

### Transdermal Delivery of Imatinib

The epidermis is the top layer of the skin, and can be further broken down into four layers. The outermost layer of the epidermis is the stratum corneum and is approximately 10-20 pm thick; it is responsible for the challenges associated with transdermal delivery. The other three layers of the epidermis can be collectively classified as the viable epidermis; the viable epidermis is 50-100 pm thick. The viable epidermis contains immune cells (langerhans cells), epithelial keratinocytes, sensory nerves (merkle cells), and networks of capillary beds, venules, and arterioles. The dermis is 1-2 mm thick and is composed of areolar tissue that is contains other types of immune cells (mast cells, lymphocytes, macrophages, neutrophils, plasma cells), fibroblasts, and various fibers (nerve fibers, collagen, elastic fibers). Additionally, la illustrates the four primary approaches that can be taken for cargo delivery across the stratum corneum. (a) the intercellular route, (b) follicular route, (c) transcellular route, and (d) removal of the stratum corneum. It is important to note that there is an increasing hydration gradient from the stratum corneum through the dermis. This gradient can provide a driving force for diffusion of various molecules into the viable epidermis and the dermis.

The stratum corneum has a “bricks and mortor” structure. The “bricks” are dead epithelial keratinocytes that are filled with keratin, sugars, and lipids. The “mortars” represent the intercellular space and are composed of ceramides, fatty acids, and cholesterol. This lipid composition confers a polarity that is similar to butanol. Due to this polarity and the overall “brick and mortar” structure, the stratum corneum is not permeable to most molecules without enhancement.

Thus, in simplist terms, the skin is composed of three primary layers, the epidermis, dermis, and subcutaneous tissues. The outermost layer (stratum corneum) is the primary component in the skin's role as a barrier. They are composed of dead epithelial keratinocytes filled with crystallized keratin, keratohyaline, and various lipids that protrude into the intercellular space. It is also composed of a variety of different lipids (i.e. ceramides, fatty acids, cholesterol) that confer a polarity similar to that of butanol. Resultant of this unique polarity, hydrogen bonding occurs in the intercellular space of the stratum corneum, which adds a second-degree of hindrance to molecules and drugs being delivered through the transdermal route. To date, there are three generations of transdermal delivery technologies. First-generation delivery systems utilize passive diffusion of lipophilic compounds with low molecular weight. Second- and third-generation delivery systems recognize that permeability of the stratum corneum is the key. The enhancement strategies of the second and third generations ablate the stratum corneum or utilize chemical enhancers, biochemical enhancers, and electromotive forces to increase permeability of the stratum corneum. The issue that arises from all enhancement strategies is finding the balance between sufficient permeability of the stratum corneum, while avoiding imitation of the deeper tissues.

The transdermal route of administration offers several benefits over the intravenous and oral routes of administration. These would include less toxicity, better tolerability and better delivery of Cargo such as chemotherapies, tyrosine kinase inhibitors and other treatments for cancer patients. The integument's circulation offers a high area for drug absorption while by-passing first-pass metabolism and adverse (drug-food, drug-pH) interactions.

Imatinib is the most commonly prescribed commercially available tyrosine kinase inhibitor. Imatinib is a weak base with a relatively low molecular weight (493 Da) and a Log P of 1.2.

We have shown that the solubility of Imatinib can easily be increased by lowering the pH. However, lowering the pH increases the ionization of the compound and molecules in the ionized state do not readily penetrate through the lipid bilayers of the skin. To enhance the intrinsic solubility (solubility of the unionized species), we evaluated several solvent and cosolvent systems (FIG. 1X2). All formulations were found to increase drug solubility over the control (water at pH 7), with highest solubility in the 10% ethanol and the DMSO formulations.

Our preliminary studies have investigated the potential of imatinib to be delivered by the transdermal route. A number of key preliminary experiments have been conducted to date. First, we determined the solubility of imatinib in water as a function of solvent pH (FIG. 1X2). Drug must be in solution in order to penetrate through the skin. However, the ionized species does not readily permeate through the stratum comewn (6). While the solubility of imatinib increased with decreasing pH, this solubility was due to the ionization of the weakly basic function groups on the chemical structure of the drug.

Next, we screened a number of cosolvents/solvent systems to increase the intrinsic solubility (solubility of the unionized species) of imatinib and dasatinib. Data from imatinib are presented in FIG. 2X2. The addition of such cosolvents to formulations is widely used to increase the solubility of poorly soluble drugs (7-9). In our previous work, ethanol, PEG 400, and DMSO were evaluated as solubility enhancers. These cosolvents are well-known to enhance the intrinsic solubility of various drugs. All cosolvent formulations and DMSO increased the solubility of imatinib compared to the control (water, pH 7). The formulation with 10% ethanol exhibited the highest solubility compared to the other formulations. Imatinib was also found to be highly soluble in DMSO.

Finally, a number of these cosolvent formulations were evaluated for their in vitro transdermal permeation properties (FIG. 3X2). Note that these cosolvents are also known to function as permeability enhancers in some formulations (10-11). For this series of experiments, human skin obtained from abdominoplastic surgery was mounted on modified Franz diffusion cells and drug permeation was determined as a function of time using high performance liquid chromatography.

The Franz diffusion cell is an essential tool in the field of transdermal drug delivery. Patient-derived skin is placed between the cell cap and the solution chamber. The cell cap is exposed to the environment allowing the stratum corneum to also be exposed to the environment. The solution chamber is filled with an isotonic diffusion buffer. Additionally the solution chamber has an injection port that allows for difhsion buffer to be removed without disturbing the setup. Finally, the solution chamber is surrounded by a water jacket that allows for temperature control. The Franz diffusion cell allows for in vitro studies of transdermal delivery to be carried out using physiological conditions. Note that penetration of any solute through the patient-derived skin into the diffusion buffer is equivalent to that solute reaching systemic circulation in an in vivo system. Protocells will be loaded with Imatinib mesylate and characterization of solute content in the diffusion buffer will be achieved using High-Performance Liquid Chromatography (HPLC). Determination of silica content in different layers of the skin will be determined using enzymatic tissue digestion, and inductively coupled plasma mass spectroscopy (ICP mass spec). Both the SLB and nanoporous particle core can be fluorescently tagged to allow for confocal microscopy. In addition, the skin samples can be microtomed after treatment and incubation with protocells so that they can be imaged using TEM.

As can be seen in FIG. 3X2, no imatinib permeated through the skin using water (pH 7) as the solvent system. The drug was able to permeate through the skin to a limited extent with other cosolvent systems evaluated. DMSO exhibited the highest permeability of the imatinib. From these data, flux (rate of permeation through the skin) was calculated and these values are shown in FIG. 4X2. Flux was increased for all the formulations compared to the control, with the DMSO formulation exhibiting the highest flux of imatinib (0.225 μg/cm2 hr).

Transdermal protocells can therefore be comprised of porous nanoparticulates that (a) are loaded with one or more pharmaceutically-active agents such as imatinib and (b) that are encapsulated by and that support a lipid bilayer which comprises one or more stratum corneum permeability-enhancers, e.g. monosaturated omega-9 fatty acids (oleic acid, elaidic acid, eicosenoic acid, mead acid, erucic acid, and nervonic acid, most preferably oleic acid), an alcohol, a diol (most preferably polyethylene glycol (PEG)), R8 peptide, and edge activators such as bile salts, polyoxyethylene esters and polyoxyethylene ethers, a single-chain surfactant (e.g. sodium deoxycholate). The protocell can have an average of between about 50 nm to about 300 nm, preferably between about 65 nm to about 75 nm.

### REFERENCES FOR EXAMPLE 2

1. “FASS.se.” Mobil.fass.se. Web. 26 Jan. 2010.
2. Benson, H. 2005. Transdermal Drug Delivery: Penetration Enhancement Techniques. Current Drug Delivery. 2: 23-33
3. Kear, C., Yang, J., Godwin, D., and Felton, L. 2008. Investigation into the Mechanism by Which Cyclodextrins Influence Transdermal Drug Delivery. Drug development and Industrial Pharmacy. 34:692-697.
4. Bany, B. W. 2001. Novel mechanisms and devices to enable successful transdermal drug delivery. European Journal of Pharmaceutical Sciences. 14101-1 14
5. Maghraby, G., Barry, W., and Williams, A. 2008. Liposomes and skin: From drug delivery to model membranes. European Journal of Pharmaceutical Science. 34203-222.
6. Singh, B., Singh, J. and Singh, B. N. 2005. Effects of ionization and penetration enhancers on the transdermal delivery of 5-fluorouracil through excised human stratum corneum. International Journal of Pharmaceutics. 298:98-107.
7. Douroumis, D., and Fahr, A. 2007. Stable carbamazepine colloidal systems using the cosolvent technique. European Journal of Pharmaceutical Science. 30:367-374.
8. Ni, N., Sanghvi, T., and Yalkowsky, S. 2002. Solubilization and prefomulation of carbendazim. International Journal of Pharmaceutics. 24499-104
9. Rubino, T. J. and Yalkowsky, H. S. 1987. Cosolvency and Cosolvent Polarity. Pharmaceutical Research. 4220-230
10. “Pharmacology of DMSO.” Dimethyl Sulfoxide (DMSO)—Dr. Stanley Jacob. Web. 30 Mar. 2010.
11. Notman, R., Otter, K. W., Noro, G. M., Briels, J. W., and Anwar, J. 2007. The Permeability Enhancing Mechanism of DSMO in Ceramide Bilayers Simulated by Molecular Dynamics. Biophysical Journal. 93:2056-2068.

### Example 3

### Apoptosis Induced by siRNA-Loaded, SP94-Targeted Protocells

### Results

Characterization of siRNA-Loaded Protocells.

Silica nanoparticles were prepared as described by Carroll, et al.³⁵ and had a BET surface area of >600 m²/g, a pore volume fraction of ˜65%, and a multimodal pore morphology composed of large (20-30 nm), surface-accessible pores interconnected by 6-12 nm pores (see FIGS. 2BX3-CX3). Silica nanoparticles were size-separated (see FIG. 2AX3) before being loaded with siRNA (or ricin toxin A-chain) as described in the Methods section. The siRNA loading capacity of protocells or lipoplexes constructed using a series of strategies is shown in FIG. 3AX3. Lipoplexes composed of the zwitterionic phospholipid, DOPC, encapsulated ˜10 nM of siRNA per 10¹⁰ particles. Construction of lipoplexes composed of the cationic lipid, DOTAP, resulted in a 5-fold increase in the siRNA cargo, presumably due to attractive electrostatic interactions between the negatively-charged nucleotide and the positively-charged lipid components. A protocell containing a negatively-charged silica core with a zwitterionic lipid bilayer had a capacity roughly equivalent to the cationic lipoplex. Modification of the silica core with the amine-containing silane, AEPTMS, increased the zeta potential from −32 mV to +12 mV and resulted in a siRNA capacity of ˜1 μM per 10¹⁰ particles. Use of DOTAP liposomes to synergistically load siRNA into negatively-charged cores³⁶ resulted in protocells with a similar capacity, more than 100-fold higher than that of the zwitterionic lipoplexes that are often utilized in particle-based therapeutic applications. The stability of DOPC and DOTAP lipoplexes, as well as DOPC protocells with AEPTMS-modified cores upon dispersion in a surrogate biological fluid is shown in FIGS. 3BX3 and 3CX3. DOPC lipoplexes rapidly release their encapsulated siRNA under both neutral and mildly acidic pH conditions, resulting in a complete loss of the nucleotide content within 4-12 hours. Although DOTAP lipoplexes were more stable than DOPC lipoplexes under neutral pH conditions, approximately 50% of their siRNA content was lost over a 72-hour period. In marked contrast to both lipoplexes, DOPC protocells with AEPTMS-modified cores retained 95% of their encapsulated RNA when exposed to the simulated body fluid for 72 hours. Under mildly acidic conditions that reflect those in the endosome/lysosome pathway, the reduced electrostatic and dipolar interactions between the siRNA-loaded, AEPTMS-modified core and the PE and PC headgroups of the supported lipid bilayer caused membrane destabilization and exposure of the core to the acidic medium. After membrane destabilization, the combined rates of cargo diffusion and core dissolution resulted in the release profile seen in FIG. 3CX3. Thus, in terms of siRNA loading capacity, particle stability, and release characteristics, protocells represent a dramatic improvement over corresponding lipoplexes.

Cytotoxicity Mediated by siRNA-Loaded Protocells:

We recently demonstrated the ability of protocells, conjugated with a targeting peptide (SP94) that binds to hepatocellular carcinomas (HCC) but not control hepatocytes, to deliver a wide variety of chemotherapeutic agents and selectively induce apoptosis in tumor cells that express the relevant surface marker.³⁴ Here we markedly expand characterization of targeted protocells loaded with macromolecular cargos, including siRNAs and protein toxins. We prepared protocells composed of AEPTMS-modified silica cores and a DOPC/DOPE/cholesterol/PEG-2000 (55:5:30:10 mass ratio) supported lipid bilayer conjugated with both SP94 to confer selective binding to HCC and an endosomolytic peptide to promote endosomal/lysosomal release. Protocells were loaded with an equimolar mixture of siRNAs that target members of the cyclin superfamily, including cyclin A2, cyclin B1, cyclin D1, and cyclin E, proteins intimately involved in the regulation of both cell cycle traverse and viability.³⁷

The concentration and time dependence of gene silencing in the HCC line, Hep3B, by siRNA-loaded, SP94-targeted DOPC protocells constructed with AEPTMS-modified cores are shown in FIG. 4X3. Panel A demonstrates that increasing concentrations of protocells and, thereby, increasing concentrations of siRNA induced a dose-dependent decrease in the protein levels of each of the targeted genes within 48 hours. The concentrations of siRNA required to repress protein expression by 90% (IC₉₀) were 125 pM, 92 pM, 149 pM and 370 pM for cyclin A2, cyclin B1, cyclin D1, and cyclin E (respectively). Panel B shows how protein levels decrease upon addition of 125 pM of siRNA loaded within targeted protocells. By 72 hours, the level of each of the targeted proteins was repressed by over 90%, with the degree of repression (cyclin E somewhat lower than the other cyclins) reflecting the differences in IC₉₀ values. FIG. 4CX3 shows the selectivity of gene silencing achievable with various types of SP94-targeted particles. DOPC protocells loaded with 125 pM of siRNA induced nearly complete repression of cyclin A2 protein following 48 hours of incubation with Hep3B but had no effect on non-transformed hepatocytes. In contrast, DOPC lipoplexes loaded with 125 pM of siRNA had little effect on cyclin protein levels in either cell line. SP94-targeted DOTAP lipoplexes loaded with 125 pM of siRNA induced a ˜60% repression of cyclin A2 expression in Hep3B but also decreased cyclin A2 levels in hepatocytes, an effect likely caused by their positive charge (ζ=+22 mV). The numbers of SP94-targeted DOPC protocells, DOPC lipoplexes, and DOTAP lipoplexes required to repress cyclin A2 expression by 90% is shown on the right axis in panel C. 10⁴-fold fewer DOPC protocells were required than analogous DOPC lipoplexes, while 300-fold fewer DOPC protocells were required than DOTAP lipoplexes. Thus, in terms of both activity and specificity, targeted protocells offer marked advantages over lipid-based nanoparticles.

Confocal fluorescence microscopy images illustrating the time-dependence of protocell distribution and cyclin A2, B1, D1, and E expression in cells exposed to siRNA-loaded, SP94-targeted protocells is shown in FIG. 5X3. As demonstrated in panel A, 1 hour after addition of protocells to Hep3B, the expression of each of the proteins remains at control levels, and the silica cores are present in a punctuate pattern, suggesting endosomal localization. By 48 hours, the silica cores are uniformly distributed throughout the cytoplasm of the Hep3B cells, and the expression of each of the targeted proteins is repressed to background levels. In comparison, an identical treatment of non-transformed hepatocytes results in neither the cellular accumulation of protocells nor the repression of protein expression (see panel B).

The ability of siRNA-loaded, SP94-targeted DOPC protocells to selectively induce cytotoxicity of HCC is demonstrated in FIG. 6X3. Protocells were loaded with 125 pM of the siRNA cocktail and added to either Hep3B or control hepatocytes. Cells in the early stages of apoptosis were identified by an increase in annexin V binding, while cells in the late stages of apoptosis were positive for both annexin V and propidium iodide staining. A selective increase in the number of apoptotic Hep3B was observed as early as 12 hours after addition of protocells (panel A), and over 90% of cells were positive for both apoptosis markers by 72 hours. In contrast, no cytotoxicity was observed in non-transformed hepatocytes, observations confirmed by the representative microscopy images shown in FIGS. 6B and 6C. Panel B demonstrates that the entire population of Hep3B became positive for surface-bound annexin V and nuclear-bound propidium iodide within 48 hours, while panel C shows that control hepatocytes remained negative for both markers of apoptosis.

Characterization of Toxin-Loaded Protocells.

Due to the presence of large (20-30 nm), surface-accessible pores, multimodal silica nanoparticles can be readily loaded with various protein toxins, including diphtheria, cholera, and ricin toxins. Furthermore, the high degree of differential specificity exhibited by DOPC protocells modified with a low density (0.015 wt %, or −6 peptides/protocell) of SP94 enables selective delivery of especially cytotoxic agents to cancer cells. Ricin toxin is found in the seeds of the castor oil plant (Ricinus communis) and is composed of a heterodimer consisting of an A and B subunit held together by disulfide bonds. The B subunit mediates entry of the toxin into cells via receptor-mediated endocytosis, while the A subunit inhibits protein synthesis by cleaving a specific glycosidic bond in the 28S rRNA.³⁸ Catalytically-active ricin toxin A-chain (RTA) has been employed as a subunit of tumor-specific immunotoxins to inhibit the growth of cancer cells in multiple model systems.^{39,40}

The capacities and release characteristics of DOPC protocells and liposomes loaded with RTA are shown in FIG. 7X3. As demonstrated by panel A, <1 nM of protein could be loaded within 10¹⁰ DOPC liposomes. In contrast, DOPC protocells with unmodified silica cores encapsulated nearly 100-fold more RTA, and modification of the cores with AEPTMS increased this capacity by a further order of magnitude. The-pH dependent stability of RTA-loaded DOPC protocells and, liposomes is shown in panels B and C. DOPC protocells released ˜5% of their encapsulated cargo when incubated in a simulated body fluid at neutral pH for up to 72 hours, and RTA was steadily released from the particle under mildly acidic (i.e. endosomal) conditions. In contrast, DOPC liposomes rapidly lost their RTA content under both neutral and acidic conditions.

### Cytotoxicity Mediated by RTA-Loaded Protocells.

As shown in FIG. 8X3, RTA encapsulated within SP94-targeted protocells caused a concentration (panel A) and time (panel B) dependent decrease in nascent protein synthesis in Hep3B cells. 48 hours after addition of RTA-loaded, SP94-targeted protocells, half-maximal inhibition of protein synthesis was achieved at a RTA concentration of ˜5 pM, and full inhibition was observed at ˜30 pM of RTA (panel A). RTA-loaded protocells caused a 50% reduction in protein synthesis within ˜24 hours and complete repression within 60 hours when added to Hep3B at a RTA concentration of 25 pM (panel B). The results shown in panel C demonstrate that RTA-loaded, SP94-targeted protocells efficiently repressed nascent protein synthesis when added to Hep3B but had little, effect on control hepatocytes under identical conditions. In contrast, SP94-targeted DOPC liposomes, when added to cells such that the final concentration of RTA was 25 pM, failed to inhibit nascent protein synthesis in either Hep3B or hepatocytes. Furthermore, as shown in the right axis of panel C, 10⁴-fold more RTA-loaded liposomes (˜60 pM of RTA) were required to repress protein biosynthesis by 90% in Hep3B cells.

Nascent protein synthesis and intracellular protocell distributions were quantified with an Alexa Fluor 488-labeled derivative of methionine and Alexa Fluor 647-labeled silica cores (respectively), as shown in FIG. 9X3. 1 hour after addition of RTA-loaded, SP94-targeted protocells to Hep3B, protein synthesis was robust, and protocells were localized in cytoplasmic vesicles (panel A). After a 48-hour incubation, protocells were dispersed throughout the cytoplasm, and protein synthesis was markedly repressed. As shown in panel B, addition of analogous protocells to non-transformed hepatocytes resulted in neither cellular accumulation of protocells nor the repression of nascent protein synthesis.

The ability of RTA-loaded protocells to selectively induce cytotoxicity in HCC but not control hepatocytes is shown in FIG. 10X3. RTA-loaded, SP94-targeted protocells induced apoptosis in Hep3B cells, as measured by the activation of caspase-9 and/or caspase-3, as early as 8 hours with 50% of the cells becoming positive by 20-28 hours (panel A). Complete cell death was seen by 48 hours. Equivalent protocell concentrations did not decrease hepatocyte viability below control levels, even after 7 days of incubation. Microscopy images showing protocell distribution and apoptosis are shown in panels B and C. 48 hours after addition of RTA-loaded, SP94-targeted protocells to Hep3B, protocells were distributed in the cytoplasm, and cells were positive for both caspase-9 and caspase-3 activation (panel B). As shown in panel C, control hepatocytes remained negative for caspase staining and particle accumulation under identical experimental conditions.

### DISCUSSION

The full potential of macromolecular therapies, including nucleic acids and toxins, which are under extensive investigation for the treatment of many diseases mediated by aberrant patterns of gene expression, remain unfulfilled due to marked deficiencies in delivery systems.^{17,18} Here, we present evidence indicating that protocells exhibit characteristics that enable efficient packaging and specific cellular delivery of both siRNAs and protein toxins.

Unmodified nucleic acids, including siRNA, cannot be systemically administered for several reasons. They are highly susceptible to plasma nucleases and have a very short circulation half-life due to efficient renal filtration.³ In addition, nucleic acids are not readily taken up by cells because of their net negative charge and large size.⁴¹ To circumvent these issues, siRNAs have been conjugated to a variety of polymers or encapsulated in nanoparticles such as liposomes. Incorporation into neutral liposomes or conjugation to cationic lipids have increased stability and circulating half-life and, in the case of cationic complexes, enhanced electrostatically-mediated delivery to cells.^{42,43} Natural products, including chitosan⁴⁴ and cyclodextran⁴⁵ have been used to form biologically-active complexes with siRNAs. Conjugation with cationic polymers, such as polyethyleneimine, has also been shown to enhance the therapeutic efficiency of siRNA by helping to prevent degradation and enhance delivery.⁴⁶

The therapeutic use of systemically-administered siRNA requires delivery to specific organs or subsets of cells to enhance efficacy and decrease non-specific toxicity. This is especially true in the case of anti-cancer therapies, where it is necessary to protect normal cells from the actions of cytotoxic siRNAs. Complications also arise if targeted cells exist at multiple locations in the body, as is the case with hematological tumors or metastatic disease where neoplastic cells are widely disseminated. To address this issue, molecules that recognize antigens differentially-expressed on the surfaces of targeted cells have been conjugated either directly to siRNAs or to particles that encapsulate the nucleotides. Receptor ligands, such as folate⁴⁷, cholesterol⁴⁸, and transferrin¹³ have been successfully used to direct the binding of siRNA complexes to cells that over-express the respective cellular receptor. Antibodies that recognize appropriate molecules on target cells have also been used to direct selective binding of particles containing siRNAs to specific classes of cells.⁴⁹ Additionally, peptides or nucleic acid aptamers, selected by a multiplex screening procedure to bind desired cellular epitopes, have been conjugated directly to siRNAs or to classes of siRNA-containing particles to enhance specific cellular interactions.⁵⁰

Despite the marked advances in some aspects of nucleic acid and protein delivery systems, including modification of their chemical structure to protect against degradation or conjugation to targeting reagents, a number of deficiencies remain. While a number of reagents that employ cationic lipids or polymers to electrostatically complex, condense, and deliver nucleic acids are commercially-available, the majority of these formulations result in the non-specific transfection of eukaryotic cells. In addition, cationic lipid/nucleic acid complexes (lipoplexes) have been found to be cytotoxic, and their transfection efficiency and colloidal stability tend to be limited in the presence of serum. Conversely, zwitterionic lipids are incapable of efficiently compacting nucleic acids, even in the presence of divalent cations. All such nanoparticle delivery systems also suffer from limited cargo capacities.

As shown by our experimental results, protocells offer significant advantages over existing delivery strategies. We have previously described their utility as targeted nanocarriers for small molecule therapeutic agents and demonstrated that their cargo capacity, stability, and cell-specific cytotoxicity are far superior to traditional liposomes. Nanoparticle-based delivery of macromolecules presents even greater challenges due to their large size, charge characteristics, and potential issues with intracellular cargo release. Here we have shown that protocells offer distinct advantages in these applications as well. Multimodal porous silica nanoparticles can be rapidly loaded with nucleic acids, toxins, and macromolecular cocktails by soaking them in solutions of the desired cargo(s). Fusion of DOPC liposomes to cargo-loaded cores results in the formation of a stabilized supported lipid bilayer (SLB) that retains cargo at neutral pH, reduces non-specific binding, improves colloidal stability, and mitigates the cytotoxicity associated with cationic liposomes and lipoplexes (see reference 34 for more details). Targeting peptides conjugated to the fluid but stable SLB interact multivalently with cell surface receptors, inducing receptor-mediated endocytosis. Within the acidified endosomal environment, SLB destabilization along with osmotic swelling and disruption of endosomes (caused by the proton sponge effect of endosomolytic peptides), result in dispersion of silica cores within the cytoplasm. Combined diffusion and silica core dissolution enable controlled, sustained cargo release for >12 hours. The combined capacity, stability, and targeting and internalization efficiency of protocells result in exceptionally low IC₉₀ values for Hep3B with practically no adverse effects on normal hepatocytes.

Protocells with 150-nm cores encapsulate, on average, ˜6×10⁷ siRNA molecules or ˜1×10⁷ ricin toxin A-chain (RTA) molecules per particle (per L) and retain nearly 100% of their cargo upon exposure to a simulated body fluid for 72 hours. In comparison, lipid and polymer nanoparticles have a 10 to 1000-fold lower capacity for macromolecular cargos and are substantially less stable at neutral pH.^{51,52} Protocells, furthermore, have a higher capacity for nucleic acid cargos than other mesoporous silica particles. S1MPs, developed by Tanaka, et al. for sustained delivery of siRNA-loaded nanoliposomes to ovarian cancer, encapsulate approximately the same amount of RNA as protocells (2.0 pg per particle vs. 1.3 pg per particle), even though their average diameter is ten times greater (1.6 μm vs. 150 nm)⁵³. Polyethyleneimine-coated mesoporous silica nanoparticles, developed by Xia, et al., complex ˜1 μg of siRNA per 10 μg of particles (10 wt %)³³; in comparison, 10 μg of protocells can be loaded with ˜6.5 μg of siRNA (65 wt %) Enhancements in capacity and stability enable siRNA-loaded protocells to silence target genes and induce apoptosis of HCC at concentrations that are 10 to 10,000 times less than values reported in the literature.^{51,52,54-58} siRNA-loaded, SP94-targeted protocells silence 90% of cyclin A2, B1, D1, and E expression at siRNA concentrations ranging from 90 pM to 370 pM (IC₉₀) and kill >90% of HCC within 48 hours at a siRNA concentration of 125 pM (LC₉₀). In comparison, targeted liposomes have IC₉₀ and LC₉₀ values of 5-500 nM, depending on the type of particle and conditions under which experiments were conducted.^{54-56,58-60} The therapeutic efficacy of siRNA-loaded, SP94-targeted protocells exceeds that of polymer-encased mesoporous nanoparticles as well. Several groups have used mesoporous silica nanoparticles encapsulated within polycationic polymers to complex siRNA; such particles result in 30-60% knockdown of reporter and endogenous gene expression within 24-48 hours at nanoparticle:siRNA (w/w) ratios of 10-20.^{33,61} Since we load siRNA within the nanopores of AEPTMS-modified silica nanoparticles, the capacity of protocells is significantly higher, resulting in complete silencing of cyclin A2, B1, D1, and E expression at a protocell:cell ratio of ˜8. In conclusion, our findings suggest that protocells might serve as universal targeted nanocarriers for multiple classes of macromolecules, including nucleic acids and toxins. The nanoporous cores can also be loaded with other disparate cargo types, including the imaging and diagnostic agents needed for the burgeoning fields of theranostics and personalized medicine.

### Materials and Methods

Materials.

Antibodies against cyclin A2 (mouse mAb), cyclin B1 (mouse mAb), cyclin D1 (mouse mAb), and cyclin E (mouse mAb) were purchased from Abcam, Inc. (Cambridge, Mass.). Silencer select siRNAs (siRNA IDs for cyclin A2, B1, D1, and E are s2513, s2515, s229, and s2526, respectively) were purchased from Applied Biosystems™ by Life Technologies Corporation (Carlsbad, Calif.). Human Hep3B (HB-8064), human hepatocytes (CRL-11233), Eagle's Minimum Essential Medium (EMEM), Dulbecco's Modified Eagle's Medium (DMEM), fetal bovine serum (FBS), and 1× trypsin-EDTA solution (0.25% trypsin with 0.53 mM EDTA) were purchased from American Type Culture Collection (ATCC; Manassas, Va.). 1,2-dioleoyl-sn-glycero-3-phosphocholine (DOPC), 1,2-dioleoyl-sn-glycero-3-phosphoethanolamine (DOPE), 1,2-dioleoyl-sn-glycero-3-phosphoethanolamine-N-[methoxy(polyethylene glycol)-2000] (18:1 PEG-2000 PE), 1,2-dioleoyl-3-trimethylammonium-propane (DOTAP), and cholesterol were purchased from Avanti Polar Lipids, Inc. (Alabaster, Ala.). CaspGLOW™ Fluorescein Active Caspase-9 Staining Kit (485/535) and CaspGLOW™ Red Active Caspase-3 Staining Kit (540/570) were purchased from BioVision, Inc. (Mountain View, Calif.). ABIL® EM 90 (cetyl PEG/PPG-10/1 dimethicone) was purchased from Evonik Industries (Essen, Germany). Hoechst 33342 (350/461), Alexa Fluor® 488 Antibody Labeling Kit (495/519), Alexa Fluor® 488 conjugate of annexin V (495/519), Click-iT® AHA Alexa Fluor® 488 Protein Synthesis HCS Assay (495/519), propidium iodide (535/617), Alexa Fluor® 647 carboxylic acid, succinimidyl ester (650/668), SlowFade® Gold antifade reagent, Image-iT® FX signal enhancer, 1× Dulbecco's phosphate-buffered saline (D-PBS), and bovine albumin fraction V solution (BSA, 7.5%) were purchased from Invitrogen Life Sciences (Carlsbad, Calif.). BEGM Bullet Kits were purchased from Lonza Group Limited (Clonetics; Walkersville, Md.). Amicon® Ultra-4 Centrifugal Filter Units (10 kDa MWCO) were purchased from Millipore (Billerica, Mass.). All peptides were synthesized by New England Peptide (Gardner, Mass.). Succinimidyl-[(N-maleimidopropionamido)-tetracosaethyleneglycol] ester (SM(PEG)₂₄) was purchased from Pierce Protein Research Products (Thermo Fisher Scientific LSR; Rockford, Ill.). Ultra pure, EM-grade formaldehyde (16%, methanol-free) was purchased from Polysciences, Inc. (Warrington, Pa.). Absolute ethanol, hydrochloric acid (37%), tetraethyl orthosilicate (TEOS, 98%), 3-[2-(2-aminoethylamino)ethylamino]propyltrimethoxysilane (AEPTMS, technical grade), hexadecyltrimethylammonium bromide (CTAB, ≧99%), sodium dodecyl sulfate (SDS, ≧98.5%), Triton X-100, hexadecane (≧99%), tert-butanol (≧99.5%), 2-mercaptoethanol (≧99.0%), DL-dithiothreitol (≧99.5%), dimethyl sulfoxide (≧99.9%), pH 5 citric acid buffer, ethylenediaminetetraacetic acid (EDTA, 99.995%), human epidermal growth factor, L-a-phosphatidylethanolamine, bovine fibronectin, bovine collagen type I, soybean trypsin inhibitor (≧98%), DMEM without phenol red, deglycosylated A-chain from Ricinus communis, and Sephadex® G-200 were purchased from Sigma-Aldrich (St. Louis, Mo.).

Cell Culture Conditions.

Hep3B and hepatocytes were obtained from ATCC and grown per manufacturer's instructions. Briefly, Hep3B was maintained in EMEM with 10% FBS. Hepatocytes were grown in flasks coated with BSA, fibronectin, and bovine collagen type I; the culture medium used was BEGM (gentamycin, amphotericin, and epinephrine were discarded from the BEGM Bullet kit) with 5 ng/mL epidermal growth factor, 70 ng/mL phosphatidylethanolamine, and 10% FBS. Cells were maintained at 37° C. in a humidified atmosphere (air supplemented with 5% CO2) and passaged with 0.05% trypsin at a sub-cultivation ratio of 1:3.

Synthesis of Multimodal Silica Nanoparticles.

The emulsion processing technique used to synthesize nanoporous silica particles with multimodal porosity has been described by Carroll, et al.³⁵ Briefly, 1.82 g of CTAB (soluble in the aqueous phase) was added to 20 g of deionized water, stirred at 40° C. until dissolved, and allowed to cool to 25° C. 0.57 g of 1.0 N HCl, 5.2 g of TEOS, and 0.22 g of NaCl were added to the CTAB solution, and the resulting sol was stirred for 1 hour. An oil phase composed of hexadecane with 3 wt % ABIL® EM 90 (a non-ionic emulsifier soluble in the oil phase) was prepared. The precursor sol was combined with the oil phase (1:3 volumetric ratio of sol:oil) in a 1000-mL round-bottom flask, stirred vigorously for 2 minutes to promote formation of a water-in-oil emulsion, affixed to a rotary evaporator (R-205; Buchi Laboratory Equipment; Switzerland), and placed in an 80° C. water bath for 30 minutes. The mixture was then boiled under a reduced pressure of 120 mbar (35 rpm for 3 hours) to remove the solvent. Particles were then centrifuged (Model Centra MP4R; International Equipment Company; Chattanooga, Tenn.) at 3000 rpm for 20 minutes, and the supernatant was decanted. Finally, the particles were calcined at 500° C. for 5 hours to remove surfactants and other excess organic matter.

To make unmodified particles more hydrophilic, they were treated with (i) 4% (v/v) ammonium hydroxide and 4% (v/v) hydrogen peroxide and (ii) 0.4 M HCl and 4% (v/v) hydrogen peroxide for 15 minutes at 80° C. Particles were then washed several times with water and re-suspended in 0.5×D-PBS at a final concentration of 25 mg/mL. Nanoporous cores were modified with the amine-containing silane, AEPTMS, by adding 25 mg of calcined particles to 1 mL of 20% AEPTMS in absolute ethanol; the particles were incubated in AEPTMS overnight at room temperature, centrifuged (5,000 rpm, 1 minute) to remove unreacted AEPTMS, and re-suspended in 1 mL of 0.5×D-PBS. AEPTMS-modified particles were fluorescently-labeled by adding 5 μL of an amine-reactive fluorophore (Alexa Fluor® 647 carboxylic acid, succinimidyl ester; 1 mg/mL in DMSO) to 1 mL of particles; the particles were kept at room temperature for 2 hours prior to being centrifuged to remove unreacted dye. Fluorescently-labeled particles were stored in 0.5×D-PBS at 4° C. Particles larger than ˜200-nm in diameter were removed via size exclusion chromatography or differential centrifugation before cargo loading and liposome fusion.

Characterization of Silica Nanoparticles.

Dynamic light scattering of nanoporous silica particles, as well as cargo-loaded protocells and liposomes, was performed using a Zetasizer Nano (Malvern; Worcestershire, United Kingdom). Samples were prepared by diluting 48 μL of silica particles (25 mg/mL) in 2.4 ml of 0.5×D-PBS. Solutions were transferred to 1 mL polystyrene cuvettes (Sarstedt; Nümbrecht, Germany) for analysis. Nitrogen sorption was performed using an ASAP 2020 Surface Area and Porosity Analyzer (Micromeritics Instrument Corporation; Norcross, Ga.). Zeta potential measurements were made using a Zetasizer Nano (Malvern; Worcestershire, United Kingdom). Silica particles were diluted 1:50 in 0.5×D-PBS and transferred to 1-mL folded capillary cells (Malvern; Worcestershire, United Kingdom) for analysis.

Liposome Fusion to Nanoporous Silica Particles.

The procedure used to synthesize protocells has been described previously^{34,36,62,63} ENREF 33 and will be mentioned only briefly. Lipids were ordered from Avanti Polar Lipids pre-dissolved in chloroform and stored at −20° C. Immediately prior to protocell synthesis, 2.5 mg of lipid was dried under a stream of nitrogen and placed in a vacuum oven (Model 1450M, VWR International, West Chester, Pa.) overnight to remove residual solvent. Lipids were re-hydrated in 0.5×D-PBS at a concentration of 2.5 mg/mL and were passed through a 100-nm filter at least 10 times using a Mini-Extruder set (Avanti Polar Lipids, Inc.; Alabaster, Ala.). Resulting liposomes (˜120-nm in diameter) were stored at 4° C. for no more than one week. Nanoporous silica cores (25 mg/mL) were incubated with a 2- to 4-fold volumetric excess of liposomes for 30-90 minutes at room temperature. Protocells were stored in the presence of excess lipid for up to 1 month at 4° C. To remove excess lipid, protocells were centrifuged at 5,000 rpm for 1 minute, washed twice, and re-suspended in 0.5×D-PBS.

Lipids were lyophilized together prior to rehydration and extrusion; for example 75 μL of DOPC (25 mg/mL), 5 pL of DOPE (25 mg/mL), 10 μL of cholesterol (75 mg/mL), and 10 μL of 18:1 PEG-2000 PE (25 mg/mL) were combined and dried to form liposomes composed of DOPC with 5 wt % DOPE, 30 wt % cholesterol, and 10 wt % PEG-2000. A DOPC:DOPE:cholesterol:18:1 PEG-2000 PE mass ratio of 55:5:30:10 was used to synthesize ‘DOPC protocells’, while a DOTAP:DOPE:cholesterol:18:1 PEG-2000 PE mass ratio of 55:5:30:10 was used to synthesize ‘DOTAP protocells’.

Conjugation of Peptides to the Supported Lipid Bilayer.

SP94 and H5WYG peptides, synthesized with C-terminal cysteine residues, were conjugated to primary amines present in the head groups of PE using the heterobifunctional crosslinker, SM(PEG)₂₄, which is reactive toward sulfhydryl and amine moieties and possesses a 9.52-nm PEG spacer arm. Protocells were first incubated with a 10-fold molar excess of SM(PEG)₂₄ for 2 hours at room temperature and centrifuged (1 minute at 5,000 rpm) to remove unreacted crosslinker. Activated protocells were then incubated with a 5-fold molar excess of SP94 for 2 hours at room temperature to attain a peptide density of 0.015 wt % (˜6 peptides/protocell) and with a 500-fold molar excess of H5WYG for 4 hours at room temperature to attain a peptide density of 0.500 wt % (˜240 peptides/protocell). Protocells were washed to remove free peptide, and average peptide density was determined by Tricine-SDS-PAGE, as described previously.³⁴

Synthesis of siRNA and Ricin Toxin A-Chain-Loaded Protocells.

Unmodified or AEPTMS-modified cores (25 mg/mL) were soaked in siRNA (250 pM in 1×D-PBS) or deglycosylated ricin toxin A-chain (100 ρM in 1×D-PBS) for 2 hours at 4° C. Unencapsulated cargo was removed via centrifugation at 5,000 rpm for 1 minute, and DOPC liposomes were immediately fused to cargo-loaded cores as described above. Unmodified cores were loaded with siRNA via the synergistic mechanism previously described by us.³⁶ Briefly, 25 μL of siRNA (1 mM) was added to 75 μL of silica nanoparticles (25 mg/mL). The solution was gently vortexed and incubated with 200 μL of DOTAP liposomes overnight at 4° C. Excess lipid and unencapsulated siRNA were removed via centrifugation immediately before use.

Synthesis of siRNA-Loaded Lipoplexes.

To prepare siRNA-loaded DOPC lipoplexes, DOPC, DOPE, cholesterol, and 18:1 PEG-2000 PE were first mixed in a 55:5:30:10 mass ratio, dried under a stream of nitrogen, and placed in a vacuum oven overnight to remove residual chloroform. The lipid film was then dissolved in tert-butanol and mixed 1:1 (v/v) with a siRNA solution (diluted in 10 mM Tris-HCl (pH 7.4) with 0.85% (w/v) NaCl and 0.25 M sucrose) such that the final DOPC:siRNA ratio was 10:1 (w/w). The mixture was vortexed, flash frozen in a bath of acetone and dry ice, and lyopholized Immediately before use, the lipoplex preparation was hydrated with an isotonic sucrose solution (10 mM Tris-HCl (pH 7.4) with 0.85% (w/v) NaCl and 0.25 M sucrose) to a final siRNA concentration of 100 μg/mL; unencapsulated siRNA was removed via centrifugal-driven filtration (10 kDa MWCO).

We prepared siRNA-loaded DOTAP lipoplexes as described by Wu, et al.,⁶⁴ with minor modifications. We replaced PEGylated ceramide with 18:1 PEG-2000 PE and used a DOTAP:DOPE:cholesterol:PEG-2000 PE ratio of 55:5:30:10. We, additionally, dissolved lyopholized lipoplexes in 10 mM Tris-HCl (pH 7.4) with 0.85% (w/v) NaCl and 0.25 M sucrose to a final siRNA concentration of 100 μg/mL and removed unencapsulated siRNA using a centrifugal filtration device (10 kDa MWCO). Lipoplexes were dissolved in 0.5×D-PBS for zeta potential analysis.

To modify DOPC and DOTAP lipoplexes with SP94 and H5WYG, they were first incubated with a 10-fold molar excess of SM(PEG)₂₄ for 2 hours at room temperature; after removal of unreacted crosslinker via centrifugal-driven filtration (10 kDa MWCO), they were incubated with a 5-fold molar excess of SP94 and a 1000-fold molar excess of H5WYG for 2 hours at room temperature. Free peptide was removed using a centrifugal filtration device (10 kDa MWCO).

Synthesis of RTA-Loaded Liposomes.

To prepare RTA-loaded DOPC liposomes, 2.5 mg of lipid (55:5:30:10 mass ratio of DOPC:DOPE:cholesterol:18:1 PEG-2000 PE) was dried under a stream of nitrogen and placed in a vacuum oven (Model 1450M, VWR International, West Chester, Pa.) overnight to remove residual solvent. Lipids were re-hydrated in 0.5×D-PBS at a concentration of 2.5 mg/mL, sonicated briefly, and mixed with an equal volume of RTA (100 μM in 0.5×D-PBS). The mixture was vortexed, flash frozen in a bath of acetone and dry ice, and lyophilized. Immediately before use, the liposome preparation was re-hydrated with the isotonic sucrose solution described above, vortex vigorously, and allowed to stand at room temperature for 2-4 hours. Liposomes were then passed through a 100-nm filter at least 10 times using a Mini-Extruder set (Avanti Polar Lipids, Inc.; Alabaster, Ala.) and passed over a Sephadex® G-200 column to remove unencapsulated RTA. RTA-loaded liposomes were modified with SP94 and H5WYG as described above.

Determination of Cargo Capacities and Release Rates.

The capacity of protocells, lipoplexes, and liposomes for siRNA and ricin toxin A-chain (RTA) was determined by incubating 1×10¹⁰ particles in 1 wt % SDS (dissolved in D-PBS) for 24 hours and centrifuging the solutions to remove protocell cores and other debris. The concentration of siRNA in the supernatant was determined by comparing the absorbance at 260 nm to a standard curve. The concentration of RTA in the supernatant was determined via SDS-PAGE by comparing band intensities to a standard curve using Image J Image Processing and Analysis software (National Institutes of Health; Bethesda, Md.).

The rate of siRNA and RTA release under neutral and acidic pH conditions was determined by suspending 1×10¹⁰ particles in 1 mL of a simulated body fluid (EMEM with 150 mM NaCl and 10% serum, pH 7.4) or citric acid buffer (pH 5.0) for various periods of time at 37° C. Particles were pelleted via centrifugation (5 minutes at 5,000×g for protocells and 30 minutes at 15,000×g for liposomes; Microfuge® 16 Centrifuge; Beckman-Coulter; Brea, Calif.). siRNA and RTA concentrations in the supernatant were determined using UV-visible spectroscopy and SDS-PAGE, as described above. The concentration of released cargo was converted into a percentage of the cargo concentration that was initially encapsulated within 10¹⁰ particles.

Quantification of Cyclin A2, B1, D1, and E Protein Expression.

To determine the concentration of siRNA necessary to silence 90% of cyclin A2, cyclin B1, cyclin D1, or cyclin E expression (IC₉₀, see FIG. 4AX3), 1×10⁶ Hep3B cells were exposed to various concentrations of siRNA loaded in SP94-targeted DOPC protocells for 48 hours at 37° C. Cells were centrifuged (1000 rpm, 1 minute) to remove excess particles, fixed with 3.7% formaldehyde (15 minutes at room temperature), and permeabilized with 0.2% Triton X-100 (5 minutes at room temperature); cells were then exposed to a 1:500 dilution of anti-cyclin A2, anti-cyclin B1, anti-cyclin D1, or anti-cyclin E, labeled using an Alexa Fluor® 488 Antibody Labeling Kit, for 1 hour at 37° C. Cells were washed three times and re-suspended in D-PBS for flow cytometry analysis (FACSCalibur). GraphPad Prism (GraphPad Software, Inc.; La Jolla, Calif.) was employed to calculate IC₉₀ values from plots of log(siRNA concentration) versus mean fluorescence intensity; the initial protein concentration was taken to be the mean fluorescence intensity of antibody-labeled cells exposed to siRNA-loaded protocells for 5 minutes.

To determine the time-dependent decrease in cyclin A2, cyclin B1, cyclin D1, and cyclin E expression (see FIG. 4BX3), siRNA-loaded, SP94-targeted DOPC protocells were mixed with 1×10⁶ Hep3B cells such that the final siRNA concentration was 125 pM; cells and protocells were incubated at 37° C. for various periods of time, and resulting protein levels were determined via immunofluorescence as described above.

To collect the data depicted in FIG. 4CX3 (left axis), a sufficient volume of siRNA-loaded, SP94-targeted DOPC protocells, DOPC lipoplexes, or DOTAP lipoplexes was added to 1×10⁶ Hep3B or hepatocytes such that the final siRNA concentration was 125 pM. Samples were incubated at 37° C. for 48 hours, and the resulting decrease in cyclin A2 expression was quantified as described above. To determine the values plotted in FIG. 4CX3 (right axis), 1×10⁶ Hep3B cells were exposed to various concentrations (particles/mL) of siRNA-loaded, SP94-targeted DOPC protocells, DOPC lipoplexes, or DOTAP lipoplexes for 48 hours at 37° C.; cyclin A2 expression was quantified using immunofluorescence, and the number of particles necessary to reduce cyclin A2 expression by 90% was calculated from a plot of particle concentration versus cyclin A2 concentration.

Cells depicted in FIG. 5 X3 were exposed to 10-fold excess of siRNA-loaded, SP94-targeted DOPC protocells with Alexa Fluor® 647-labeled cores for either 1 hour or 48 hours at 37° C. Cells were washed 3 times with D-PBS, labeled with Hoechst 33342 per manufacturer's instructions, fixed with 3.7% formaldehyde (15 minutes at room temperature), permeabilized with 0.2% Triton X-100 (5 minutes at room temperature), and blocked with Image-iT® FX signal enhancer (30 minutes, room temperature). Cells were then exposed to Alexa Fluor® 488-labeled antibodies against cyclin A2, B1, D1, or E (diluted 1:500 in 1% BSA) overnight at 4° C., washed 3 times in D-PBS, and mounted with SlowFade® Gold.

Quantification of Apoptosis Induced by siRNA-Loaded, SP94-Targeted Protocells.

The time-dependent viability of Hep3B and hepatocytes (see FIG. 6AX3) exposed to siRNA-loaded, SP94-targeted protocells was determined by incubating 1×10⁶ cells with 125 pM of siRNA for various periods of time at 37° C. Cells were centrifuged (1000 rpm, 1 minute) to remove excess protocells and stained with Alexa Fluor 488®-labeled annexin V and propidium iodide. The number of viable (double-negative) and non-viable (single- or double-positive) cells was determined via flow cytometry (FACSCalibur).

Cells shown in FIGS. 6BX3 and 6CX3 were exposed to a 10-fold excess of siRNA-loaded, SP94-targeted protocells with Alexa Fluor® 647-labeled cores for either 1 hour or 48 hours at 37° C. Cells were then washed 3 times with D-PBS, stained with Hoechst 33342, Alexa Fluor® 488-labeled annexin V, and propidium iodide per manufacturer's instructions, fixed (3.7% formaldehyde for 10 minutes at room temperature), and mounted with SlowFade® Gold.

Quantification of Nascent Protein Synthesis.

The IC₉₀ value of RTA-loaded, SP94-targeted DOPC protocells (see FIG. 8A X3) was determined by incubating 1×10⁶ Hep3B cells with various concentrations of protocell-encapsulated RTA for 48 hours at 37° C. The resultant decrease in nascent protein synthesis was detected using the Click-iT® AHA Alexa Fluor® 488 Protein Synthesis HCS Assay (per manufacturer's instructions) and quantified via flow cytometry (FACSCalibur). The mean fluorescence intensity of each sample was plotted against log(toxin concentration), and the IC₉₀ value was determined using GraphPad Prism.

The time-dependent decline in nascent protein synthesis (see FIG. 8BX3) was measured by incubating RTA-loaded, SP94-targeted protocells ([RTA]=25 pM) with 1×10⁶ Hep3B cells for various periods of time at 37° C.; nascent protein synthesis was assayed for as described above.

To collect the data shown in FIG. 8CX3 (left axis), a sufficient volume of RTA-loaded, SP94-targeted DOPC protocells or liposomes was added to 1×10⁶ Hep3B or hepatocytes such that the final RTA concentration was 25 pM. Samples were incubated at 37° C. for 48 hours, and the resulting decrease in nascent protein synthesis was quantified as described above. To determine the values plotted in FIG. 8C (right axis), 1×10⁶ Hep3B cells were exposed to various concentrations (particles/mL) of RTA-loaded, SP94-targeted DOPC protocells or liposomes for 48 hours at 37° C.; protein biosynthesis was quantified using the Click-iT® AHA Alexa Fluor® 488 Protein Synthesis HCS Assay, and the number of particles necessary to reduce nascent protein synthesis by 90% was calculated from a plot of particle concentration versus nascent protein concentration.

Cells shown in FIG. 9X3 were exposed to a 10-fold excess of RTA-loaded, SP94-targeted DOPC protocells with Alexa Fluor® 647-labeled cores for 1 hour or 48 hours at 37° C. Newly synthesized proteins were labeled using the Click-iT® AHA Alexa Fluor® 488 Protein Synthesis HCS Assay (per manufacturer's instructions). Cells were then stained with Hoechst 33342 per manufacturer's instructions, fixed with 3.7% formaldehyde (10 minutes at room temperature), and mounted using SlowFade® Gold.

Quantification of Apoptosis Induced by RTA-Loaded, SP94-Targeted Protocells.

The time-dependent activation of caspase-9 and caspase-3 (see FIG. 10AX3) was determined by exposing 1×10⁶ Hep3B and hepatocytes to RTA-loaded, SP94-targeted DOPC protocells ([RTA]=25 pM) for various periods of time at 37° C. The degree of caspase activation was quantified using the CaspGLOW™ Fluorescein Active Caspase-9 and CaspGLOW™ Red Active Caspase-3 Staining Kits; flow cytometry (FACSCalibur) was employed to determine the number of cells expressing green fluorescence (FL1) and/or red fluorescence (FL2) at levels 100-times higher than that of the background (viable Hep3B cells). Apoptotic cells were defined as those positive for caspase-9 and/or caspase-3.

Cells shown in FIGS. 10BX3 and 10CX3 were exposed to a 10-fold excess of RTA-loaded, SP94-targeted DOPC protocells with Alexa Fluor® 647-labeled cores for 48 hours at 37° C. Active caspase-9 and active caspase-3 were labeled using the CaspGLOW™ Fluorescein Active Caspase-9 and CaspGLOW™ Red Active Caspase-3 Staining Kits (respectively). Cells were then washed 3 times in D-PBS, stained with Hoechst 33342 per manufacturer's instructions, fixed (3.7% formaldehyde for 10 minutes at room temperature), and mounting using SlowFade® Gold.

Flow Cytometry Equipment and Settings.

For FIGS. 4AX3-4CX3, 6DX3, 8AX3-8CX3, and 10AX3, cell samples were analyzed with a FACSCalibur flow cytometer (Becton Dickinson; Franklin Lakes, N.J.) equipped with BD CellQuest™ software, version 5.2.1. Samples were acquired with the fsc channel in linear mode and all other channels in log mode. Events were triggered based upon forward light scatter, and a gate was placed on the forward scatter-side scatter plot that excluded cellular debris. Alexa Fluor® 488 and fluorescein were excited using the 488-nm laser source, and emission intensity was collected in the FL1 channel (530/30 filter/bandpass). Propidium iodide and sulfo-rhodamine (CaspGLOW™ Red Active Caspase-3 Staining Kit) were excited using the 488-nm laser source, and emission intensity was collected in the FL2 channel (585/42). Mean fluorescence intensity was determined using FlowJo Software, version 6.4 (Tree Star, Inc.; Ashland, Oreg.). All plots were generated using Sigma Plot, version 11.0 (Systat Software, Inc.; San Jose, Calif.).

Confocal Fluorescence Microscopy Equipment and Settings.

Three- and four-color images were acquired using a Zeiss LSM510 META (Carl Zeiss MicroImaging, Inc.; Thornwood, N.Y.) operated in Channel mode of the LSM510 software; a 63X, 1.4-NA oil immersion objective was employed in all imaging. Typical laser power settings were: 30% transmission for the 405-nm diode laser, 5% transmission (60% output) for the 488-nm Argon laser, 100% transmission for the 543-nm HeNe laser, and 85% transmission for the 633-nm HeNe laser. Gain and offset were adjusted for each channel to avoid saturation and were typically maintained at 500-700 and −0.1, respectively. 8-bit z-stacks with 1024×1024 resolution were acquired with a 0.7 to 0.9-μm optical slice. LSM510 software was used to overlay channels and to create collapsed projections of z-stack images. All fluorescence images are collapsed projections.

For all microscopy experiments, cells were grown in culture flasks to 70-80% confluence, harvested (0.05% trypsin, 10 minutes), centrifuged at 4000 rpm for 2 minutes, and re-suspended in complete growth medium. 1×10⁴-1×10⁶ cells/mL were seeded on sterile coverslips (25-mm, No. 1.5) coated with 0.01% poly-L-lysine (150-300 kDa) and allowed to adhere for 4-24 hours at 37° C. before being exposed to protocells. 48-hour samples were spun back onto coverslips using a Cytopro® Centrifuge, model 7620 (Wescor, Inc.; Logan, Utah).

### REFERENCES FOR EXAMPLE 3

1. Peer D, Karp J M, Hong S, Farokhzad O C, Margalit R, Langer R. Nanocarriers as an emerging platform for cancer therapy. Nat Nano. 2007; 2(12):751-760.
2. Petros R A, DeSimone J M. Strategies in the design of nanoparticles for therapeutic applications. Nat Rev Drug Discov. 2010; 9(8):615-627.
3. Wang M, Thanou M. Targeting nanoparticles to cancer. Pharmacological Research. 2010; 62(2):90-99.
4. Meister G, Tuschl T. Mechanisms of gene silencing by double-stranded RNA. Nature. 2004; 431(7006):343-349.
5. Rana T M. Illuminating the silence: understanding the structure and function of small RNAs. Nat Rev Mol Cell Biol. 2007; 8(1):23-36.
6. Davidson B L, McCray P B. Current prospects for RNA interference-based therapies. Nat Rev Genet. 2011; 12(5):329-340.
7. Lares M R, Rossi J J, Ouellet D L. RNAi and small interfering RNAs in human disease therapeutic applications. Trends in Biotechnology. 2010; 28(11):570-579.
8. Bumcrot D, Manoharan M, Koteliansky V, Sah D W Y. RNAi therapeutics: a potential new class of pharmaceutical drugs. Nat Chem Biol. 2006; 2(12):711-719.
9. Aagaard L, Rossi J J. RNAi therapeutics: Principles, prospects and challenges. Advanced Drug Delivery Reviews. 2007; 59(2-3):75-86.
10. Ozpolat B, Sood A K, Lopez-Berestein G. Nanomedicine based approaches for the delivery of siRNA in cancer. Journal of Internal Medicine. 2010; 267(1):44-53.
11. Petrocca F, Lieberman J. Promise and Challenge of RNA Interference-Based Therapy for Cancer. Journal of Clinical Oncology. Feb. 20, 2011; 29(6):747-754.
12. de Fougerolles A, Vornlocher H-P, Maraganore J, Lieberman J. Interfering with disease: a progress report on siRNA-based therapeutics. Nat Rev Drug Discov. 2007; 6(6):443-453.
13. Davis M E, Zuckerman J E, Choi C H J, et al. Evidence of RNAi in humans from systemically administered siRNA via targeted nanoparticles. Nature. 2010; 464(7291):1067-1070.
14. Jackson A L, Burchard J, Leake D, et al. Position-specific chemical modification of siRNAs reduces “off-target” transcript silencing. RNA. Jul. 1, 2006; 12(7):1197-1205.
15. Judge A D, Sood V, Shaw J R, Fang D, McClintock K, MacLachlan I. Sequence-dependent stimulation of the mammalian innate immune response by synthetic siRNA. Nat Biotech. 2005; 23(4):457-462.
16. Soutschek J, Akinc A, Bramlage B, et al. Therapeutic silencing of an endogenous gene by systemic administration of modified siRNAs. Nature. 2004; 432(7014):173-178.
17. Pecot C V, Calin G A, Coleman R L, Lopez-Berestein G, Sood A K. RNA interference in the clinic: challenges and future directions. Nat Rev Cancer. 2011; 11(1):59-67.
18. Whitehead K A, Langer R, Anderson DG. Knocking down barriers: advances in siRNA delivery. Nat Rev Drug Discov. 2009; 8(2):129-138.
19. Edbrooke M, Clarke N. RNAi Therapeutics: addressing targets? Eur Pharm Rev. August 2008; 4:11-17.
20. Alderson R F, Kreitman R J, Chen T, et al. CAT-8015: A Second-Generation

Pseudomonas Exotoxin A-Based Immunotherapy Targeting CD22-Expressing Hematologic Malignancies. Clinical Cancer Research. Feb. 1, 2009; 15(3):832-839.
21. Wayne A S, Kreitman R J, Findley H W, et al. Anti-CD22 Immunotoxin RFB4(dsFv)-PE38 (BL22) for CD22-Positive Hematologic Malignancies of Childhood: Preclinical Studies and Phase I Clinical Trial. Clinical Cancer Research. Mar. 15, 2010; 16(6):1894-1903.
22. Pastan I, Hassan R, FitzGerald D J, Kreitman R J. Immunotoxin therapy of cancer. Nat Rev Cancer. 2006; 6(7):559-565.
23. Cauda V, Argyo C, Bein T. Impact of different PEGylation patterns on the long-term bio-stability of colloidal mesoporous silica nanoparticles. Journal of Materials Chemistry. 2010; 20(39):8693-8699.
24. Cauda V, Argyo C, Schlossbauer A, Bein T. Controlling the delivery kinetics from colloidal mesoporous silica nanoparticles with pH-sensitive gates. Journal of Materials Chemistry. 2010; 20(21):4305-4311.
25. Cauda V, Engelke H, Sauer A, et al. Colchicine-Loaded Lipid Bilayer-Coated 50 nm Mesoporous Nanoparticles Efficiently Induce Microtubule Depolymerization upon Cell Uptake. Nano Letters. 2010 Jul. 14 2010; 10(7):2484-2492.
26. Cauda V, Muehistein L, Onida B, Bein T. Tuning drug uptake and release rates through different morphologies and pore diameters of confined mesoporous silica. Microporous Mesoporous Mater. 2009; 118:435-442.
27. Cauda V, Schlossbauer A, Bein T. Bio-degradation study of colloidal mesoporous silica nanoparticles: Effect of surface functionalization with organo.silanes and poly(ethylene glycol). Microporous Mesoporous Mater. 2010; 132:60-71.
28. Horn C, Lu J, Liong M, et al. Mesoporous Silica Nanoparticles Facilitate Delivery of siRNA to Shutdown Signaling Pathways in Mammalian Cells. Small. 2010; 6(11):1185-1190.
29. Liong M, Lu J, Kovochich M, et al. Multifunctional Inorganic Nanoparticles for Imaging, Targeting, and Drug Delivery. ACS Nano. 2008 May 1 2008; 2(5):889-896.
30. Meng H, Liong M, Xia T, et al. Engineered Design of Mesoporous Silica Nanoparticles to Deliver Doxorubicin and P-Glycoprotein siRNA to Overcome Drug Resistance in a Cancer Cell Line. ACS Nano. 2010 Aug. 24 2010; 4(8):4539-4550.
31. Meng H, Xue M, Xia T, et al. Autonomous in Vitro Anticancer Drug Release from Mesoporous Silica Nanoparticles by pH-Sensitive Nanovalves. Journal of the American Chemical Society. 2010 Sep. 15 2010; 132(36):12690-12697.
32. Sauer A M, Schlossbauer A, Ruthardt N, Cauda V, Bein T, Braüchle C. Role of Endosomal Escape for Disulfide-Based Drug Delivery from Colloidal Mesoporous Silica Evaluated by Live-Cell Imaging. Nano Letters. 2010 Sep. 8 2010; 10(9):3684-3691.
33. Xia T, Kovochich M, Liong M, et al. Polyethyleneimine Coating Enhances the Cellular Uptake of Mesoporous Silica Nanoparticles and Allows Safe Delivery of siRNA and DNA Constructs. ACS Nano. 2009 Oct. 27 2009; 3(10):3273-3286.
34. Ashley C E, Carnes E C, Phillips G K, et al. The targeted delivery of multicomponent cargos to cancer cells by nanoporous particle-supported lipid bilayers. Nat Mater. 2011; 10(5):389-397.
35. Carroll N J, Pylypenko S, Atanassov P B, Petsev D N. Microparticles with Bimodal Nanoporosity Derived by Microemulsion Templating. Langmuir. 2009; 25:13540-13544.
36. Liu J, Stace-Naughton A, Jiang X, Brinker C J. Porous Nanoparticle Supported Lipid Bilayers (Protocells) as Delivery Vehicles. Journal of the American Chemical Society. 2009 Feb. 4 2009; 131(4):1354-1355.
37. Pines J. Cyclins: wheels within wheels. Cell Growth Differ. Jun. 1, 1991; 2(6):305-310.
38. Lord M J, Jolliffe N A, Marsden C J, et al. Ricin: Mechanisms of Cytotoxicity. Toxicological Reviews. 2003; 22(1):53-64.
39. Herrera L, Bostrom B, Gore L, et al. A Phase 1 Study of Combotox in Pediatric Patients With Refractory B-lineage Acute Lymphoblastic Leukemia. Journal of Pediatric Hematology/Oncology. 2009; 31(12):936-941 910.1097/MPH.1090b1013e3181bdf1211.
40. Herrera L, Yarbrough S, Ghetie V, Aquino D B, Vitetta E S. Treatment of SCID//human B cell precursor ALL with anti-CD19 and anti-CD22 immunotoxins. Leukemia. 2003; 17(2):334-338.
41. Grimm D, Kay M A. Therapeutic application of RNAi: is mRNA targeting finally ready for prime time? The Journal of Clinical Investigation. 2007; 117(12):3633-3641.
42. Chen Y, Bathula S R, Li J, Huang L. Multifunctional Nanoparticles Delivering Small Interfering RNA and Doxorubicin Overcome Drug Resistance in Cancer. Journal of Biological Chemistry. Jul. 16, 2010; 285(29):22639-22650.
43. Li S-D, Chen Y-C, Hackett M J, Huang L. Tumor-targeted Delivery of siRNA by Self-assembled Nanoparticles. Mol Ther. 2007; 16(1):163-169.
44. Howard K A, Rahbek U L, Liu X, et al. RNA Interference in Vitro and in Vivo Using a Chitosan/siRNA Nanoparticle System. Mol Ther. 2006; 14(4):476-484.
45. Hu-Lieskovan S, Heidel J D, Bartlett D W, Davis M E, Triche T J. Sequence-Specific Knockdown of EWS-FLI1 by Targeted, Nonviral Delivery of Small Interfering RNA Inhibits Tumor Growth in a Murine Model of Metastatic Ewing's Sarcoma. Cancer Research. Oct. 1, 2005; 65(19):8984-8992.
46. Schiffelers R M, Ansari A, Xu J, et al. Cancer siRNA therapy by tumor selective delivery with ligand-targeted sterically stabilized nanoparticle. Nucleic Acids Research. Jan. 1, 2004; 32(19):e149.
47. Cheng H, Zhu J-L, Zeng X, Jing Y, Zhang X-Z, Zhuo R-X. Targeted Gene Delivery Mediated by Folate-polyethylenimine-block-poly(ethylene glycol) with Receptor Selectivity. Bioconjugate Chemistry. 2009 Mar. 18 2009; 20(3):481-487.
48. DiFiglia M, Sena-Esteves M, Chase K, et al. Therapeutic silencing of mutant huntingtin with siRNA attenuates striatal and cortical neuropathology and behavioral deficits. Proceedings of the National Academy of Sciences. Oct. 23, 2007; 104(43):17204-17209.
49. Chen Y, Zhu X, Zhang X, Liu B, Huang L. Nanoparticles Modified With Tumor-targeting scFv Deliver siRNA and miRNA for Cancer Therapy. Mol Ther. 2010; 18(9):1650-1656.
50. Orava E W, Cicmil N, Gariépy J. Delivering cargoes into cancer cells using DNA aptamers targeting internalized surface portals. Biochim Biophys Acta. 2010; 1798(12):2190-2200.
51. Becker A L, Orlotti N I, Folini M, et al. Redox-Active Polymer Microcapsules for the Delivery of a Survivin-Specific siRNA in Prostate Cancer Cells. ACS Nano. 2011 Feb. 22 2011; 5(2):1335-1344.
52. MacDiarmid J A, Amaro-Mugridge N B, Madrid-Weiss J, et al. Sequential treatment of drug-resistant tumors with targeted minicells containing siRNA or a cytotoxic drug. Nat Biotech. 2009; 27(7):643-651.
53. Tanaka T, Mangala L S, Vivas-Mejia P E, et al. Sustained Small Interfering RNA Delivery by Mesoporous Silicon Particles. Cancer Research. May 1, 2010; 70(9):3687-3696.
54. Li J, Chen Y-C, Tseng Y-C, Mozumdar S, Huang L. Biodegradable calcium phosphate nanoparticle with lipid coating for systemic siRNA delivery. Journal of Controlled Release. 2010; 142(3):416-421.
55. Li S-D, Huang L. Targeted Delivery of Antisense Oligodeoxynucleotide and Small Interference RNA into Lung Cancer Cells. Molecular Pharmaceutics. 2006 Oct. 1 2006; 3(5):579-588.
56. Pirollo K F, Rait A, Zhou Q, et al. Materializing the Potential of Small Interfering RNA via a Tumor-Targeting Nanodelivery System. Cancer Research. Apr. 1, 2007; 67(7):2938-2943.
57. Sun T-M, Du J-Z, Yao Y-D, et al. Simultaneous Delivery of siRNA and Paclitaxel via a “Two-in-One” Micelleplex Promotes Synergistic Tumor Suppression. ACS Nano. 2011 Feb. 22 2011; 5(2):1483-1494.
58. Xiong X-B, Lavasanifar A. Traceable Multifunctional Micellar Nanocarriers for Cancer-Targeted Co-delivery of MDR-1 siRNA and Doxorubicin. ACS Nano. 2011 Jun. 28 2011; 5(6):5202-5213.
59. Chen Y, Bathula S R, Yang Q, Huang L. Targeted Nanoparticles Deliver siRNA to Melanoma. J Invest Dermatol. 2010; 130(12):2790-2798.
60. Chono S, Li S-D, Conwell C C, Huang L. An efficient and low immunostimulatory nanoparticle formulation for systemic siRNA delivery to the tumor. Journal of Controlled Release. 2008; 131(1):64-69.
61. Bhattarai S, Muthuswamy E, Wani A, et al. Enhanced Gene and siRNA Delivery by Polycation-Modified Mesoporous Silica Nanoparticles Loaded with Chloroquine. Pharmaceutical Research. 2010; 27(12):2556-2568.
62. Liu J W, Jiang X M, Ashley C, Brinker C J. Electrostatically Mediated Liposome Fusion and Lipid Exchange with a Nanoparticle-Supported Bilayer for Control of Surface Charge, Drug Containment, and Delivery. Journal of the American Chemical Society. June 2009; 131(22):7567-7569.
63. Liu J W, Stace-Naughton A, Brinker C J. Silica nanoparticle supported lipid bilayers for gene delivery. Chemical Communications. 2009(34):5100-5102.
64. Wu S Y, Singhania A, Burgess M, et al. Systemic delivery of E6/7 siRNA using novel lipidic particles and its application with cisplatin in cervical cancer mouse models. Gene Ther. 2011; 18(1):14-22.
65. Lo A, Lin C-T, Wu H-C. Hepatocellular carcinoma cell-specific peptide ligand for targeted drug delivery. Molecular Cancer Therapeutics. Mar. 1, 2008; 7(3):579-589.
66. Moore N M, Sheppard C L, Barbour T R, Sakiyama-Elbert S E. The effect of endosomal escape peptides on in vitro gene delivery of polyethylene glycol-based vehicles. The Journal of Gene Medicine. 2008; 10(10):1134-1149.

### Example 4

### Targeted Delivery of Therapeutic RNA and DNA to Host Cells ‘Infected’ with NipahVirus via Mesoporous Silica Nanoparticle-Supported Lipid Bilayers (MSN-SLBs)

Nipah virus (NiV), a highly pathogenic member of the Paramyxoviridae family, has been classified as a BSL-4 select agent due to its numerous routes of transmission and the high mortality rates associated with infection. Despite recent advances in understanding the cellular tropism of NiV, however, treatment remains primarily supportive. To this end, we have developed mesoporous silica nanoparticle-supported lipid bilayers (MSN-SLBs; see Nature Materials (2011) 10: 389-397) that specifically deliver high concentrations of therapeutic RNA and DNA to model host cells transfected with a NiV gene. MSN-SLBs are formed via fusion of liposomes (DOPC with 5 wt % DOPE for peptide and PEG conjugation) to 100-nm mesoporous silica nanoparticles. Due to its high surface area (>1000 m²/g) and large (20-25 nm), surface-accessible pores, the mesoporous silica core can be rapidly loaded with high concentrations (˜1 μM per 10¹⁰ particles) of siRNA that induces sequence-specific degradation of NiV nucleocapsid protein (NiV-N) mRNA. Liposome fusion to siRNA-loaded cores results in a supported lipid bilayer (SLB) that promotes long-term (>3 months) cargo retention and provides a fluid interface for ligand display. MSN-SLB bilayers are modified with multiple copies of a targeting peptide, a peptide (R8) that induces macropinocytosis, and PEG to enable cytosolic delivery of siRNA to model host cells.

Using phage display, we have identified peptides that bind to ephrin B2 (EB2), a transmembrane-anchored ligand of the EphB2, EphB3, and EphB4 tyrosine kinases that is expressed by human endothelial cells and neurons and that acts as the primary receptor for NiV entry via macropinocytosis; TGAILHP (SEQ ID NO:18) was the predominant sequence after five rounds of affinity selection against CHO-K1 cells transfected to express human EB2 and counter-selections against both parental CHO-K1 and CHO-K1 cells transfected to express human ephrin B1. Using flow cytometry, we found that TGAILHP-targeted MSN-SLBs have a nanomolar affinity for EB2-positive cells (HEK 293) at both high (1.5 wt % or ˜500 peptides/particle) and low (0.015 wt % or ˜5 peptides/particle) peptide valencies. Importantly, MSN-SLBs modified with 0.015 wt % of TGAILHP (SEQ ID NO:18) and 10 wt % PEG-2000, which promotes colloidal stability and reduces non-specific interactions, have a 10³-fold higher affinity for HEK 293 cells than for EB2-negative cells (parental CHO-K1). Using confocal fluorescence microscopy, we determined that MSN-SLBs modified with 0.015 wt % of TGAILHP (SEQ ID NO:18) and 0.500 wt % of R8 are rapidly (t_{1/2}=5 minutes) internalized by HEK 293 and that pre-treatment of cells with various macropinocytosis inhibitors reduces uptake by 60-80%. Acidification of macropinosomes (1) destabilizes the SLB, which triggers release of encapsulated siRNA and (2) protonates the R8 peptide, which disrupts macropinosomal membranes via the proton-sponge mechanism, both of which enable cytosolic distribution of siRNA.

Selective binding and internalization, followed by macropinosome escape enable TGAILHP-targeted, siRNA-loaded MSN-SLBs to silence 90% of NiV-N mRNA in HEK 293 at a siRNA concentration of ˜5 pM without affecting NiV-N levels in parental CHO-K1 cells. However, siRNA-mediated RNAi is transient, and NiV-N mRNA levels start to increase 5 days post-treatment. We, therefore, designed a plasmid that encodes a small hairpin RNA (shRNA) specific for NiV-N, packaged the plasmid with histones, and modified the resulting 18-nm complex with a nuclear localization sequence (NLS) before loading it within the silica core. MSN-SLBs have a 100-fold higher capacity for histone-packaged plasmids (4.5 kbp) than corresponding lipoplexes formed from a 50:50 molar ratio of DOTAP and DOPE. Furthermore, plasmid-loaded MSN-SLBs modified with 0.015 wt % of TGAILHP (SEQ ID NO:18) and 0.500 wt % of R8 silence 90% of NiV-N mRNA in HEK 293 at a particle:cell ratio of ˜1:20 (˜1750 plasmids/cell) and induce long-term RNAi; the concentration of NiV-N mRNA remains at <10% of its initial value for 4 weeks. Due to their enormous cargo capacity, as well as their stability and specificity, MSN-SLBs show promise as delivery vehicles for therapeutic agents capable of preventing viral replication and transmission.

### Example 5

### Transdermal Protocells

Two experiments were performed to test whether or not Protocells could be engineered to facilitate SC permeation enhancement and transdermal delivery. In the first experiment, the goal was to determine if it was possible for a standard formulation of Protocells to cross the skin either by passive diffusion across the stratum corneum or via bypassing the skin. To accomplish this, a vertical Franz diffusion apparatus, full thickness skin obtained from abdominoplasties, and inductively coupled plasma mass spectroscopy (ICP-MS) was used. The full experimental methods are described in the following section, but briefly the SC was removed from half the samples using a tape stripping method, and left intact on the remaining samples. Protocells were made using silica particles with a mean diameter of 90 nm and a pore-size diameter of 2.5 nm, and liposomes with a mean diameter of 120 nm and a bilayer composition formulated with 55 wt % DOPC, 30 wt % Chol, and 15 wt % DOPE-PEG-2000.

Table 1 shows the name, abbreviation, and relevant physical properties for all lipids. A modified Franz diffusion cell was used for diffusion experiments by filling the receptacle, placing the skin sample on and clamping the donor cap down. Controls from each group (SC Intact, SC Removed) were treated with 0.5×PBS, while the remaining samples were treated with 8.125 mg of Protocells for 24 hours. The remaining sample in the donor cap, skin samples, and receptacle fluid were then collected. Only the receptacle fluids were analyzed with ICP-MS due to the high cost/sample. FIG. 3aX5 shows the total amount of SiO₂ in the receptacle fluid, per group (n=3), as reported by ICP-MS, demonstrating that Protocells are able to penetrate the SC and diffuse across the skin. Nearly 4× the amount of Protocells were able to diffuse across skin samples that had the SC removed in comparison to those with an intact SC, however, due to the high degree of error within each group these values are not statistically significant therefore these data only confirm the feasibility of the proposed work. The next experiment served two purposes, first, due to the high cost/sample for ICP-MS, to develop a cost effective method for quantifying the transdermal flux, and second, to determine the effect of Protocell's SLB composition and formulation on the transdermal kinetics. Spectrafluorimetry was chosen to quantify flux due to its high sensitivity, facile access to a fluorimeter, and the ease with which the core can be fluorescently labeled. FIG. 3bX5 is a schematic that illustrates how the Protocell core can be fluorescently labeled through functionalization of the cores using the 1^{o}-amine-containing organosilane, 3-aminopropyltriethoxy silane (APTES), followed by incubation with an amine-reactive fluorophore. The skin itself is highly autofluorescent at all visible wavelengths, but far-red wavelengths exhibit the least amount of autofluorescence as demonstrated by spectrafluorimetry and confocal laser scanning microscopy (CLSM). Therefore, Alexa Flour 633 (ex: 632, em: 647) was chosen for this experiment and will be used in all subsequent experiments. The fluorimeter sensitivity for the 633-labeled cores in receptacle buffer ranged from ˜195 ng/ml-500 ng/ml depending on the skin's degree of autofluorescence. In this experiment Protocells with fluorescently labeled cores were constructed using three basic SLB compositions with a total of six formulations based on differences in lipid transition temperature, degree of saturation/un-saturation, and head group: 1.) 70 wt % DOPC/30 wt % Chol, 2.) 55 wt % DOPC/30 wt % Chol/15 wt % DOPE-PEG-2000, 3.) 70 wt % DSPC/30 wt % Chol, 4.) 55 wt % DSPC/30w % Chol/15 wt % DSPE-PEG-2000, 5.) 45 wt % DOPC/30 wt % Chol/25 wt % DOPE, and 6.) 30 wt % DOPC/30 wt % Chol/25 wt % DOPE/15 wt % DOPE. The SC was left intact on all samples and controls were treated with 0.5×PBS while each sample was treated with 8 mg of Protocells for 24 hours. FIG. 3cX5 summarizes the results and illustrates that SLB composition and formulation drastically affects the transdermal kinetics of Protocells. This is consistent with the literature, which suggests that lipids with lower transition temperatures diffuse deeper into full thickness skin and lipids with higher transition temperatures remain localized in the stratum corneum.⁵¹ Collectively, preliminary data demonstrates the feasibility of the proposed work and its high potential for success. Additionally, a cost effective fluorimetry protocol has been developed to quantify the transdermal kinetics of Protocells.

### Approach

### Protocell Synthesis and Characterization:

Nanoporous particle cores are synthesized using different evaporation induced self-assembly (EISA) approaches either in a colloidal solution or via aersolization. EISA uses amphiphilic surfactant and block-copolymers as structure directing agents in conjunction with soluble sol-gel precursors (i.e. acid or base, H₂O or EtOH, and some kind of organosilane) to promote self-assembly of spherical nanosized silica (SiO₂) particles with highly ordered/uniform pore sizes through simple solvent evaporation.^{56, 57} Once particle synthesis is complete the structure directing agent is removed using solvent extraction or calcination at 500° C. Particle size (30-1000 nm), porosity, pore size (2.5-20 nm), dissolution kinetics, and surface chemistry can be controlled by tailoring concentrations and through the choice of structured directing agents. Additionally, post synthesis functionalizations can be made (FIG. 3bX5) using the procedure described above. SLBs are formed via extrusion, a process in which an aqueous lipid solution is passed through a porous polycarbonate membrane with uniform pores multiple times to yield a monodisperse liposome solution. Lipids are purchased as 25 mg/ml stocks solutions stored in chloroform so they must be extracted and dried prior to extrusion. Lipids are dispensed into a single scintillation vial formulated in different ratio such that the final mass is 2.5 mg. The choice of lipid composition and formulation allows for one level of precise control over the SLB's physical and chemical properties, an additional level of control comes from subsequent SLB modifications once it has been fused to the core (Table 1). Chloroform is removed under a vacuum and the lipid is rehydrated with 0.5×PBS to a final concentration of 2.5 mg/ml and extruded, or immediately stored at −20° C. for <6 months.

| Abbreviation | Lipid Name | T_{M}(° C.) | MW (g/mol) |
|---|---|---|---|
| DOPC | 1,2-dioleoyl-sn-glycero-3- | −20 | 786.113 |
| | phosphocholine | | |
| DPPC | 1,2-dipalmitoyl-sn-glycero- | 41 | 733.562 |
| | 3-phosphocholine | | |
| DSPC | 1,2-distearoyl-sn-glycero- | 55 | 790.145 |
| | 3-phosphocholine | | |
| DOPE | 1,2-dioleoyl-sn-glycero- | −16, 10 | 744.034 |
| | 3-phosphoethanolamine | | |
| DPPE | 1,2-dipalmitoyl-sn-glycero- | 63, 118 | 692 |
| | 3-phosphoethanolamine | | |
| DSPE | 1,2-distearoyl-sn-glycero- | 74, 100 | 748.065 |
| | 3-phosphochoethanolamine | | |
| DOPE-PEG | 1,2-dioleoyl-sn-glycero-3- | −63.5 | 2801.465 |
| | phosphoethanolamine-N- | | |
| | [methoxy(polyethylene glycol)- | | |
| | 2000] (ammonium salt) | | |
| DPPE-PEG | 1,2-dipalmitoyl-sn-glycero- | −63.5 | 2749.391 |
| | 3-phosphoethanolamine-N- | | |
| | [methoxy(polyethylene glycol)- | | |
| | 2000] (ammonium salt) | | |
| DSPE-PEG | 1,2-distearoyl-sn-glycero-3- | −63.5 | N/A |
| | phosphoethanolamine-N- | | |
| | [methoxy(polyethylene glycol)- | | |
| | 2000] (ammonium salt) | | |
| Chol | Cholesterol | 147-149 | 386.65 |
| Chol-S | Sodium Cholesteryl Sulfate | 178-180 | 488.7 |
| Cer18:1 | N-oleoyl-D-erythro-sphingosine | N/A | 563.938 |
| Cer18 | N-stearoyl-D-erythro- | N/A | 567.97 |
| | sphinganine | | |
| Acyl-Cer | 1-oleoyl-N-heptadecanoyl-D- | N/A | 816.373 |
| | erythro-sphingosine | | |

Table 1 shows the names and physical properties of the lipids to be used. Data from: www.avantilipids.com

Note that liposomes are extruded just above the highest Tₘ in the formulation to ensure that all lipids are fluid therefore it is often necessary to place the extruder on a hot plate. Protocells are made by adding liposomes to the cores in volumetric excess using a 3:1 (v/v) ratio and letting them incubate with agitation at room temperature for 30-60 minutes. Next, further bilayer modifications are made (i.e. conjugation of peptides), using heterobifunctional crosslinkers, then the Protocell solution is concentrated to the desired working concentration (<20 mg/ml). Characterization of Protocells and their components consists of transmission electron microscopy (TEM) to qualitatively assess pore and particle structure and to visually and statistically quantify, particle diameter and distribution, dynamic light scattering (DLS) to obtain a hydrodynamic radius, nitrogen sorption (NS) to quantify Brunauer-Emmett-Teller (BET) surface-area and Barret-Joyner-Halenda (BJH) pore-size distribution, zeta potential g) to assess colloidal stability and surface charge, and absorbance or fluorescence to assess cargo-loading capacity.⁹⁻¹¹ Protocell cores are subject to all the fore mentioned before and after any modification is made. Liposomes and Protocells are only subject to 4-potential and DLS before and after any modifications.

### Skin Preparation and Franz Diffusion Apparatus:

Skin preparation and proper handling is important because it can directly impact the skin's structure. Full thickness human skin obtained from abdominoplasties is donated in accordance with local regulations. Upon receipt of the skins, they are double-bagged and stored at −20° C. for <6 months. The skin's barrier function has been shown to remain intact with multiple freeze-thaw cycles.^{58,59} As needed, the frozen skin is thawed in a 30° C. oven and subcutaneous fat is removed using a scalpel, then the skin samples are sectioned into 1-cm² pieces. The samples are then rinsed with DI H₂O and the SC side is blotted dry. In some cases the SC will need to be removed or isolated; this can be accomplished using tape stripping or enzymatic tissue digestion, respectively. When the experiment is complete, skin samples should be washed in 10 ml 0.5×PBS, blotted dry, individually doubled-bagged, wrapped in foil, and frozen until it can be analyzed. Professor Linda Felton's laboratory, located in the Multidisciplinary Research Facility (MRF) houses a vertical, modified Franz diffusion apparatus that is equipped with 9 water-jacketed diffusion cells, heating/cooling circulator, built in stir plate, sampling port and a donor surface area of 0.64 cm², and a receptacle volume of 5.1 mL To prepare an experiment, the receptacle is filled with 0.5×PBS (or some other isotonic buffer) and the temperature is set to 37° C. Next, the skins are stretched over the receptacle, careful to avoid forming of any air bubbles, and the donor cap is clamped into place and covered to prevent dehydration. The skins are then allowed to equilibrate for 1 hour, the receptacle fluid is then replaced and the skins are re-equilibrated for another 30 minutes. Before the experiment starts, 400 ul of receptacle fluid is removed from the sampling port and kept as a 0 hour blank for the receptacle it came from. 400 ul samples are taken from the sampling port at desired time points, and then 400 ul of diffusion buffer is replenished to maintain constant volume and avoid the formation of air bubbles at the skin-fluid interface.

### Spectrafluorimetry:

Quantification of all transdermal Protocell diffusion experiments will be quantified using a PTI QuantaMaster-40 spectrafluorimeter equipped with FelixGX software, two PMT detectors, optical filters, and a sample carousal that can accommodate 4 cuvettes. Skin is highly heterogeneous and highly autofluorescent, characteristics that are usually transferred to the receptacle fluid. This protocol was developed such that these issues can be accounted for during analysis. A standard curve is made from the receptacle fluid of the control sample (24-hour time point) over the concentration range 0.16-1.95×10⁻⁵ mg/ml using half dilutions. In addition, 380 ul are pulled from the control and placed in a cuvette to serve as a blank. This blank remains in the carousal for the duration of the experiment, which allows only three samples to be analyzed at a time. The standards are run 3 times, averaged and reported with a 95% confidence interval, and plotted on a log-log scale to obtain a linear equation. All samples are analyzed a minimum of 3 times and a maximum of 9 for statistical relevance. Once all samples have been analyzed the file is saved, exported as a text file, and manually entered into an excel spreadsheet. The mean of all blank-values are averaged and a 95% confidence interval is calculated. The mean fluorescence intensity (MFI) is individually calculated for all samples at all time points. Linear regression analysis is used to calculate the unknown concentrations. A correction value for each of the 8 samples is determined through the addition/subtraction of each 0-hour MFI to/from the blank MFI in order to normalize everything to the standard curve. The correction value is then added/subtracted to the MFI at each time point to give a corrected MFI. The log of each MFI is taken and using the equation obtained from the standard curve the concentration is calculated. Finally, the concentration values calculated for each 0-hour time point are subtracted from every other time point to give the absolute concentration. Note that all standard curves follow polynomial trends over the entire concentration range; in order to obtain linear curves only the relevant concentration/intensity range is plotted, and fit to linear trend lines (R²>0.9300).

Specific Aim 1—Investigate the parameters of the Protocell's supported lipid bilayer (SLB) and Nanoporous Silica-Particle Core that Influence Transdermal Penetration Kinetics In Vitro and Determine if the SLB Dissociates from the Core.

To accomplish this specific aim, a systematic manipulation of each of the Protocell's biophysical and biochemical properties will be performed. First, each individual property of the SLB will be investigated, followed by independently assessing each of the core's properties. Spectrafluorimetry will be used to quantify flux, and skins will be imbedded in paraffin wax, histologically³² sectioned and imaged using dual-channel CLSM²³ to qualitatively assess Protocell partitioning into the skin. If CLSM is insufficient for this, either TEM⁵¹ or multi-photon microscopy⁴² (SNL-CINT) will be employed. For the experiments concerned with the SLB, all cores will be fluorescently labeled with Alexa Fluor 633 and synthesized via aersolization and templated with cetyl trimethylammonium bromide (CTAB), the standard core optimized for the targeted-Protocell. These particles have a ζ=−20 mV, uniform BJH pore size=2.5 nm, particle size distribution=90±60, and a BET surface-area=1000 g/m². For the experiments concerning the core, the SLB formulation determined to result in the highest overall flux will be used and will be held constant. Finally, once the SLB and core properties have been optimized the fate of the SLB will be determined. The first set of experiments will identify which neutrally charged phospholipid (DOPC, DPPC, and DSPC) alone yields the greatest overall flux over the course of 24 hours, based on transition temperature/fluidity. Van den Bergh et al have shown that fluid lipids (Tₘ<37° C.) diffuse deeper into the skin, while non-fluid lipids (Tₘ>37° C.) remain localized in the SC.⁵¹ Preliminary results support those findings, however an innovative property of Protocells is simultaneously enhanced SLB fluidity and stability due to the nanoporous support conferred by the core and a corresponding decrease in the apparent transition temperature of SLB lipids, as confirmed by temperature-dependent FRAP. This property is especially interesting in the case of DPPC where the apparent T_{M} decreases from 41° C. to 37° C.⁹ If the flux for DPPC Protocells falls between those of DOPC and DSPC Protocells, then subsequent base compositions of Protocells will only use DOPC and DSPC to draw the comparison between Protocells with fluid and non-fluid SLBs, since those results will be consistent with liposomal literature. However, if the overall flux of DPPC Protocells is outside that range, due to SLB-core interactions, then all three SLB base compositions will be used in subsequent experiments in order to further investigate the effects of these interactions. The second experiment will look at the effects of cholesterol, cholesterol-sulfate, and ceramides on the overall flux over the course of 24 hours. The SC is composed of cholesterol, cholesterol sulfate, fatty acids, and ceramides.^{1, 33, 60, 61} Therefore, in an attempt to increase the solubility of Protocells in the skin incorporation of these SC lipids into the SLB will be performed to elucidate any effects on permeation. The effects of each of these lipids will be studied independently and collectively. Preliminary results demonstrate that PEG-2000 has a large effect on flux. Additionally, PEG-400 is a common permeation enhancer in many commercially available topical and transdermal drug formulations.⁶²⁻⁶⁴ The concentration of PEG-2000 will first be varied in order to determine the optimal PEG formulation; followed by constant PEG concentration and varying PEG length. The fourth set of experiments will be to modify the optimized SLB formulation with an arginine-rich peptide (i.e. R8).—Arginine-rich peptides have been shown to increase cellular internalization⁶⁵, while conjugation of hepta-arginine peptides to cyclosporin-A demonstrates enhanced transdermal kinetics.²⁸ The next task will be to determine how the core properties affect the transdermal kinetics. Keeping the SLB formulation constant, the effects of core size and surface functionalization will be determined. Alvarez-Román et al demonstrated that polystyrene beads preferentially accumulate in different locations of the skin in a size-dependent fashion.²³ Additionally, Rancan et al showed that mesoporous stober silica particles are taken up by skin cells and able to diffuse across skin with a modified SC, both in a size-dependent fashion.⁶⁶ Verma et al. reported significantly enhanced penetration of liposomes, which are deformable, with a diameter of 120 nm, and maximal enhancement of the stratum corneum using liposomes with a diameter of 70 nm.²⁰ These studies illustrate the importance of particle size in addition to chemical and physical surface properties on transdermal kinetics. Three monodisperse sizes of particles (30 nm, 100 nm, and 200 nm) will be synthesized and characterized using a colloidal synthesis, in addition to the broad distribution created by aerosol-assisted EISA. The sixth set of experiments will investigate the effects of core functionalization/core charge. Unmodified silica has a strongly negative ζ-potential (−40 to −15 mV) and can be functionalized to alter the ζ-potential.⁵⁶ Using the optimal core size, particles will be functionalized to have a strongly positive charge (>10 mV) or methylated to confer hydrophobicity. The seventh set of experiments will be to fluorescently label the SLB and perform fluorescence co-localization experiments to determine the fate of the SLB. Finally, using the optimized transderm-PC, the time-dependent flux will be determined.

Specific Aim 2—Elucidate the Mechanisms by which SLB Formulation, Composition, and Functionalization Affect Transdermal Kinetics.

A simple iteration of Fick's 1^{st} law of diffusion relates transdermal flux (J) to SC permeability (P) based on the concentration difference between the receptacle (c_{R}) and the donor (c_{D}) and the thickness of the SC^{1, 17}, allowing for a direct correlation between Protocell SLB formulation, through changes in overall flux and permeability. Permeability coefficients will be calculated from experimental data, however, experimental determination of flux and permeability only reveal information on the kinetics of transdermal diffusion, but nothing about the mechanism of permeation enhancement^{17, 30}, which will be an important parameter to understand for transderm-PC cargo delivery. The standard means of characterizing SC permeation in pharmaceutics is through analysis of decreases in the T_{M}'s of SC lipids via DSC.^{17, 26, 29-32, 35} There are three T_{M} peaks typically associated with human SC lipids.^{32, 59} The first, at 75° C., is due to a change in lipid structure from lamellar to disordered, 90° C., which is associated with transition of protein-associated lipids from the gel to liquid state, and 120° C. indicating protein-associated lipids have been denatured. In SC samples that have been treated with various permeation enhancers, marked decreases in the T_{M}, and decreased peak intensities have been extensively reported.³² However, DSC only gives information on the SC macrostructure so further characterization is needed to fully understand how the SLB enhances permeation. XRD is a materials science characterization technique that gives information on a crystalline structure based on x-ray scattering patterns from fixed angles. Kim et al and many others have previously used small- and wide-angle XRD to characterize the SC's structure.^{26, 32, 33} For small-angle XRD, two peaks have been associated with scattering due to ceramides (d=6.13 nm) and crystalline cholesterol (d=3.38 nm). For wide-angle XRD, one peak at 16.7 Å is associated with crystalline cholesterol.³² FTIR spectroscopy can also be used to characterize changes in the SC structure by measuring changes in the carbon-hydrogen and carbon-oxygen stretching frequencies associated with SC lipid stretching (2850 cm⁻¹ & 2920 cm⁻¹) and change in the structure of SC keratin molecules (1650 cm⁻¹).^{32, 35, 67} The final method of characterization that will be performed is histology/microscopy. Standard H&E staining will be used to investigate any macroscopic changes in the SC's structure, and fluorescence microscopy will be used to qualitatively assess particle distribution in the skin. The biggest challenge with this specific aim will be isolating the SC samples for DSC, XRD, and FTIR without damaging its structure. Once this is accomplished, the skin samples generated in Specific aim 1, will be characterized to correlate how each SLB formulation alters the SC's structure.

Specific Aim 3—Assess the Delivery Efficacy of Transderm-PCs In Vitro Using Nicotine and Ibuprofen, Drugs with Physical and Chemical Properties that Favor or Disfavor Transdermal Diffusion.

Nicotine patches are one of the most commonly used transdermal patches in the country. The chemical and physical properties of Nicotine (K_{o/w}=15.85, miscible in H₂O, 162.234 Da, Tₘ=−7.9° C.) make it ideal for transdermal delivery. On the other hand, the chemical and physical properties of ibuprofen are K_{o/w}=9332.54, insoluble in H₂O, 206.28 Da, Tₘ=74-77° C. As evidenced by its poor aqueous solubility and extremely lipophilic K_{o/w}, its transdermal kinetics are poorly favored as it preferentially partitions in the SC and does not diffuse into deeper tissues.¹⁷ The first experiment will be to determine the loading capacities for both drugs using the optimized core particle, then loading and fusing the optimized SLB for transdermal delivery. Loading capacities and drug release kinetics will be determined using UV spectroscopy. Additionally, the aqueous solubility and K_{o/w} of Ibuprofen loaded-cores will need to be determined to assess how protocells can mask the apparent chemical behavior of a drug. The second experiment will be to deliver nicotine and ibuprofen transdermally, as free drugs and using Protocells. The drug flux will then be calculated using HPLC in order to determine the efficacy of transdermal delivery using protocells and to give insight into the Protocell's drug release profile in the skin.^{58, 59} The final experiment will be to determine if it is possible to deliver combinations of drugs with different chemical and physical properties. This will be accomplished by loading different ratios of these two drugs into the protocell core. The ability to deliver personalized drug combinations, which favor different transdermal behaviors, across the skin using nanoparticles would be an innovation not yet demonstrated. A potentially problematic issue is the fact that most HPLC columns use silica beads, therefore the sample pH will have to be titrated up to dissolve the particles prior to HPLC analysis.

Specific Aim 4—Determine the Basic Pharmacologic Properties of Transderm-PCs Loaded with Nicotine or Ibuprofen In Vivo Using a NU/NU Nude Mouse Model.

This mouse model is hairless, athymic and therefore lacks a functional adaptive immune system, however they have a functional NK innate immune system, making them well suited. In these preliminary in vivo studies, we will administer transderm-PCs topically using a band aide to prevent leakage and water evaporation. After application we will monitor the serum levels of nicotine and ibuprofen as a function of time, and assess biodistribution, pharmacokinetics, and excretion of transderm-PCs. Additionally, we will examine skin for any signs of irritation or damage. Analysis will be performed using HPLC, fluorescence spectral imaging, histology, and ICP-MS.

### REFERENCES FOR EXAMPLE 5

### Bibliography and References Cited

1. Roberts, M. S., Targeted drug delivery to the skin and deeper tissues: role of physiology, solute structure and disease. Clinical and Experimental Pharmacology and Physiology 1997, 24, 874-879.
2. 2010 Census Interactive Population Search. http://2010.census.gov/2010census/popmap/ipmtext.php?fl=35:3514950.
3. New Mexico Department of Public Health, New Mexico Substance Abuse Epidemiology Profile. http://nmhealth.org/erd/HealthData/substance_abuse.shtml, 2011.
4. B. W, B., Novel mechanisms and devices to enable successful transdermal drug delivery. European Journal of Pharmaceutical Sciences 2001, 14, 101−114.
5. Prausnitz, M. R.; Langer, R., Transdermal drug delivery. Nat Biotech 2008, 26, 1261-1268.
6. Thomas, B. J.; Finnin, B. C., The transdermal revolution. Drug Discovery Today 2004, 9, 697-703.
7. Prausnitz, M. R.; Mitragotri, S.; Langer, R., Current status and future potential of transdermal drug delivery. Nat Rev Drug Discov 2004, 3, 115-124.
8. Brod, J., Characterization and physiological role of epidermal lipids. Int J Dermatol 1991, 30, 84-90.
9. Ashley, C. E.; Carnes, E. C.; Phillips, G. K.; Padilla, D.; Durfee, P. N.; Brown, P. A.; Hanna, T. N.; Liu, J. W.; Phillips, B.; Carter, M. B.; Carroll, N. J.; Jiang, X. M.; Dunphy, D. R.; Willman, C. L.; Petsev, D. N.; Evans, D. G.; Parikh, A. N.; Chackerian, B.; Wharton, W.; Peabody, D. S.; Brinker, C. J., The targeted delivery of multicomponent cargos to cancer cells by nanoporous particle-supported lipid bilayers. Nature Materials 2011, 10, 389-397.
10. Epler, K.; Padilla, D.; Phillips, G.; Crowder, P.; Castillo, R.; Wilkinson, D.; Wilkinson, B.; Burgard, C.; Kalinich, R.; Townson, J.; Chackerian, B.; Willman, C.; Peabody, D.; Wharton, W.; Brinker, C. J.; Ashley, C.; Carnes, E., Delivery of Ricin Toxin A-Chain by Peptide-Targeted Mesoporous Silica Nanoparticle-Supported Lipid Bilayers. Advanced Healthcare Materials 2012, n/a-n/a.
11. Ashley, C. E.; Carnes, E. C.; Epler, K. E.; Padilla, D. P.; Phillips, G. K.; Castillo, R. E.; Wilkinson, D. C.; Wilkinson, B. S.; Burgard, C. A.; Kalinich, R. M.; Townson, J. L.; Chackerian, B.; Willman, C. L.; Peabody, D. S.; Wharton, W.; Brinker, C. J., Delivery of Small Interfering RNA by Peptide-Targeted Mesoporous Silica Nanoparticle-Supported Lipid Bilayers. ACS Nano 2012, 6, 2174-2188.
12. Liu, J. W.; Jiang, X. M.; Ashley, C.; Brinker, C. J., Electrostatically Mediated Liposome Fusion and Lipid Exchange with a Nanoparticle-Supported Bilayer for Control of Surface Charge, Drug Containment, and Delivery. Journal of the American Chemical Society 2009, 131, 7567-7570.
13. Liu, J. W.; Stace-Naughton, A.; Jiang, X. M.; Brinker, C. J., Porous Nanoparticle Supported Lipid Bilayers (Protocells) as Delivery Vehicles. Journal of the American Chemical Society 2009, 131, 1354-1357.
14. Cevc, G.; Vierl, U., Nanotechnology and the transdermal route: A state of the art review and critical appraisal. Journal of Controlled Release 2010, 141, 277-299.
15. Kushner, J.; Deen, W.; Blankschtein, D.; Langer, R., First-principles, structure-based transdermal transport model to evaluate lipid partition and diffusion coefficients of hydrophobic permeants solely from stratum corneum permeation experiments. Journal of Pharmaceutical Sciences 2007, 96, 3236-3251.
16. Marianna, F., Non-invasive administration of drugs through the skin: challenges in delivery system design. Pharmaceutical Science &amp; Technology Today 2000, 3, 417-425.
17. Barry, B. W., Lipid-Protein-Partitioning theory of skin penetration enhancement. Journal of Controlled Release 1991, 15, 237-248.
18. Kirjavainen, M.; Monkkonen, J.; Saukkosaari, M.; Valjakka-Koskela, R.; Kiesvaara, J.; Urtti, A., Phospholipids affect stratum corneum lipid bilayer fluidity and drug partitioning into the bilayers. Journal of Controlled Release 1999, 58, 207-214.
19. Sinico, C.; Manconi, M.; Peppi, M.; Lai, F.; Valenti, D.; Fadda, A. M., Liposomes as carriers for dermal delivery of tretinoin: in vitro evaluation of drug permeation and vesicle-skin interaction. Journal of Controlled Release 2005, 103, 123-136.
20. Verma, D. D.; Verma, S.; Blume, G.; Fahr, A., Particle size of liposomes influences dermal delivery of substances into skin. International Journal of Pharmaceutics 2003, 258, 141-151.
21. Kirjavainen, M.; Urtti, A.; Valjakka-Koskela, R.; Kiesvaara, J.; Monkkonen, J., Liposome-skin interactions and their effects on the skin permeation of drugs. European Journal of Pharmaceutical Sciences 1999, 7, 279-286.
22. Ashley, C.; Carnes, E.; Epler, K.; Padilla, D.; Phillips, G.; Castillo, R.; Wilkinson, D.; Wilkinson, B.; Burgard, C.; Sewell, R.; Townson, J.; Chackerian, B.; Willman, C.; Peabody, D.; Petsev, D.; Wharton, W.; Brinker, C. J., Delivery of Therapeutic Macromolecular Cargos by Targeted Protocells ACS Nano (In Review), 2011.
23. Alvarez-Román, R.; Naik, A.; Kalia, Y. N.; Guy, R. H.; Fessi, H., Skin penetration and distribution of polymeric nanoparticles. Journal of Controlled Release 2004, 99, 53-62.
24. Williams, A. C.; Barry, B. W., Penetration enhancers. In Adv Drug Deliv Rev, Netherlands, 2004; Vol. 56, pp 603-18.
25. Martin, B. C.; Morgan, T. M., Transdermal penetration enhancers: Applications, limitations, and potential. Journal of Pharmaceutical Sciences 1999, 88, 955-958.
26. Cornwell, P. A.; Barry, B. W.; Bouwstra, J. A.; Goons, G. S., Modes of action of terpene penetration enhancers in human skin; Differential scanning calorimetry, small-angle X-ray diffraction and enhancer uptake studies. International Journal of Pharmaceutics 1996, 127, 9-26.
27. Akimoto, T.; Nagase, Y., Novel transdermal drug penetration enhancer: synthesis and enhancing effect of alkyldisiloxane compounds containing glucopyranosyl group. Journal of Controlled Release 2003, 88, 243-252.
28. Rothbard, J. B.; Garlington, S.; Lin, Q.; Kirschberg, T.; Kreider, E.; McGrane, P. L.; Wender, P. A.; Khavari, P. A., Conjugation of arginine oligomers to cyclosporin A facilitates topical delivery and inhibition of inflammation. Nat Med 2000, 6, 1253-1257.
29. Golden, G. M.; Guzek, D. B.; Harris, R. R.; McKie, J. E.; Potts, R. 0., Lipid thermotropic transitions in human stratum corneum. J Invest Dermatol 1986, 86, 255-9.
30. Leopold, C. S.; Lippold, B. C., An attempt to clarify the mechanism of the penetration enhancing effects of lipophilic vehicles with differential scanning calorimetry (DSC). J Pharm Pharmacol 1995, 47, 276-81.
31. Al-Saidan, S. M.; Barry, B. W.; Williams, A. C., Differential scanning calorimetry of human and animal stratum corneum membranes. International Journal of Pharmaceutics 1998, 168, 17-22.
32. Kim, Y.-C.; Ludovice, P. J.; Prausnitz, M. R., Transdermal delivery enhanced by magainin pore-forming peptide. Journal of Controlled Release 2007, 122, 375-383.
33. Schreiner, V.; Gooris, G. S.; Pfeiffer, S.; Lanzendorfer, G.; Wenck, H.; Diembeck, W.; Proksch, E.; Bouwstra, J., Barrier characteristics of different human skin types investigated with X-ray diffraction, lipid analysis, and electron microscopy imaging. In J Invest Dermatol, United States, 2000; Vol. 114, pp 654-60.
34. Guan, T. T.; Miao, Y. Q.; Xu, L. S.; Yang, S. S.; Wang, J.; He, H. B.; Tang, X.; Cai, C. F.; Xu, H., Injectable nimodipine-loaded nanoliposomes: Preparation, lyophilization and characteristics. International Journal of Pharmaceutics 2011, 410, 180-187.
35. Chen, H.-C.; Mendelsohn, R.; Rerek, M. E.; Moore, D. J., Fourier transform infrared spectroscopy and differential scanning calorimetry studies of fatty acid homogeneous ceramide 2. Biochimica et Biophysica Acta (BBA)—Biomembranes 2000, 1468, 293-303.
36. Hadgraft, J.; Pugh, W. J., The selection and design of topical and transdermal agents: a review. J. Invest. Dermatol. Symp. Proc. 1998, 3, 131-135.
37. Lombry, C.; Dujardin, N.; Preat, V., Transdermal Delivery of Macromolecules Using Skin Electroporation. Pharmaceutical Research 2000, 17, 32-37.
38. Schmook, F. P.; Stutz, A.; Reinhardt, J., Penetration of Sandimmune (cyclosporin A) in rat skin in vitro. Effects of penetration enhancers and solvents. Skin Pharmacol. 1993, 6, 116-124.
39. Bach, M.; Lippold, B. C., Percutaneous penetration enhancement and its quantification. Eur. J. Pharm. Biopharm. 1998, 46, 1-13.
40. Chen, Y.; Shen, Y.; Guo, X.; Zhang, C.; Yang, W.; Ma, M.; Liu, S.; Zhang, M.; Wen, L.-P., Transdermal protein delivery by a coadministered peptide identified via phage display. Nat Biotech 2006, 24, 455-460.
41. Mitragotri, S.; Kost, J., Low-frequency sonophoresis: a review. In Adv Drug Deliv Rev, Netherlands, 2004; Vol. 56, pp 589-601.
42. Kushner, J. I. V.; Kim, D.; So, P. T. C.; Blankschtein, D.; Langer, R. S., Dual-Channel Two-Photon Microscopy Study of Transdermal Transport in Skin Treated with Low-Frequency Ultrasound and a Chemical Enhancer. J Invest Dermatol 2007, 127, 2832-2846.
43. Kushner, J.; Blankschtein, D.; Langer, R., Evaluation of the porosity, the tortuosity, and the hindrance factor for the transdermal delivery of hydrophilic permeants in the context of the aqueous pore pathway hypothesis using dual-radiolabeled permeability experiments. Journal of Pharmaceutical Sciences 2007, 96, 3263-3282.
44. Kushner, J.; Blankschtein, D.; Langer, R., Evaluation of hydrophilic permeant transport parameters in the localized and non-localized transport regions of skin treated simultaneously with low-frequency ultrasound and sodium lauryl sulfate. Journal of Pharmaceutical Sciences 2008, 97, 906-918.
45. Kushner, J.; Blankschtein, D.; Langer, R., Heterogeneity in skin treated with low-frequency ultrasound. Journal of Pharmaceutical Sciences 2008, 97, 4119-4128.
46. Benson, H. A. E.; Namjoshi, S., Proteins and peptides: Strategies for delivery to and across the skin. Journal of Pharmaceutical Sciences 2008, 97, 3591-3610.
47. Denet, A. R.; Vanbever, R.; Preat, V., Skin electroporation for transdermal and topical delivery. In Adv Drug Deliv Rev, Netherlands, 2004; Vol. 56, pp 659-74.
48. Prausnitz, M. R., Microneedles for transdermal drug delivery. In Adv Drug Deliv Rev, Netherlands, 2004; Vol. 56, pp 581-7.
49. Mark, K., Engineering of needle-free physical methods to target epidermal cells for DNA vaccination. Vaccine 2006, 24, 4651-4656.
50. Oh, J.-H.; Park, H.-H.; Do, K.-Y.; Han, M.; Hyun, D.-H.; Kim, C.-G.; Kim, C.-H.; Lee, S. S.; Hwang, S.-J.; Shin, S.-C.; Cho, C.-W., Influence of the delivery systems using a microneedle array on the permeation of a hydrophilic molecule, calcein. European Journal of Pharmaceutics and Biopharmaceutics 2008, 69, 1040-1045.
51. van den Bergh, B. A. I.; Salomons-de Vries, I.; Bouwstra, J. A., Interactions between liposomes and human stratum corneum studied by freeze-substitution electron microscopy. International Journal of Pharmaceutics 1998, 167, 57-67.
52. Wagner, A.; Vorauer-Uhl, K., Liposome Technology for Industrial Purposes. Journal of Drug Delivery 2011, 2011.
53. Ashley, C. E.; Carnes, E. C.; Phillips, G. K.; Durfee, P. N.; Buley, M. D.; Lino, C. A.; Padilla, D. P.; Phillips, B.; Carter, M. B.; Willman, C. L.; Brinker, C. J.; Caldeira, J. D.; Chackerian, B.; Wharton, W.; Peabody, D. S., Cell-Specific Delivery of Diverse Cargos by Bacteriophage MS2 Virus-like Particles. Acs Nano 2011, 5, 5729-5745.
54. Mei, Z.; Chen, H.; Weng, T.; Yang, Y.; Yang, X., Solid lipid nanoparticle and microemulsion for topical delivery of triptolide. European Journal of Pharmaceutics and Biopharmaceutics 2003, 56, 189-196.
55. Santander-Ortega, M. J.; Stauner, T.; Loretz, B.; Ortega-Vinuesa, J. L.; Bastos-Gonzalez, D.; Wenz, G.; Schaefer, U. F.; Lehr, C. M., Nanoparticles made from novel starch derivatives for transdermal drug delivery. Journal of Controlled Release 2010, 141, 85-92.
56. Brinker, C. J.; Scherer, G. W., Sol-Gel Science: the physics and chemistry of sol-gel processing. 1st ed.; Gulf Professional Publishing: 1990; p 908.
57. Zhang, H.; Dunphy, D.; Jiang, X.; Meng, H.; Sun, B.; Tarn, D.; Xue, M.; Wang, X.; Lin, S.; Ji, Z.; Ling, R.; Garcia, F.; Yang, J.; Kirk, M.; Xia, T.; Zink, J.; Nel, A.; Brinker, C. J., Processing pathway dependence of amorphous silica nanoparticle toxicity—colloidal vs. pyrolytic. 2012. MANUSCRIPT IN PREPARATION
58. Felton, L. A.; Wiley, C. J.; Godwin, D. A., Influence of Hydroxypropyl-β-Cyclodextrin on the Transdermal Permeation and Skin Accumulation of Oxybenzone. Drug Development and Industrial Pharmacy 2002, 28, 1117-1124.
59. Kear, C. L.; Yang, J.; Godwin, D. A.; Felton, L. A., Investigation into the Mechanism by Which Cyclodextrins Influence Transdermal Drug Delivery. Drug Development and Industrial Pharmacy 2008, 34, 692-697.
60. Kitagawa, S.; Sawada, M.; Hirata, H., Fluorescence analysis with diphenylhexatriene and its ionic derivatives of the fluidity of liposomes constituted from stratum corneum lipids: Contribution of each lipid component and effects of long-chain unsaturated fatty acids. International Journal of Pharmaceutics 1993, 98, 203-208.
61. Khazanov, E.; Priev, A.; Shillemans, J. P.; Barenholz, Y., Physicochemical and Biological Characterization of Ceramide-Containing Liposomes: Paving the Way to Ceramide Therapeutic Application. Langmuir 2008, 24, 6965-6980.
62. Akimoto, T.; Aoyagi, T.; Minoshima, J.-i.; Nagase, Y., Polymeric percutaneous drug penetration enhancer: Synthesis and enhancing property of PEG/PDMS block copolymer with a cationic end group. Journal of Controlled Release 1997, 49, 229-241.
63. Mitragotri, S., Synergistic Effect of Enhancers for Transdermal Drug Delivery. Pharmaceutical Research 2000, 17, 1354-1359.
64. Mura, P.; Faucci, M. T.; Bramanti, G.; Corti, P., Evaluation of transcutol as a clonazepam transdermal permeation enhancer from hydrophilic gel formulations. European Journal of Pharmaceutical Sciences 2000, 9, 365-372.
65. Suzuki, T.; Futaki, S.; Niwa, M.; Tanaka, S.; Ueda, K.; Sugiura, Y., Possible Existence of Common Internalization Mechanisms among Arginine-rich Peptides. Journal of Biological Chemistry 2002, 277, 2437-2443.
66. Rancan, F.; Gao, Q.; Graf, C.; Troppens, S.; Hadam, S.; Hackbarth, S.; Kembuan, C.; Blume-Peytavi, U.; Ruhl, E.; Lademann, J.; Vogt, A., Skin Penetration and Cellular Uptake of Amorphous Silica Nanoparticles with Variable Size, Surface Functionalization, and Colloidal Stability. ACS Nano 2012.
67. Takeuchi, Y.; Yasukawa, H.; Yamaoka, Y.; Kato, Y.; Morimoto, Y.; Fukumori, Y.; Fukuda, T., Effects of fatty acids, fatty amines and propylene glycol on rat stratum corneum lipids and proteins in vitro measured by fourier transform infrared/attenuated total reflection (FT-IR/ATR) spectroscopy. Chem Pharm Bull (Tokyo) 1992, 40, 1887-92.

### Example 6

### A Modular Nanoparticle Platform for the Treatment of Emerging Viral Pathogens

### 1. Overview/Abstract

### 1.1 Problem Statement:

Anti-viral drugs must typically be administered in large, frequent doses to effectively treat viral infections, including those caused by emerging and engineered viruses. High doses can, however, cause toxic side-effects to the host and, if taken improperly, can accelerate the evolution of drug resistant pathogens. There is, therefore, a need to develop biocompatible nanoparticle delivery vehicles in order to reduce the number, frequency, duration, and dosage of treatment, delay treatment beyond the current limit, and prevent recurrent disease. Most state-of-the-art nanocarriers, including liposomes and polymeric nanoparticles, suffer from low capacity, poor stability, and minimal uptake by target cells, however. This proposal seeks to address these limitations by designing a modular, highly adaptable nanocarrier, termed a ‘protocell,’⁷⁻⁹ which synergistically combines advantages of liposomes and mesoporous silica nanoparticles.

1.2 Protocells are comprised of a mesoporous silica nanoparticle core encased within a supported lipid bilayer and simultaneously exhibit extremely high loading capacities (>1000-fold higher than comparable liposomes) for chemically disparate therapeutic and diagnostic agents, long-term stability in complex biological fluids, and sub-nanomolar affinities for target cells at low ligand densities. Our ability to precisely control loading, release, stability, and targeting specificity, as well as our ability to engineer the particle size, shape, charge, and surface modification(s) allow us to dramatically reduce dosage and off-target effects, mitigate immunogenicity, maximize biocompatibility and biodegradability, and control biodistribution and persistence. As we reported in the May 2011 cover article of Nature Materials,⁸ protocells, due to their unique biophysical properties, are one-million times more effective at treating human liver cancer than state-of-the-art liposomes. In this proposal, we seek to extend the utility of protocells to emerging viruses that have relevance as potential biothreats and will assess the prophylactic and therapeutic potential of protocells loaded with traditional and novel anti-viral agents and targeted to both potential host cells and already infected cells.

### 2. Experimental Approach

### 2.1 Technical Approach:

Viral infections are treated using small molecule drugs that inhibit entry, fusion, replication, or budding processes¹ and, more recently, therapeutic nucleic acids, such as small interfering RNA (siRNA) that silence expression of specific viral genes or, if tolerated by the host, cellular receptor(s) for viral entry.²⁻³ Many anti-viral agents, however, suffer from a plethora of shortcomings that limit their therapeutic efficacy, including: (1) hypersensitivity and allergic reactions, as well as a variety of other deleterious side effects; (2) the increasing prevalence of drug resistant pathogens and the potential for engineered resistance; and (3) the necessity for large doses and frequent administration in order to promote sufficient accumulation at sites of infection, which is, in turn, caused by poor bioavailability, rapid clearance, limited solubility, incomplete adsorption, and off-target accumulation.⁴ Therapeutic siRNAs can be designed to reduce off-target effects but have limited stability in serum, short half-lives, poor penetration into tissues and cells, and induce innate immune responses.⁵ There is, therefore, a need for biocompatible nanoparticle delivery systems (‘nanocarriers’) that can improve the pharmacokinetics and pharmacodynamics of traditional and novel anti-virals. Numerous nanocarriers, including liposomes, polymeric nanoparticles, dendrimers, carbon nanotubes, and porous, inorganic nanoparticles have been developed for a variety of in vivo diagnostic and therapeutic applications.⁶ While substantial progress has been made toward improving biocompatibility, increasing circulation times, reducing immunogenicity, and minimizing off-target interactions, the therapeutic efficacy of most state-of-the-art nanocarriers is still, however, restricted by low loading capacity, poor targeting specificity, and limited stability under physiological conditions. To this end, we have developed mesoporous silica nanoparticle-supported lipid bilayers (‘protocells’),⁷⁻⁹ which synergistically combine the advantages of two promising nanoparticle delivery vehicles: liposomes and mesoporous silica nanoparticles (MSNPs).

### Protocells Combine Advantages of Both Liposomes and Mesoporous Silica Nanoparticles.

Protocells (see FIG. 1X6) are comprised of a spherical MSNP core encased within a supported lipid bilayer (SLB). MSNPs have an extremely high surface area (>1200 m²/g) and can, therefore, be loaded with high concentrations of various therapeutic and diagnostic agents by simply soaking them in a solution of the cargo(s) of interest. Furthermore, since the aerosol-assisted evaporation-induced self-assembly (EISA) process¹⁰ we use to synthesize MSNPs is compatible with a wide range of structure-directing surfactants and post-synthesis processing of resulting particles, the pore size can be varied from 2.5-nm to 25-nm, and the pore walls can be modified with cationic or hydrophobic silanes, both of which enable facile encapsulation of a variety of chemically disparate cargos, including small molecule drugs (acidic, basic, and hydrophobic) and drug cocktails, siRNAs, proteins, and DNA vectors that encode small hairpin RNAs (shRNAs), as well as diagnostic agents like quantum dots and iron oxide nanoparticles, if desired. We have shown that protocells have a loading capacity of up to 50 wt % for small molecule drugs, which is 5-fold higher than other MSNP-based delivery vehicles¹¹ and 1000-fold higher than similarly-sized liposomes.⁸ Release rates can be tailored by controlling the core's degree of silica condensation and, therefore, its dissolution rate under physiological conditions; thermal calcination maximizes condensation and results in particles with sustained release profiles (7-10% release per day for up to 2 weeks), while use of acidified ethanol to extract surfactants enhances particle solubility and results in burst release of encapsulated drugs (100% release within 12 hours). Liposome fusion to cargo-loaded MSNPs results in the formation of a coherent SLB that provides a stable, fluid, biocompatible interface for display of functional molecules, such as polyethylene glycol (PEG) and targeting ligands. We have demonstrated that protocells stably encapsulate small molecule drugs for up to 4 weeks when dispersed in complex biological fluids (e.g. complete growth medium and blood), regardless of whether the SLB is composed of lipids that are fluid or non-fluid at body temperature; in contrast, liposomes rapidly leak their encapsulated drugs, even when their bilayers are composed of fully saturated lipids, which have a high packing density and should, therefore, limit diffusion of drugs across the bilayer.⁸ The fluid, yet stable SLB enables us to achieve exquisitely high targeting specificities at low ligand densities, which, in turn, reduces immunogenicity and non-specific interactions; we have shown that protocells modified with an average of just 5 targeting peptides per particle have a 10,000-fold higher affinity for target cells than for non-target cells when the SLB is composed of the fluid, zwitterionic lipid, 1,2-dioleoyl-sn-glycero-3-phosphocholine (DOPC).⁸ We have, furthermore, shown that incorporation of peptides that trigger endocytosis and endosomal escape on the protocell SLB enables cytosolic dispersion of encapsulated cargos and that, by modifying cargo molecules with targeting moieties, such as a nuclear localization sequence (NLS), we can effect intracellular accumulation of cargos within specific organelles.⁸ Due to their high capacity for disparate cargos, high targeting specificity at low ligand densities, and long-term bilayer stability, protocells loaded with a cocktail of chemotherapeutic drugs and targeted to human liver cancer are one million times more efficacious than comparable liposomes.⁸ In the proposed R&D, we will engineer protocells for targeted delivery of therapeutics to cells infected by intracellular pathogens with the goal of realizing a therapeutic efficacy that is similarly superior to free drugs and drug-loaded liposomes.

### The Flexible, Modular Nature of Protocells Enables Various In Vivo Challenges to be Addressed.

In order to promote accumulation of anti-viral agents within potential or already infected host cells, protocells must: (1) subsist in the circulation for a sufficient period of time without causing toxicity to the host; (2) accumulate within target tissue(s); (3) selectively bind to and become internalized by target cell(s); (4) release their encapsulated drugs with the necessary kinetics and within the appropriate intracellular compartment(s); and (5) degrade into biocompatible monomers that can be readily excreted. As discussed above, we have shown that PEGylated protocells modified with low densities of targeting ligands readily bind to and become internalized by target cells and stably encapsulate, drugs until endosome acidification destabilizes the SLB, thereby exposing the core and driving either sustained or burst release of encapsulated drugs (steps 3 and 4 above).⁸ During the course of the proposed R&D, we will re-assess the in vitro performance of protocells targeted to virally-infected cells and loaded with anti-viral agents as described below but will also characterize the biodistribution, biocompatibility, and biodegradability (steps 1, 2, and 5 above) of protocells in mouse and avian embryo models. Our preliminary in vivo studies indicate that protocells are highly biocompatible and can be engineered for broad distribution and persistence within target tissues. As shown in FIG. 2AX6, Balb/c mice injected with 200 mg/kg doses of PEGylated protocells three times each week for 3 weeks show no signs of gross toxicity or weight loss; given their high loading capacity, this result indicates that protocells can deliver at least 900 mg/kg of small molecule drugs with either burst or sustained release kinetics. Furthermore, as demonstrated by FIG. 2BX6, PEGylated protocells 20-200 nm in diameter remain broadly distributed for 48 hours when injected in Balb/c mice at a dose of 200 mg/kg, which provides a sufficient period of time for targeted protocells to accumulate within target tissues. We have also shown that, by controlling the size and surface modification(s), we can promote accumulation of protocells within the bone and liver of Balb/c and Nu/Nu mice for treatment of acute lymphoblastic leukemia and hepatocellular carcinoma, respectively, and that protocells, even when loaded with a therapeutically relevant dose of the chemotherapeutic, doxorubicin, persist in the target tissue for up to 4 weeks with no signs of gross or histological toxicity, as determined by organ weight and pathology, respectively (unpublished data). Additionally, our collaborators at the UCLA Center for Environmental Implications of Nanotechnology have shown that MSNPs are biodegradable and are ultimately excreted in the urine and feces as silicic acid.¹² Finally, we have shown that protocells modified with high densities (up to 10 wt %) of peptides 7-12 amino acids in length induce neither IgG nor IgM responses when injected in C57B1/6 mice at a total dose of 400 mg/kg (unpublished data). Depending upon the biodistribution required for a specific application, we can control the MSNP size and shape (spherical, disk-shaped, and rod-shaped¹³) and the SLB charge and surface modification(s), making the protocell a highly modular, flexible nanoparticle delivery system.

Synthesis of Protocells Loaded with Anti-Viral Agents and Targeted to Uninfected and Infected Host Cells.

In the proposed R&D, we will engineer protocells for targeted delivery of siRNA and small molecule anti-virals to cells infected with Nipah virus (NiV), a BSL-4 paramyxovirus for which no approved vaccines or effective therapeutics exist, with the ultimate goals of minimizing the number, frequency, duration, and dosage of treatment, delaying treatment beyond the current limit, and preventing recurrent disease compared to what is achievable with free drug or liposomal drug. We selected NiV as a model emerging virus due to its well-characterized structure and cellular tropism, as well as its relevance as a biothreat.¹⁴ We have previously reported the utility of protocells in delivery of siRNA to the cytosol of target cells; however, the MSNPs we used in these studies were synthesized using a water-in-oil emulsion technique¹⁵ that suffers from high batch-to-batch variation in particle size, size distribution, and yield. Therefore, we will begin by adapting the aerosol-assisted EISA process, which enables production of large quantities of particles with reproducible properties, to generate MSNPs suitable for encapsulation and delivery of siRNA. These MSNPs must have positively-charged pores large enough to accommodate negatively-charged siRNA (13-15 kDa) and should be <200-nm in diameter to minimize accumulation in the liver and spleen and reduce uptake by monocytes/macrophages of the reticuloendothelial system (RES);⁶ maximizing surface area and pore connectivity will also be important to maximize loading capacity. To generate particles with these properties, we will investigate two synthesis strategies. In the first strategy, we will use a binary surfactant system to generate monophasic particles; specifically, we will employ a large pore-forming surfactant, such as Pluronic® F127, in combination with a surfactant that normally forms high-surface-area mesophases with a high degree of connectivity, such as cetyl trimethylammonium bromide (CTAB). If we can form a stable, tertiary phase mixture of these surfactants in the silica precursor sol, it should be possible to generate particles with >5-nm pores. In the second strategy, we will pre-form a stable, worm-like mesophase by polymerizing a large pore-forming surfactant (e.g. F127) with benzoic acid; this hybrid surfactant will then be added to the silca precursor sol, along with a polymeric swelling agent (e.g. polypropylene glycol) to obtain surface-accessible pores up to 20-nm in size. Once the pore size and geometry have been optimized, we will react particles with aminated silanes, such as 3-aminopropyl triethoxysilane (APTES), to dramatically increase the zeta potential of the particle with minimal impact on the pore structure. Finally, we will investigate ways to modify the aerosol-assisted EISA process, which normally results in a broad distribution of particles (from 50-nm to >1 μm), in order to reduce the particle size and size distribution; reducing the viscosity of the precursor sol by diluting it with ethanol or heating it prior to aerosolization should shift the distribution of resulting particles to <200-nm. The size and size distribution, zeta potential, surface area, and pore size distribution of all MSNPs will be characterized using dynamic light scattering (DLS), electron microscopy, and nitrogen sorption. Once we have generated MSNPs with the appropriate properties, we will test their siRNA loading capacities and pH-dependent release rates using previously reported techniques; although we will initially employ particles capable of burst release, we can adapt the release rate depending on the results of the ex ovo studies described below. We will then fuse liposomes composed of 65 wt % DOPC, 5 wt % 1,2-dioleoyl-sn-glycero-3-phosphoethanolamine (DOPE), and 30 wt % cholesterol to siRNA-loaded cores and modify the resulting SLB with single-chain antibody fragments (scFvs) or peptides (synthesized with C-terminal cysteine residues to facilitate conjugation) using commercially-available crosslinkers that react with primary amine moieties in DOPE and with the sulfhydryl moiety in cysteine. We will also modify the SLB with 10 wt % of PEG-2000, which has been shown to reduce adsorption of serum proteins to nanocarrier surfaces in vivo and to minimize uptake by the RES⁶ and characterize the average ligand and PEG densities using mass spectrometry. FIG. 1X6 shows a schematic of the protocell we propose to develop.

### In Vitro Optimization of the Binding, Internalization, and Cargo Delivery Properties of Targeted, Drug-Loaded Protocells.

We have used phage display to identify peptides that bind to ephrin B2, the entry receptor for NiV,¹⁶ by panning against Chinese hamster ovary (CHO) cells transfected to express human ephrin B2 and conducting subtractive panning against parental CHO cells and CHO cells transfected to express human ephrin B1. After five rounds of selection, the predominant sequence was the 7-mer, TGAILHP (SEQ ID NO:18), which binds well to several ephrin B2-positive cell lines, as determined by an enzyme-linked immunosorbent assay (unpublished data). We will measure dissociation constants (K_{d}) of protocells modified with high and low densities of the TGAILHP peptide for various ephrin B2-positive and negative cells using flow cytometry or surface plasmon resonance and compare these values with the affinity of protocells that display an ephrin B2-specific scFv;¹⁷ targeting peptides are preferable to scFvs, given that protocells modified with up to 10 wt % of a heptapeptide are non-immunogenic. We will also modify protocells with a scFv that binds to the NiV attachment glycoprotein (G),¹⁸ which is expressed on the surfaces on infected cells, in order to target both host cells (i.e. cells that express ephrin B2) and infected cells (i.e. cells transfected to express NiV-G in initial studies). If ligands that bind to ephrin B2 or NiV-G are insufficient to achieve the desired affinities, we will conduct phage display to identify additional ligands. We will then use confocal fluorescence microscopy to determine whether peptide and scFv-targeted protocells are internalized by target cells and, if so, to assess their intracellular fate(s). If targeting ligands do not naturally trigger internalization, we will further modify the SLB with a peptide (octaarginine, or R8) known to trigger both macropinocytosis and macropinosome escape when displayed on nanoparticles in high densities.¹⁹⁻²⁰ To assess the therapeutic efficacy of siRNA-loaded protocells, we will first design and validate siRNAs specific for a far red fluorescent reporter protein (mKATE), NiV nucleocapsid protein (N), and NiV matrix protein (M). We will then use real-time PCR to determine expression levels in: (1) Vero and/or human embryonic kidney (HEK) cells, pre-infected with a NiV-G/F pseudotyped vesicular stomatitis virus (NiVpp¹⁸) that encodes mKATE and exposed to ephrin B2-targeted protocells loaded with mKATE-specific siRNA(s); (2) Vero and/or HEK cells, pre-transfected with NiV-N and NiV-M and exposed to ephrin B2-targeted protocells loaded with NiV-N and M-specific siRNA(s); and (3) Vero and/or HEK cells, pre-infected with NiVpp that encodes both mKATE and surface expression of NiV-G and exposed to G-targeted protocells loaded with mKATE-specific siRNA. In parallel, we will provide NiV-N and NiV-M siRNAs to A. Freiberg at the University of Texas Medical Branch (UTMB) for validation against live NiV; if any N or M-specific siRNA(s) inhibit viral replication in vitro, we will test the efficacy of siRNA-loaded, ephrin B2-targeted protocells as well. If siRNA is insufficient to silence target genes for a sustained (>72 hours) period of time, we will design, load, and deliver minicircle DNA vector(s)²¹ that encode shRNA(s) specific for mKATE, NiV-N, and/or NiV-M. We will also determine whether channel rhodopsin²² and other light-gated ion channels can be engineered for transmission of small molecule anti-virals and incorporated within the protocell SLB to enable triggered delivery. Use of Avian Embryos to Assess the In Vivo Therapeutic Potential of Protocells. Once we have optimized the binding, internalization, and cargo delivery properties of peptide or scFv-targeted protocells in vitro, we will assess their in vivo therapeutic potential. To do so, we will employ avian embryos as a model in vivo system since NiV does not cause disease in common small animal models (i.e. mice and rats).¹⁴ Furthermore, avian embryos have been used to study NiV pathogenesis²³ and are amenable to intravital imaging techniques capable of single-cell resolution. Finally, avian embryos cost one-tenth to one-hundredth as much as common small animal models and are not subject to Institutional Animal Care and Use Committee (IACUC) regulations, making them ideal for cost-effective, high-throughput screening of nanoparticles. We will first optimize the embryo age and NiVpp concentration in order to maximize expression of NiVpp-encoded proteins while minimizing toxicity to the embryo. We will then determine the silencing efficacy of protocells loaded with mKATE-specific siRNA(s) and targeted to NiV-G using embryos pre-infected with NiVpp that encodes mKATE and induces surface expression of NiV-G on infected cells. Finally, we will assess the ability of protocells to deliver complex combinations of anti-virals, including siRNA (or minicircle DNA, as appropriate), traditional anti-viral agents (e.g. ribavirin), and novel, broad-spectrum anti-virals (e.g. LJ001²⁴) to embryos that have been transfected to express human ephrin B2 and infected with the NiVpp that encodes mKATE and NiV-G.

### REFERENCES FOR EXAMPLE 6

1. Clercq, E. D., Nat Rev Drug Discov, 6, 941-941 (2007).
2. Ge, Q., L. Filip, et al., Proceedings of the National Academy of Sciences of the United States of America, 101, 8676-8681 (2004).
3. Novina, C. D., M. F. Murray, et al., Nature Medicine, 8, 681-686 (2002).
4. Lembo, D., R. Cavalli, Antiviral Chemistry and Chemotherapy, 21, 53-70 (2010).
5. Gavrilov, K., W. M. Saltzman, Yale Journal of Biology and Medicine, 85, 187-200 (2012).
6. Peer, D., J. M. Karp, et al., Nat Nano, 2, 751-760 (2007).
7. Ashley, C. E., E. C. Carnes, et al., ACS Nano, 6, 2174-2188 (COVER) (2012).
8. Ashley, C. E., E. C. Carnes, et al., Nat Mater, 10, 389-397 (COVER) (2011).
9. Epler, K., . . . C. E. Ashley, E. C. Carnes, Advanced Healthcare Materials, 1, 241-241 (COVER) (2012).
10. Lu, Y. F., H. Y. Fan, et al., Nature, 398, 223-226 (1999).
11. Meng, H., M. Liong, et al., ACS Nano, 4, 4539-4550 (2010).
12. Lu, J., M. Liong, et al., Small, 6, 1794-1805 (2010).
13. Meng, H., S. Yang, et al., ACS Nano, 5, 4434-4447 (2011).
14. Bossart, K., J. Bingham, D. Middleton, The Open Virology Journal, 1, 14-25 (2007).
15. Carroll, N. J., S. Pylypenko, P. B. Atanassov, D. N. Petsev, Langmuir, 25, 13540-13544 (2009).
16. Negrete, 0. A., E. L. Levroney, et al., Nature, 436, 401-405 (2005).
17. Gu, X., Y. Vedvyas, et al., PLoS ONE, 7, e30680 (2012).
18. Negrete, 0. A., D. Chu, H. C. Aguilar, B. Lee, Journal of Virology, 81, 10804-10814 (2007).
19. Khalil, I. A., K. Kogure, S. Futaki, H. Harashima, Journal of Biological Chemistry, 281, 3544-3551 (2006).
20. El-Sayed, A., I. A. Khalil, et al., Journal of Biological Chemistry, 283, 23450-23461 (2008).
21. Chen, Z.-Y., C.-Y. He, A. Ehrhardt, M. A. Kay, Mol Ther, 8, 495-500 (2003).
22. Kleinlogel, S., K. Feldbauer, et al., Nat Neurosci, 14, 513-518 (2011).
23. Tanimura, N., T. Imada, Y. Kashiwazaki, S. H. Sharifah, Journal of Comparative Pathology, 135, 74-82 (2006).
24. Wolf, M. C., A. N. Freiberg, et al., Proceedings of the National Academy of Sciences (2010).
25. Gao, F., P. Botella, et al., The Journal of Physical Chemistry B, 113, 1796-1804 (2009).

### Example 7

### Biodistribution and Toxicity of Untargeted Protocells

Preliminary biodistribution and toxicity of untargeted protocells has been evaluated. Using live animal fluorescence imaging with Bulb/c or Nu/Nu mice, untargeted protocells modified with a fluorescent core are found to be systemic distributed following IV injection at a maximum dose of 4 mg per mouse (200 mg/kg) [FIG. 1X7A]. This period of systemic circulation would provide time for accumulation of targeted protocells within specified cells independent of the location of the cells. Over the course of 24-48 hours, remaining protocells can be seen accumulated in the liver and spleen. Following 3 doses at 200 mg/kg, a sizable concentration of particles remain in the liver for at least 2 weeks (FIGS. 1X7B and D). This accumulation and retention in the liver does not result in any gross liver (FIG. 2X7) toxicity, as determined via pathology and liver weight. Therefore, untargeted protocells may serve as ideal reservoirs for delivering large, sustained doses of antivirals and siRNA, in addition to their potential to be used in targeted delivery. Furthermore, after 3 weeks of tri-weekly 200 mg/kg doses (total silica dose of 36 mg per mouse) no toxicity or decline in weight gain was observed (FIG. 1X7C). Even at this exceedingly high dose, protocells appear to have minimal to no toxicity.

### Example 8

### Transdermal Protocell Diffusion

Approach: Using standard protocell formulation (DOPC (Tm=−20° C.) 55 wt %, Cholesterol 30 wt %, DOPE-PEG 15 wt %) and expose them to skin samples where stratum corneum is left intact and stratum corneum is removed. Analyze using ICP mass spec.

Adipose tissue was removed from the skins and they were cut into 0.64 cm×0.64 cm squares. The skins were then placed on the Fraz diffusion cell and allowed to equilibrate 45 minutes. After equilibration, the diffusion buffer was removed and replaced with clean diffusion buffer. Once again the skins were allowed to equilibrate for 45 minutes. 8.125 mg (650 ul) of protocells were added into the cell cap. After 24 hours, the fluid in the cell cap was collected. The skins were then dabbed dry, and washed. Receptor fluid was also collected. The skins and the receptor were analyzed with ICP mass spec. The SC was left intact on three of the samples and removed using tape on the other three samples. Controls were skin samples with the SC removed and intact, treated with 0.5×PBS. Data above shows the ICP Mass spec results for the receptacle fluid from each sample. ICP for receptacle fluid was taken on 10-27-2011. This data was averaged and the standard deviation was determined. FIGS. 1X8, 2X8, 3X8. ICP mass spec of donor cap samples was determined. FIG. 4X8.

Preliminary data suggested that a small percentage of protocells are able to diffuse through both full and partial thickness skin, suggesting that protocell surface modifications could influence the skin's permeability and subsequent diffusion of protocells through the skin.

To identify a quick way to quantify the amount of protocells that diffuse through the skin, SiO₂ cores fluorescently labeled with Alexa Fluor 633 are made spectrafluorimetry is used to determine the concentration of SiO₂ in the receptacle fluid. This can also be extended to determine the amount of SiO2 left in cell donor cap. See FIG. 6X8, 7X8, 8X8, 9X8.

Core functionalization is shown in FIG. 5X8.

Positive control showed that fluorescently-tagged particles in the skin can be imaged while taking advantage of the skin's autofluorescence. FIG. 10X8.

### Example 9

### Transdermal SiO₂ Nanoparticles

### Fluorimeter Settings

Intensity units=counts/second

### Excitation: 632 nm

Emission scan: 644 nm-650 nm; *All values calculated at 647 nm*
Step size=1 nm
Slit size=2 nm
Integration time=1 sec
ASOC Sampling frequency=0.2 kHz

### Assumptions and Known Variables:

All particles were fluorescently tagged using Dylight 633 with a 10 ug:1 mg of dye to NH2-silica
Emission maximum for Dylight 633 is 647 nm with an excitation maximum at 632 nm
All blanks were taken from same stock solution composed of receptacle fluid and therefore values were averaged
Each sample was run a minimum of 3 times and a maximum of 9 times
   Sample was mixed prior to each run
   All error bars represent 95% confidence interval; standard deviation from the mean was calculated then used to calculate standard error and multiplied by 1.96 to obtain 95% confidence
   Standard curves were generated using 24 hour receptacle fluid from control receptacle (denoted S1)
      Standard curves began at a [starting] of 0.16 mg/ml with 1:2 dilutions down to 1.953125E-5 mg/ml
   Standard curves follow 2nd order polynomial equations (R2>0.99), however linear regression analysis can be applied by using the linear portion of the curve over the relevant concentration ranges (R2>0.93); Standard curves plotted Log Mean FI vs. Log [SiO2]
   Skin exhibits a high degree of heterogeneity in autofluorescence, therefore 0 hour samples were pulled prior to administering protocells in the donor cap and the difference in autofluorescence was established between the 24 hour blank (S1)
      This difference was then added or subtracted from the 0 hour samples as a correction value to standardize all remaining receptacle fluids (S2-S9) to the control (S1)
   Equation from linear portion of curve was used to calculate unknown concentrations from the corrected mean fluorescence intensities at 0, 4, and 24 hour time points
      The concentration obtained from the 0 hour time point was subtracted from the 4 and 24 hour time point to determine the actual SiO2 content in the receptacle.

A modified Franz diffusion cell was used in all experiments. After removal of subcutaneous tissue, the donated abdominal skin was cut into ˜2 cm2 pieces and placed over the 5.1 ml receptacle while avoiding the formation of air bubbles and allowed to equilibrate for 60 minutes. Receptacle fluid was kept at 37° C. After 60 minutes the skin was removed, the receptacle fluid was replaced and the skins were allowed to re-equilibrate for 30 minutes. After 30 minutes, the 0 hour sample was pulled (˜400 ul) and replaced with fresh diffusion buffer. Various protocell formulations were administered (500 ul of 16 mg/ml in 0.5×PBS) with n=4 for each formulation. 1 skin (S1) from each experiment was treated with 0.5×PBS. Standard curves were generated within the concentration range of 0.16 mg/ml—1.953125E-5 mg/ml using a 1:2 dilution from the S1 24 hour receptacle fluid. All standard curves followed the same general 2nd order polynomial trend and are plotted on a Log vs. Log scale. The red line denotes the mean blank value (S1 24 hr) with 95% confidence. FIG. 1X9.

Linear regression analysis was conducted in conjunction with spectrafluorimetry was used to discern the unknown concentrations in each receptacle at the 4 hour and 24 hour time points. A linear trendline was applied to the relevant concentration/intensity ranges to obtain an equation with the form y=mx+b; all R2>0.9300. 10× was solved for to obtain each concentration at 0, 4, and 24 hour times. This value was then multiplied by 5.1 to give the total mg of SiO2 at each time point. The final amount was obtained by subtracting out the value obtained from the 0 hour sample. FIG. 2X9.

PCs with 9 different bilayer formulations and SiO2 cores with no supported lipid bilayer (SLB) were investigated. SiO2 cores w/o the SLB show the highest fluorescence intensity with the most variance, however since these were administered in 0.5×PBS the intensities seen are most like due to a dissolution event and not intact cores. DOPC protocells with 30 wt % cholesterol show the most consistent diffusion with the least amount of variation, followed by DSPC protocells with 30 wt % cholesterol. Protocells with 25 wt % DOPE, 30 wt % cholesterol and 45 wt % DOPC showed ug quantities of SiO2 at the 24 hour time point. Finally, protocells with 25 wt % DOPE, 30 wt % cholesterol, 30 wt % DOPC, and 15 wt % PEG showed a significant increase in transdermal diffusion over DOPC/cholesterol formulated PCs, but the statistical variance between each sample was high. The results indicate that the SLB formulation can drastically affect transdermal diffusion. In addition, an interesting trend was observed regarding formulations with PEG. FIG. 3X9.

DOPC (Tm=−20)/cholesterol protocells showed about double the amount of SiO2 at the 24 hour time point when compared to DSPC (Tm=55)/cholesterol protocells. This is consistent with the liposome literature that suggests lipids with lower transition temperatures diffuse deeper into full thickness skin and lipids with higher transition temperatures remain localized in the stratum corneum. The addition of PEG to the DOPC/chol and DSPC/chol formulations significantly decreased transdermal diffusion. PEG, an hydrophilic polymer, has previously been used as a penetration enhancer. I am hypothesizing that the decreased diffusion is due to interactions between the aqueous portions of intercellular lamellae that do not disrupt the intercellular structures and therefore hinder diffusion. The introduction of DOPE into the DOPC PC formulation shows increased diffusion over other formulations tested (precious slide). The addition of both DOPE and PEG shows significant increase in transdermal diffusion (with high statistical variance) suggesting that the combination of ethanolamine and PEG can favorably increase transdermal diffusion. This trend will be further investigated using DSC, small angle XRD, confocal microscopy, and possibly FTIR. FIG. 4X9.

FIGS. 5X9 and 6X9 show the individual increase in the corrected mean fluorescence intensities as a function of time. In all graphs, S1 denotes the blank values at the 0, 4, and 24 hour time points. Autofluorescence from the blanks varies across the board and either remains the same or increases to the “24 hour blank value” over time. Some of the literature suggests that the autofluorescence seen in the receptacle fluid decreases as a function of time but that has not been observed here. In some instances the slope of intensity vs. time is much more steep within the first 4 hours and decreases over time, other cases the slope is less prominent in the first 4 hours and becomes more steep as time progresses, and in some cases the slope remains constant over time. This is not surprising due to the heterogeneity of the skin. FIGS. 7X9, 8X9 and 9X9 illustrate the effect of formulation on kinetics.

1-90. (canceled)

91. A transdermal protocell comprising a porous nanoparticulate core (a) that is loaded with one or more pharmaceutically-active agents and (b) that is encapsulated by and that supports a lipid bilayer, wherein the lipid bilayer comprises one or more stratum corneum permeability-enhancers selected from the group consisting of a monosaturated omega-9 fatty acid, an alcohol, a diol, a solvent, a co-solvent, R8 peptide, and an edge activator.

92. The transdermal protocell of claim 91, wherein the one or more stratum corneum permeability-enhancers is a monosaturated omega-9 fatty acid selected from the group consisting of oleic acid, elaidic acid, eicosenoic acid, mead acid, erucic acid, and nervonic acid, most preferably oleic acid, and mixtures thereof.

93. The transdermal protocell of claim 91, wherein the one or more stratum corneum permeability-enhancers is an alcohol is selected from the group consisting of methanol, ethanol, propanol, and butanol, and mixtures thereof; or a solvent or co-solvent selected from the group consisting of PEG 400 and DMSO.

94. The transdermal protocell of claim 91, wherein the one or more stratum corneum permeability-enhancers is a diol selected from the group consisting of ethylene glycol and polyethylene glycol, and mixtures thereof.

95. The transdermal protocell of claim 91, wherein the one or more stratum corneum permeability-enhancers is an edge activator selected from the group consisting of bile salts, polyoxyethylene esters and polyoxyethylene ethers, and a single-chain surfactant, and mixtures thereof.

96. The transdermal protocell of claim 91, wherein the one or more stratum corneum permeability-enhancers is an edge activator, and the edge activator is sodium deoxycholate.

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. The transdermal protocell of claim 91, wherein the nanoparticulate core is comprised of one or more compositions selected from the group consisting of silica, a biodegradable polymer, a solgel, a metal and a metal oxide.

111. (canceled)

112. (canceled)

113. (canceled)

114. The transdermal protocell of claim 91, wherein the lipid bilayer is comprised of lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures thereof.

115. A method of treating a subject with a disease, the method comprising transdermally administering to the subject a pharmaceutically-effective amount of a transdermal protocell composition, the transdermal protocell composition comprising a plurality of transdermal protocells, the transdermal protocells comprising a porous nano particulate core (a) that is loaded with one or more pharmaceutically active agents and (b) that is encapsulated by and that supports a lipid bilayer, wherein the laid bilayer comprises one or more stratum corneum permeability-enhancers selected from the group consisting of a monosaturated omega-9 fatty acid, an alcohol, a diol, a solvent, a co-solvent, R8 peptide, and an edge activator.

116. (canceled)

117. (canceled)

118. A transdermal pharmaceutical composition comprising a pharmaceutically-effective amount of transdermal protocells of claim 91, and a pharmaceutically-acceptable excipient.

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. A protocell comprising a porous, nanoparticulate silica core that:
(a) is modified with an amine-containing silane selected from the group consisting of (1) a primary amine, a secondary amine a tertiary amine, each of which is functionalized with a silicon atom, (2) a monoamine or a polyamine (3) N-(2-aminoethyl)-3-aminopropyltrimethoxysilane (AEPTMS) (4) 3-aminopropyltrimethoxysilane (APTMS), (5) 3-aminopropyltriethoxysilane (APTS), (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, or quaternary alkyl amines, or combinations thereof;
(b) are loaded with a siRNA or a protein toxin; and
(c) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bilayer comprises a cationic lipid and one or more zwitterionic phospholipids.

138. (canceled)

139. (canceled)

140. (canceled)

141. (canceled)

142. (canceled)

143. A method of treating a subject with a disease comprising administering to the subject a pharmaceutically-effective amount of a protocells of claim 137.

144. (canceled)

145. A pharmaceutical composition comprising a pharmaceutically-effective amount of a protocells of claim 137, and a pharmaceutically-acceptable excipient.

146. The protocell of claim 137, wherein the core is loaded with an siRNA that induces sequence-specific degradation of NiV nucleocapsid protein (NiV-N) mRNA.

147. The protocell of claim 137, wherein the core is loaded with an siRNA that targets a member of the cyclin superfamily selected from the group consisting of cyclin A2, cyclin B1, cyclin D1, and cyclin E.

148. The transdermal protocell of claim 91, wherein the porous nanoparticulate core is loaded with at least one anticancer agent.

149. A plurality of transdermal protocells according to claim 148, wherein the protocells have an average flux of the anticancer agent of around 0.20 to about 0.30 μg/cm² hr.

150. The pharmaceutical composition of claim 145, wherein the pharmaceutical compositions is co-administered with one or more anticancer agents.

151. A protocell comprising a porous nanoparticulate silica core that:
(a) is modified with an amine-containing silane selected from the group consisting of (1) a primary amine, a secondary amine, or a tertiary amine, each of which is functionalized with a silicon atom, (2) a monoamine or a polyamine, (3) 3-aminopropyltrimethoxysilane (APTMS), (4) 3-aminopropyltriethoxysilane (APTS), (5) an amino-functional trialkoxysilane, and (6) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, or combinations thereof; and
(b) is encapsulated by and that supports a lipid bilayer comprising one of more lipids.

152. A transdermal protocell composition comprising a plurality of transdermal protocells, the plurality of transdermal protocells comprising a porous nanoparticulate core (a) that is loaded with one or more pharmaceutically-active agents and (b) that is encapsulated by and that support a lipid bilayer, wherein the lipid bilayer comprises one or more stratum corneum permeability-enhancers selected from the group consisting of a monosaturated omega-9 fatty acid, an alcohol, a diol, a solvent, a co-solvent, R8 peptide, and an edge activator.

153. The transdermal protocell composition according to claim 152, wherein the protocells have an average diameter of between about 50 nm and about 300 nm.

154. The transdermal protocell composition according to claim 152, wherein the protocells have an average diameter of between about 55 nm and about 270 nm.

155. The transdermal protocell composition according to claim 152, wherein the protocells have an average diameter of between about 60 nm and about 240 nm.

156. The transdermal protocell composition according to claim 152, wherein the protocells have an average diameter of between about 65 nm and about 210 nm.

157. The transdermal protocell composition according to claim 152, wherein the protocells have an average diameter of between about 65 nm and about 190 nm.

158. The transdermal protocell composition according to claim 152, wherein the protocells have an average diameter of between about 65 nm and about 160 nm.

159. The transdermal protocell composition according to claim 152, wherein the protocells have an average diameter of between about 65 nm and about 130 nm.
